(19) United States
(12) Patent Application Publication
(10) Pub. No.: US 2026/0315492 A1
(43) Pub. Date: Oct. 15, 2026

(54) 5,6-FUSED BICYCLIC AMIDES AND COMPOSITIONS FOR USE AS 15-PROSTAGLANDIN DEHYDROGENASE MODULATORS

(71) Applicant: AMGEN INC., Thousand Oaks, CA (US)

(72) Inventors: Kevin Lloyd GREENMAN, Thousand Oaks, CA (US); Kexue LI, Newbury Park, CA (US); Albert K. AMEGADZIE, Westlake Village, CA (US); Hui-Ling WANG, Thousand Oaks, CA (US); Nicholas Anthony WEIRES, Thousand Oaks, CA (US); John G. ALLEN, Newbury Park, CA (US); Matthew P. BOURBEAU, Woodland Hills, CA (US)

(73) Assignee: AMGEN INC., Thousand Oaks, CA (US)

(21) Appl. No.: 19/481,979

(22) PCT Filed: May 7, 2024
(86) PCT No.: PCT/US2024/028185

§ 371 Date: Nov. 6, 2025

Related U.S. Application Data
(60) Provisional application No. 63/501,062, filed on May 9, 2023.

(51) Int. Cl. A61K 31/553; A61K 31/437; A61K 31/4545; A61K 31/4725; A61K 31/4985; A61K 31/506; A61K 31/519; A61K 31/5377; A61K 31/5386; A61K 31/541; C07D 409/04; C07D 471/04; C07D 495/04; C07D 513/04; C07D 519/00

(52) U.S. Cl. CPC A61K 31/553; A61K 31/437; A61K 31/4545; A61K 31/4725; A61K 31/4985; A61K 31/506; A61K 31/519; A61K 31/5377; A61K 31/5386; A61K 31/541; C07D 409/04; C07D 471/04; C07D 495/04; C07D 513/04; C07D 519/00

(57) ABSTRACT

The present disclosure provides novel 15-hydroxy-prostaglandin dehydrogenase inhibitors, pharmaceutical compositions comprising such compounds, and methods of using such compounds and compositions in treating 15-hydroxy-prostaglandin dehydrogenase-mediated disease. Also provided are methods of making such compounds and intermediates thereof. The compounds have a structure of Formula (I). Further, the disclosure provides intermediates useful in the synthesis of compounds of Formula (I).

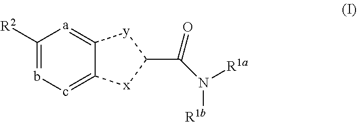

### CROSS-REFERENCE TO RELATED APPLICATIONS

This application claims the benefit of priority to U.S. Provisional Patent Application No. 63/501,062, filed May 9, 2023, which is hereby incorporated by reference in its entirety for all purposes as if fully set forth herein.

### FIELD

Provided herein are novel compounds, pharmaceutical compositions comprising such compounds, and methods of using such compounds and compositions in treating 15-hydroxy-prostaglandin dehydrogenase-mediated disease. Also provided are methods of making such compounds and intermediates thereof.

### BACKGROUND

Short-chain dehydrogenases (SCDs) are a family of dehydrogenases that are involved in synthesis and degradation of fatty acids, steroids, and some prostaglandins. They are therefore implicated in a variety of disorders such as lipid storage disease, myopathy, SCD deficiency, and certain genetic disorders. The SCD 15-hydroxy-prostaglandin dehydrogenase (15-PGDH) (also identified as 15-prostaglandin dehydrogenase or hydroxyprostaglandin dehydrogenase 15-(nicotinamide adeninedinucleotide)), represents a key enzyme in the inactivation of a number of active prostaglandins, leukotrienes and hydroxyeicosatetraenoic acids (HETEs). Recent studies suggest that inhibitors of 15-PGDH and activators of 15-PGDH could be therapeutically valuable. 15-PGDH is responsible for the inactivation of prostaglandin E2 (PGE2), which is a downstream product of cyclooxygenase-2 (COX-2) metabolism. PGE2 has been shown to be beneficial in a variety of biological processes, such as hair density, dermal wound healing, and bone formation.

### SUMMARY

Several embodiments disclosed herein provide novel compounds. In several embodiments, these compounds comprise a 5,6-fused bicyclic thieno-aryl ring system, a 5,6-fused bicyclic thieno-heteroaryl, ring system, or a 5,6-fused bicyclic thieno-heterocyclyl ring system as a core (see, e.g., Formula (I)). In several embodiments, the compounds disclosed herein are useful in modulating SCD and, more specifically, in modulating the activity of and/or in inhibiting the activity of 15-PDGH. In several embodiments, these 15-PGDH inhibiting compounds are useful in modulating tissue prostaglandin levels and treating diseases, disorders, or conditions that are related to 15-PGDH activity. In several embodiments, the compounds disclosed herein can be administered to a subject in an amount effective to inhibit the activity of a 15-PGDH and/or to treat a 15-PGDH mediated disease, disorder, or condition.

Several embodiments disclosed herein pertain to a compound of Formula (I):

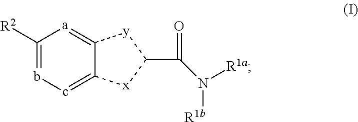

or a pharmaceutically acceptable salt thereof;
wherein

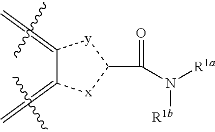

is

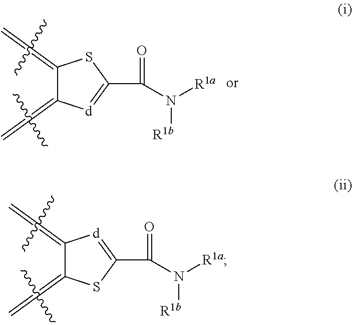

a is N, —N⁺—O⁻, or CH;
b is N or CH;
c is N, CH, or C—R³;
d is N, CH, or C—R⁴;
R^{1a} is —H, alkyl, haloalkyl, or heteroalkyl;
   wherein R^{1a} may be unsubstituted or substituted with one or more substituents, each of which independently is halogen, —OH, —N(R^{b})₂, alkyl, or alkoxy;
R^{1b} is alkyl, haloalkyl, heteroalkyl, carbocyclyl, or heterocyclyl;
   wherein R^{1b} may be unsubstituted or substituted with one or more substituents, each of which independently is halogen, —OH, —N(R^{b})₂, alkyl, or alkoxy;
or, alternatively, R^{1a} and R^{1b} together form a heterocyclyl;
   wherein the heterocyclyl formed by R^{1a} and R^{1b} may be unsubstituted or substituted with one or more substituents, each of which independently is halogen, —OH, —CN, —N(R^{b})₂, alkyl, haloalkyl, alkoxy, heteroalkyl, cycloalkyl, or heterocyclyl;
R² is heterocyclyl, heteroaryl, or, alternatively, when a is —N⁺—O⁻, then R² may be H;
   wherein R² may be unsubstituted or substituted with one or more instances of R⁵;
   wherein each instance of R⁵ independently is oxo, CN, alkyl, cycloalkyl, haloalkyl, alkoxy, C(O)N(R⁶)₂, —N(R⁶)₂, —N(R⁶)(alkylene-OH), or heterocyclyl;
      wherein each instance of R⁵ may be unsubstituted or substituted one or more instances of R⁶;
      wherein each instance of R⁶ independently is —H, alkyl, C(O)alkyl, C(O)₂alkyl, alkylene-OH, or S(O)ₙalkyl, where n is 0, 1, or 2;
         wherein R⁶ may be unsubstituted or substituted with one or more substituents each of which independently is —F, —OH, alkoxy;
R³ is —H, alkyl, cycloalkyl, aryl, heteroaryl, or heterocyclyl;
   wherein R³ may be unsubstituted or substituted with one or more substituents each of which independently is halogen, —OH, oxo, —N(R^{b})₂, alkyl, haloalkyl, alkoxy, alkylene-alkoxy, or heteroalkyl;
R⁴ is —H, alkyl, CN, —F, —Cl, or —N(R⁷)₂ where each instance of R⁷, where present, independently is —H or alkyl; or, alternatively, two R⁷ substituents together form a heterocyclyl;
   wherein R⁷ may be unsubstituted or substituted with one or more substituents each of which independently is —F, —OH, alkyl, or alkoxy; and
wherein each instance of R^{b}, where present, independently is —H or alkyl; or, alternatively, two R^{b} substituents together form a heterocyclyl;
wherein R^{b} may be unsubstituted or substituted with one or more substituents each of which independently is —F, —OH, alkyl, or alkoxy.

In several embodiments, d is CH or C—R⁴. In several embodiments, d is CH.

In several embodiments, R^{1a} is —H, C₁₋₆ alkyl, C₁₋₆ haloalkyl, or C₁₋₆ heteroalkyl. In several embodiments, R^{1a} may be unsubstituted or substituted with one or more substituents, each of which independently is halogen, —OH, —N(R^{b})₂, C₁₋₃ alkyl, or C₁₋₃ alkoxy.

In several embodiments, R^{1b} is C₁₋₆ alkyl, C₁₋₆ haloalkyl, C₁₋₆ heteroalkyl, C₃₋₁₀ cycloalkyl, or heterocyclyl having 3 to 10 ring members. In several embodiments, R^{1b} may be unsubstituted or substituted with one or more substituents, each of which independently is halogen, —OH, —N(R^{b})₂, C₁₋₃ alkyl, or C₁₋₃ alkoxy.

In several embodiments, R^{1a} and R^{1b} together form a heterocyclyl having 3 to 10 ring members. In several embodiments, the heterocyclyl formed by R^{1a} and R^{1b} may be unsubstituted or substituted with one or more substituents, each of which independently is halogen, —OH, —CN, —N(R^{b})₂, C₁₋₆ alkyl, C₁₋₆ haloalkyl, C₁₋₆alkoxy, C₁₋₆heteroalkyl, C₃₋₆cycloalkyl, or heterocyclyl having 3 to 6 ring members.

In several embodiments, R² is heterocyclyl having 5 to 10 ring members, heteroaryl having 5 to 10 ring members or, alternatively, when a is —N⁺—O⁻, then R² may be H. In several embodiments, R² may be unsubstituted or substituted with one or more instances of R⁵; wherein each instance of R⁵ independently is oxo, CN, C₁₋₆ alkyl, C₃₋₆ cycloalkyl, C₁₋₆ haloalkyl, C₁₋₆ alkoxy, C(O)N(R⁶)₂, —N(R⁶)₂, —N(R⁶)(C₁₋₆ alkylene-OH), or heterocyclyl having 3 to 10 members. In several embodiments, R⁵ may be unsubstituted or substituted one or more instances of R⁶. In several embodiments, R⁶ may be unsubstituted or substituted with one or more substituents each of which independently is —F, —OH, C₁₋₃ alkoxy.

In several embodiments, R³ is —H, C₁₋₆ alkyl, C₃₋₁₀ cycloalkyl, C₆₋₁₀ aryl, heteroaryl having 5 to 10 ring members, or heterocyclyl having 3 to 10 ring members. In several embodiments, R³ may be unsubstituted or substituted with one or more substituents each of which independently is halogen, —OH, oxo, —N(R^{b})₂, C₁₋₃ alkyl, C₁₋₃ haloalkyl, C₁₋₃ alkoxy, C₁₋₃ alkylene-C₁₋₃ alkoxy, or C₁₋₆ heteroalkyl.

In several embodiments, R⁴ is —H, alkyl, or —N(R⁷)₂. In several embodiments, R⁴ is —H, C₁₋₆ alkyl, or —N(R⁷)₂ where each instance of R⁷, where present, independently is —H or C₁₋₆ alkyl; or, alternatively, two R⁷ substituents together form a heterocyclyl having 3 to 6 ring members. In several embodiments, R⁷ may be unsubstituted or substituted with one or more substituents each of which independently is —F, —OH, C₁₋₃ alkyl, or C₁₋₃ alkoxy. In several embodiments, R⁷ may be unsubstituted or substituted with one or more substituents each of which independently is —F, —OH, or C₁₋₃ alkoxy.

In several embodiments, each instance of R^{b}, where present, independently is —H or C₁₋₆ alkyl; or, alternatively, two R^{b} substituents together form a heterocyclyl having 3 to 6 ring members. In several embodiments, R^{b} may be unsubstituted or substituted with one or more substituents each of which independently is —F, —OH, C₁₋₃ alkyl, or C₁₋₃ alkoxy. In several embodiments, R^{b} may be unsubstituted or substituted with one or more substituents each of which independently is —F, —OH, or C₁₋₃ alkoxy.

Several embodiments pertain to a pharmaceutical composition comprising a compound or salt of Formula (I) and a pharmaceutically acceptable excipient.

Several embodiments pertain to a compound or salt of Formula (I) or a pharmaceutical composition comprising a compound of Formula (I) for use as a medicament.

Several embodiments pertain to a compound or salt of Formula (I) or a pharmaceutical composition comprising a compound of Formula (I) for use in treating a 15-PGDH mediated disease or disorder.

Several embodiments pertain to a compound or salt of Formula (I) or a pharmaceutical composition comprising a compound of Formula (I) for use in treating inflammatory bowel disease.

Several embodiments pertain to a compound or salt of Formula (I) or a pharmaceutical composition comprising a compound of Formula (I) for use in treating ulcerative colitis.

Several embodiments pertain to a compound or salt of Formula (I) or a pharmaceutical composition comprising a compound of Formula (I) for use in treating Crohn's disease.

Several embodiments pertain to a compound or salt of Formula (I) or a pharmaceutical composition comprising a compound of Formula (I) for use in treating a fibrotic disease, disorder or condition.

Several embodiments pertain to a method of treating a 15-PGDH mediated disease, disorder, or condition in a subject in need thereof, the method comprising administering to the subject therapeutically an effective amount of a compound or salt of Formula (I) or a pharmaceutical composition comprising a compound of Formula (I).

Several embodiments pertain to a method of treating intestinal, gastrointestinal, or bowel disorders in a subject in need thereof, the method comprising administering to the subject therapeutically an effective amount of a compound or salt of Formula (I) or a pharmaceutical composition comprising a compound of Formula (I).

Further aspects and advantages will be apparent to those of ordinary skill in the art from a review of the following detailed description. The description hereafter includes specific cases, embodiments, and examples with the understanding that the disclosure is illustrative and is not intended to limit the embodiments of the present disclosure to the specific cases, embodiments, and examples described herein. To the contrary, reference to embodiments of the present disclosure is intended to cover alternatives, modifications, and equivalents as may be included within the spirit and scope of the embodiments of the present disclosure as defined by the appended claims.

### DETAILED DESCRIPTION

Several embodiments provide compounds and compositions, methods of use thereof, and methods of manufacture thereof. In several embodiments, the compounds disclosed herein are useful for inhibiting SCD and/or 15-PGDH and/or for treating SCD and/or 15-PGDH diseases, disorders, and conditions. In several embodiments, the compounds disclosed herein have a 5,6-fused bicyclic thieno-aryl ring system, a 5,6-fused bicyclic thieno-heteroaryl, ring system, or a 5,6-fused bicyclic thieno-heterocyclyl ring system core structure. In several embodiments, the compounds disclosed herein have a 5,6-fused bicyclic thieno-aryl ring system core structure. In several embodiments, the compounds disclosed herein have a 5,6-fused bicyclic thieno-heteroaryl ring system core structure. In several embodiments, the compounds disclosed herein have a 5,6-fused bicyclic thieno-heterocyclyl ring system core structure. In several embodiments, the compound comprises an amido moiety located alpha to the 2-position of a 5,6-fused bicyclic thieno-aryl ring system. In several embodiments, the compound comprises an amido moiety located alpha to the 2-position of a 5,6-fused bicyclic thieno-heteroaryl ring system. In several embodiments, the compound comprises an amido moiety located alpha to the 2-position of a 5,6-fused bicyclic thieno-heterocyclyl ring system. Elsewhere herein, these compounds may be referred to as 2-amido-5,6-fused-thieno-aryl(s) or 2-amido-5,6-fused-thieno-heterocyclyl(s) for brevity. These compounds and any other compounds disclosed herein may be generally referred to as 15-PGDH inhibitors.

The following description provides context and examples, but should not be interpreted to limit the scope of the inventions covered by the claims that follow in this specification or in any other application that claims priority to this specification. No single component or collection of components is essential or indispensable. The section headings used herein are for organizational purposes only and are not to be construed as limiting the subject matter described. Features disclosed under one heading (such as a composition or combination) can be combined with features disclosed under a different heading (a method of treating).

### Definitions

Unless defined otherwise, all technical and scientific terms used herein have the same meaning as is commonly understood by one of ordinary skill in the art to which this disclosure belongs. In the event that there is a plurality of definitions for a term herein, those in this section prevail unless stated otherwise.

As used herein, “C_{a-b}” (or similar wording, such as, Cₐ to C_{b}) in which “a” and “b” are integers refer to the number of carbon atoms in an alkyl, alkylene, alkenyl, alkenylene, alkynyl, alkynylene, haloalkyl, alkoxy, thioalkyl, heteroalkyl, cycloalkyl, cycloalkenyl, cycloalkynyl, aryl, heteroaryl, or other group. That is, the alkyl, alkenyl, alkynyl, ring of the cycloalkyl, ring of the cycloalkenyl, ring of the cycloalkynyl, ring of the aryl, or the ring of the heteroaryl can contain from “a” to “b”, inclusive, carbon atoms. Thus, for example, a “C₁₋₄ alkyl” group (or C₁ to C₄ alkyl) refers to all alkyl groups having from 1 to 4 carbons (e.g., 1, 2, 3, or 4), that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. A “C₁₋₆ alkyl” group refers to all alkyl groups having from 1 to 6 carbons (e.g., 1, 2, 3, 4, 5, or 6). If no “a” and “b” are designated with regard to a group, the ranges described in these definitions are envisioned. As will be appreciated in view of the preceding disclosure, where a range is disclosed (for alkyl groups or other groups disclosed herein), each individual member within that disclosed range is also envisioned and disclosed. Thus, where a “C₁₋₆ alkyl” is disclosed, all alkyl groups having from 1 to 6 carbons and any alkyl groups containing 1 carbon, 2 carbons, 3 carbons, 4 carbons, 5 carbons, or 6 carbons, or combinations of the foregoing (e.g., 1, 2, 3, or 5 carbons; 2, 3, 4, or 6 carbons; etc.) are provided. Likewise, where a “C₁₋₃ alkyl” is disclosed, all alkyl groups having from 1 to 3 carbons and any alkyl groups containing 1 carbon, 2 carbons, or 3 carbons, or combinations of the foregoing (e.g., 1 or 3; 1 or 2; 2 or 3; etc.) are provided.

The term “alkyl” refers to a straight or branched hydrocarbon chain that is fully saturated (i.e., contains no double or triple bonds). Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 12 carbon atoms. The “alkyl” group could also be a lower alkyl having 1 to 6 carbon atoms. A C₁₋₅ alkyl includes C₅ alkyls, C₄ alkyls, C₃ alkyls. C₂ alkyls and C₁ alkyl (i.e., methyl). A C₁₋₆ alkyl includes all moieties described above for C₁₋₅ alkyls but also includes C₆ alkyls. A C₁₋₁₀ alkyl includes all moieties described above for C₁₋₅ alkyls and C₁₋₆ alkyls, but also includes C₇, C₈, C₉ and C₁₀ alkyls. Similarly, a C₁₋₁₂ alkyl includes all the foregoing moieties, but also includes C₁₁ and C₁₂ alkyls. By way of example only, “C₁₋₄ alkyl” indicates that there are one to four carbon atoms in the alkyl chain, i.e., the alkyl chain is methyl, ethyl, propyl, iso-propyl, n-butyl, iso-butyl, sec-butyl, or t-butyl. Non-limiting examples of C₁₋₁₂ alkyl include, but are in no way limited to, methyl (“Me” or —CH₃), ethyl, n-propyl, i-propyl, sec-propyl, n-butyl, i-butyl, sec-butyl, t-butyl, n-pentyl, neo-pentyl, n-hexyl, n-heptyl, n-octyl, n-nonyl, n-decyl, n-undecyl, and n-dodecyl.

The term “alkylene” or “alkylene chain” refers to a fully saturated, straight or branched divalent hydrocarbon chain radical, and having from one to twelve carbon atoms. Examples of alkylene groups include, but are not limited to, methylene, ethylene, propylene, butylene, pentylene, hexylene, heptylene and octylene. A C₁₋₁₂ alkylene includes C₁₂ alkylenes, C₁₁ alkylenes, C₁₀ alkylenes, C₉ alkylenes, C₈ alkylenes, C₇ alkylenes, C₆ alkylenes, C₅ alkylenes, C₄ alkylenes, C₃ alkylenes, and C₂ alkylenes, and C₁ alkylene (i.e., methylene). An alkylene group may be a lower alkylene having 1 to 6 carbon atoms. A lower alkylene includes C₆ alkylenes, C₅ alkylenes. C₄ alkylenes, C₃ alkylenes, and C₂ alkylenes, and C₁ alkylene. It also is to be understood that certain diradical naming conventions can include either mono-radical or di-radical naming conventions, depending on the context. For example, where a position of a substituent within a molecule requires two points of attachment to the rest of the molecule, it is understood that the substituent is a di-radical. For example, a substituent identified as alkyl but that requires two points of attachment includes alkylene di-radicals such as —CH₂—, —CH₂CH₂—, —CH₂CH(CH₃)CH₂—, and the like.

The term “alkenyl” or “alkenyl group” refers to a straight or branched hydrocarbon chain radical having from two to twelve carbon atoms and having one or more carbon-carbon double bonds. An alkenyl group comprising up to 12 carbon atoms is a C₂₋₁₂ alkenyl, an alkenyl comprising up to 10 carbon atoms is a C₂₋₁₀ alkenyl, an alkenyl group comprising up to 6 carbon atoms is a C₂₋₆ alkenyl and an alkenyl comprising up to 5 carbon atoms is a C₂₋₅ alkenyl. A C₂₋₅ alkenyl includes C₅ alkenyls, C₄ alkenyls, C₃ alkenyls, and C₂ alkenyls. A C₂₋₆ alkenyl includes all moieties described above for C₂₋₅ alkenyls but also includes C₆ alkenyls. A C₂₋₁₀ alkenyl includes all moieties described above for C₂₋₅ alkenyls and C₂₋₆ alkenyls, but also includes C₇, C₈, C₉ and C₁₀ alkenyls. A C₂₋₁₂ alkenyl includes all the foregoing moieties, but also includes C₁₁ and C₁₂ alkenyls. Non-limiting examples of C₂₋₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl.

The term “alkynyl” or “alkynyl group” refers to a straight or branched hydrocarbon chain radical having from two to twelve carbon atoms and having one or more carbon-carbon triple bonds. An alkynyl group comprising up to 12 carbon atoms is a C₂₋₁₂ alkynyl, an alkynyl comprising up to 10 carbon atoms is a C₂₋₁₀ alkynyl, an alkynyl group comprising up to 6 carbon atoms is a C₂₋₆ alkynyl and an alkynyl comprising up to 5 carbon atoms is a C₂₋₅ alkynyl. A C₂₋₅ alkynyl includes C₅ alkynyls, C₄ alkynyls, C₃ alkynyls, and C₂ alkynyls. A C₂₋₆ alkynyl includes all moieties described above for C₂₋₅ alkynyls but also includes C₆ alkynyls. A C₂₋₁₀ alkynyl includes all moieties described above for C₂₋₅ alkynyls and C₂₋₆ alkynyls, but also includes C₇, C₈, C₉ and C₁₀ alkynyls. A C₂₋₁₂ alkynyl includes all the foregoing moieties, but also includes C₁₁ and C₁₂ alkynyls. Non-limiting examples of C₂₋₁₂ alkynyl include ethynyl, propynyl, butynyl, pentynyl and the like.

The term “halogen” or “halo” means fluoro (—F), chloro (—Cl), bromo (—Br), or iodo (—I).

The term “haloalkyl” refers to a straight- or branched-chain alkyl group, substituting one or more or all hydrogens with halogens. Examples of haloalkyl groups include, but are not limited to, —CF₃, —CHF₂, —CH₂F, —CH₂CF₃, —CH₂CHF₂, —CH₂CH₂F, —CH₂CH₂Cl, —CH₂CF₂CF₃ and other groups that in light of the ordinary skill in the art and the teachings provided herein, would be considered equivalent to any one of the foregoing examples. The haloalkyl may be a lower haloalkyl. The haloalkyl may be perhalogenated (e.g., perfluorinated).

The term “alkoxy” refers to the formula —OR wherein R is an alkyl group as defined elsewhere herein. For illustration, a “C₁₋₉ alkoxy” includes but is not limited to methoxy, ethoxy, n-propoxy, 1-methylethoxy (isopropoxy), n-butoxy, iso-butoxy, sec-butoxy, tert-butoxy, and the like.

The term “heteroalkyl” refers to a straight or branched hydrocarbon chain (e.g., alkyl) containing one or more heteroatoms. A heteroatom is given its plain and ordinary meaning in organic chemistry, which includes an element other than carbon, including but not limited to, nitrogen (e.g., amino, etc.), oxygen (e.g., alkoxy, ether, hydroxyl, etc.), sulfur, and halogens. The heteroalkyl group may have 1 to 12 carbon atoms although the present definition also covers the occurrence of the term “heteroalkyl” where no numerical range is designated. The heteroalkyl group could also be a lower heteroalkyl having 1 to 6 carbon atoms. In various embodiments, the heteroalkyl may have from 1 to 4 heteroatoms, from 1 to 3 heteroatoms, 1 or 2 heteroatoms, or 1 heteroatom. The heteroalkyl group of the compounds may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain. As will be understood from the above, in embodiments having one heteroatom, the heteroatom may be found anywhere along the alkyl portion of the heteroalkyl group, including in the first position (e.g., the heteroatom of the heteroalkyl may serve as the atom that directly attaches the alkyl to the rest of the molecule).

The term “aryl” refers to a hydrocarbon ring system radical comprising hydrogen and at least one aromatic ring. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. For example, the aryl group can be a C₆₋₁₄ aryl group, a C₆₋₁₀ aryl group, or a C₆ aryl group. For purposes of this invention, the aryl radical can be a monocyclic, bicyclic, tricyclic or tetracyclic ring system, which can include fused or bridged ring systems, wherein at least one ring in the system is aromatic. Examples of aryl groups include, but are not limited to, phenyl, aceanthrylenyl, acenaphthylenyl, acephenanthrylenyl, anthracenyl, azulenyl, chrysenyl, fluoranthenyl, fluorenyl, as-indacene-yl, s-indacene-yl, indanyl, indenyl, naphthalenyl, phenalenyl, phenanthrenyl, pleiadenyl, pyrenyl, and triphenylenyl.

The term “carbocyclyl,” “carbocyclic,” or “carbocycle” means a non-aromatic cyclic ring or ring system containing only carbon atoms in the ring system backbone and comprising 3 to 20 ring members (3 to 20 carbon atom ring members; C₃₋₂₀). When the carbocyclyl is a ring system, two or more rings may be joined together in a fused, bridged or spiro-connected fashion. Carbocyclyls include cycloalkyls, cycloalkenyls, and cycloalkynyls. The carbocyclyl group may be a medium size carbocyclyl having 3 to 10 carbon atoms. The carbocyclyl group could have 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

The term “cycloalkyl” refers to a non-aromatic monocyclic or polycyclic fully saturated hydrocarbon radical consisting solely of carbon and hydrogen atoms, which can include fused, bridged, or spiral (spiro) ring systems, having from 3 to 20 carbon atom ring members (e.g., having from 3 to 10 ring atoms, 3 to 8 ring atoms, etc.) and which is attached to the rest of the molecule by a single bond. No ring in a cycloalkyl ring or ring system is aromatic. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like.

The term “cycloalkenyl” refers to a monocyclic or polycyclic hydrocarbon radical consisting solely of carbon and hydrogen atoms, having one or more carbon-carbon double bonds, which can include fused, bridged, or spiral (e.g., spiro) ring systems, having from 3 to 20 carbon atom ring members (e.g., having from 3 to 10 ring atoms, 4 to 10 ring atoms, etc.) and which is attached to the rest of the molecule by a single bond. No ring in a cycloalkenyl ring or ring system is aromatic. An example is cyclohexenyl, cycloalkenyl groups can contain 4 to 10 atoms in the ring(s). Monocyclic cycloalkenyl radicals include, for example, cyclopentenyl, cyclohexenyl, cycloheptenyl, cyclooctenyl, and the like. Polycyclic cycloalkenyl radicals include, for example, bicyclo[2.2.1]hept-2-enyl and the like.

The term “cycloalkynyl” refers to a monocyclic or polycyclic hydrocarbon radical consisting solely of carbon and hydrogen atoms, having one or more carbon-carbon triple bonds, which can include fused, bridged, or spiral (e.g., spiro) ring systems, having from 8 to 20 carbon atom ring members (e.g., having from 8 to 10 ring atoms, etc.) and which is attached to the rest of the molecule by a single bond. No ring in a cycloalkynyl ring or ring system is aromatic. Monocyclic cycloalkynyl radicals include, for example, cycloheptynyl, cyclooctynyl, and the like.

The term “heterocyclyl” refers to three-, four-, five-, six-, seven-, eight-, nine-, ten-, up to 20-membered monocyclic, bicyclic, and tricyclic ring systems wherein carbon atoms together with from 1 to 5 heteroatoms (each of which independently is nitrogen (e.g., N, NH, N—R, etc.), oxygen, or sulfur (e.g., S, S(O), S(O)₂)) constitute said ring system. Heterocyclyl or heterocyclic rings include non-heteroaryl ring systems (e.g., those cycles comprising heteroatoms as ring members that do not fall within the definition of a “heteroaryl”). Unless stated otherwise specifically in the specification, the heterocyclyl radical can be a monocyclic, bicyclic, tricyclic or tetracyclic ring system, which can include fused, bridged, and spiro ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, aziridinyl, 1,3-dioxin-yl, 1,3-dioxanyl, 1,4-dioxanyl, 1,3-oxathianyl, 1,3-oxathiolanyl, 1,3-oxathiolanyl, 1,3-dithiolyl, 1,3-dithiolanyl, 1,4-oxathianyl, tetrahydro-1,4-thiazinyl, dioxolanyl, decahydroisoquinolyl, imidazolidinyl, isothiazolidinyl, isoxazolidinyl, maleimidyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, succinimidyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, and the like. The point of attachment of the heterocyclyl, heterocyclic ring, or heterocycle to the rest of the molecule by a single bond is through a ring member atom, which can be carbon or nitrogen. A heterocycle may optionally contain one or more unsaturated bonds situated in such a way, however, that a fully delocalized pi-electron system does not occur in any ring of the ring system. The heterocyclyl group may be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations.

The term “heteroaryl” refers to a 5- to 20-membered ring system radical with 1 to 19 carbon atoms and 1 to 6 heteroatoms (each of which independently is nitrogen (e.g., N, NH, N—R, etc.), oxygen, or sulfur) as the ring members. The heteroaryl radical can be a monocyclic, bicyclic, tricyclic or tetracyclic ring system, which can include fused or bridged ring systems, wherein at least one ring is aromatic. The nitrogen, carbon or sulfur atoms in the heteroaryl radical can be optionally oxidized and the nitrogen atom can be optionally quaternized. The heteroaryl group can contain 5 to 14 ring members (atoms in the ring(s)), 5 to 10 ring members (atoms in the ring(s)), 5 to 9 ring members (atoms in the ring(s)), 5 to 7 ring members (atoms in the ring(s)), 5 to 6 ring members (atoms in the ring(s)). The heteroaryl group may be a medium size heteroaryl having 5 to 10 ring members. The heteroaryl group could also be a heteroaryl having 5 to 6 ring members. The heteroaryl group could also be a heteroaryl having 6 to 9 ring members. The heteroaryl group could also have 6 ring members. The heteroaryl group could also have 9 ring members. In various embodiments, a heteroaryl contains from 1 to 6 heteroatoms, from 1 to 5 heteroatoms, from 1 to 4 heteroatoms, from 1 to 3 heteroatoms, from 1 to 2 heteroatoms, or 1 heteroatom. For example, in various embodiments, a heteroaryl contains 1 to 5 nitrogen atoms, 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, 1 sulfur or oxygen atom, etc. Examples include, but are not limited to, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, isothiazolyl, imidazolyl, imidazo[1,2-a]pyridinyl, indazolyl, indolyl, indazolyl, isoindolyl, indolinyl, isoindolinyl, isoquinolyl, indolizinyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrazolopyridinyl, pyridine-onyl, quinazolinyl, quinoxalinyl, quinolinyl, isoquinolinyl, tetrahydroquinolinyl, thiazolyl, thiadiazolyl, triazolyl, tetrazolyl, triazinyl, and thiophene-yl (i.e., thienyl).

The term “multicyclic” refers to a ring system having more than one ring (e.g., bicyclic, tricyclic or tetracyclic), which can include fused, bridged, and spiral (e.g., spiro) ring systems.

The term “hydroxy” (or “hydroxyl”) refers to a —OH group.

The term “cyano” group refers to a “—CN” group.

The term “oxo” refers to a ═O substituent.

The term “amino” refers to a NH₂ group.

The term “alkylamino” refers to a “—NR_{A}H” group in which R_{A} is alkyl.

The term “dialkylamino” refers to a “—NR_{A}R_{B}” group in which R_{A} and R_{B} are each independently alkyl. R_{A} and R_{B} may be taken together with the nitrogen to which they are attached to provide a heteroaryl or heterocyclyl.

The term “aminoalkyl” refers to an amino group connected via an alkylene group. An aminoalkyl can be unsubstituted or substituted.

As used herein, any “R” group(s) such as, without limitation, R¹, R², R³, etc., represent substituents that can be attached to the indicated atom. An R group may be unsubstituted or substituted. If two “R” groups are described as being “taken together” (or similar language), the R groups and the atoms they are attached to can form a cycle (e.g., cycloalkyl, aryl, heteroaryl, heterocyclyl). When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

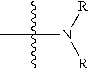

and each instance of R is defined as being independently hydrogen or alkyl, or each instance of R together with the nitrogen to which they are attached form a heterocyclyl, it is meant that each instance of R can be independently hydrogen or alkyl, or alternatively, the substructure has structure:

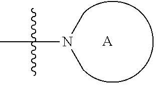

where ring A is a heterocyclyl ring containing the depicted nitrogen. As further illustration, without limitation, if R_{A} and R_{B} of an NR_{A} R_{B} group are indicated to be “taken together,” it means that they are covalently bonded to one another to form a ring:

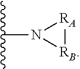

A cyclic structure may be shown using provided using the following structure (or a similar structure with a different ring, heteroatoms, unsaturated bonds, etc.):

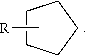

When a cyclic structure is depicted using this type of illustration, what is meant is that the R group may be attached to any position of the ring by replacing an —H of the ring with —R. For example, for the following ring:

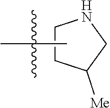

it is meant to include any of the following structures:

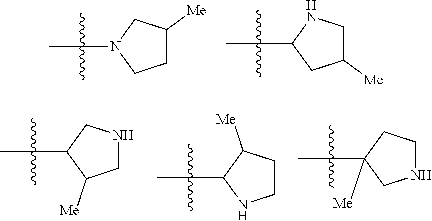 [SMILES: *N1CCC(C)C1 ; *C1CC(C)CN1 ; *C1CNCC1C ; *C1NCCC1C ; *C1(C)CCNC1]

where [symbol] indicates a bond to a remaining portion of the structure. Likewise, for the following structure:

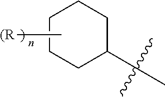

where [symbol] indicates a bond to a remaining portion of the structure and n is 1 to 6, any of the following structures are envisioned or other variations (as would be readily appreciated by the one of skill in the art):

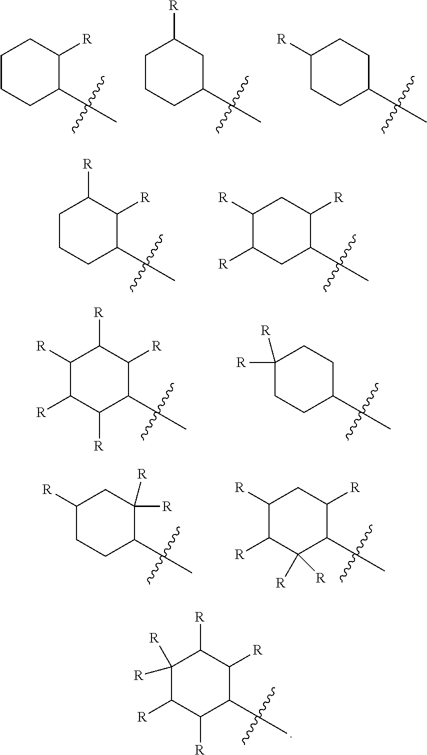

Additionally, where a nitrogen atom is present in the cycle, a hydrogen atom may be removed from the nitrogen to provide a substitution. Thus, for the following structure:

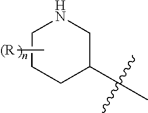

where [symbol] indicates a bond to a remaining portion of the structure and n is 1 to 6, any of the following structures are envisioned or other variations (as would be readily appreciated by the one of skill in the art):

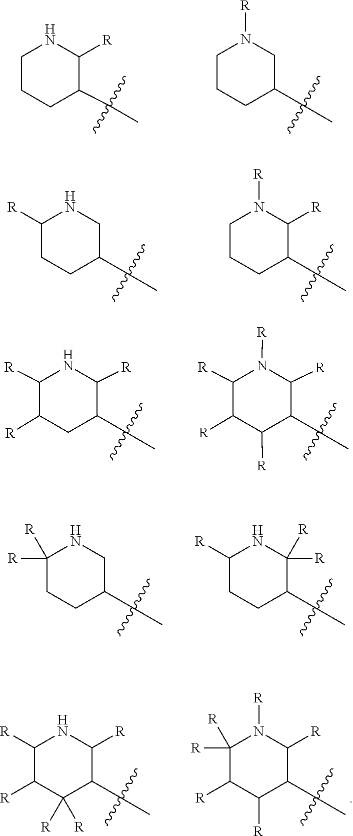

Where specified, a nitrogen atom in the cycle may provide a cation. Thus, for the following structure:

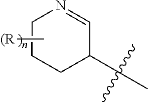

where [symbol] indicates a bond to a remaining portion of the structure and n is 1 to 6, any of the following structures are envisioned or other variations (as would be readily appreciated by the one of skill in the art):

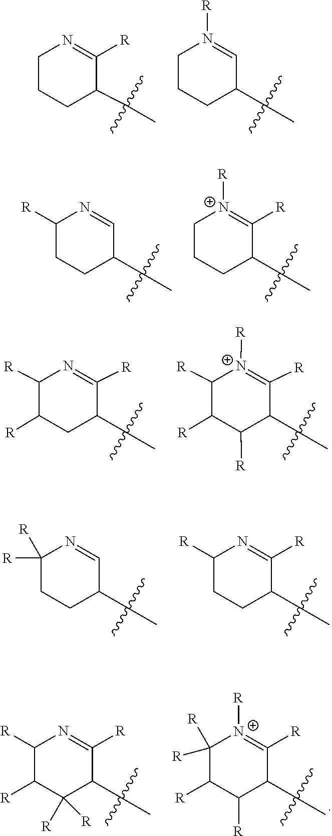

When two “adjacent” R groups are said to form a ring “together with the atoms to which they are attached,” it is meant that the collective unit of the atoms, intervening bonds, and the two R groups are the recited ring. For example, when the following substructure is present:

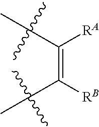

and R^{A} and R^{B} are each independently hydrogen or alkyl, or R¹ and R² together with the atoms to which they are attached form an aryl or carbocyclyl, it is meant that R¹ and R² can be selected from hydrogen or alkyl, or alternatively, the substructure has structure:

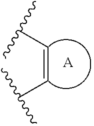

where A is an aryl ring or a carbocyclyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

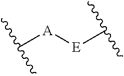

includes the substituent being oriented such that the “A” is attached at the leftmost attachment point of the molecule as well as the case in which “A” is attached at the rightmost attachment point of the molecule.

As noted in the definition for alkylene, it also is to be understood that certain radical naming conventions can include either a mono-radical or a di-radical, depending on the context. For example, where a substituent (e.g., in a genus structure) requires two points of attachment to the rest of the molecule, it is understood that the substituent is a di-radical. For example, a substituent identified as aminoalkyl that requires two points of attachment includes di-radicals such as —NHCH₂—, —NHCH₂CH₂—, —NHCH₂CH(CH₃)CH₂—, and the like. Other examples a substituent may require two points of attachment include alkoxy, aryl, heteroaryl, carbocyclyl, heterocyclyl, etc.

As used herein, a radical indicates species with a single, unpaired electron such that the species containing the radical can be covalently bonded to another species. Hence, in this context, a radical is not necessarily a free radical. Rather, a radical indicates a specific portion of a larger molecule.

Where a particular group (e.g., an R group) is indicated as being substituted, it is understood that such substitutions occur where valency allows. Thus, if the group is R and R is —H or alkyl with R is indicated as being unsubstituted or substituted, then it will be understood that the “—H” atom is not substituted because valency does not allow it to be. On the other hand, where R is alkyl, it may be unsubstituted or substituted.

“Solvate” refers to the compound formed by the interaction of a solvent and a compound described herein, a metabolite, or salt thereof. A solvate means solvent addition forms that contain either stoichiometric or non-stoichiometric amounts of solvent. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate. Non-limiting examples of hydrates include monohydrates, dihydrates, etc. Nonlimiting examples of solvates include ethanol solvates, acetone solvates, etc. The scope of the instant disclosure is to be understood to encompass all solvents of the compounds disclosed herein and the stereoisomers, tautomers and isotopically-labelled forms thereof or a pharmaceutically acceptable salt of any of the foregoing.

The term “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included.

The term “pharmaceutically acceptable salt” refers to salts that retain the biological effectiveness and properties of a compound, which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be prepared using and/or formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, camphorsulfonic acid, maleic acid, malonic acid, succinic acid, fumaric acid, formic acid, tartaric acid, citric acid, benzoic acid, cinnamic acid, mandelic acid, methanesulfonic acid, ethanesulfonic acid, p-toluenesulfonic acid, salicylic acid, carbonic acid, and the like. Pharmaceutically acceptable base addition salts can be prepared using and/or formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Organic bases from which salts can be derived also include, for example, ethylenediamine, N-methyl-glucamine, lysine, arginine, ornithine, choline, N,N-dibenzylethylenediamine, chloroprocaine, diethanolamine, procaine, N-benzylphenethylamine, piperazine, tris-(hydroxymethyl)-aminomethane, tetramethylammonium hydroxide, dibenzylamine, ephenamine, dehydroabietylamine, N-ethylpiperidine, benzylamine, tetramethylammonium, tetraethylammonium, methylamine, dimethylamine, ethylamine, basic amino acids, and the like. Other salts are known in the art, as described in International Publication No. WO87/05297. Additionally, the salts of the compounds described herein, can exist in either hydrated or unhydrated (the anhydrous) form or as solvates with other solvent molecules.

As used herein, “intermediate” compounds, includes structures produced from the synthetic procedures described, whether isolated or generated in-situ and not isolated, prior to obtaining the finally desired compound. These intermediates are included in the scope of this disclosure. Exemplary embodiments of such intermediate compounds are set forth elsewhere herein.

The compounds of the present disclosure may contain, for example, double bonds, one or more asymmetric carbon atoms, and bonds with a hindered rotation, and therefore, may exist as stereoisomers, such as double-bond isomers (i.e., geometric isomers (E/Z)), enantiomers, diastereomers, and atropoisomers. Accordingly, the scope of the instant disclosure is to be understood to encompass all possible stereoisomers of the illustrated compounds, including the stereoisomerically pure form (for example, geometrically pure, enantiomerically pure (e.g., (R), (S), (R)(R), (S)(S), etc.), diastereomerically pure, and atropoisomerically pure) and stereoisomeric mixtures (e.g., both (R) and (S), etc., diastereomers, and atropoisomers, or mixture of any of the foregoing) of any chemical structures disclosed herein (in whole or in part), unless the stereochemistry is specifically identified.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rats and mice but also includes many other species.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the standard deviation found in their respective testing measurements.

When referring to numerical values, the terms “or ranges including and/or spanning the aforementioned values” (and variations thereof) is meant to include any range that includes or spans the aforementioned values. For example, when the temperature of a reaction is expressed as “20° C., 30° C., 40° C., 50° C., or ranges including and/or spanning the aforementioned values,” this includes the particular temperature provided (e.g., 20° C., 30° C., 40° C., or 50° C.) or temperature ranges extending between 20° C. to 50° C., 20° C. to 40° C., 20° C. to 30° C., 30° C. to 50° C., 30° C. to 40° C., or 40° C. to 50° C.

### Compounds

Provided herein as Embodiment 1 is a compound of Formula (I)

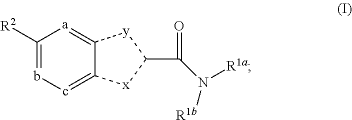

or a pharmaceutically acceptable salt of said compound;
wherein

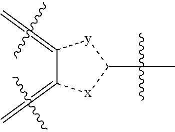

is

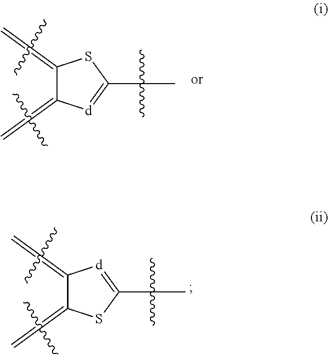

a is N, —N⁺—O⁻, or CH;
b is N or CH;
c is N, CH, or C—R³;
d is N, CH, or C—R⁴;
R^{1a} is —H, C₁₋₆ alkyl, C₁₋₆ haloalkyl, or C₁₋₆ heteroalkyl; wherein R^{1a} may be unsubstituted or substituted with one or more substituents, each of which independently is halogen, —OH, —N(R^{b})₂, C₁₋₃ alkyl, or C₁₋₃ alkoxy;
R^{1b} is C₁₋₆ alkyl, C₁₋₆ haloalkyl, C₁₋₆ heteroalkyl, C₃₋₁₀ cycloalkyl, or heterocyclyl having 3 to 10 ring members; wherein R^{1b} may be unsubstituted or substituted with one or more substituents, each of which independently is halogen, —OH, —N(R)₂, C₁₋₃ alkyl, or C₁₋₃ alkoxy; or, alternatively, R^{1a} and R^{1b} together form a heterocyclyl having 3 to 10 ring members;
   wherein the heterocyclyl formed by R^{1a} and R^{1b} may be unsubstituted or substituted with one or more substituents, each of which independently is halogen, —OH, —CN, —N(R)₂, C₁₋₆ alkyl. C₁₋₆ haloalkyl, C₁₋₆ alkoxy, C₁₋₆ heteroalkyl, C₃₋₆ cycloalkyl, or heterocyclyl having 3 to 6 ring members;
R² is heterocyclyl having 5 to 20 ring members, heteroaryl having 5 to 20 ring members or, alternatively, when a is —N⁺—O⁻, then R² may be H;
   wherein R² may be unsubstituted or substituted with one or more instances of R⁵;
   wherein each instance of R⁵ independently is oxo, CN, halogen, —OH, C₁₋₆ alkyl, C₃₋₆ cycloalkyl, C₁₋₆ haloalkyl, C₁₋₆ alkoxy, C(O)N(R⁶)₂, —N(R⁶)₂, —N(R⁶)(C₁₋₆ alkylene-OH), or heterocyclyl having 3 to 10 members;
      wherein each instance of R⁵ may be unsubstituted or substituted one or more instances of R⁶;
      wherein each instance of R⁶ independently is —H, C₁₋₆ alkyl, C(O)C₁₋₆ alkyl, C(O)₂C₁₋₆ alkyl, C₁₋₆ alkylene-OH, or S(O)ₙC₁₋₆alkyl, where n is 0, 1, or 2;
         wherein R⁶ may be unsubstituted or substituted with one or more substituents each of which independently is —F, —OH, C₁₋₃ alkoxy;
R³ is —H, C₁₋₆ alkyl, C₃₋₁₀ cycloalkyl, C₆₋₁₀ aryl, heteroaryl having 5 to 10 ring members, or heterocyclyl having 3 to 10 ring members;
   wherein R³ may be unsubstituted or substituted with one or more substituents each of which independently is halogen, —OH, oxo, —N(R^{b})₂, C₁₋₃ alkyl, C₁₋₃ haloalkyl, C₁₋₃ alkoxy, C₁₋₃ alkylene-C₁₋₃ alkoxy, or C₁₋₆ heteroalkyl;
R⁴ is —H, C₁₋₆ alkyl, or —N(R⁷)₂ where each instance of R⁷, where present, independently is —H or C₁₋₆ alkyl; or, alternatively, two R⁷ substituents together form a heterocyclyl having 3 to 6 ring members;
   wherein R⁷ may be unsubstituted or substituted with one or more substituents each of which independently is —F, —OH, C₁₋₃ alkyl, or C₁₋₃ alkoxy; and
wherein each instance of R^{b}, where present, independently is —H or C₁₋₆ alkyl; or, alternatively, two R^{b} substituents together form a heterocyclyl having 3 to 6 ring members;
   wherein R^{b} may be unsubstituted or substituted with one or more substituents each of which independently is —F, —OH, C₁₋₃ alkyl, or C₁₋₃ alkoxy.

For example, if and only if a is —N⁺—O⁻, then R² may be H. In several embodiments, R² is not thiophenyl. In several embodiments, R⁴ is not —N(R⁷)₂. In several embodiments, R⁴ is —H or C₁₋₆ alkyl. In several embodiments, if R³ is phenyl, then R⁴ is —H or C₁₋₆ alkyl. In several embodiments, if R³ is unsubstituted phenyl, then R⁴ is —H or C₁₋₆ alkyl. In several embodiments, R^{1a} is —H, C₁₋₃ alkyl, C₁₋₃ heteroalkyl, or C₁₋₃ haloalkyl. In several embodiments, R^{1a} is C₁₋₆ alkyl, C₁₋₆ heteroalkyl, or C₁₋₆ haloalkyl. In several embodiments, R^{1a} is C₁₋₃ alkyl, C₁₋₃ heteroalkyl, or C₃ haloalkyl. In several embodiments, R^{1a} is C₁₋₆ alkyl or C₁₋₆haloalkyl. In several embodiments, R^{1a} is —H or C₁₋₆alkyl. In several embodiments, R^{1a} is —H or C₁₋₆ haloalkyl. In several embodiments, R^{1a} is —H or C₁₋₆heteroalkyl. In several embodiments, R^{1a} is C₁₋₆alkyl. In several embodiments, R^{1a} is C₁₋₆haloalkyl. In several embodiments, R^{1a} is C₁₋₆heteroalkyl. In several embodiments, R^{1a} is —H. In several embodiments, R^{1b} is C₁₋₆ alkyl, C₁₋₆ haloalkyl, C₁₋₆ heteroalkyl, C₃₋₁₀ cycloalkyl, or heterocyclyl having 3 to 10 ring members. In several embodiments, R^{1b} is C₁₋₆ alkyl, C₁₋₆ haloalkyl, or C₁₋₆ heteroalkyl. In several embodiments, R^{1b} is propyl. In several embodiments, R^{1b} is C₃₋₁₀ cycloalkyl or heterocyclyl having 3 to 10 ring members. In several embodiments, R^{1b} is haloalkyl. In several embodiments, R^{1b} is heterocyclyl having 3 to 10 ring members. In several embodiments, R^{1a} and R^{1b} together form a heterocyclyl having 5 to 12-ring members. In several embodiments, R^{1a} and R^{1b} together form a heterocyclyl having 6 to 12-ring members. In several embodiments, R^{1a} and R^{1b} together form a heterocyclyl having 5 to 12-ring members. In several embodiments, R^{1a} and R^{1b} together form a heterocyclyl having 6 to 12-ring members. In several embodiments, R^{1a} and R^{1b} together form a heterocyclyl having 3 to 10 ring members. In several embodiments, R¹ and R^{1b} together form a heterocyclyl having 4 to 6 ring members. In several embodiments, R² is heteroaryl having 5 to 10 ring members or heterocyclyl having 3 to 10 ring members. In several embodiments, R² is heteroaryl having 5 to 10 ring members. In several embodiments, R³ is C₁₋₆ alkyl, C₁₋₆ haloalkyl, C₃₋₆ cycloalkyl, C₆₋₁₀ aryl, heteroaryl having 5 to 10 ring members, or heterocyclyl having 5 to 10 ring members. In several embodiments, R³ is C₁₋₆ alkyl, C₁₋₆ haloalkyl, or heterocyclyl having 5 to 10 ring members. In several embodiments, R³ is C₃₋₆ cycloalkyl. In several embodiments, R³ is heteroaryl having 5 to 10 ring members. In several embodiments, a haloalkyl as provided in a variable of Embodiment 1 is a perhalogenated, where each —H has been exchanged with a halogen. In several embodiments, the haloalkyl as provided in a variable of Embodiment 1 is not perhalogenated and one or more instances C—H occur (e.g., 1, 2, 3, 4, 5, 6, 7, 8, or more). In several embodiments, C—H instances occurring within the haloalkyl as provided in a variable of Embodiment 1 may occur on the same carbon (e.g., providing CH₂ or CH₃). The haloalkyl as provided in a variable of Embodiment 1 includes at least one and optionally more instances of a halogen (e.g., 1, 2, 3, 4, 5, 6, 7, 8, 9, 10, 11, more instances, or perhalo). In several embodiments, each halogen independently is —F, —Cl, —Br, or —I. In several embodiments, a heteroalkyl as provided in a variable of Embodiment 1 is includes a heteroatom within the alkyl backbone. In several embodiments, each heteroatom of a heteroalkyl as provided in a variable of Embodiment 1 independently is nitrogen, oxygen, or sulfur (e.g., S, SO, or SO₂). In several embodiments, a heteroalkyl as provided in a variable of Embodiment 1 may have from 1 to 4 heteroatoms (e.g., 1, 2, 3, or 4), from 1 to 3 heteroatoms (e.g., 1, 2, or 3), 1 or 2 heteroatoms, or 1 heteroatom. In several embodiments, R⁷ may be unsubstituted or substituted with one or more substituents each of which independently is —F, —OH, or C₁₋₃ alkoxy. R^{b} may be unsubstituted or substituted with one or more substituents each of which independently is —F, —OH, or C₁₋₃ alkoxy.

Provided herein as Embodiment 2 is the compound or salt of Embodiment 1, wherein, when R^{1a} is H, then a is CH, b is CH, c is C—R³, d is C—R⁴, R² is thienyl, R³ is phenyl, R⁴ is NH₂, R^{1b} is C₁₋₃ alkyl, wherein R^{1b} may be unsubstituted or substituted with C₁₋₃ alkoxy. In several embodiments, R³ is not —H. In several embodiments, R⁴ is not —H. In several embodiments, R⁶ is not —H.

Provided herein as Embodiment 3 is the compound or salt of Embodiment 1 or 2, wherein, when R^{1a} is C₁₋₆ alkyl, C₁₋₆ haloalkyl, or C₁₋₆ heteroalkyl, then d is CH.

Provided herein as Embodiment 4 is the compound or salt of any one of Embodiments 1 to 3, wherein when R² is triazolyl, tetrahydropyranyl, or oxetanyl, then R^{1b} is not a unsubstituted or substituted bridged [2.2.2]heterocyclyl with 8 ring members.

Provided herein as Embodiment 5 is the compound or salt of any one of Embodiments 1 to 4, wherein, when R² is heteroaryl having 5 ring members or heterocyclyl having 4 to 6 ring members, then R^{1b} is not a unsubstituted or substituted bridged [2.2.2]heterocyclyl with 8 ring members.

Provided herein as Embodiment 6 is the compound or salt of any one of Embodiments 1 to 5, wherein, R^{1b} is not a unsubstituted or substituted bridged [2.2.2]heterocyclyl with 8 ring members.

Provided herein as Embodiment 7 is the compound or salt of any one of Embodiments 1 to 6, wherein, when R^{1a} is H or Me and R^{1b} is C₁₋₃ alkyl or C₁₋₃ haloalkyl, then R³ is not unsubstituted or substituted morpholinyl.

Provided herein as Embodiment 8 is the compound or salt of any one of Embodiments 1 to 7, wherein, when R^{1a} is H, R³ is not unsubstituted or substituted morpholinyl.

Provided herein as Embodiment 9 is the compound or salt of any one of Embodiments 1 to 8, wherein, when R^{1a} and R^{1b} together provide unsubstituted or substituted piperidinyl or unsubstituted or substituted piperizinyl, then R³ is not unsubstituted or substituted morpholinyl.

Provided herein as Embodiment 10 is the compound or salt of any one of Embodiments 1 to 8, wherein, R³ is not unsubstituted or substituted morpholinyl.

Provided herein as Embodiment 11 is the compound or salt of any one of Embodiments 1 to 10, wherein R² is a heteroaryl that does not include S as a ring member.

Provided herein as Embodiment 12 is the compound or salt of any one of Embodiments 1 to 11, wherein R² is not

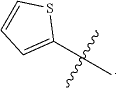 [SMILES: *c1cccs1]

Provided herein as Embodiment 13 is the compound or salt of any one of Embodiments 1 to 12, wherein R^{1a} is not —H.

Provided herein as Embodiment 14 is the compound or salt of any one of Embodiments 1 to 13, wherein R^{1b} is not propyl.

Provided herein as Embodiment 15 is the compound or salt of any one of Embodiments 1 to 14, wherein, when d is C—R⁴, then R⁴ is not —N(R)₂.

Provided herein as Embodiment 16 is the compound or salt of any one of Embodiments 1 to 15, wherein, when d is C—R⁴, then R⁴ is not —NH₂.

Provided herein as Embodiment 17 is the compound or salt of any one of Embodiments 1 to 16, wherein R³ is not

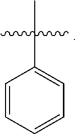 [SMILES: *c1ccccc1]

Provided herein as Embodiment 18 is the compound or salt of any one of Embodiments 1 to 17, wherein the compound of Formula (I) is not:

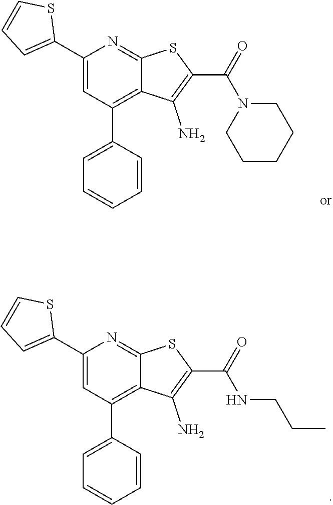 [SMILES: Nc1c(C(=O)N2CCCCC2)sc2nc(-c3cccs3)cc(-c3ccccc3)c12 ; CCCNC(=O)c1sc2nc(-c3cccs3)cc(-c3ccccc3)c2c1N]

Provided herein as Embodiment 19 is the compound or salt of any one of Embodiments 1 to 18, wherein R² is not

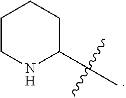 [SMILES: *C1CCCCN1]

Provided herein as Embodiment 20 is the compound or salt of any one of Embodiments 1 to 19, wherein the compound of Formula (I) is not 3-amino-N-methyl-6-(2-piperidinyl)thieno[2,3-b]pyridine-2-carboxamide.

Provided herein as Embodiment 21 is the compound or salt of any one of Embodiments 1 to 20, wherein the compound of Formula (I) is not: 1-cyclopentyl-4-[[6-(2-furanyl)-4-(trifluoromethyl)thieno[2,3-b]pyridin-2-yl]carbonyl]-2-piperazinone, or 1-cyclopentyl-4-[[6-(2-thienyl)-4-(trifluoromethyl)thieno[2,3-b]pyridin-2-yl]carbonyl]-2-piperazinone. For example, in several embodiments, the compound of Formula (I) is not 1-cyclopentyl-4-[[6-(2-furanyl)-4-(trifluoromethyl)thieno[2,3-b]pyridin-2-yl]carbonyl]-2-piperazinone. In several embodiments, the compound of Formula (I) is not 1-cyclopentyl-4-[[6-(2-thienyl)-4-(trifluoromethyl)thieno[2,3-b]pyridin-2-yl]carbonyl]-2-piperazinone.

Provided herein as Embodiment 22 is the compound or salt of any one of Embodiments 1 to 21, wherein the compound of Formula (I) is not: 2-(1H-indazol-4-yl)-N-methyl-4-(morpholin-4-yl)thieno[2,3-d]pyrimidine-6-carboxamide, N-methoxy-N-methyl-2-(4-morpholinyl)thieno[2,3-d]pyrimidine-6-carboxamide. N-Ethyl-2-(1H-indazol-4-yl)-4-(morpholin-4-yl)thieno[2,3-d]pyrimidine-6-carboxamide, 2-(1H-indazol-4-yl)-N-(1-methylethyl)-4-(4-morpholinyl)thieno[2,3-d]pyrimidine-6-carboxamide, N-(2-hydroxyethyl)-2-(1H-indazol-4-yl)-4-(morpholin-4-yl)thieno[2,3-d]pyrimidine-6-carboxamide, 2-(1H-indazol-4-yl)-4-(4-morpholinyl)-N-(2,2,2-trifluoroethyl)thieno[2,3-d]pyrimidine-6-carboxamide, 2-(1H-indazol-4-yl)-N,N-dimethyl-4-(morpholin-4-yl)thieno[2,3-d]pyrimidine-6-carboxamide, [2-(1H-Indazol-4-yl)-4-(morpholin-4-yl)thieno[2,3-d]pyrimidin-6-yl](4-methylpiperazin-1-yl)methanone, or [2-(1H-Indazol-4-yl)-4-(morpholin-4-yl)thieno[2,3-d]pyrimidin-6-yl](4-acetylpiperazin-1-yl)methanone. For example, in several embodiments, the compound of Formula (I) is not 2-(1H-indazol-4-yl)-N-methyl-4-(morpholin-4-yl)thieno[2,3-d]pyrimidine-6-carboxamide. In several embodiments, the compound of Formula (I) is not N-methoxy-N-methyl-2-(4-morpholinyl)thieno[2,3-d]pyrimidine-6-carboxamide. In several embodiments, the compound of Formula (I) is not N-Ethyl-2-(1H-indazol-4-yl)-4-(morpholin-4-yl)thieno[2,3-d]pyrimidine-6-carboxamide. In several embodiments, the compound of Formula (I) is not 2-(1H-indazol-4-yl)-N-(1-methylethyl)-4-(4-morpholinyl)thieno[2,3-d]pyrimidine-6-carboxamide. In several embodiments, the compound of Formula (I) is not N-(2-hydroxyethyl)-2-(1H-indazol-4-yl)-4-(morpholin-4-yl)thieno[2,3-d]pyrimidine-6-carboxamide. In several embodiments, the compound of Formula (I) is not 2-(1H-indazol-4-yl)-4-(4-morpholinyl)-N-(2,2,2-trifluoroethyl)thieno[2,3-d]pyrimidine-6-carboxamide. In several embodiments, the compound of Formula (I) is not 2-(1H-indazol-4-yl)-N,N-dimethyl-4-(morpholin-4-yl)thieno[2,3-d]pyrimidine-6-carboxamide. In several embodiments, the compound of Formula (I) is not [2-(1H-Indazol-4-yl)-4-(morpholin-4-yl)thieno[2,3-d]pyrimidin-6-yl](4-methylpiperazin-1-yl)methanone. In several embodiments, the compound of Formula (I) is not [2-(1H-Indazol-4-yl)-4-(morpholin-4-yl)thieno[2,3-d]pyrimidin-6-yl](4-acetylpiperazin-1-yl)methanone.

Provided herein as Embodiment 23 is the compound or salt of any one of Embodiments 1 to 22, wherein, when R³ is heterocyclyl comprising an N ring member, then R³ is not connected to the Formula (I) structure though an N ring member of the heterocyclyl.

Provided herein as Embodiment 24 is the compound or salt of any one of Embodiments 1 to 23, wherein the compound of Formula (I) is not: 2-(2-amino-5-pyrimidinyl)-N-methyl-4-(4-morpholinyl)thieno[3,2-d]pyrimidine-6-carboxamide, 2-(1H-indazol-4-yl)-N-methyl-4-(4-morpholinyl)thieno[3,2-d]pyrimidine-6-carboxamide, N-Ethyl-2-(1H-indazol-4-yl)-4-(4-morpholinyl)thieno[3,2-d]pyrimidine-6-carboxamide, 2-(1H-Indol-4-yl)-N-(2-methoxyethyl)-4-(4-morpholinyl)thieno[3,2-d]pyrimidine-6-carboxamide, N-(2-Hydroxyethyl)-2-(1H-indazol-4-yl)-4-(4-morpholinyl)thieno[3,2-d]pyrimidine-6-carboxamide, 2-(1H-Indazol-4-yl)-N-(2-methylpropyl)-4-(4-morpholinyl)thieno[3,2-d]pyrimidine-6-carboxamide, N-(2-Hydroxy-1-methylethyl)-2-(1H-indazol-4-yl)-4-(4-morpholinyl)thieno[3,2-d]pyrimidine-6-carboxamide, 2-(1H-Indazol-4-yl)-N-(2-methoxyethyl)-4-(4-morpholinyl)thieno[3,2-d]pyrimidine-6-carboxamide, 2-(2-Amino-5-pyrimidinyl)-4-(4-morpholinyl)-N-[2-(1-piperidinyl)ethyl]thieno[3,2-d]pyrimidine-6-carboxamide, 2-(1H-Indazol-4-yl)-4-(4-morpholinyl)-N-[2-(4-morpholinyl)ethyl]thieno[3,2-d]pyrimidine-6-carboxamide, 2-(1H-Indazol-4-yl)-4-(4-morpholinyl)-N-[2-(1-piperidinyl)ethyl]thieno[3,2-d]pyrimidine-6-carboxamide, 2-(1H-Indazol-4-yl)-4-(4-morpholinyl)-N-(2,2,2-trifluoroethyl)thieno[3,2-d]pyrimidine-6-carboxamide, [2-(2-Amino-5-pyrimidinyl)-4-(4-morpholinyl)thieno[3,2-d]pyrimidin-6-yl]-4-morpholinylmethanone, 2-(1H-Indazol-4-yl)-N,N-dimethyl-4-(4-morpholinyl)thieno[3,2-d]pyrimidine-6-carboxamide, [2-(2-Amino-5-pyrimidinyl)-4-(4-morpholinyl)thieno[3,2-d]pyrimidin-6-yl](4-methyl-1-piperazinyl)methanone, [2-(2-Amino-5-pyrimidinyl)-4-(4-morpholinyl)thieno[3,2-d]pyrimidin-6-yl][4-(2-hydroxyethyl)-1-piperazinyl]methanone, [2-(1H-Indazol-4-yl)-4-(4-morpholinyl)thieno[3,2-d]pyrimidin-6-yl]-4-morpholinylmethanone, N,N-Bis(2-hydroxyethyl)-2-(1H-indazol-4-yl)-4-(4-morpholinyl)thieno[3,2-d]pyrimidine-6-carboxamide. [2-(1H-Indazol-4-yl)-4-(4-morpholinyl)thieno[3,2-d]pyrimidin-6-yl](4-methyl-1-piperazinyl)methanone, [2-(1H-Indazol-4-yl)-4-(4-morpholinyl)thieno[3,2-d]pyrimidin-6-yl][3-(methylamino)-1-pyrrolidinyl]methanone, (4-Hydroxy-1-piperidinyl)[2-(1H-indazol-4-yl)-4-(4-morpholinyl)thieno[3,2-d]pyrimidin-6-yl]methanone, [4-(2-Hydroxyethyl)-1-piperazinyl][2-(1H-indazol-4-yl)-4-(4-morpholinyl)thieno[3,2-d]pyrimidin-6-yl]methanone, or [2-(1H-Indazol-4-yl)-4-(4-morpholinyl)thieno[3,2-d]pyrimidin-6-yl][4-(1-pyrrolidinyl)-1-piperidinyl]methanone. For example, in several embodiments, the compound of Formula (I) is not 2-(2-amino-5-pyrimidinyl)-N-methyl-4-(4-morpholinyl)thieno[3,2-d]pyrimidine-6-carboxamide. In several embodiments, the compound of Formula (I) is not 2-(1H-indazol-4-yl)-N-methyl-4-(4-morpholinyl)thieno[3,2-d]pyrimidine-6-carboxamide. In several embodiments, the compound of Formula (I) is not N-Ethyl-2-(1H-indazol-4-yl)-4-(4-morpholinyl)thieno[3,2-d]pyrimidine-6-carboxamide. In several embodiments, the compound of Formula (I) is not 2-(1H-Indol-4-yl)-N-(2-methoxyethyl)-4-(4-morpholinyl)thieno[3,2-d]pyrimidine-6-carboxamide. In several embodiments, the compound of Formula (I) is not N-(2-Hydroxyethyl)-2-(1H-indazol-4-yl)-4-(4-morpholinyl)thieno[3,2-d]pyrimidine-6-carboxamide. In several embodiments, the compound of Formula (I) is not 2-(1H-Indazol-4-yl)-N-(2-methylpropyl)-4-(4-morpholinyl)thieno[3,2-d]pyrimidine-6-carboxamide. In several embodiments, the compound of Formula (I) is not N-(2-Hydroxy-1-methylethyl)-2-(1H-indazol-4-yl)-4-(4-morpholinyl)thieno[3,2-d]pyrimidine-6-carboxamide. In several embodiments, the compound of Formula (I) is not 2-(1H-Indazol-4-yl)-N-(2-methoxyethyl)-4-(4-morpholinyl)thieno[3,2-d]pyrimidine-6-carboxamide. In several embodiments, the compound of Formula (I) is not 2-(2-Amino-5-pyrimidinyl)-4-(4-morpholinyl)-N-[2-(1-piperidinyl)ethyl]thieno[3,2-d]pyrimidine-6-carboxamide. In several embodiments, the compound of Formula (I) is not 2-(1H-Indazol-4-yl)-4-(4-morpholinyl)-N-[2-(4-morpholinyl)ethyl]thieno[3,2-d]pyrimidine-6-carboxamide. In several embodiments, the compound of Formula (I) is not 2-(1H-Indazol-4-yl)-4-(4-morpholinyl)-N-[2-(1-piperidinyl)ethyl]thieno[3,2-d]pyrimidine-6-carboxamide. In several embodiments, the compound of Formula (I) is not 2-(1H-Indazol-4-yl)-4-(4-morpholinyl)-N-(2,2,2-trifluoroethyl)thieno[3,2-d]pyrimidine-6-carboxamide. In several embodiments, the compound of Formula (I) is not [2-(2-Amino-5-pyrimidinyl)-4-(4-morpholinyl)thieno[3,2-d]pyrimidin-6-yl]-4-morpholinylmethanone. In several embodiments, the compound of Formula (I) is not 2-(1H-Indazol-4-yl)-N,N-dimethyl-4-(4-morpholinyl)thieno[3,2-d]pyrimidine-6-carboxamide. In several embodiments, the compound of Formula (I) is not [2-(2-Amino-5-pyrimidinyl)-4-(4-morpholinyl)thieno[3,2-d]pyrimidin-6-yl](4-methyl-1-piperazinyl)methanone. In several embodiments, the compound of Formula (I) is not [2-(2-Amino-5-pyrimidinyl)-4-(4-morpholinyl)thieno[3,2-d]pyrimidin-6-yl][4-(2-hydroxyethyl)-1-piperazinyl]methanone. In several embodiments, the compound of Formula (I) is not [2-(1H-Indazol-4-yl)-4-(4-morpholinyl)thieno[3,2-d]pyrimidin-6-yl]-4-morpholinylmethanone. In several embodiments, the compound of Formula (I) is not N,N-Bis(2-hydroxyethyl)-2-(1H-indazol-4-yl)-4-(4-morpholinyl)thieno[3,2-d]pyrimidine-6-carboxamide. In several embodiments, the compound of Formula (I) is not [2-(1H-Indazol-4-yl)-4-(4-morpholinyl)thieno[3,2-d]pyrimidin-6-yl](4-methyl-1-piperazinyl)methanone. In several embodiments, the compound of Formula (I) is not [2-(1H-Indazol-4-yl)-4-(4-morpholinyl)thieno[3,2-d]pyrimidin-6-yl][3-(methylamino)-1-pyrrolidinyl]methanone. In several embodiments, the compound of Formula (I) is not (4-Hydroxy-1-piperidinyl)[2-(1H-indazol-4-yl)-4-(4-morpholinyl)thieno[3,2-d]pyrimidin-6-yl]methanone. In several embodiments, the compound of Formula (I) is not [4-(2-Hydroxyethyl)-1-piperazinyl][2-(1H-indazol-4-yl)-4-(4-morpholinyl)thieno[3,2-d]pyrimidin-6-yl]methanone. In several embodiments, the compound of Formula (I) is not [2-(1H-Indazol-4-yl)-4-(4-morpholinyl)thieno[3,2-d]pyrimidin-6-yl][4-(1-pyrrolidinyl)-1-piperidinyl]methanone.

Provided herein as Embodiment 25 is the compound or salt of any one of Embodiments 1 to 10, wherein, when R^{1a} is H and R^{1b} is C₁₋₃ alkyl, then R² is not heteroaryl having 10 ring members and substituted with NH₂.

Provided herein as Embodiment 26 is the compound or salt of any one of Embodiments 1 to 25, wherein, when R² is pyrrolyl and R^{1b} is methyl, then R^{1a} is not H or methyl.

Provided herein as Embodiment 27 is the compound or salt of any one of Embodiments 1 to 26, wherein

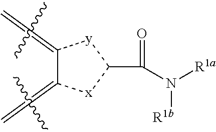

is

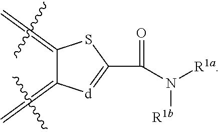

Provided herein as Embodiment 28 is the compound or salt of any one of Embodiments 1 to 26, wherein

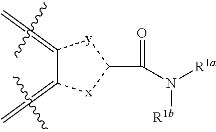

is

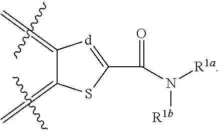

Provided herein as Embodiment 29 is the compound or salt of any one of Embodiments 1 to 28, wherein a is N.

Provided herein as Embodiment 30 is the compound or salt of any one of Embodiments 1 to 28, wherein a is CH.

Provided herein as Embodiment 31 is the compound or salt of any one of Embodiments 1 to 30, wherein b is N.

Provided herein as Embodiment 32 is the compound or salt of any one of Embodiments 1 to 30, wherein b is CH.

Provided herein as Embodiment 33 is the compound or salt of any one of Embodiments 1 to 32, wherein c is N.

Provided herein as Embodiment 34 is the compound or salt of any one of Embodiments 1 to 32, wherein c is CH.

Provided herein as Embodiment 35 is the compound or salt of any one of Embodiments 1 to 32, wherein c is C—R³.

Provided herein as Embodiment 36 is the compound or salt of any one of Embodiments 1 to 35, wherein d is N.

Provided herein as Embodiment 37 is the compound or salt of any one of Embodiments 1 to 35, wherein d is CH.

Provided herein as Embodiment 38 is the compound or salt of any one of Embodiments 1 to 35, wherein d is C—R⁴.

Provided herein as Embodiment 39 is the compound or salt of any one of Embodiments 1 to 28, wherein the

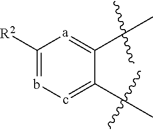

group is

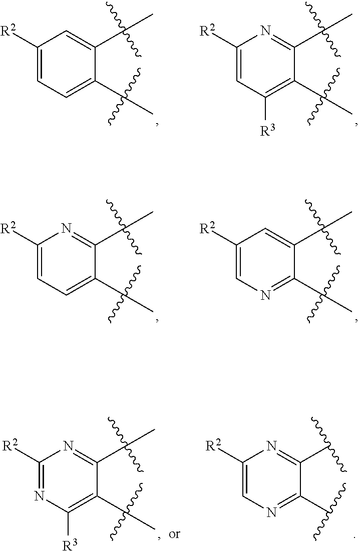

For example, in several embodiments, the

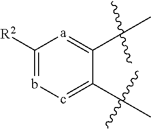

group is

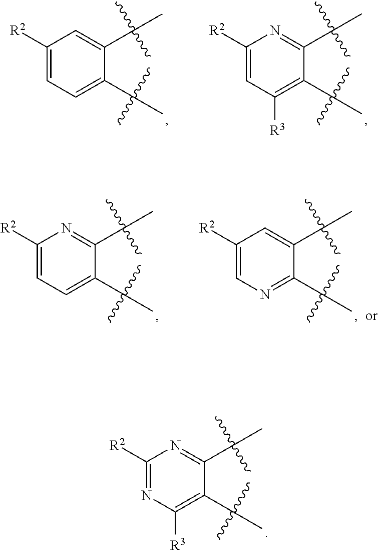

In several embodiments, the

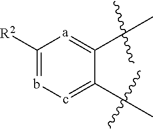

group is

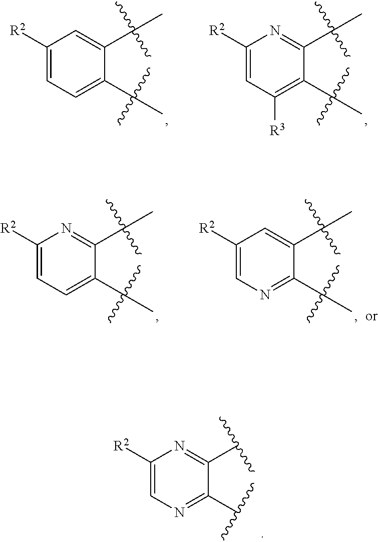

In several embodiments, the

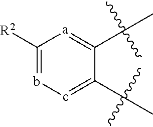

group is

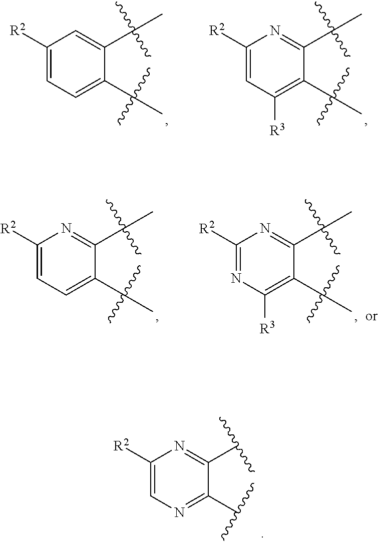

In several embodiments, the

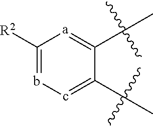

group is,

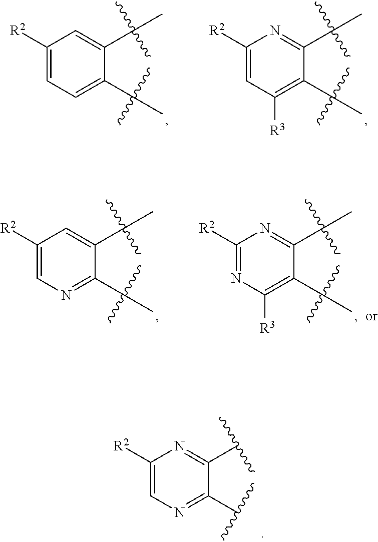

In several embodiments, the

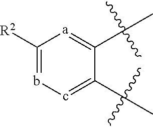

group is

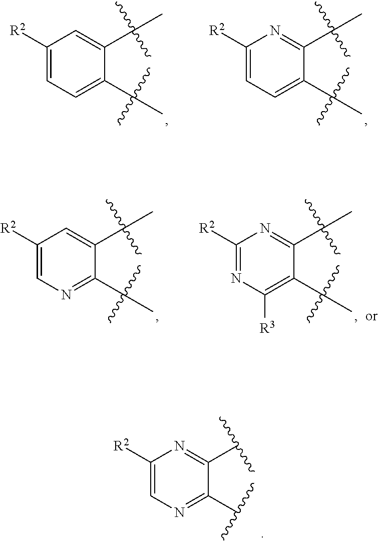

In several embodiments, the

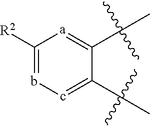

group is

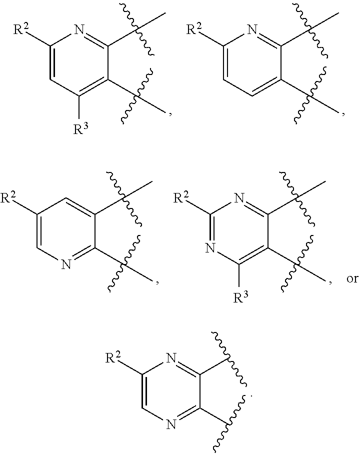

In several embodiments, the

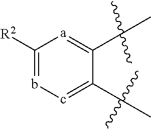

group is

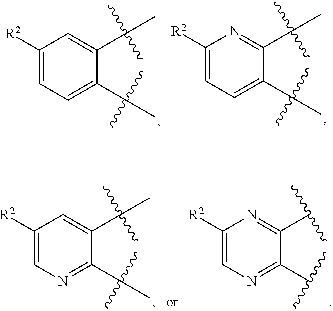

In several embodiments, the

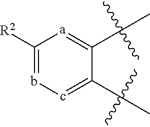

group is

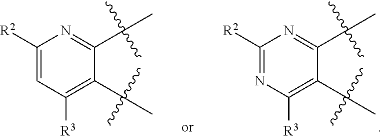

In several embodiments, the

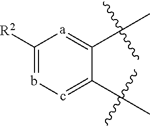

group is

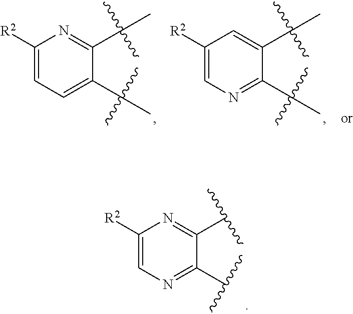

Provided herein as Embodiment 40 is the compound or salt of any one of Embodiments 1 to 28 or 39, wherein the

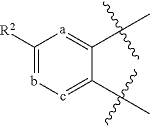

group is

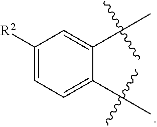

Provided herein as Embodiment 41 is the compound or salt of any one of Embodiments 1 to 28 or 39, wherein the

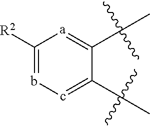

group is

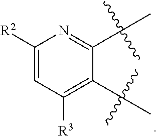

Provided herein as Embodiment 42 is the compound or salt of any one of Embodiments 1 to 28 or 39, wherein the

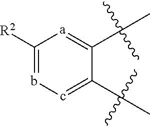

group is

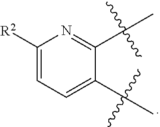

Provided herein as Embodiment 43 is the compound or salt of any one of Embodiments 1 to 28 or 39, wherein the

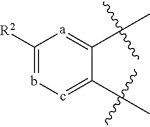

group is

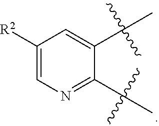

Provided herein as Embodiment 44 is the compound or salt of any one of Embodiments 1 to 28 or 39, wherein the

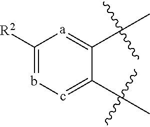

group is

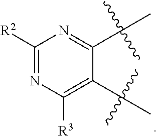

Provided herein as Embodiment 45 is the compound or salt of any one of Embodiments 1 to 28 or 39, wherein the

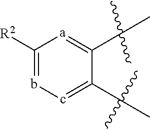

group is

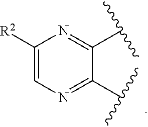

Provided herein as Embodiment 46 is the compound or salt of any one of Embodiments 1 to 45, wherein not more than two instances of a, b, and c are N.

Provided herein as Embodiment 47 is the compound or salt of any one of Embodiments 1 to 46, wherein not more than two instances of a, b, c, and d are N.

Provided herein as Embodiment 48 is the compound or salt of any one of Embodiments 1 to 47, wherein only one of a, b, and c is N.

Provided herein as Embodiment 49 is the compound or salt of any one of Embodiments 1 to 48, wherein only one of a, b, c, and d is N.

Provided herein as Embodiment 50 is the compound or salt of any one of Embodiments 1 to 49, wherein R^{1a} is —H, C₁₋₃ alkyl, or C₁₋₃ haloalkyl. For example, in several embodiments, R^{1a} is —H or C₁₋₃ haloalkyl. In several embodiments, R^{1a} is C₁₋₃alkyl or C₁₋₃haloalkyl. In several embodiments, R^{1a} is —H or C₁₋₃alkyl. In several embodiments, R^{1a} is C₁₋₂ alkyl. In several embodiments, R^{1a} is methyl. In several embodiments, R^{1a} is C₂alkyl. In several embodiments, R^{1a} is C₃ alkyl. In several embodiments, R^{1a} is C₁₋₂ haloalkyl. In several embodiments, R^{1a} is C₁ haloalkyl. In several embodiments, R^{1a} is C₂haloalkyl. In several embodiments, R^{1a} is C₃ haloalkyl.

Provided herein as Embodiment 51 is the compound or salt of any one of Embodiments 1 to 50, wherein R^{1a} is alkyl. For example, in several embodiments, R^{1a} is C₁₋₆ alkyl. In several embodiments, R^{1a} is C₁₋₅ alkyl. In several embodiments, R^{1a} is C₁₋₄ alkyl. In several embodiments, R^{1a} is C₁₋₃ alkyl. In several embodiments, R^{1a} is C₁₋₂ alkyl. In several embodiments, R^{1a} is C₂₋₃ alkyl. In several embodiments, R^{1a} is C₁ alkyl. In several embodiments, R^{1a} is C₂ alkyl. In several embodiments, R^{1a} is C₃ alkyl.

Provided herein as Embodiment 52 is the compound or salt of any one of Embodiments 1 to 50, wherein R^{1a} is haloalkyl. For example, in several embodiments, R^{1a} is C₁₋₆ haloalkyl. In several embodiments, R^{1a} is C₁₋₅ haloalkyl. In several embodiments, R^{1a} is C₁₋₄haloalkyl. In several embodiments, R^{1a} is C₁₋₃ haloalkyl. In several embodiments, R^{1a} is C₁₋₂haloalkyl. In several embodiments, R^{1a} is C₂₋₃ haloalkyl. In several embodiments, R^{1a} is C₁ haloalkyl. In several embodiments, R^{1a} is C₂haloalkyl. In several embodiments, R^{1a} is C₃ haloalkyl.

Provided herein as Embodiment 53 is the compound or salt of any one of Embodiments 1 to 52, wherein R^{1a} is substituted with 1, 2, 3, or 4 substituents. For example, in several embodiments, R^{1a} is substituted with 1 to 4 substituents. In several embodiments, R^{1a} is substituted with 1 to 3 substituents. In several embodiments, R^{1a} is substituted with 1 to 2 substituents. In several embodiments, R^{1a} is substituted with 2 to 4 substituents. In several embodiments, R^{1a} is substituted with 3 to 4 substituents. In several embodiments, R^{1a} is substituted with 2 to 3 substituents.

Provided herein as Embodiment 54 is the compound or salt of any one of Embodiments 1 to 53, wherein R^{1a} is substituted with 1, 2, or 3 substituents.

Provided herein as Embodiment 55 is the compound or salt of any one of Embodiments 1 to 54, wherein R^{1a} is substituted with 1 or 2 substituents.

Provided herein as Embodiment 56 is the compound or salt of any one of Embodiments 1 to 55, wherein R^{1a} is substituted with 1 substituent.

Provided herein as Embodiment 57 is the compound or salt of any one of Embodiments 1 to 56, wherein, when R^{1a} is substituted, each R^{1a} substituent independently is halogen, —OH, C₁₋₃ alkyl, or C₁₋₃ alkoxy. For example, in several embodiments, R^{1a} is substituted with —F or —OH. In several embodiments, R^{1a} is substituted with —OH or C₁₋₃ alkoxy. In several embodiments, R^{1a} is substituted with —F or C₁₋₃ alkoxy. In several embodiments, R^{1a} is substituted with —F, —OH, C₁ alkyl, C₂ alkyl, C₃ alkyl, C₁ alkoxy, C₂ alkoxy, and/or C₃ alkoxy. In several embodiments, R^{1a} is substituted with methyl, C₂ alkyl, and/or C₃ alkyl. In several embodiments, R^{1a} is substituted with —C₁ alkoxy, C₂ alkoxy, or C₃ alkoxy. In several embodiments, R^{1a} is substituted with —C₁ alkoxy. In several embodiments, R^{1a} is substituted with C₂ alkoxy. In several embodiments, R^{1a} is substituted with C₃ alkoxy. In several embodiments, R^{1a} is substituted with —F. In several embodiments, R^{1a} is substituted with —OH. In several embodiments, R^{1a} is substituted with methyl.

Provided herein as Embodiment 58 is the compound or salt of any one of Embodiments 1 to 57, wherein, when R^{1a} is substituted, each R^{1a} substituent independently is —F, —OH, or C₁₋₃alkoxy.

Provided herein as Embodiment 59 is the compound or salt of any one of Embodiments 1 to 52, wherein R^{1a} is unsubstituted.

Provided herein as Embodiment 60 is the compound or salt of any one of Embodiments 1 to 50, wherein R^{1a} is —H or —CH₃.

Provided herein as Embodiment 61 is the compound or salt of any one of Embodiments 1 to 12 and 14 to 50, wherein R^{1a} is —H.

Provided herein as Embodiment 62 is the compound or salt of any one of Embodiments 1 to 50, wherein R^{1a} is —CH₃.

Provided herein as Embodiment 63 is the compound or salt of any one of Embodiments 1 to 62, wherein R^{1b} is C₁₋₃alkyl, C₁₋₃haloalkyl, C₃₋₆cycloalkyl, or heterocyclyl having 3- to 6-ring members. For example, in several embodiments, R^{1b} is C₁₋₃ haloalkyl, C₃₋₆ cycloalkyl, or heterocyclyl having 3- to 6-ring members. In several embodiments, R^{1b} is C₁₋₃ alkyl, C₃₋₆ cycloalkyl, or heterocyclyl having 3- to 6-ring members. In several embodiments, R^{1b} is C₁₋₃ alkyl, C₁₋₃ haloalkyl, or C₃₋₆ cycloalkyl. In several embodiments, R^{1b} is C₁₋₃ alkyl or C₃₋₆ cycloalkyl. In several embodiments, R^{1b} is C₁₋₃ alkyl or C₁₋₃ haloalkyl. In several embodiments, R^{1b} is C₃₋₆ cycloalkyl or heterocyclyl having 3- to 6-ring members. In several embodiments, R^{1b} is C₁₋₃ haloalkyl or heterocyclyl having 3- to 6-ring members. In several embodiments, R^{1b} is C₁₋₃ haloalkyl or C₃₋₆ cycloalkyl. In several embodiments, where R^{1b} is alkyl, R^{1b} is C₁₋₂ alkyl. In several embodiments, where R^{1b} is alkyl, R^{1b} is C₂₋₃ alkyl. In several embodiments, where R^{1b} is alkyl, R^{1b} is C₁ alkyl. In several embodiments, where R^{1b} is alkyl, R^{1b} is C₂ alkyl. In several embodiments, where R^{1b} is alkyl, R^{1b} is C₃ alkyl. In several embodiments, where R^{1b} is haloalkyl, R^{1b} is C₁₋₂haloalkyl. In several embodiments, where R^{1b} is haloalkyl, R^{1b} is C₂₋₃ haloalkyl. In several embodiments, where R^{1b} is haloalkyl, R^{1b} is C₁haloalkyl. In several embodiments, where R^{1b} is haloalkyl, R^{1b} is C₂haloalkyl. In several embodiments, where R^{1b} is haloalkyl, R^{1b} is C₃ haloalkyl. In several embodiments, where R^{1b} is cycloalkyl, R^{1b} is C₄₋₆ cycloalkyl. In several embodiments, where R^{1b} is cycloalkyl, R^{1b} is C₃-5 cycloalkyl. In several embodiments, where R^{1b} is cycloalkyl, R^{1b} is C₃ cycloalkyl. In several embodiments, where R^{1b} is cycloalkyl, R^{1b} is C₄cycloalkyl. In several embodiments, where R^{1b} is cycloalkyl, R^{1b} is C₅ cycloalkyl. In several embodiments, where R^{1b} is cycloalkyl, R^{1b} is C₆ cycloalkyl. In several embodiments, where R^{1b} is heterocyclyl, R^{1b} is heterocyclyl having 4 to 6-ring members. In several embodiments, where R^{1b} is heterocyclyl, R^{1b} is heterocyclyl having 3 to 5-ring members. In several embodiments, where R^{1b} is heterocyclyl, R^{1b} is heterocyclyl having 3-ring members. In several embodiments, where R^{1b} is heterocyclyl, R^{1b} is heterocyclyl having 4-ring members. In several embodiments, where R^{1b} is heterocyclyl, R^{1b} is heterocyclyl having 5-ring members. In several embodiments, where R^{1b} is heterocyclyl, R^{1b} is heterocyclyl having 6-ring members.

Provided herein as Embodiment 64 is the compound or salt of any one of Embodiments 1 to 63, wherein R^{1b} is heterocyclyl.

Provided herein as Embodiment 65 is the compound or salt of any one of Embodiments 1 to 64, wherein R^{1b} is heterocyclyl and includes 1, 2, 3, or 4 heteroatom ring members each of which independently is nitrogen, oxygen, or —S(O)ₓ—, where x is 0, 1, or 2. For example, in several embodiments, when R^{1b} is heterocyclyl, the R^{1b} heterocyclyl includes 1 or 3 heteroatom ring members. In several embodiments, when R^{1b} is heterocyclyl, the R^{1b} heterocyclyl includes 2 or 3 heteroatom ring members.

Provided herein as Embodiment 66 is the compound or salt of any one of Embodiments 1 to 65, wherein R^{1b} is heterocyclyl and has 1, 2, or 3 heteroatom ring members.

Provided herein as Embodiment 67 is the compound or salt of any one of Embodiments 1 to 66, wherein R^{1b} is heterocyclyl and has 1 or 2 heteroatom ring members.

Provided herein as Embodiment 68 is the compound or salt of any one of Embodiments 1 to 67, wherein R^{1b} is heterocyclyl and has 1 heteroatom ring member.

Provided herein as Embodiment 69 is the compound or salt of any one of Embodiments 1 to 68, wherein R^{1b} is heterocyclyl and the heteroatom ring member or ring members of R^{1b} comprise or consist of nitrogen.

Provided herein as Embodiment 70 is the compound or salt of any one of Embodiments 1 to 69, wherein R^{1b} is heterocyclyl and the heteroatom ring member or heteroatom ring members of R^{1b} comprise or consist of oxygen.

Provided herein as Embodiment 71 is the compound or salt of any one of Embodiments 1 to 63, wherein R^{1b} is alkyl.

Provided herein as Embodiment 72 is the compound or salt of any one of Embodiments 1 to 63, wherein R^{1b} is haloalkyl.

Provided herein as Embodiment 73 is the compound or salt of any one of Embodiments 1 to 63, wherein R^{1b} is cycloalkyl.

Provided herein as Embodiment 74 is the compound or salt of any one of Embodiments 1 to 70, wherein R^{1b} is 3- to 6-ring member heterocyclyl.

Provided herein as Embodiment 75 is the compound or salt of any one of Embodiments 1 to 74, wherein R^{1b} is substituted with 1, 2, 3, or 4 substituents. For example, in several embodiments, R^{1b} is substituted with 1 to 4 substituents. In several embodiments, R^{1b} is substituted with 1 to 3 substituents. In several embodiments, R^{1b} is substituted with 1 to 2 substituents. In several embodiments, R^{1b} is substituted with 2 to 4 substituents. In several embodiments, R^{1b} is substituted with 3 to 4 substituents. In several embodiments, R^{1b} is substituted with 2 to 3 substituents.

Provided herein as Embodiment 76 is the compound or salt of any one of Embodiments 1 to 75, wherein R^{1b} is substituted with 1, 2, or 3 substituents.

Provided herein as Embodiment 77 is the compound or salt of any one of Embodiments 1 to 76, wherein R^{1b} is substituted with 1 or 2 substituents.

Provided herein as Embodiment 78 is the compound or salt of any one of Embodiments 1 to 77, wherein R^{1b} is substituted with 1 substituent.

Provided herein as Embodiment 79 is the compound or salt of any one of Embodiments 1 to 78, wherein, when R^{1b} is substituted, each R^{1b} substituent independently is halogen, C₁₋₃alkyl, or C₁₋₃alkoxy. For example, in several embodiments, when R^{1b} is substituted, each R^{1b} substituent independently is halogen or C₁₋₃alkoxy. In several embodiments, when R^{1b} is substituted, each R^{1a} substituent independently is halogen or C₁₋₃alkyl. In several embodiments, when R^{1b} is substituted, each R^{1b} substituent independently is C₁₋₃alkyl or C₁₋₃alkoxy. In several embodiments, the substituent is —F, —Cl, or —Br. In several embodiments, the substituent is —F or —Cl. In several embodiments, the substituent is —F. In several embodiments, the substituent is C₁₋₂ alkyl. In several embodiments, the substituent is C₂₋₃ alkyl. In several embodiments, the substituent is Methyl. In several embodiments, the substituent is C₂ alkyl. In several embodiments, the substituent is C₃ alkyl. In several embodiments, the substituent is C₁₋₂ alkoxy. In several embodiments, the substituent is C₂₋₃ alkoxy. In several embodiments, the substituent is C₁ alkoxy. In several embodiments, the substituent is C₂ alkoxy. In several embodiments, the substituent is C₃ alkoxy.

Provided herein as Embodiment 80 is the compound or salt of any one of Embodiments 1 to 78, wherein each R^{1b} substituent independently is —F, —OH, methyl, or —OMe. For example, in several embodiments, the substituent is —OH, methyl, or —OMe. In several embodiments, the substituent is —F, methyl, or —OMe. In several embodiments, the substituent is —F, —OH, or —OMe. In several embodiments, the substituent is —F, —OH, or methyl. In several embodiments, the substituent is —F or —OH. In several embodiments, the substituent is —F or methyl. In several embodiments, the substituent is —F or —OMe. In several embodiments, the substituent is —OH or methyl. In several embodiments, the substituent is —OH or —OMe. In several embodiments, the substituent is methyl or —OMe.

Provided herein as Embodiment 81 is the compound or salt of any one of Embodiments 1 to 80, wherein R^{1b} is substituted with —OMe.

Provided herein as Embodiment 82 is the compound or salt of any one of Embodiments 1 to 74, wherein R^{1b} is unsubstituted.

Provided herein as Embodiment 83 is the compound or salt of any one of Embodiments 1 to 63 or 82, wherein R^{1b} is —CH₃, —CH₂CH₂—O—CH₃, or unsubstituted cyclobutyl.

Provided herein as Embodiment 84 is the compound or salt of any one of Embodiments 1 to 49, wherein

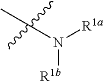

is represented by:

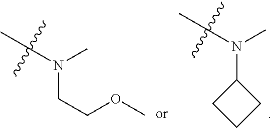

Provided herein as Embodiment 85 is the compound or salt of any one of Embodiments 1 to 49, wherein

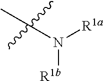

is represented by:

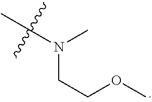 [SMILES: *N(C)CCOC]

Provided herein as Embodiment 86 is the compound or salt of any one of Embodiments 1 to 49, wherein R^{1a} and R^{1b} together form a 3 to 10 membered heterocyclyl. For example, in several embodiments, R^{1a} and R^{1b} together form a heterocyclyl having 3 to 6-ring members. In several embodiments, R^{1a} and R^{1b} together form a heterocyclyl having 4 to 6-ring members. In several embodiments, R^{1a} and R^{1b} together form a heterocyclyl having 5 to 6-ring members. In several embodiments, R^{1a} and R^{1b} together form a heterocyclyl having 3 to 5-ring members. In several embodiments, R^{1a} and R^{1b} together form a heterocyclyl having 3 to 4-ring members. In several embodiments, R^{1a} and R^{1b} together form a heterocyclyl having 4 to 5-ring members. In several embodiments, R^{1a} and R^{1b} together form a heterocyclyl having 5 to 10-ring members. In several embodiments, R^{1a} and R^{1b} together form a heterocyclyl having 6 to 10-ring members. In several embodiments, R^{1a} and R^{1b} together form a heterocyclyl having 5 to 9-ring members. In several embodiments, R^{1a} and R^{1b} together form a heterocyclyl having 6 to 9-ring members. In several embodiments, R^{1a} and R^{1b} together form a heterocyclyl having 3-ring members. In several embodiments, R^{1a} and R^{1b} together form a heterocyclyl having 4-ring members. In several embodiments, R^{1a} and R^{1b} together form a heterocyclyl having 5-ring members. In several embodiments, R^{1d} and R^{1b} together form a heterocyclyl having 6-ring members. In several embodiments, R^{1a} and R^{1b} together form a heterocyclyl having 7-ring members. In several embodiments, R^{1a} and R^{1b} together form a spirocyclic heterocyclyl. In several embodiments, R^{1a} and R^{1b} together form a heterocyclyl having a fused ring system. In several embodiments, R^{1a} and R^{1b} together form a heterocyclyl having a bridged ring system. In several embodiments, R^{1a} and R^{1b} together form a heterocyclyl having two rings in a ring system.

Provided herein as Embodiment 87 is the compound or salt of any one of Embodiments 1 to 49 and 86, wherein R^{1a} and R^{1b} together form a heterocyclyl and each heteroatom ring member of the R^{1a} and R^{1b} heterocyclyl independently is nitrogen, oxygen, or —S(O)ₓ—, where x is 0, 1, or 2.

Provided herein as Embodiment 88 is the compound or salt of any one of Embodiments 1 to 49 and 86 to 87, wherein R^{1a} and R^{1b} together form a heterocyclyl having 1, 2, or 3 heteroatom ring members. For example, in several embodiments, R^{1a} and R^{1b} together form a heterocyclyl having 1 or 3 heteroatom ring members. In several embodiments, R^{1a} and R^{1b} together form a heterocyclyl having 2 or 3 heteroatom ring members.

Provided herein as Embodiment 89 is the compound or salt of any one of Embodiments 1 to 49 and 86 to 88, wherein R^{1a} and R^{1b} together form a heterocyclyl having 1 or 2 heteroatom ring members.

Provided herein as Embodiment 90 is the compound or salt of any one of Embodiments 1 to 49 and 86 to 89, wherein R^{1a} and R^{1b} together form a heterocyclyl having 1 heteroatom ring member.

Provided herein as Embodiment 91 is the compound or salt of any one of Embodiments 87 to 90, wherein R^{1a} and R^{1b} together form a heterocyclyl and the heteroatom or heteroatoms of the R^{1a} and R^{1b} heterocyclyl comprise or consist of nitrogen.

Provided herein as Embodiment 92 is the compound or salt of any one of Embodiments 87 to 91, wherein R^{1a} and R^{1b} together form a heterocyclyl and the heteroatom or heteroatoms of the R^{1a} and R^{1b} heterocyclyl comprise oxygen.

Provided herein as Embodiment 93 is the compound or salt of any one of Embodiments 1 to 49 and 86 to 92, wherein R^{1a} and R^{1b} together form a heterocyclyl that is substituted with 1, 2, 3, 4, or 5 substituents.

Provided herein as Embodiment 94 is the compound or salt of any one of Embodiments 1 to 49 and 86 to 93, wherein R^{1a} and R^{1b} together form a heterocyclyl that is substituted with 1, 2, 3, or 4 substituents.

Provided herein as Embodiment 95 is the compound or salt of any one of Embodiments 1 to 49 and 86 to 94, wherein R^{1a} and R^{1b} together form a heterocyclyl that is substituted with 1, 2, or 3 substituents.

Provided herein as Embodiment 96 is the compound or salt of any one of Embodiments 1 to 49 and 86 to 95, wherein R^{1a} and R^{1b} together form a heterocyclyl that is substituted with 1 or 2 substituents.

Provided herein as Embodiment 97 is the compound or salt of any one of Embodiments 1 to 49 and 86 to 96, wherein R^{1a} and R^{1b} together form a heterocyclyl that is substituted with 1 substituent.

Provided herein as Embodiment 98 is the compound or salt of any one of Embodiments 1 to 49 and 86 to 97, wherein, when R^{1a} and R^{1b} together form a heterocyclyl that is substituted, each substituent of the heterocyclyl independently is halogen, —CN, C₁₋₆ alkyl, C₁₋₆ haloalkyl, C₁₋₆alkoxy, C₁₋₆heteroalkyl, or C₃₋₆ cycloalkyl. For example, in several embodiments, each instance of a substituent of the R^{1a} and R^{1b} heterocyclyl independently is halogen, CN, C₁₋₆ alkyl, C₁₋₆ heteroalkyl, C₁₋₆ alkoxy, or C₁₋₆ haloalkyl. In several embodiments, an instance of a substituent of the R^{1a} and R^{1b} heterocyclyl is —OH. In several embodiments, an instance of a substituent of the R^{1a} and R^{1b} heterocyclyl is —NH₂. In several embodiments, an instance of a substituent of the R^{1a} and R^{1b} heterocyclyl is halogen. In several embodiments, an instance of a substituent of the R^{1a} and R^{1b} heterocyclyl is —F, —Cl, or —Br. In several embodiments, an instance of a substituent of the R^{1a} and R^{1b} heterocyclyl is —F or —Cl. In several embodiments, an instance of a substituent of the R^{1a} and R^{1b} heterocyclyl is —F. In several embodiments, an instance of a substituent of the R^{1a} and R^{1b} heterocyclyl is C₁₋₃alkyl. In several embodiments, an instance of a substituent of the R^{1a} and R^{1b} heterocyclyl is C₁₋₂ alkyl. In several embodiments, an instance of a substituent of the R^{1a} and R^{1b} heterocyclyl is C₂₋₃ alkyl. In several embodiments, an instance of a substituent of the R^{1a} and R^{1b} heterocyclyl is methyl. In several embodiments, an instance of a substituent of the R^{1a} and R^{1b} heterocyclyl is C₂ alkyl. In several embodiments, an instance of a substituent of the R^{1a} and R^{1b} heterocyclyl is C₃ alkyl. In several embodiments, an instance of a substituent of the R^{1a} and R^{1b} heterocyclyl is C₁₋₃ haloalkyl. In several embodiments, an instance of a substituent of the R^{1a} and R^{1b} heterocyclyl is C₁₋₂ haloalkyl. In several embodiments, an instance of a substituent of the R^{1a} and R^{1b} heterocyclyl is C₂₋₃ haloalkyl. In several embodiments, an instance of a substituent of the R^{1a} and R^{1b} heterocyclyl is C₁ haloalkyl. In several embodiments, an instance of a substituent of the R^{1a} and R^{1b} heterocyclyl is C₂haloalkyl. In several embodiments, an instance of a substituent of the R^{1a} and R^{1b} heterocyclyl is C₃ haloalkyl. In several embodiments, an instance of a substituent of the R^{1a} and R^{1b} heterocyclyl is C₁₋₃ heteroalkyl. In several embodiments, an instance of a substituent of the R^{1a} and R^{1b} heterocyclyl is C₁₋₂heteroalkyl. In several embodiments, an instance of a substituent of the R^{1a} and R^{1b} heterocyclyl is C₂₋₃ heteroalkyl. In several embodiments, an instance of a substituent of the R^{1a} and R^{1b} heterocyclyl is C₁ heteroalkyl. In several embodiments, an instance of a substituent of the R^{1a} and R^{1b} heterocyclyl is C₂heteroalkyl. In several embodiments, an instance of a substituent of the R^{1a} and R^{1b} heterocyclyl is C₃ heteroalkyl. In several embodiments, an instance of a substituent of the R^{1a} and R^{1b} heterocyclyl is C₁₋₂ alkoxy. In several embodiments, an instance of a substituent of the R^{1a} and R^{1b} heterocyclyl is C₂₋₃ alkoxy. In several embodiments, an instance of a substituent of the R^{1a} and R^{1b} heterocyclyl is methoxy. In several embodiments, an instance of a substituent of the R^{1a} and R^{1b} heterocyclyl is C₂ alkoxy. In several embodiments, an instance of a substituent of the R^{1a} and R^{1b} heterocyclyl is C₃ alkoxy.

Provided herein as Embodiment 99 is the compound or salt of any one of Embodiments 1 to 49 and 86 to 97, wherein, when R^{1a} and R^{1b} together form a heterocyclyl that is substituted, at least one substituent is —CF₃, —CF₂CF₃, or —CF₂CF₂CF₃.

Provided herein as Embodiment 100 is the compound or salt of any one of Embodiments 1 to 49 and 86 to 97, wherein, when R^{1a} and R^{1b} together form a heterocyclyl that is substituted, each substituent of the heterocyclyl independently is —F, —OH, oxo, -Me, —CF₃, —CN, or methoxy. In several embodiments, when R^{1a} and R^{1b} together form a heterocyclyl that is substituted, a substituent of the heterocyclyl is —F. In several embodiments, when R^{1a} and R^{1b} together form a heterocyclyl that is substituted, a substituent of the heterocyclyl is —OH. In several embodiments, when R^{1a} and R^{1b} together form a heterocyclyl that is substituted, a substituent of the heterocyclyl is oxo. In several embodiments, when R^{1a} and R^{1b} together form a heterocyclyl that is substituted, a substituent of the heterocyclyl is -Me. In several embodiments, when R^{1a} and R^{1b} together form a heterocyclyl that is substituted, a substituent of the heterocyclyl is —CF₃. In several embodiments, when R^{1a} and R^{1b} together form a heterocyclyl that is substituted, a substituent of the heterocyclyl is —CN. In several embodiments, when R^{1a} and R^{1b} together form a heterocyclyl that is substituted, a substituent of the heterocyclyl is methoxy.

Provided herein as Embodiment 101 is the compound or salt of any one of Embodiments 1 to 49 and 86 to 92, wherein R^{1a} and R^{1b} together form a heterocyclyl that is unsubstituted.

Provided herein as Embodiment 102 is the compound or salt of any one of Embodiments 1 to 49 and 86 to 101, wherein, when R^{1a} and R^{1b} together form a heterocyclyl, the heterocyclyl is a bicyclic fused ring system.

Provided herein as Embodiment 103 is the compound or salt of any one of Embodiments 1 to 49 and 86 to 101, wherein, when R^{1a} and R^{1b} together form a heterocyclyl, the heterocyclyl is a bicyclic spiro ring system.

Provided herein as Embodiment 104 is the compound or salt of any one of Embodiments 1 to 49 and 86 to 101, wherein, when R^{1a} and R^{1b} together form a heterocyclyl, the heterocyclyl is a bridged ring system.

Provided herein as Embodiment 105 is the compound or salt of any one of Embodiments 1 to 49 and 86 to 90, wherein

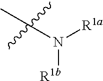

is:

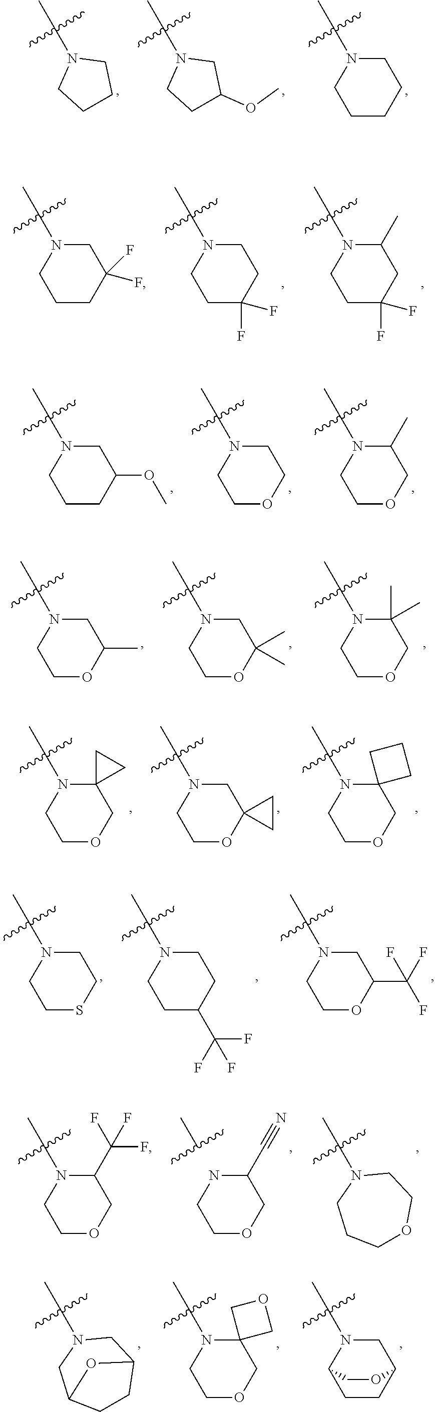

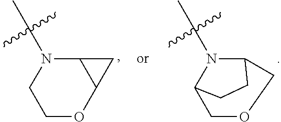 [SMILES: *N1CCOC2CC21 ; *N1C2CCC1COC2]

For example, in several embodiments,

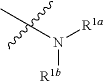

is represented by

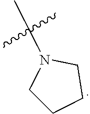 [SMILES: *N1CCCC1]

In several embodiments,

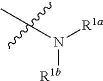

is represented by

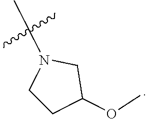 [SMILES: *N1CCC(OC)C1]

In several embodiments,

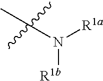

is represented by

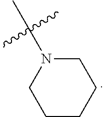 [SMILES: *N1CCCCC1]

In several embodiments,

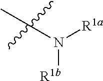

is represented by

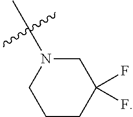 [SMILES: *N1CCCC(F)(F)C1]

In several embodiments,

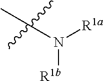

is represented by

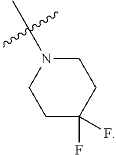 [SMILES: *N1CCC(F)(F)CC1]

In several embodiments,

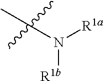

is represented by

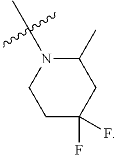 [SMILES: *N1CCC(F)(F)CC1C]

In several embodiments,

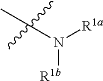

is represented by

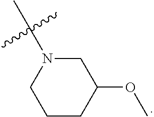 [SMILES: *N1CCCC(OC)C1]

In several embodiments,

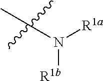

is represented by

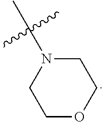 [SMILES: *N1CCOCC1]

In several embodiments,

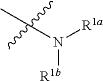

is represented by

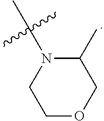 [SMILES: *N1CCOCC1C]

In several embodiments,

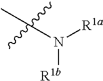

is represented by

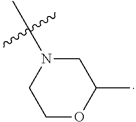 [SMILES: *N1CCOC(C)C1]

In several embodiments,

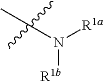

is represented by

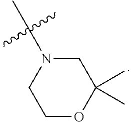 [SMILES: *N1CCOC(C)(C)C1]

In several embodiments,

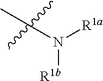

is represented by

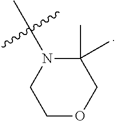 [SMILES: *N1CCOCC1(C)C]

In several embodiments,

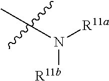

is represented by

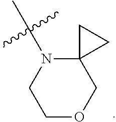 [SMILES: *N1CCOCC12CC2]

In several embodiments,

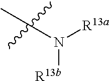

is represented by

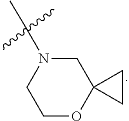 [SMILES: *N1CCOC2(CC2)C1]

In several embodiments,

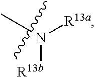

is represented by

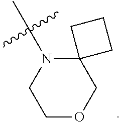 [SMILES: *N1CCOCC12CCC2]

In several embodiments,

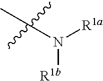

is represented by

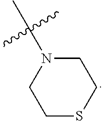 [SMILES: *N1CCSCC1]

In several embodiments,

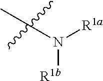

is represented by

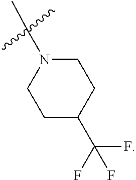 [SMILES: *N1CCC(C(F)(F)F)CC1]

In several embodiments,

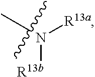

is represented by

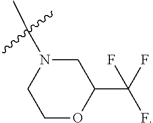 [SMILES: *N1CCOC(C(F)(F)F)C1]

In several embodiments,

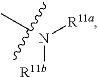

is represented by

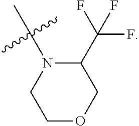 [SMILES: *N1CCOCC1C(F)(F)F]

In several embodiments,

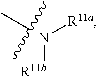

is represented by

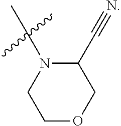 [SMILES: *N1CCOCC1C#N]

In several embodiments,

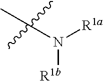

is represented by

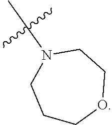 [SMILES: *N1CCCOCC1]

In several embodiments,

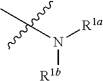

is represented by

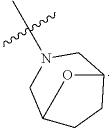 [SMILES: *N1CC2CCC(C1)O2]

In several embodiments,

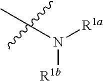

is represented by

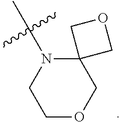 [SMILES: *N1CCOCC12COC2]

In several embodiments,

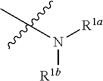

is represented by

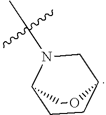 [SMILES: *N1C[C@H]2CC[C@@H]1CO2]

In several embodiments,

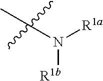

is represented by

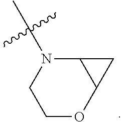 [SMILES: *N1CCOC2CC21]

In several embodiments,

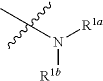

is represented by

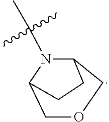 [SMILES: *N1C2CCC1COC2]

Provided herein as Embodiment 106 is the compound or salt of any one of Embodiments 1 to 49 and 86 to 90, wherein

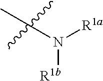

is:

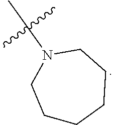 [SMILES: *N1CCCCCC1]

Provided herein as Embodiment 107 is the compound or salt of any one of Embodiments 1 to 49 and 86 to 90, wherein,

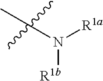

is:

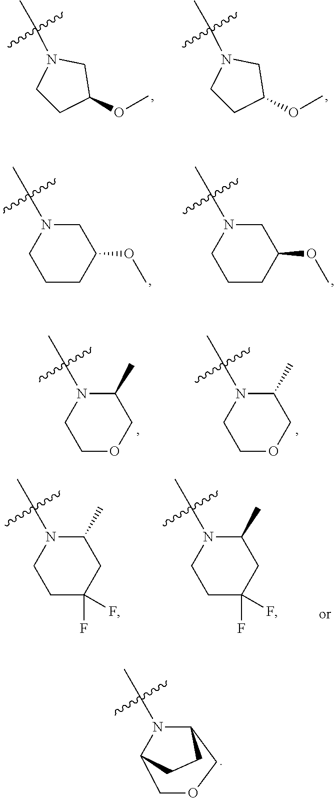

Provided herein as Embodiment 108 is the compound or salt of any one of Embodiments 1 to 107, wherein R² is heterocyclyl.

Provided herein as Embodiment 109 is the compound or salt of any one of Embodiments 1 to 107, wherein R² is heteroaryl.

Provided herein as Embodiment 110 is the compound or salt of any one of Embodiments 1 to 109, wherein the R² has 1, 2, 3, 4, or 5 heteroatom ring members. For example, in several embodiments, R² includes 5 heteroatom ring members. In several embodiments, R² includes 4 heteroatom ring members. In several embodiments, R² includes 3 heteroatom ring members. In several embodiments, R² includes 2 heteroatom ring members. In several embodiments, R² includes 1 heteroatom ring member. In several embodiments, R² comprises 1 to 5 heteroatom ring members. In several embodiments, R² comprises 1 to 4 heteroatom ring members. In several embodiments. R² comprises 1 to 3 heteroatom ring members. In several embodiments, R² comprises 1 or 2 heteroatom ring members. In several embodiments, R² comprises 2 to 5 heteroatom ring members. In several embodiments, R² comprises 3 to 5 heteroatom ring members. In several embodiments, R² comprises 3 to 4 heteroatom ring members. In several embodiments, R² comprises nitrogen atoms as the only ring heteroatoms. In several embodiments, R² comprises one nitrogen atom as a ring heteroatom. In several embodiments, R² comprises two nitrogen atoms as a ring heteroatoms. In several embodiments, R² comprises three nitrogen atoms as a ring heteroatoms. In several embodiments, R² comprises four nitrogen atoms as a ring heteroatoms. In several embodiments, R² comprises five nitrogen atoms as a ring heteroatoms. In several embodiments, R² comprises one oxygen ring heteroatom and the remaining ring heteroatoms are nitrogen atoms. In several embodiments, R² comprises oxygen atoms as the only ring heteroatoms.

Provided herein as Embodiment 111 is the compound or salt of any one of Embodiments 1 to 110, wherein R² has 1, 2, 3, or 4 heteroatom ring members.

Provided herein as Embodiment 112 is the compound or salt of any one of Embodiments 1 to 111, wherein R² has 1, 2, or 3 heteroatom ring members.

Provided herein as Embodiment 113 is the compound or salt of any one of Embodiments 1 to 112, wherein R² has 1 or 2 heteroatom ring members.

Provided herein as Embodiment 114 is the compound or salt of any one of Embodiments 1 to 113, wherein R² has 1 heteroatom ring member.

Provided herein as Embodiment 115 is the compound or salt of any one of Embodiments 1 to 114, wherein each ring member heteroatom of R² independently is nitrogen, oxygen, or —S(O)_{y}—, where y is 0, 1, or 2.

Provided herein as Embodiment 116 is the compound or salt of any one of Embodiments 1 to 115, wherein each heteroatom of the R² ring independently is nitrogen or oxygen.

Provided herein as Embodiment 117 is the compound or salt of any one of Embodiments 1 to 116, wherein each ring heteroatom of the R² is nitrogen.

Provided herein as Embodiment 118 is the compound or salt of any one of Embodiments 1 to 116, wherein each ring heteroatom of R² is oxygen.

Provided herein as Embodiment 119 is the compound or salt of any one of Embodiments 1 to 118, wherein R² is a fused bicyclic group. In several embodiments, the fused bicyclic R² comprises a 6-membered ring. In several embodiments, the fused bicyclic R² comprises a 5-membered ring. In several embodiments, the fused bicyclic R² is a 5,6-member fused ring. In several embodiments, the fused bicyclic R² is a 6,6-member fused ring.

Provided herein as Embodiment 120 is the compound or salt of any one of Embodiments 1 to 119, wherein R² comprises 5 to 10 ring-members.

Provided herein as Embodiment 121 is the compound or salt of any one of Embodiments 1 to 120, wherein R² comprises 5 to 9 ring-members.

Provided herein as Embodiment 122 is the compound or salt of any one of Embodiments 1 to 120, wherein R² has 10 ring members.

Provided herein as Embodiment 123 is the compound or salt of any one of Embodiments 1 to 121, wherein R² has 9 ring members.

Provided herein as Embodiment 124 is the compound or salt of any one of Embodiments 1 to 118, wherein R² has 6 ring members.

Provided herein as Embodiment 125 is the compound or salt of any one of Embodiments 1 to 109 and 111 to 118, wherein R² has 5 ring members.

Provided herein as Embodiment 126 is the compound or salt of any one of Embodiments 1 to 125, wherein R² is substituted with 1, 2, 3, 4, or 5 instances of R⁵. For example, in several embodiments, R² is substituted with 2 to 5 R² substituents. In several embodiments, R² is substituted with 3 to 4 R⁵ substituents. In several embodiments, R⁵ is substituted with 3 to 5 R⁵ substituents. In several embodiments, R² is substituted with 4 to 5 R⁵ substituents. In several embodiments, R² is substituted with 2, 3, 4, or 5 R⁵ substituents. In several embodiments R² is substituted with 1, 2, or 4 R⁵ substituents. In several embodiments, R² is substituted 1 or 3 R⁵ substituents.

Provided herein as Embodiment 127 is the compound or salt of any one of Embodiments 1 to 126, wherein R² is substituted with 1, 2, 3, or 4 instances of R⁵.

Provided herein as Embodiment 128 is the compound or salt of any one of Embodiments 1 to 127, wherein R² is substituted with 1, 2, or 3 instances of R⁵.

Provided herein as Embodiment 129 is the compound or salt of any one of Embodiments 1 to 128, wherein R² is substituted with 1 or 2 instances of R⁵.

Provided herein as Embodiment 130 is the compound or salt of any one of Embodiments 1 to 129, wherein R² is substituted with 1 instance of R⁵.

Provided herein as Embodiment 131 is the compound or salt of any one of Embodiments 1 to 130, wherein R⁵ is substituted with 1, 2, or 3 instances of R⁶.

Provided herein as Embodiment 132 is the compound or salt of any one of Embodiments 1 to 129 and 145, wherein R⁵ is substituted with 1 or 2 instances of R⁶.

Provided herein as Embodiment 133 is the compound or salt of any one of Embodiments 1 to 132, wherein R⁵ is substituted with 1 instance of R⁶.

Provided herein as Embodiment 134 is the compound or salt of any one of Embodiments 1 to 133, wherein, each instance of R⁵ independently is oxo, halogen, —OH, CN, C₁₋₃ alkyl, C₃₋₆ cycloalkyl, C₁₋₃ haloalkyl, C₁₋₃ alkoxy, C(O)N(R⁶)₂, —N(R⁶)₂, —N(R⁶)(C₁₋₃ alkylene-OH), or heterocyclyl having 3 to 10 members. For example, in several embodiments, each R⁵ independently is oxo, CN, C₁₋₃ alkyl, C₃₋₆ cycloalkyl, C₁₋₃ haloalkyl, C₁₋₃ alkoxy, C(O)N(R⁶)₂, —N(R⁶)₂, —N(R⁶)(C₁₋₃ alkylene-OH), or heterocyclyl having 3 to 10 members. In several embodiments, each R⁵ independently is halogen, —OH, oxo, or C₁₋₃alkyl. In several embodiments, each R⁵ independently is halogen, —OH, oxo, C₁₋₃alkyl, or C₁₋₃alkoxy. In several embodiments, each R⁵ independently is halogen, —OH, oxo, or C₁₋₃alkoxy. In several embodiments, each R⁵ independently is halogen, —OH, C₁₋₃alkyl, or C₁₋₃alkoxy. In several embodiments, each R⁵ independently is halogen, oxo, C₁₋₃alkyl, or C₁₋₃alkoxy. In several embodiments, each R independently is —OH, oxo, C₁₋₃alkyl, or C₁₋₃alkoxy. In several embodiments, each R⁵ independently is oxo, C₁₋₃alkyl, or C₁₋₃alkoxy. In several embodiments, each R⁵ independently is halogen, C₁₋₃alkyl, or C₁₋₃alkoxy. In several embodiments, each R⁵ independently is halogen, —OH, or C₁₋₃alkoxy. In several embodiments, each R⁵ independently is halogen, —OH, or oxo. In several embodiments, each R⁵ independently is halogen, —OH, oxo, C₁₋₃alkyl, or C₁₋₃alkoxy. In several embodiments, each R⁵ independently is —OH, oxo, C₁₋₃alkyl, or C₁₋₃ haloalkyl. In several embodiments, each R⁵ independently is oxo or C₁₋₃alkyl. In several embodiments, each R⁵ independently is C₁₋₃alkoxy or C₁₋₃alkyl. In several embodiments, each R⁵ independently is oxo or C₁₋₃ alkyl. In several embodiments, each R⁵ independently is halogen or oxo. In several embodiments, an instance of R⁵ is halogen. In several embodiments, a halogen R⁵ substituent (or one or more substituents independently) is —F, −Cl, or −Br. In several embodiments, a halogen R⁵ substituent (or one or more substituents independently) is —F or —Cl. In several embodiments, a halogen R⁵ substituent (or one or more substituents) is —F. In several embodiments, an instance of R⁵ is C₁₋₃alkyl. In several embodiments, an R⁵ alkyl is C₁₋₂ alkyl. In several embodiments, an R⁵ alkyl is C₂₋₃ alkyl. In several embodiments, an R⁵ alkyl is methyl. In several embodiments, an R⁵ alkyl is C₂alkyl. In several embodiments, an R⁵ alkyl is C₃ alkyl. In several embodiments, an R⁵ alkoxy is C₁₋₂ alkoxy. In several embodiments, an R⁵ alkoxy is C₂₋₃ alkoxy. In several embodiments, an R⁵ alkoxy is methoxy. In several embodiments, an R⁵ alkoxy is C₂ alkoxy. In several embodiments, an R⁵ alkoxy is C₃ alkoxy.

Provided herein as Embodiment 135 is the compound or salt of any one of Embodiments 1 to 133, wherein, each instance of R⁵ independently is oxo, CN, C₁₋₃ alkyl, C₃₋₆ cycloalkyl, C₁₋₃ haloalkyl, or C₁₋₃ alkoxy.

Provided herein as Embodiment 136 is the compound or salt of any one of Embodiments 1 to 133, wherein, each instance of R⁵ independently is —N(R⁶)₂, —N(R⁶)(C₁₋₃ alkylene-OH), or heterocyclyl having 3 to 10 members.

Provided herein as Embodiment 137 is the compound or salt of any one of Embodiments 1 to 136, wherein, each instance of R⁶ independently is —H, C₁₋₃ alkyl, C(O)C₁₋₃ alkyl, C(O)₂C₁₋₃ alkyl, C₁₋₃ alkylene-OH, or S(O)ₙC₁₋₃ alkyl.

Provided herein as Embodiment 138 is the compound or salt of any one of Embodiments 1 to 136, wherein each instance of R⁶ independently is —H, -Me, —C(O)OtBu, or —S(O)₂ C₁₋₆alkyl

Provided herein as Embodiment 139 is the compound or salt of any one of Embodiments 1 to 136, wherein, each instance of R⁶ independently is —H or C₁₋₃ alkyl.

Provided herein as Embodiment 140 is the compound or salt of any one of Embodiments 1 to 136, wherein, each instance of R⁶ is C₁₋₃ alkyl.

Provided herein as Embodiment 141 is the compound or salt of any one of Embodiments 1 to 136, wherein, each instance of R⁶ is H.

Provided herein as Embodiment 142 is the compound or salt of any one of Embodiments 1 to 130, wherein each instance of R⁵ independently is —F, methyl, ethyl, or isopropyl.

Provided herein as Embodiment 143 is the compound or salt of any one of Embodiments 1 to 130, wherein each instance of R⁵ is methyl.

Provided herein as Embodiment 144 is the compound or salt of any one of Embodiments 1 to 130 and 134 to 136, wherein each instance of R⁵ is unsubstituted.

Provided herein as Embodiment 145 is the compound or salt of any one of Embodiments 1 to 125, wherein R² is unsubstituted.

Provided herein as Embodiment 146 is the compound or salt of any one of Embodiments 1 to 145, wherein R² is

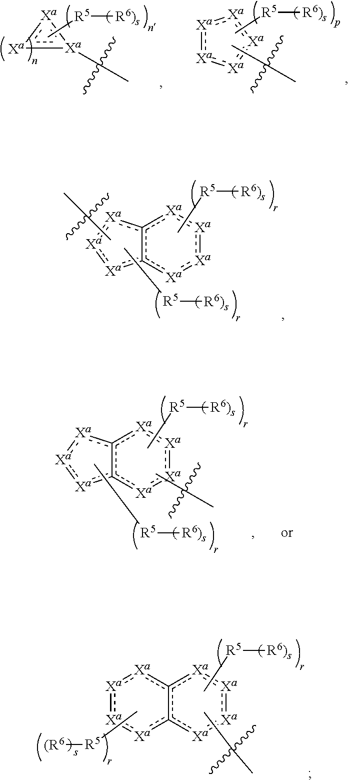

where
“ [symbol] ” indicates a double bond or single bond may be present;
each instance of X^{a} independently is nitrogen, oxygen, or carbon;
each instance of n, where present, is an integer selected from 1, 2, 3, 4, or 5;
each instance of n′, where present, is an integer selected from 1, 2, 3, 4, or 5;
each instance of p, where present, is an integer selected from 0, 1, 2, 3, or 4;
each instance of s, where present, is an integer selected from 0, 1, 2, or 3;
each instance of r, where present, independently is an integer selected from 0, 1, 2, 3, 4, or 5, wherein where two instances of r occur on the same R² group, the total of both r values combined does not exceed 5;
each instance of R⁵, when present and attached to an N ring member, independently is C₁₋₆alkyl, C₁₋₆haloalkyl, C₃₋₆ cycloalkyl, or heterocyclyl having 3 to 10 members; and
each instance of R⁵, when present and attached to a C ring member, independently is oxo, CN, C₁₋₆ alkyl, C₃₋₆ cycloalkyl, C₁₋₆ haloalkyl, C₁₋₆ alkoxy, C(O)N(R⁶)₂, —N(R⁶)₂, —N(R⁶)(C₁₋₆ alkylene-OH), heterocyclyl having 3 to 10 members;
each instance of R⁶, where present, independently is —H, C₁₋₃ alkyl, C(O)C₁₋₃ alkyl, C(O)₂ C₁₋₃ alkyl, C₁₋₃ alkylene-OH, or S(O)ₙC₁₋₃alkyl;
wherein a —N═ ring nitrogen can be bonded to an R⁵ to provide a cation.

Provided herein as Embodiment 147 is the compound or salt of any one of Embodiments 1 to 118 and 146, wherein R² is

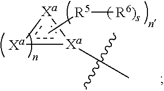

where
“ [symbol] ” indicates a double bond or single bond may be present;
each instance of X^{a} independently is nitrogen, oxygen, or carbon;
each instance of n, where present, is an integer selected from 1, 2, 3, 4, or 5;
each instance of n′, where present, is an integer selected from 1, 2, 3, 4, or 5;
each instance of s, where present, is an integer selected from 0, 1, 2, or 3;
each instance of R⁵, when present and attached to an N ring member, independently is C₁₋₆alkyl, C₁₋₆haloalkyl, C₃₋₆ cycloalkyl, or heterocyclyl having 3 to 10 members; and
each instance of R⁵, when present and attached to a C ring member, independently is oxo, CN, C₁₋₆ alkyl, C₃₋₆ cycloalkyl, C₁₋₆ haloalkyl, C₁₋₆ alkoxy, C(O)N(R⁶)₂, —N(R⁶)₂, —N(R⁶)(C₁₋₆ alkylene-OH), heterocyclyl having 3 to 10 members;
each instance of R⁶, where present, independently is —H, C₁₋₃ alkyl, C(O)C₁₋₃ alkyl, C(O)₂ C₁₋₃ alkyl, C₁₋₃ alkylene-OH, or S(O)ₙC₁₋₃alkyl;
wherein a —N═ ring nitrogen can be bonded to an R⁵ to provide a cation.

Provided herein as Embodiment 148 is the compound or salt of any one of Embodiments 1 to 118, 127 to 133, and 146, wherein R² is

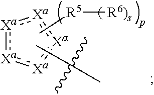

where
“ [symbol] ” indicates a double bond or single bond may be present;
each instance of X^{a} independently is nitrogen, oxygen, or carbon;
each instance of p, where present, is an integer selected from 0, 1, 2, 3, or 4;
each instance of s, where present, is an integer selected from 0, 1, 2, or 3;
each instance of R⁵, when present and attached to an N ring member, independently is C₁₋₆alkyl, C₁₋₆haloalkyl, C₃₋₆ cycloalkyl, or heterocyclyl having 3 to 10 members; and
each instance of R⁵, when present and attached to a C ring member, independently is oxo, CN, C₁₋₆ alky, C₃₋₆ cycloalkyl, C₁₋₆ haloalkyl, C₁₋₆ alkoxy, C(O)N(R⁶)₂, —N(R⁶)₂, —N(R⁶)(C₁₋₆ alkylene-OH), heterocyclyl having 3 to 10 members;
each instance of R⁶, where present, independently is —H, C₁₋₃ alkyl, C(O)C₁₋₃ alkyl, C(O)₂ C₁₋₃ alkyl, C₁₋₃ alkylene-OH, or S(O)ₙC₁₋₃alkyl;
wherein a —N═ ring nitrogen can be bonded to an R⁵ to provide a cation.

Provided herein as Embodiment 149 is the compound or salt of any one of Embodiments 1 to 118 and 146, wherein R² is

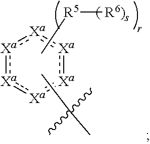

where “ [symbol] ” indicates a double bond or single bond may be present; each instance of X^{a} independently is nitrogen, oxygen, or carbon; each instance of s, where present, is an integer selected from 0, 1, 2, or 3; each instance of r, where present, independently is an integer selected from 0, 1, 2, 3, 4, or 5, wherein where two instances of r occur on the same R² group, the total of both r values combined does not exceed 5; each instance of R⁵, when present and attached to an N ring member, independently is C₁₋₆alkyl, C₁₋₆haloalkyl, C₃₋₆ cycloalkyl, or heterocyclyl having 3 to 10 members; and each instance of R⁵, when present and attached to a C ring member, independently is oxo, CN, C₁₋₆ alkyl, C₃₋₆ cycloalkyl, C₁₋₆ haloalkyl, C₁₋₆ alkoxy, C(O)N(R⁶)₂, —N(R⁶)₂, —N(R⁶)(C₁₋₆ alkylene-OH), heterocyclyl having 3 to 10 members; each instance of R⁶, where present, independently is —H, C₁₋₃ alkyl, C(O)C₁₋₃ alkyl, C(O)₂ C₁₋₃ alkyl, C₁₋₃ alkylene-OH, or S(O)ₙC₁₋₃alkyl; wherein a —N═ ring nitrogen can be bonded to an R⁵ to provide a cation. In several embodiments, one instance of X^{a} is N and five instances of X^{a} are C (e.g., CH, CR⁵, etc.). In several embodiments, two instances of X^{a} are N (independently N, NH, or N—R⁵) and four instances of X^{a} are C (e.g., CH, CR⁵, etc.).

Provided herein as Embodiment 150 is the compound or salt of any one of Embodiments 1 to 118 and 146, wherein R² is

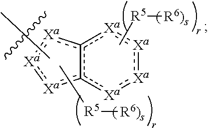

where
“ [symbol] ” indicates a double bond or single bond may be present;
each instance of X⁸ independently is nitrogen, oxygen, or carbon;
each instance of s, where present, is an integer selected from 0, 1, 2, or 3;
each instance of r, where present, independently is an integer selected from 0, 1, 2, 3, or 4, wherein where two instances of r occur on the same R² group, the total of both r values combined does not exceed 5;
each instance of R⁵, when present and attached to an N ring member, independently is C₁₋₆alkyl, C₁₋₆haloalkyl, C₃₋₆ cycloalkyl, or heterocyclyl having 3 to 10 members; and
each instance of R⁵, when present and attached to a C ring member, independently is oxo, CN, C₁₋₆ alkyl, C₃₋₆ cycloalkyl, C₁₋₆ haloalkyl, C₁₋₆ alkoxy, C(O)N(R⁶)₂, —N(R⁶)₂, —N(R⁶)(C₁₋₆ alkylene-OH), heterocyclyl having 3 to 10 members;
each instance of R⁶, where present, independently is —H, C₁₋₃ alkyl, C(O)C₁₋₃ alkyl, C(O)₂ C₁₋₃ alkyl, C₁₋₃ alkylene-OH, or S(O)ₙC₁₋₃alkyl;
wherein a —N═ ring nitrogen can be bonded to an R⁵ to provide a cation.

Provided herein as Embodiment 151 is the compound or salt of any one of Embodiments 1 to 118 and 146, wherein R² is

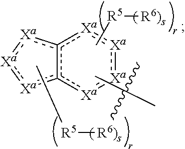

where
“ [symbol] ” indicates a double bond or single bond may be present;
each instance of X^{a} independently is nitrogen, oxygen, or carbon;
each instance of s, where present, is an integer selected from 0, 1, 2, or 3;
each instance of r, where present, independently is an integer selected from 0, 1, 2, or 3, wherein where two instances of r occur on the same R² group, the total of both r values combined does not exceed 5;
each instance of R⁵, when present and attached to an N ring member, independently is C₁₋₆alkyl, C₁₋₆haloalkyl, C₃₋₆ cycloalkyl, or heterocyclyl having 3 to 10 members; and
each instance of R⁵, when present and attached to a C ring member, independently is oxo, CN, C₁₋₆ alky, C₃₋₆ cycloalkyl, C₁₋₆ haloalkyl, C₁₋₆ alkoxy, C(O)N(R⁶)₂, —N(R⁶)₂, —N(R⁶)(C₁₋₆ alkylene-OH), heterocyclyl having 3 to 10 members;
each instance of R⁶, where present, independently is —H, C₁₋₃ alkyl, C(O)C₁₋₃ alkyl, C(O)₂ C₁₋₃ alkyl, C₁₋₃ alkylene-OH, or S(O)ₙC₁₋₃alkyl;
wherein a —N═ ring nitrogen can be bonded to an R⁵ to provide a cation.

Provided herein as Embodiment 152 is the compound or salt of any one of Embodiments 1 to 118 and 146, wherein R² is

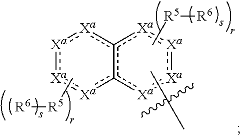

where
“ [symbol] ” indicates a double bond or single bond may be present;
each instance of X^{a} independently is nitrogen, oxygen, or carbon;
each instance of s, where present, is an integer selected from 0, 1, 2, or 3;
each instance of r, where present, independently is an integer selected from 0, 1, 2, 3, or 4, wherein where two instances of r occur on the same R² group, the total of both r values combined does not exceed 5;
each instance of R⁵, when present and attached to an N ring member, independently is C₁₋₆alkyl, C₁₋₆haloalkyl, C₃₋₆ cycloalkyl, or heterocyclyl having 3 to 10 members; and
each instance of R⁵, when present and attached to a C ring member, independently is oxo, CN, C₁₋₆ alkyl, C₃₋₆ cycloalkyl, C₁₋₆ haloalkyl, C₁₋₆ alkoxy, C(O)N(R⁶)₂, —N(R⁶)₂, —N(R⁶)(C₁₋₆ alkylene-OH), heterocyclyl having 3 to 10 members;
each instance of R⁶, where present, independently is —H, C₁₋₃ alkyl, C(O)C₁₋₃ alkyl, C(O)₂ C₁₋₃ alkyl, C₁₋₃ alkylene-OH, or S(O)ₙC₁₋₃alkyl;
wherein a —N═ ring nitrogen can be bonded to an R⁵ to provide a cation.

Provided herein as Embodiment 153 is the compound or salt of any one of Embodiments 1 to 109, wherein R² is

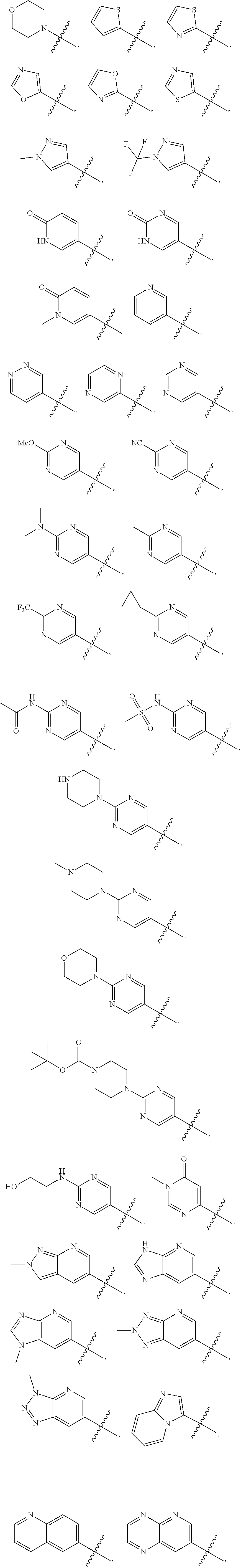

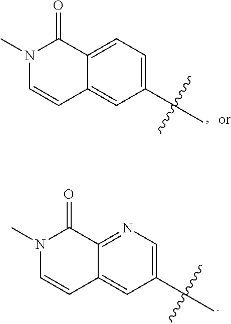 [SMILES: *c1ccc2c(=O)n(C)ccc2c1 ; *c1cnc2c(=O)n(C)ccc2c1]

For example, in several embodiments, R² is

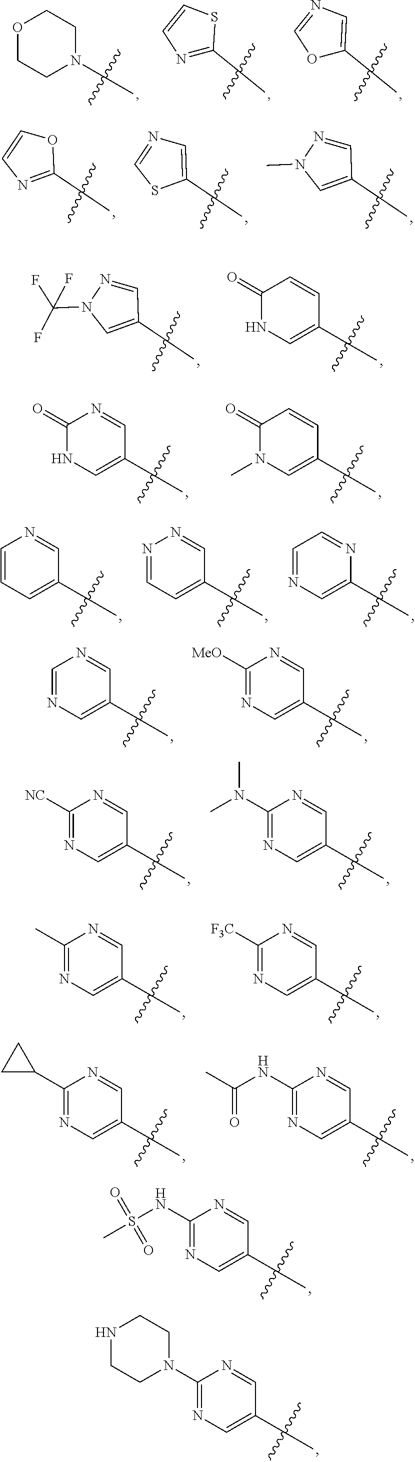

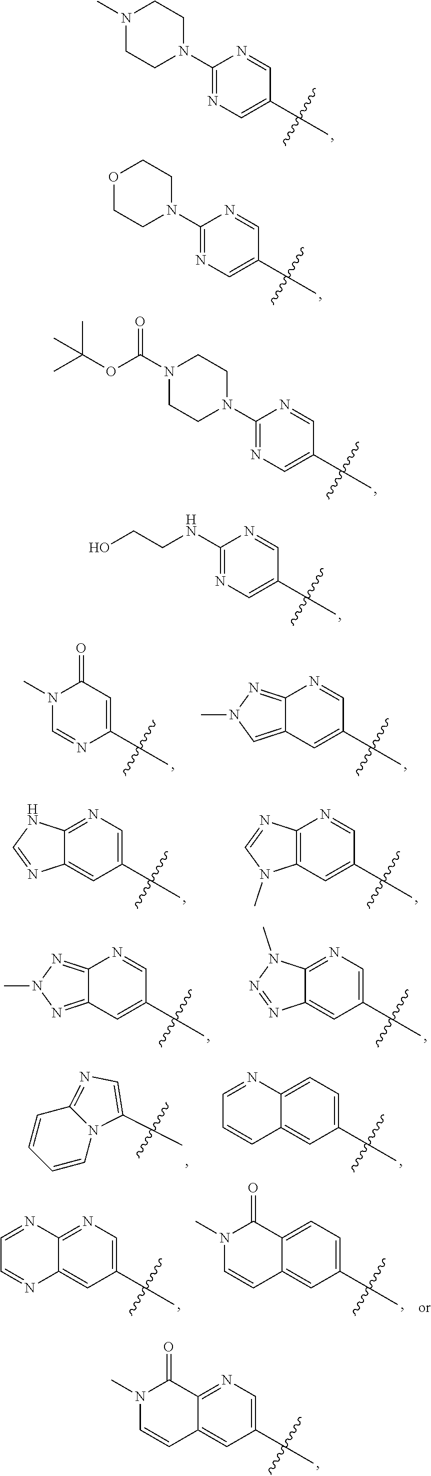

In several embodiments, R² is

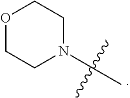 [SMILES: *N1CCOCC1]

In several embodiments, R² is

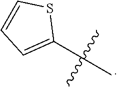 [SMILES: *c1cccs1]

In several embodiments, R² is

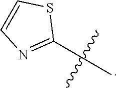 [SMILES: *c1nccs1]

In several embodiments, R² is

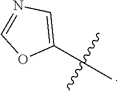 [SMILES: *c1cnco1 ; "."]

In several embodiments, R² is

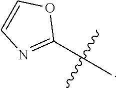 [SMILES: *c1ncco1]

In several embodiments, R² is

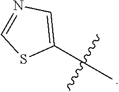 [SMILES: *c1cncs1]

In several embodiments, R² is

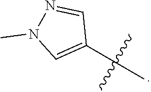 [SMILES: *c1cnn(C)c1]

In several embodiments, R² is

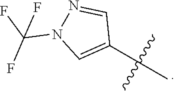 [SMILES: *c1cnn(C(F)(F)F)c1 ; "."]

In several embodiments, R²

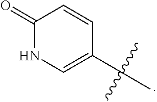 [SMILES: *c1ccc(=O)[nH]c1]

In several embodiments, R² is

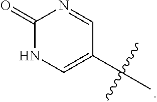 [SMILES: *c1cnc(=O)[nH]c1]

In several embodiments, R² is

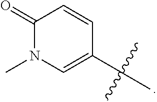 [SMILES: *c1ccc(=O)n(C)c1]

In several embodiments, R² is

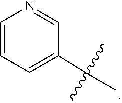 [SMILES: *c1cccnc1]

In several embodiments, R² is

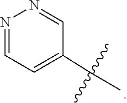 [SMILES: *c1ccnnc1]

In several embodiments, R² is

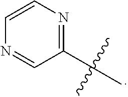 [SMILES: *c1cnccn1]

In several embodiments, R² is

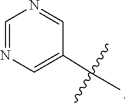 [SMILES: *c1cncnc1]

In several embodiments, R² is

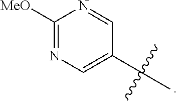 [SMILES: *c1cnc(OC)nc1]

In several embodiments, R² is

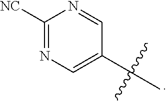 [SMILES: *c1cnc(C#N)nc1]

In several embodiments, R² is

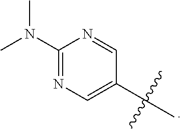 [SMILES: *c1cnc(N(C)C)nc1]

In several embodiments, R² is

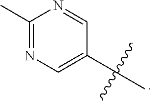 [SMILES: *c1cnc(C)nc1]

In several embodiments, R² is

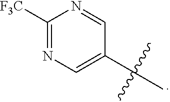 [SMILES: *c1cnc(C(F)(F)F)nc1]

In several embodiments, R² is

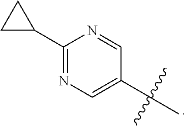 [SMILES: *c1cnc(C2CC2)nc1]

In several embodiments, R² is

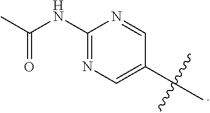 [SMILES: *c1cnc(NC(C)=O)nc1]

In several embodiments, R² is

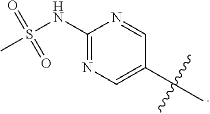 [SMILES: *c1cnc(NS(C)(=O)=O)nc1]

In several embodiments, R² is

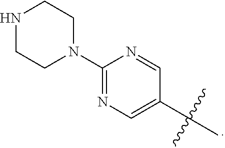 [SMILES: *c1cnc(N2CCNCC2)nc1]

In several embodiments, R² is

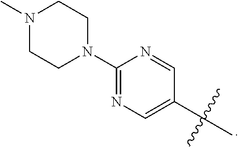 [SMILES: *c1cnc(N2CCN(C)CC2)nc1]

In several embodiments, R² is

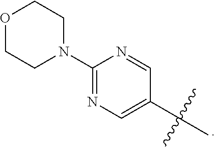 [SMILES: *c1cnc(N2CCOCC2)nc1]

In several embodiments, R² is

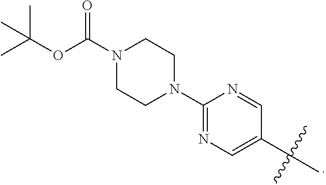 [SMILES: *c1cnc(N2CCN(C(=O)OC(C)(C)C)CC2)nc1]

In several embodiments, R² is

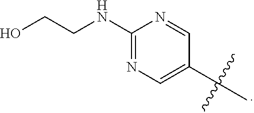 [SMILES: *c1cnc(NCCO)nc1]

In several embodiments, R² is

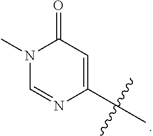 [SMILES: *c1cc(=O)n(C)cn1]

In several embodiments, R² is

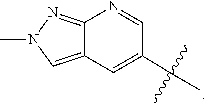 [SMILES: *c1cnc2nn(C)cc2c1]

In several embodiments, R² is

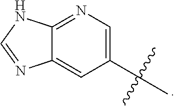 [SMILES: *c1cnc2[nH]cnc2c1]

In several embodiments, R² is

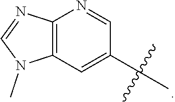 [SMILES: *c1cnc2ncn(C)c2c1]

In several embodiments, R² is

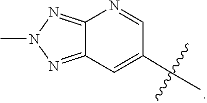 [SMILES: *c1cnc2nn(C)nc2c1]

In several embodiments, R² is

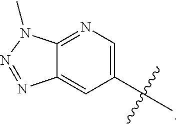

In several embodiments, R² is

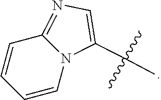 [SMILES: *c1cnc2ccccn12]

In several embodiments, R² is

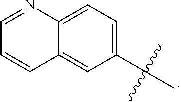 [SMILES: *c1ccc2ncccc2c1]

In several embodiments, R² is

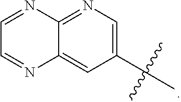 [SMILES: *c1cnc2nccnc2c1]

In several embodiments, R² is

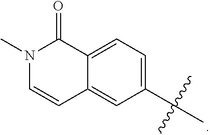 [SMILES: *c1ccc2c(=O)n(C)ccc2c1]

In several embodiments, R² is

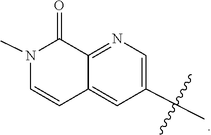 [SMILES: *c1cnc2c(=O)n(C)ccc2c1]

Provided herein as Embodiment 154 is the compound or salt of any one of Embodiments 1 to 107, wherein R² is

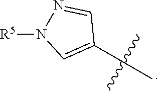

Provided herein as Embodiment 155 is the compound or salt of any one of Embodiments 1 to 107, wherein R² is

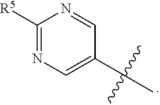

Provided herein as Embodiment 156 is the compound or salt of any one of Embodiments 1 to 130, 132, and 146 to 152, wherein each instance of R⁵ independently is —N(R⁶)₂, —OMe, —CF₃, —CN,

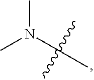 [SMILES: *N(C)C]

### —NC(O)Me, or

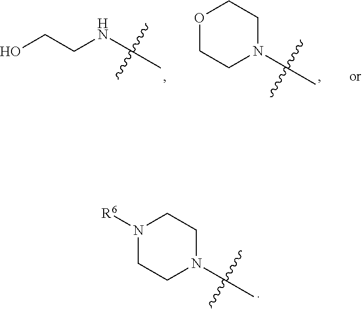

Provided herein as Embodiment 157 is the compound or salt of any one of Embodiments 1 to 130, 132, and 146 to 152, wherein each instance of R⁵ independently is —OMe, —CF₃, or —CH₃.

Provided herein as Embodiment 158 is the compound or salt of any one of Embodiments 1 to 157, wherein R³ is C₁₋₆ alkyl, C₃₋₁₀ cycloalkyl, C₆₋₁₀ aryl, heteroaryl having 5 to 10 ring members, heterocyclyl having 3 to 10 ring members. For example, in several embodiments, R³ is C₁₋₆ alkyl, C₃₋₆ cycloalkyl, C₆₋₁₀ aryl, heteroaryl having 5 to 10 ring members, or heterocyclyl having 5 to 10 ring members. In several embodiments, R³ is C₁₋₆ alkyl, heteroaryl having 5 to 10 ring members, or heterocyclyl having 5 to 10 ring members. In several embodiments, R³ is C₁₋₆ alkyl, C₃₋₆ cycloalkyl, or heteroaryl having 5 to 10 ring members. In several embodiments, R³ is heteroaryl having 5 to 9 ring members.

Provided herein as Embodiment 159 is the compound or salt of any one of Embodiments 1 to 158, wherein R³ is C₃₋₆ cycloalkyl, heteroaryl having 5 to 10 ring members, or heterocyclyl having 3 to 10 ring members.

Provided herein as Embodiment 160 is the compound or salt of any one of Embodiments 1 to 159, wherein R³ is heteroaryl or heterocyclyl and the R³ heteroaryl or heterocyclyl includes 1, 2, 3, 4, or 5 heteroatom ring members. For example, in several embodiments, R³ includes 4 heteroatom ring members. In several embodiments, R³ includes 3 heteroatom ring members. In several embodiments, R³ includes 2 heteroatom ring members. In several embodiments, R³ includes 1 heteroatom ring member. In several embodiments, R³ comprises 1 to 4 heteroatom ring members. In several embodiments, R³ comprises 1 to 3 heteroatom ring members. In several embodiments. R³ comprises 1 or 2 heteroatom ring members. In several embodiments, R³ comprises 3 to 4 heteroatom ring members. In several embodiments, R³ comprises nitrogen atoms as the only ring heteroatoms. In several embodiments, R³ comprises one nitrogen atom as a ring heteroatom. In several embodiments, R³ comprises two nitrogen atoms as a ring heteroatoms. In several embodiments, R³ comprises three nitrogen atoms as a ring heteroatoms. In several embodiments, R³ comprises four nitrogen atoms as a ring heteroatoms. In several embodiments, R³ comprises one oxygen ring heteroatom and the remaining ring heteroatoms are nitrogen atoms. In several embodiments, R³ comprises oxygen atoms as the only ring heteroatoms.

Provided herein as Embodiment 161 is the compound or salt of any one of Embodiments 1 to 160, wherein R³ is heteroaryl or heterocyclyl and the R³ heteroaryl or heterocyclyl includes 1, 2, 3, or 4 heteroatom ring members.

Provided herein as Embodiment 162 is the compound or salt of any one of Embodiments 1 to 161, wherein R³ is heteroaryl or heterocyclyl and the R³ heteroaryl or heterocyclyl includes 1, 2, or 3 heteroatom ring members.

Provided herein as Embodiment 163 is the compound or salt of any one of Embodiments 1 to 162, wherein R³ is heteroaryl or heterocyclyl and the R³ heteroaryl or heterocyclyl includes 1 or 2 heteroatom ring members.

Provided herein as Embodiment 164 is the compound or salt of any one of Embodiments 1 to 163, wherein R³ is heteroaryl or heterocyclyl and the R³ heteroaryl or heterocyclyl includes 1 heteroatom ring member.

Provided herein as Embodiment 165 is the compound or salt of any one of Embodiments 1 to 164, wherein R³ is heteroaryl or heterocyclyl and each heteroatom of the R³ ring independently is nitrogen, oxygen, or —S(O)_{z}—, where z is 0, 1, or 2.

Provided herein as Embodiment 166 is the compound or salt of any one of Embodiments 1 to 165, wherein R³ is heteroaryl or heterocyclyl and each heteroatom of the R³ ring independently is nitrogen or oxygen.

Provided herein as Embodiment 167 is the compound or salt of any one of Embodiments 1 to 166, wherein R³ is heteroaryl or heterocyclyl and each heteroatom of the R³ ring is nitrogen.

Provided herein as Embodiment 168 is the compound or salt of any one of Embodiments 1 to 159 and 162 to 166, wherein R³ is heteroaryl or heterocyclyl and each heteroatom of the R³ ring is oxygen.

Provided herein as Embodiment 169 is the compound or salt of any one of Embodiments 1 to 168, wherein R³ is fused bicyclic heteroaryl or fused bicyclic heterocyclic.

Provided herein as Embodiment 170 is the compound or salt of any one of Embodiments 1 to 169, wherein R³ is heteroaryl.

Provided herein as Embodiment 171 is the compound or salt of any one of Embodiments 1 to 169, wherein R³ is heterocyclic.

Provided herein as Embodiment 172 is the compound or salt of any one of Embodiments 1 to 171, wherein R³ comprises 5 to 10 ring-members.

Provided herein as Embodiment 173 is the compound or salt of any one of Embodiments 1 to 172, wherein R³ comprises 5 to 9 ring-members.

Provided herein as Embodiment 174 is the compound or salt of any one of Embodiments 1 to 173, wherein R³ has 9-ring members.

Provided herein as Embodiment 175 is the compound or salt of any one of Embodiments 1 to 159 and 162 to 168 and 170 to 173, wherein R³ has 6-ring members.

Provided herein as Embodiment 176 is the compound or salt of any one of Embodiments 1 to 159 and 161 to 168 and 170 to 173, wherein R³ has 5-ring members.

Provided herein as Embodiment 177 is the compound or salt of any one of Embodiments 1 to 158, wherein R³ is C₃₋₄cycloalkyl.

Provided herein as Embodiment 178 is the compound or salt of any one of Embodiments 1 to 158, wherein R³ is C₁₋₆ alkyl.

Provided herein as Embodiment 179 is the compound or salt of any one of Embodiments 1 to 158, wherein R³ is C₆₋₁₀ aryl.

Provided herein as Embodiment 180 is the compound or salt of any one of Embodiments 1 to 158, wherein R³ is C₆ aryl.

Provided herein as Embodiment 181 is the compound or salt of any one of Embodiments 1 to 180, wherein R³ is substituted with 1, 2, 3, 4, or 5 substituents. For example, in several embodiments, R³ is substituted with 2 to 5 substituents. In several embodiments, R³ is substituted with 3 to 4 substituents. In several embodiments, R³ is substituted with 3 to 5 substituents. In several embodiments, R³ is substituted with 4 to 5 substituents. In several embodiments, R³ is substituted with 2, 3, 4, or 5 substituents. In several embodiments R³ is substituted with 1, 2, or 4 substituents. In several embodiments, R³ is substituted 1 or 3 substituents.

Provided herein as Embodiment 182 is the compound or salt of any one of Embodiments 1 to 181, wherein R³ is substituted with 1, 2, 3, or 4 substituents.

Provided herein as Embodiment 183 is the compound or salt of any one of Embodiments 1 to 182, wherein R³ is substituted with 1, 2, or 3 substituents.

Provided herein as Embodiment 184 is the compound or salt of any one of Embodiments 1 to 183, wherein R³ is substituted with 1 or 2 substituents.

Provided herein as Embodiment 185 is the compound or salt of any one of Embodiments 1 to 184, wherein R³ is substituted with 1 substituent.

Provided herein as Embodiment 186 is the compound or salt of any one of Embodiments 1 to 185, wherein, when R³ is substituted, each R³ substituent independently is halogen, —OH, C₁₋₃alkyl, C₁₋₃alkoxy, or C₁₋₃ alkylene-C₁₋₃ alkoxy. For example, in several embodiments, each substituent of R³ independently is C₁₋₃alkyl, C₁₋₃alkoxy, or C₁₋₃ alkylene-C₁₋₃ alkoxy. In several embodiments, R³ is substituted with —F or —OH. In several embodiments, R³ is substituted with —OH or C₁₋₃ alkoxy. In several embodiments, R³ is substituted with —F or C₁₋₃ alkoxy. In several embodiments, R³ is substituted with —F, —OH, C₁ alkyl, C₂ alkyl, C₃ alkyl, C₁ alkoxy, C₂ alkoxy, and/or C₃ alkoxy. In several embodiments, R³ is substituted with methyl, C₂ alkyl, and/or C₃ alkyl. In several embodiments, R³ is substituted with —C₁ alkoxy, C₂ alkoxy, or C₃ alkoxy. In several embodiments, R³ is substituted with —C₁ alkoxy. In several embodiments, R³ is substituted with C₂alkoxy. In several embodiments, R³ is substituted with C₃ alkoxy. In several embodiments, R³ is substituted with —F. In several embodiments, R³ is substituted with —OH. In several embodiments, R³ is substituted with methyl.

Provided herein as Embodiment 187 is the compound or salt of any one of Embodiments 1 to 185, wherein each R³ substituent independently is methyl, ethyl, or isopropyl.

Provided herein as Embodiment 188 is the compound or salt of any one of Embodiments 1 to 158, wherein R³ is

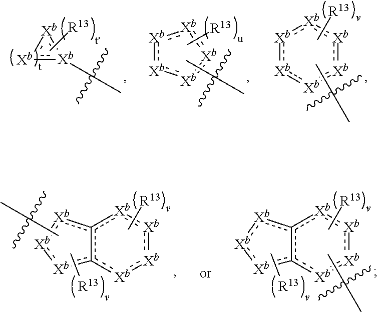

where
“ [symbol] ” indicates a double bond or single bond may be present;
each instance of X^{b} independently is nitrogen, oxygen, or carbon;
each instance of t, where present, is an integer selected from 1, 2, 3, 4, or 5;
each instance of t′, where present, is an integer selected from 1, 2, 3, 4, or 5;
each instance of u, where present, is an integer selected from 1, 2, 3, 4, or 5;
each instance of v, where present, is an integer selected from 0, 1, 2, 3, 4, or 5, wherein where two instances of v occur on the same R³ group, the total of both v values combined does not exceed 5;
each instance of R¹³, when present and attached to an N ring member, independently is C₁₋₃alkyl, C₁₋₃haloalkyl, or C₁₋₃ alkylene-C₁₋₃ alkoxy; and
each instance of R¹³, when present and attached to a C ring member, independently is halogen, —OH, oxo, —N(R^{b})₂, C₁₋₃alkyl, C₁₋₃haloalkyl, or C₁₋₃alkylene-O—C₁₋₃alkyl;
wherein a —N═ ring nitrogen can be bonded to an R¹³ to provide a cation.

Provided herein as Embodiment 189 is the compound or salt of any one of Embodiments 1 to 158 and 188, wherein R³ is:

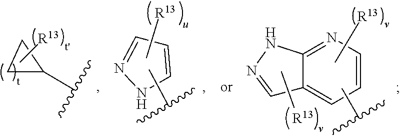

where
each instance of t, where present, is an integer selected from 1, 2, 3, 4, or 5;
each instance of t′, where present, is an integer selected from 1, 2, 3, 4, or 5;
each instance of v, where present, is an integer selected from 0, 1, 2, 3, 4, or 5, wherein where two instances of v occur on the same R³ group, the total of both v values combined does not exceed 5;
each instance of R¹³, when present and attached to an N ring member, independently is C₁₋₃alkyl, C₁₋₃haloalkyl, or C₁₋₃ alkylene-C₁₋₃ alkoxy; and
each instance of R¹³, when present and attached to a C ring member, independently is halogen, —OH, oxo, C₁₋₃alkyl, C₁₋₃haloalkyl, or C₁₋₃alkylene-O—C₁₋₃alkyl;
or, alternatively, two instances of R¹³ together form a C₃₋₆ cycloalkyl or heterocyclyl having 3 to 6 ring members, which may provided a fused ring system or spiro ring system when R³ is a ring structure;
wherein a —N═ ring nitrogen can be bonded to an R¹³ to provide a cation.

Provided herein as Embodiment 190 is the compound or salt of any one of Embodiments 1 to 158, wherein R³ is:

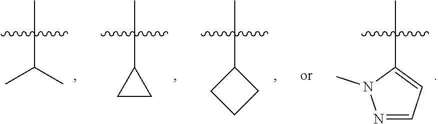

Provided herein as Embodiment 191 is the compound or salt of any one of Embodiments 1 to 158, wherein R³ is:

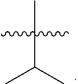 [SMILES: *C(C)C]

Provided herein as Embodiment 192 is the compound or salt of any one of Embodiments 1 to 158, wherein R³ is:

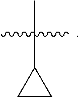 [SMILES: *C1CC1]

Provided herein as Embodiment 193 is the compound or salt of any one of Embodiments 1 to 158, wherein R³ is:

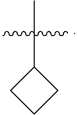 [SMILES: *C1CCC1]

Provided herein as Embodiment 194 is the compound or salt of any one of Embodiments 1 to 16 and 17 to 158, wherein R³ is:

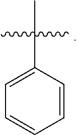 [SMILES: *c1ccccc1]

Provided herein as Embodiment 195 is the compound or salt of any one of Embodiments 1 to 158, wherein R³ is:

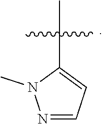 [SMILES: *c1ccnn1C]

Provided herein as Embodiment 196 is the compound or salt of any one of Embodiments 1 to 157, wherein R³ is —H.

Provided herein as Embodiment 197 is the compound or salt of any one of Embodiments 1 to 196, wherein R⁴ is C₁₋₆ alkyl. For example, in several embodiments, R⁴ is C₁₋₅ alkyl. In several embodiments, R⁴ is C₁₋₄ alkyl. In several embodiments, R⁴ is C₁₋₃ alkyl. In several embodiments, R⁴ is C₁₋₂alkyl. In several embodiments, R⁴ is C₂₋₃ alkyl. In several embodiments, R⁴ is C₁ alkyl (e.g., methyl). In several embodiments, R⁴ is C₂ alkyl. In several embodiments, R⁴ is C₃ alkyl.

Provided herein as Embodiment 198 is the compound or salt of any one of Embodiments 1 to 197, wherein R⁴ is C₁₋₃ alkyl.

Provided herein as Embodiment 199 is the compound or salt of any one of Embodiments 1 to 14 and 16 to 196, wherein R⁴ is —N(R⁷)₂.

Provided herein as Embodiment 200 is the compound or salt of any one of Embodiments 1 to 14, 16 to 196, and 199, wherein at least one instance of R⁷ is C₁₋₃alkyl. For example, in several embodiments, at least one instance of R⁷ is C₁₋₂ alkyl. In several embodiments, at least one instance of R⁷ is C₂₋₃ alkyl. In several embodiments, at least one instance of R⁷ is C₁ alkyl. In several embodiments, at least one instance of R⁷ is C₂ alkyl. In several embodiments, at least one instance of R⁷ is C₃ alkyl. In several embodiments, both instances of R⁷ are the same. In several embodiments, each instance of R⁷ is different.

Provided herein as Embodiment 201 is the compound or salt of any one of Embodiments 1 to 14, 16 to 196 and 199, wherein at least one instance of R⁷ is -Me.

Provided herein as Embodiment 202 is the compound or salt of any one of Embodiments 1 to 14, 16 to 196 and 199, wherein at least one instance of R⁷ is H.

Provided herein as Embodiment 203 is the compound or salt of any one of Embodiments 1 to 14, 17 to 196 and 199, wherein each instance of R⁷ is H.

Provided herein as Embodiment 204 is the compound or salt of any one of Embodiments 1 to 196, wherein R⁴ is —H.

Provided herein as Embodiment 205 is the compound or salt of any one of Embodiments 1 to 27, 30, 32, 34, 38 to 40, 46, 47, 50 to 157, 197, and 198, wherein the compound of Formula (I) is a compound of Formula (Ia):

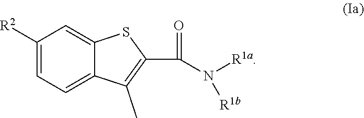

Provided herein as Embodiment 206 is the compound or salt of any one of Embodiments 1 to 14, 16 to 27, 29, 32, 34, 38, 39, 41, 46 to 196, 199, 202, and 203, wherein the compound of Formula (I) is a compound of Formula (Ib):

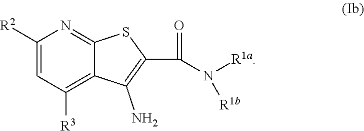

Provided herein as Embodiment 207 is the compound or salt of any one of Embodiments 1 to 27, 29, 32, 34, 37, 39, 42, 46 to 157, 196, and 204, wherein the compound of Formula (I) is a compound of Formula (Ic):

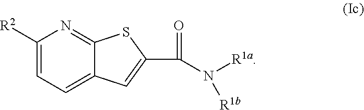

Provided herein as Embodiment 208 is the compound or salt of any one of Embodiments 1 to 27, 29, 32, 35, 37, 39, 41, 46 to 196, and 204, wherein the compound of Formula (I) is a compound of Formula (Id):

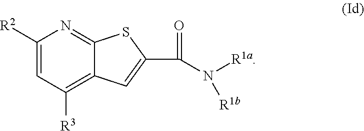

Provided herein as Embodiment 209 is the compound or salt of any one of Embodiments 1 to 26, 28, 30, 32, 33, 37, 39, 43, 46 to 157, and 204, wherein the compound of Formula (I) is a compound of Formula (Ie):

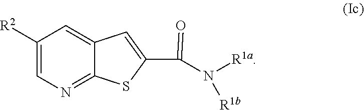

Provided herein as Embodiment 210 is the compound or salt of any one of Embodiments 1 to 27, 29, 31, 35, 37, 39, 44, 46, 47, 50 to 196, and 204, wherein the compound of Formula (I) is a compound of Formula (If):

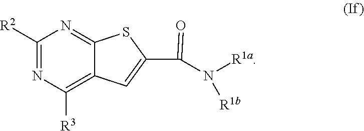

Provided herein as Embodiment 211 is the compound or salt of any one of Embodiments 1 to 27, 29, 32, 33, 37, 39, 45, 46, 47, 50 to 157, and 204, wherein the compound of Formula (I) is a compound of Formula (Ig):

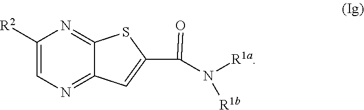

Provided herein as Embodiment 212 is the compound or salt of any one of Embodiments 1 to 26, 28, 29, 32, 33, 37, 39, 45, 46, 47, 50 to 157, and 204, wherein the compound of Formula (I) is a compound of Formula (Ih):

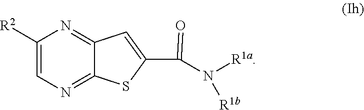

Provided herein as Embodiment 213 is the compound or salt of any one of Embodiments 1 to 26, 28, 29, 32, 34, 37, 39, 42, 46 to 157, and 204, wherein the compound of Formula (I) is a compound of Formula (Ii):

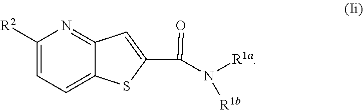

Provided herein as Embodiment 214 is the compound or salt of any one of Embodiments 1 to 26, 28, 29, 32, 34, 38, 39, 42, 46 to 157, and 197 to 204, wherein the compound of Formula (I) is a compound of Formula (Ij):

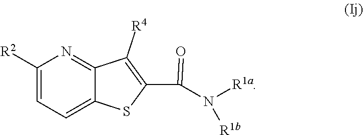

Provided herein as Embodiment 215 is the compound or salt of any one of Embodiments 1 to 26, 28, 29, 31, 35, 37, 39, 44, 46, 47, 50 to 196, and 204, wherein the compound of Formula (I) is a compound of Formula (Ik):

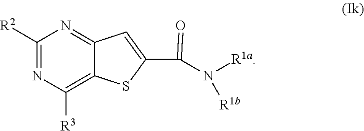

Provided herein as Embodiment 216 is the compound or salt of any one of Embodiments 1 to 27, 29, 32, 34, 36, 39, 42, 46, 47, 48, and 50 to 157, wherein the compound of Formula (I) is a compound of Formula (Il):

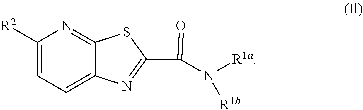

In several embodiments, the compound or salt of Formula (I) (e.g., as provided in Embodiment 1) may be further represented by a compound any one or more of Formulae (Ia), (Ib), (Ic), (Id), (Ie), (If), (Ig), (Ih), (Ii), (Ij), (Ik), and/or (Il). For example, in several embodiments, the compound or salt of Formula (I) (e.g., as provided in Embodiment 1) may be further represented by all of, or any sub-combination of, Formulae (Ia), (Ib), (Ic), (Id), (Ie), (If), (Ig), (Ih), (Ii), (Ij), and/or (Ik) (e.g., excluding Formula (Il)). For example, in several embodiments, the compound or salt of Formula (I) (e.g., as provided in Embodiment 1) may be further represented by Formulae (Ia), (Ib), (Ic), (Id), (Ie), (If), (Ig), (Ih), (Ii), (Ij), and/or (Il) (e.g., excluding Formula (Ik)). In several embodiments, the compound or salt of Formula (I) (e.g., as provided in Embodiment 1) may be further represented by Formulae (Ia), (Ib), (Ic), (Id), (Ie), (If), (Ig), and/or (Ii) (e.g., excluding Formulae (Ih), (Ij), (Ik), and (Il)). In several embodiments, the compound or salt of Formula (I) (e.g., as provided in Embodiment 1) may be further represented by Formulae (Ia), (Ib), (Ic), (Id), (Ie), (If), (Ih), and/or (Ii). In several embodiments, the compound or salt of Formula (I) (e.g., as provided in Embodiment 1) may be further represented by Formulae (Ia), (Ib), (Ic), (Id), (Ie), (Ig), (Ih), and/or (Ii). In several embodiments, the compound or salt of Formula (I) (e.g., as provided in Embodiment 1) may be further represented by Formulae (Ia), (Ib), (Ic), (Id), (If), (Ig), (Ih), and/or (Ii). In several embodiments, the compound or salt of Formula (I) (e.g., as provided in Embodiment 1) may be further represented by Formulae (Ia), (Ib), (Ic), (Ie), (If), (Ig), (Ih), and/or (Ii). In several embodiments, the compound or salt of Formula (I) (e.g., as provided in Embodiment 1) may be further represented by Formulae (Ia), (Ib), (Id), (Ie), (If), (Ig), (Ih), and/or (Ii). In several embodiments, the compound or salt of Formula (I) (e.g., as provided in Embodiment 1) may be further represented by Formulae (Ia), (Ic), (Id), (Ie), (If), (Ig), (Ih), and/or (Ii). In several embodiments, the compound or salt of Formula (I) (e.g., as provided in Embodiment 1) may be further represented by Formulae (Ia), (Ib), (Ic), (Id), (Ie), (If), (Ig), (Ih), and/or (Ii). In several embodiments, the compound or salt of Formula (I) (e.g., as provided in Embodiment 1) may be further represented by Formulae (Ib), (Ic), (Id), (Ie), (If), (Ig), (Ih), and/or (Ii). In several embodiments, the compound or salt of Formula (I) (e.g., as provided in Embodiment 1) may be further represented by Formulae (Ia), (Ib), (Ic). (Id), (Ie), (If), and/or (Ig). In several embodiments, the compound or salt of Formula (I) (e.g., as provided in Embodiment 1) may be further represented by Formulae (Ia), (Ib), (Ic), (Id), (Ie), (If), and/or (Ii). In several embodiments, the compound or salt of Formula (I) (e.g., as provided in Embodiment 1) may be further represented by Formulae (Ia), (Ib), (Ic), (Id), (Ic), (Ih), and/or (Ii). In several embodiments, the compound or salt of Formula (I) (e.g., as provided in Embodiment 1) may be further represented by Formulae (Ia), (Ib), (Ic), (Id), (Ig), (Ih), and/or (Ii). In several embodiments, the compound or salt of Formula (I) (e.g., as provided in Embodiment 1) may be further represented by Formulae (Ia), (Ib), (Ic), (If), (Ig), (Ih), and/or (Ii). In several embodiments, the compound or salt of Formula (I) (e.g., as provided in Embodiment 1) may be further represented by Formulae (Ia), (Ib), (Ie), (If), (Ig), (Ih), and/or (Ii). In several embodiments, the compound or salt of Formula (I) (e.g., as provided in Embodiment 1) may be further represented by Formulae (Ia), (Id), (Ie), (If), (Ig), (Ih), and/or (Ii). In several embodiments, the compound or salt of Formula (I) (e.g., as provided in Embodiment 1) may be further represented by Formulae (Ia), (Ic), (Id), (Ie), (If), (Ig), (Ih), and/or (Ii). In several embodiments, the compound or salt of Formula (I) (e.g., as provided in Embodiment 1) may be further represented by Formulae (Ib), (Ic), (Id), (Ie), (If), (Ig), (Ih), and/or (Ii). In several embodiments, the compound or salt of Formula (I) (e.g., as provided in Embodiment 1) may be further represented by Formulae (Ia), and/or (Ib). In several embodiments, the compound or salt of Formula (I) (e.g., as provided in Embodiment 1) may be further represented by Formulae (Ic), (Id), and/or (Ie). In several embodiments, the compound or salt of Formula (I) (e.g., as provided in Embodiment 1) may be further represented by Formulae (If), (Ig), (Ih), and/or (Ii). In several embodiments, the compound or salt of Formula (I) (e.g., as provided in Embodiment 1) may be further represented by Formulae (Ij), (Ik), and/or (Il). As will be appreciated, any other combinations of formulae are also envisioned. In several embodiments, provided herein is a compound or salt of any one or more of Embodiments 205 to 216 (including any sub-combination of Embodiments 205 to 216) as further defined in any one or more of Embodiments 1 to 204.

Provided herein as Embodiment 217 is the compound or salt of any one of Embodiments 1 to 27, 32, 34, 37, and 50 to 107, wherein the compound of Formula (I) is a compound of Formula (Im):

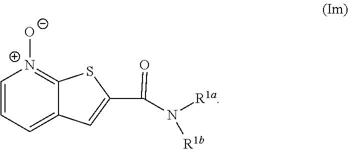

Provided herein as Embodiment 218 is the compound or salt of any one of Embodiments 1 to 27, 32, 34, 37, and 50 to 107, wherein when a is N⁺—O⁻, then

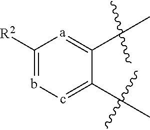

group is

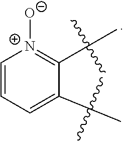 [SMILES: *c1ccc[n+]([O-])c1*]

Provided herein as Embodiment 219 is the compound or salt of Embodiment 1, wherein the compound is:

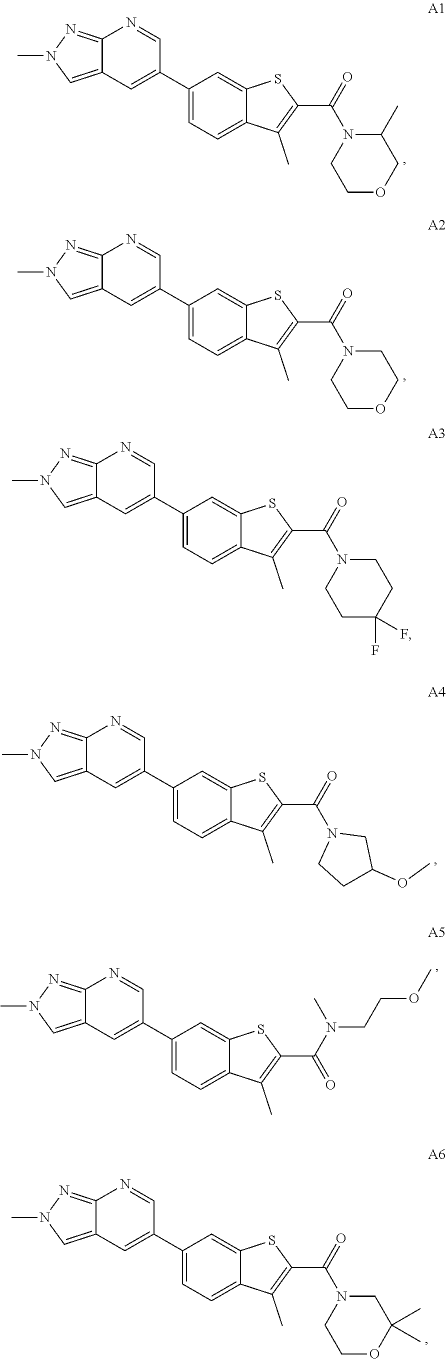

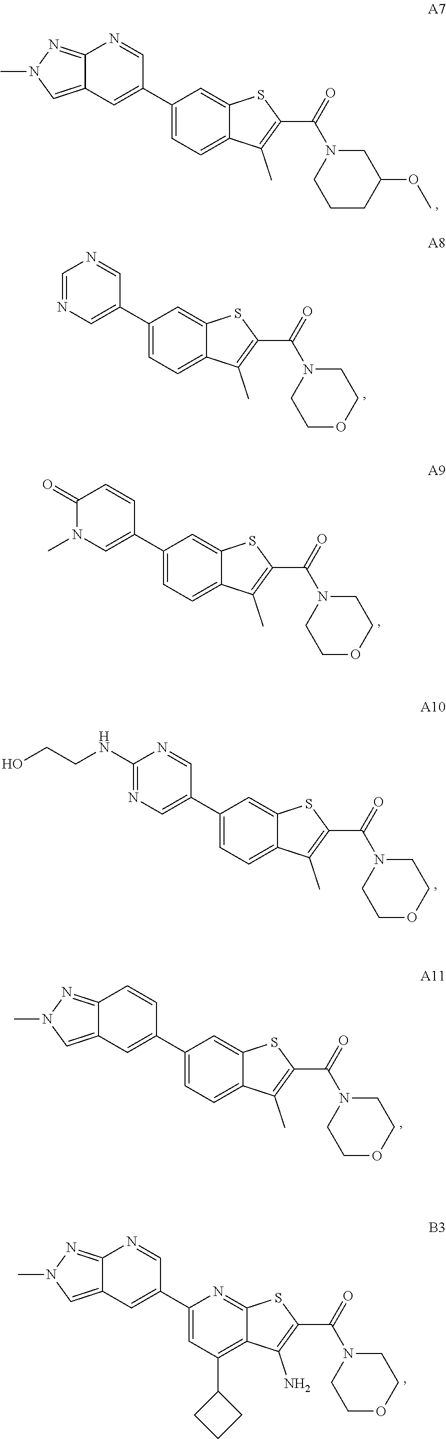

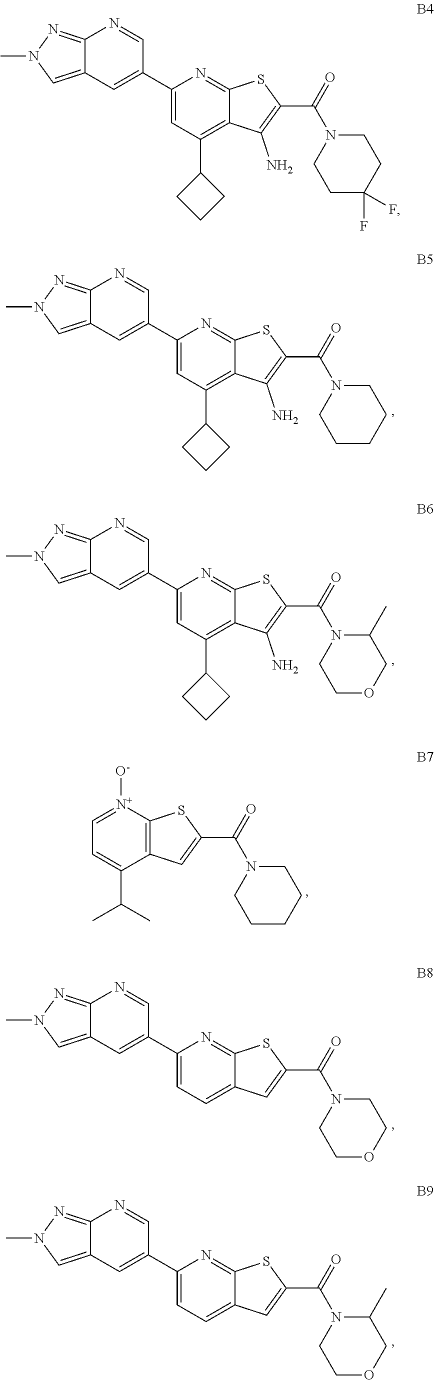

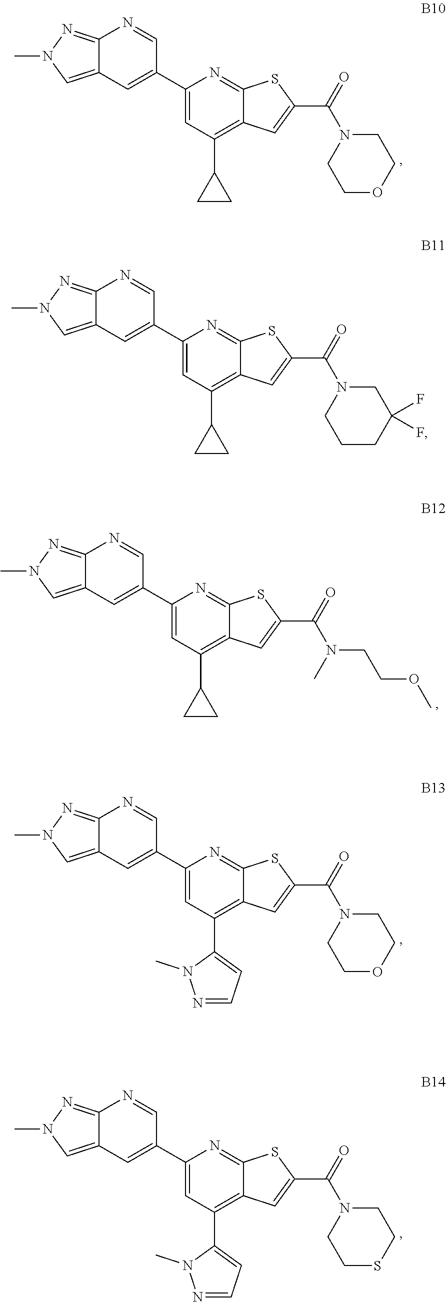

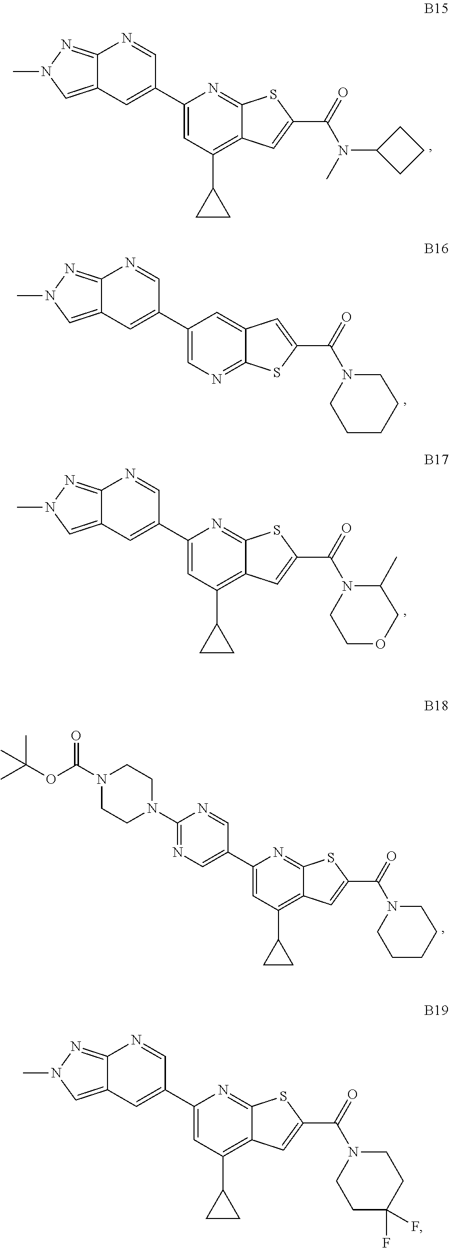 [SMILES: CN(C(=O)c1cc2c(C3CC3)cc(-c3cnc4nn(C)cc4c3)nc2s1)C1CCC1 ; Cn1cc2cc(-c3cnc4sc(C(=O)N5CCCCC5)cc4c3)cnc2n1 ; CC1COCCN1C(=O)c1cc2c(C3CC3)cc(-c3cnc4nn(C)cc4c3)nc2s1 ; CC(C)(C)OC(=O)N1CCN(c2ncc(-c3cc(C4CC4)c4cc(C(=O)N5CCCCC5)sc4n3)cn2)CC1 ; Cn1cc2cc(-c3cc(C4CC4)c4cc(C(=O)N5CCC(F)(F)CC5)sc4n3)cnc2n1]

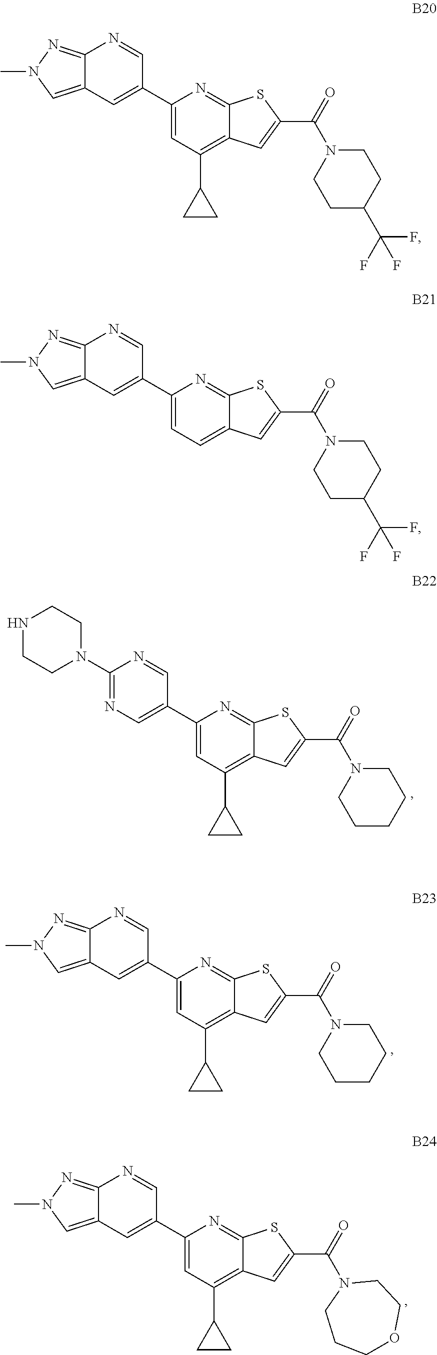 [SMILES: Cn1cc2cc(-c3cc(C4CC4)c4cc(C(=O)N5CCC(C(F)(F)F)CC5)sc4n3)cnc2n1 ; Cn1cc2cc(-c3ccc4cc(C(=O)N5CCC(C(F)(F)F)CC5)sc4n3)cnc2n1 ; O=C(c1cc2c(C3CC3)cc(-c3cnc(N4CCNCC4)nc3)nc2s1)N1CCCCC1 ; Cn1cc2cc(-c3cc(C4CC4)c4cc(C(=O)N5CCCCC5)sc4n3)cnc2n1 ; Cn1cc2cc(-c3cc(C4CC4)c4cc(C(=O)N5CCCOCC5)sc4n3)cnc2n1]

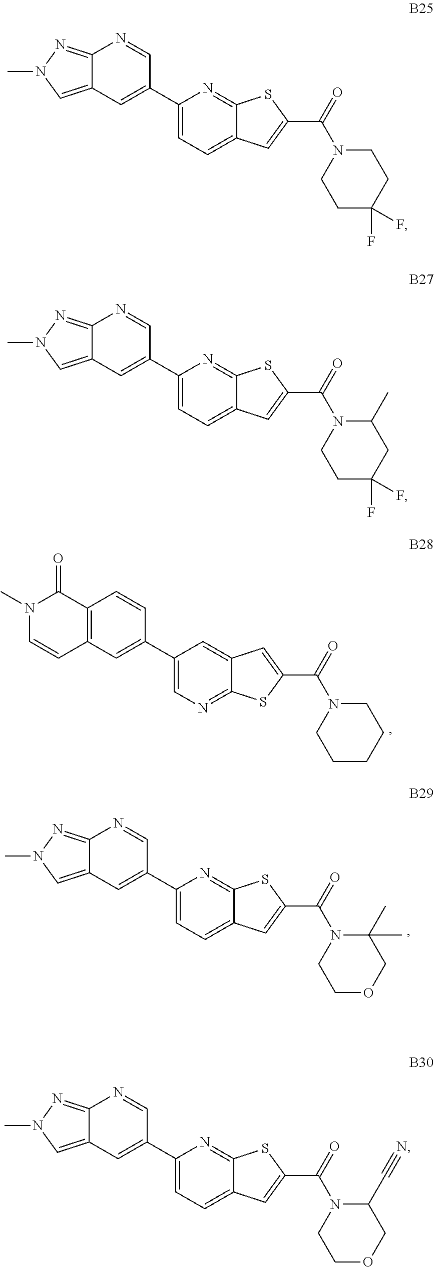

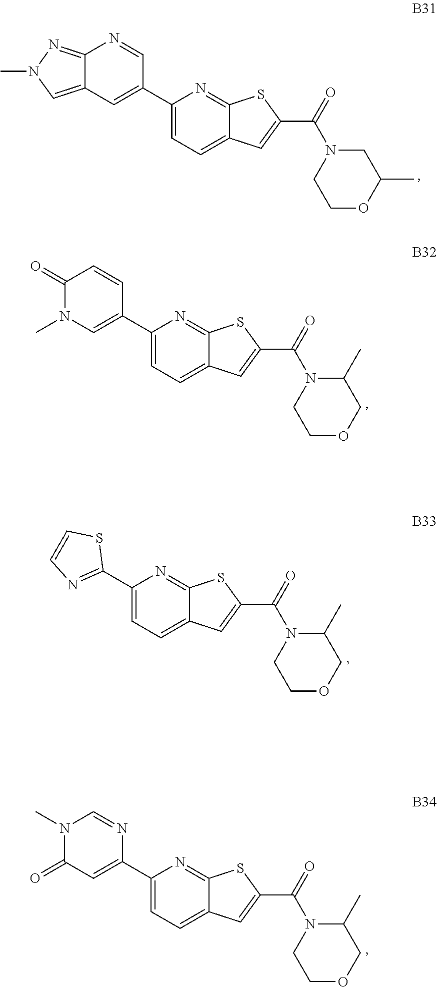

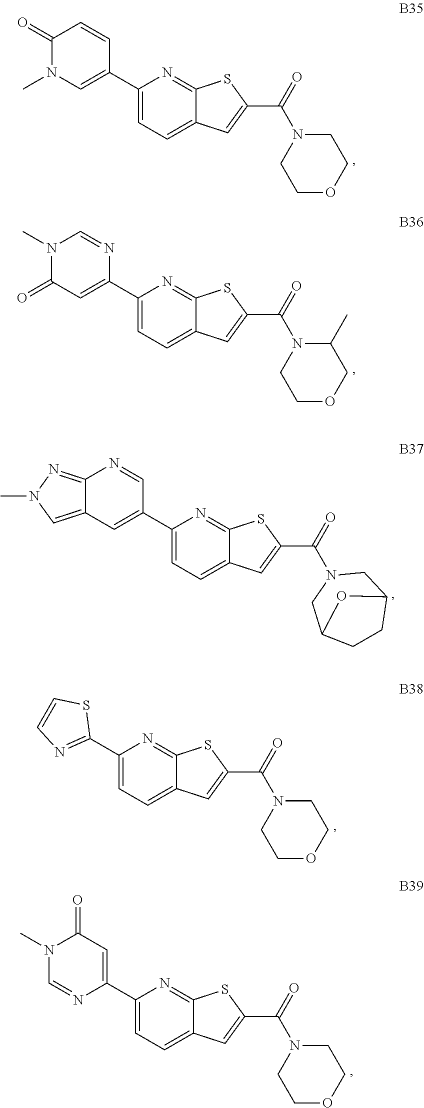

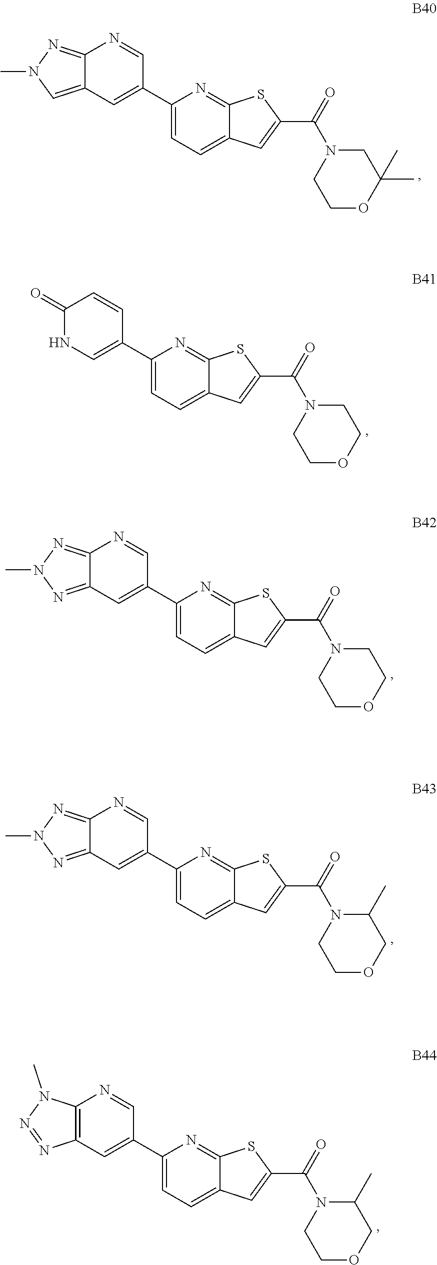

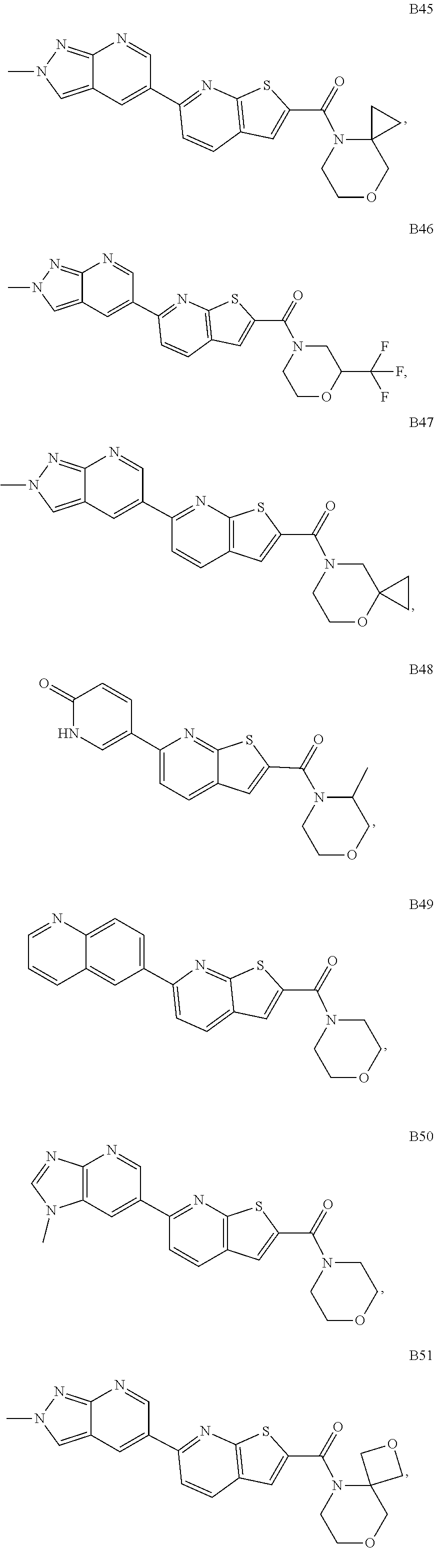

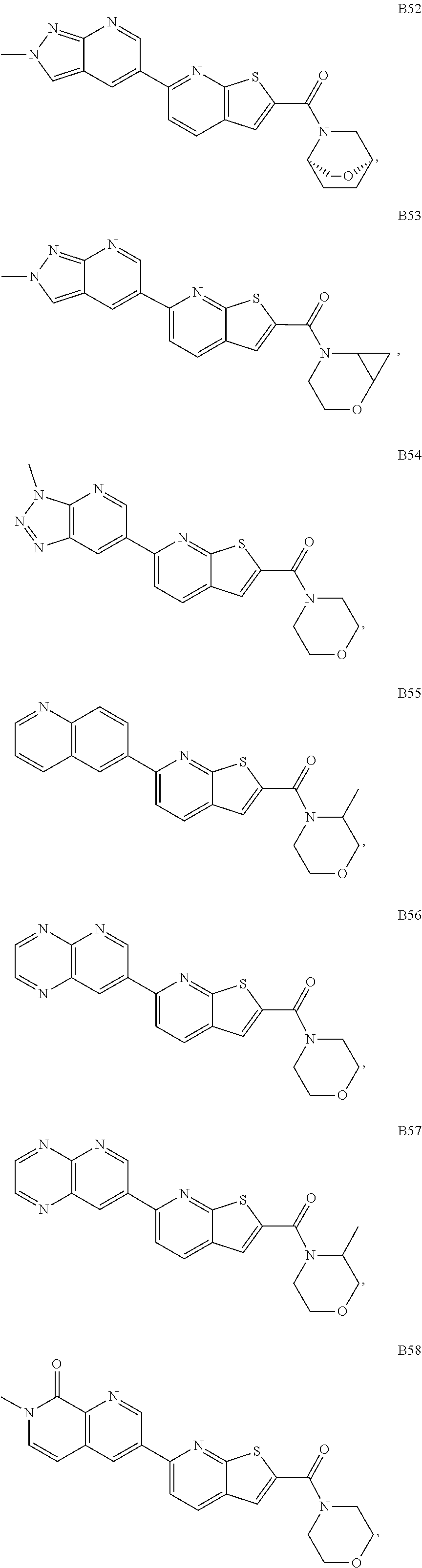

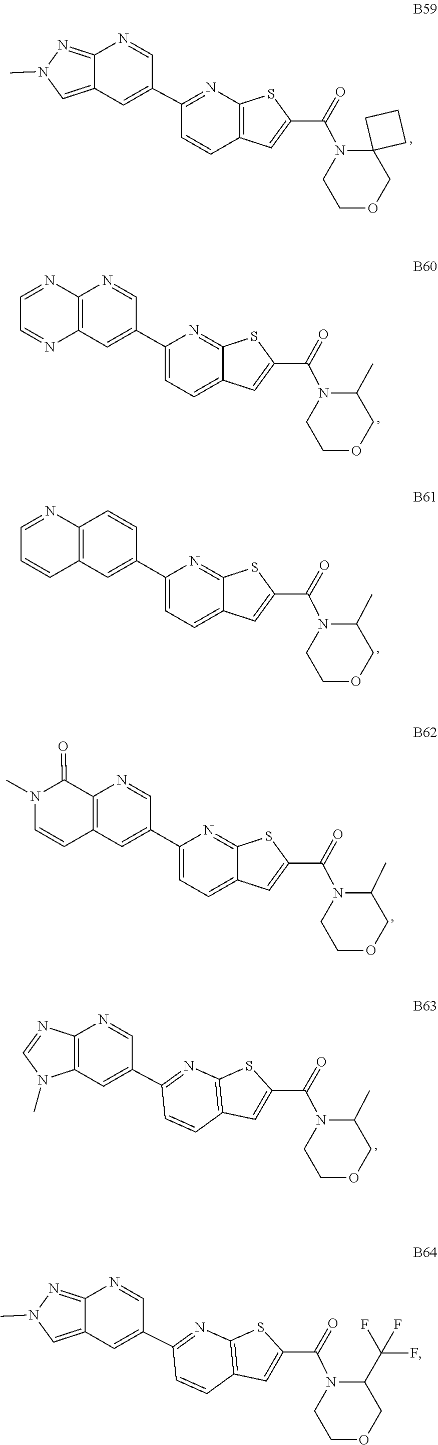 [SMILES: Cn1cc2cc(-c3ccc4cc(C(=O)N5CCOCC56CCC6)sc4n3)cnc2n1 ; CC1COCCN1C(=O)c1cc2ccc(-c3cnc4nccnc4c3)nc2s1 ; CC1COCCN1C(=O)c1cc2ccc(-c3ccc4ncccc4c3)nc2s1 ; CC1COCCN1C(=O)c1cc2ccc(-c3cnc4c(=O)n(C)ccc4c3)nc2s1 ; CC1COCCN1C(=O)c1cc2ccc(-c3cnc4ncn(C)c4c3)nc2s1 ; Cn1cc2cc(-c3ccc4cc(C(=O)N5CCOCC5C(F)(F)F)sc4n3)cnc2n1]

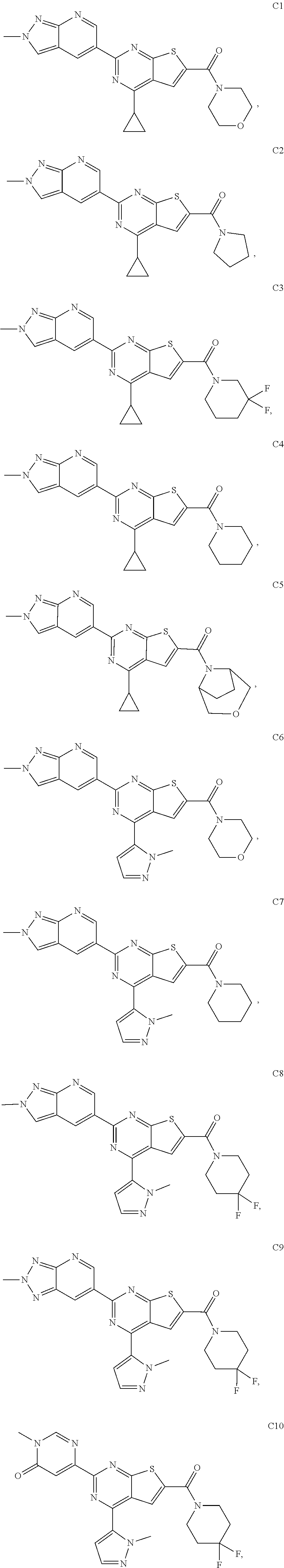

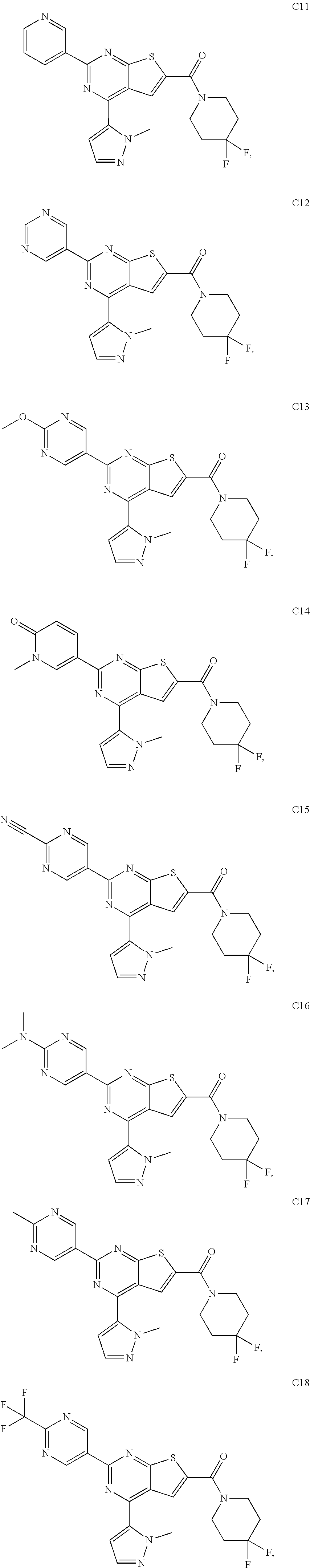

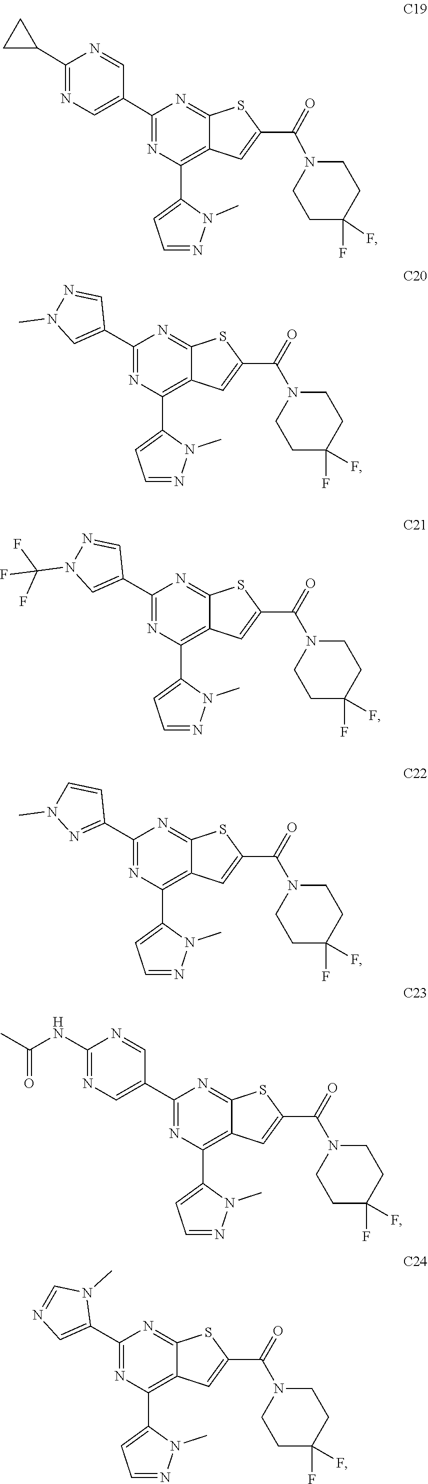 [SMILES: Cn1nccc1-c1nc(-c2cnc(C3CC3)nc2)nc2sc(C(=O)N3CCC(F)(F)CC3)cc12 ; Cn1cc(-c2nc(-c3ccnn3C)c3cc(C(=O)N4CCC(F)(F)CC4)sc3n2)cn1 ; Cn1nccc1-c1nc(-c2cnn(C(F)(F)F)c2)nc2sc(C(=O)N3CCC(F)(F)CC3)cc12 ; Cn1ccc(-c2nc(-c3ccnn3C)c3cc(C(=O)N4CCC(F)(F)CC4)sc3n2)n1 ; CC(=O)Nc1ncc(-c2nc(-c3ccnn3C)c3cc(C(=O)N4CCC(F)(F)CC4)sc3n2)cn1 ; Cn1cncc1-c1nc(-c2ccnn2C)c2cc(C(=O)N3CCC(F)(F)CC3)sc2n1]

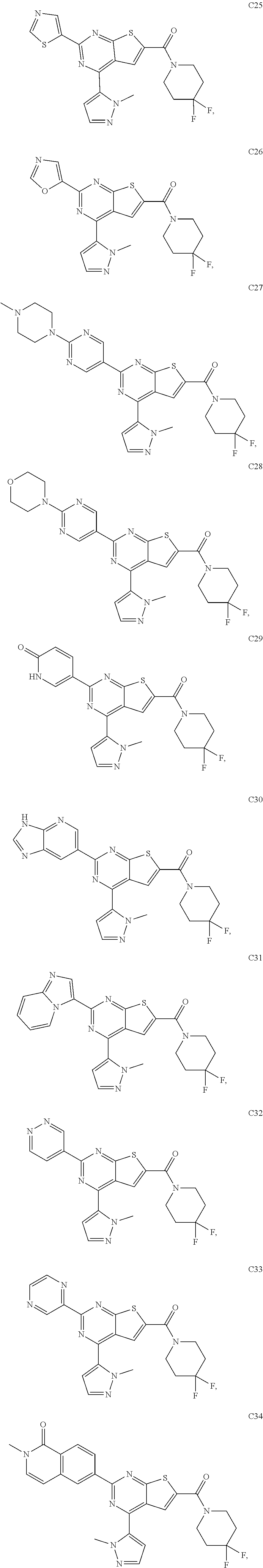

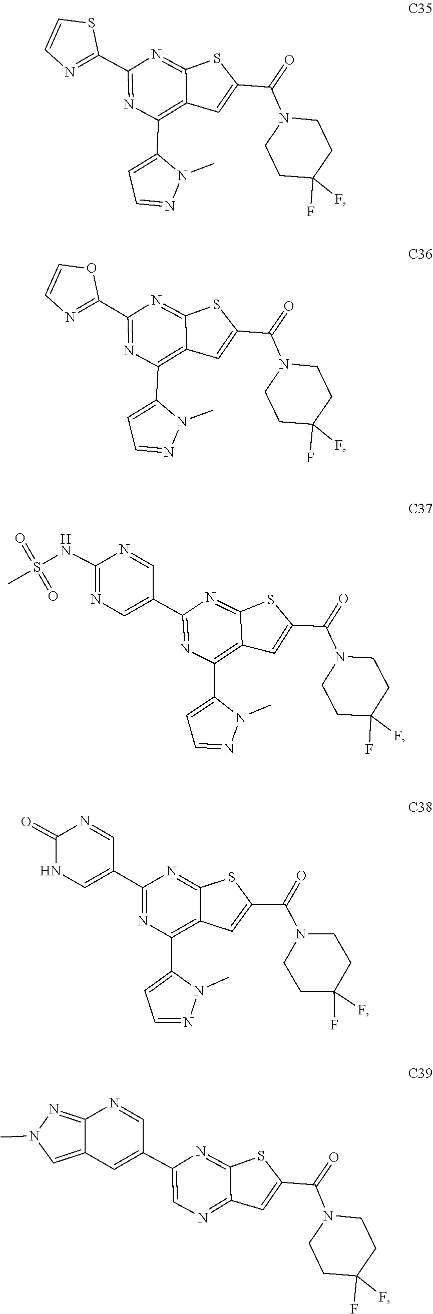 [SMILES: Cn1nccc1-c1nc(-c2nccs2)nc2sc(C(=O)N3CCC(F)(F)CC3)cc12 ; Cn1nccc1-c1nc(-c2ncco2)nc2sc(C(=O)N3CCC(F)(F)CC3)cc12 ; Cn1nccc1-c1nc(-c2cnc(NS(C)(=O)=O)nc2)nc2sc(C(=O)N3CCC(F)(F)CC3)cc12 ; Cn1nccc1-c1nc(-c2cnc(=O)[nH]c2)nc2sc(C(=O)N3CCC(F)(F)CC3)cc12 ; Cn1cc2cc(-c3cnc4cc(C(=O)N5CCC(F)(F)CC5)sc4n3)cnc2n1]

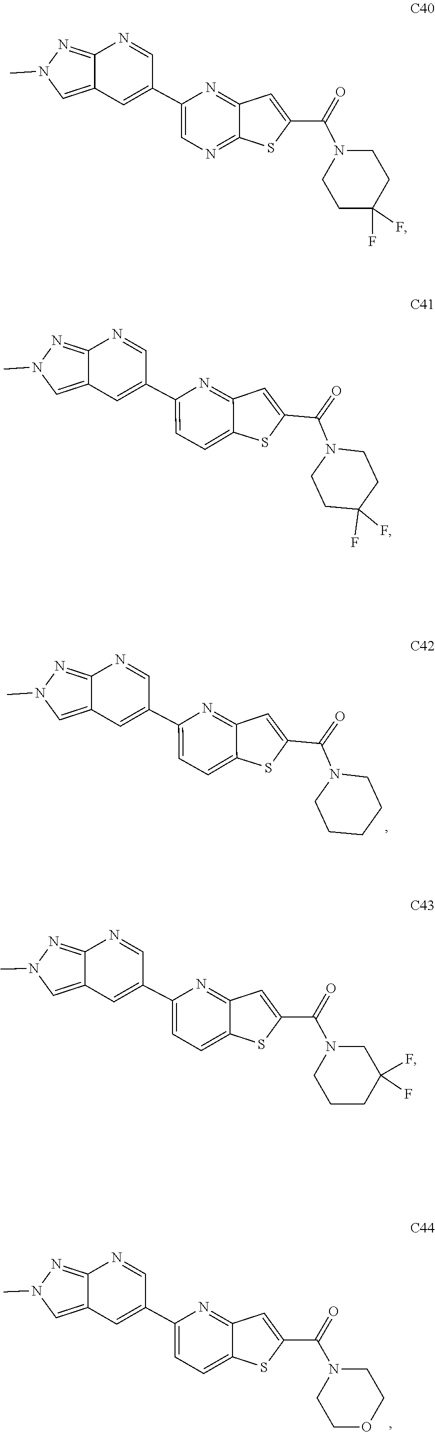 [SMILES: Cn1cc2cc(-c3cnc4sc(C(=O)N5CCC(F)(F)CC5)cc4n3)cnc2n1 ; Cn1cc2cc(-c3ccc4sc(C(=O)N5CCC(F)(F)CC5)cc4n3)cnc2n1 ; Cn1cc2cc(-c3ccc4sc(C(=O)N5CCCCC5)cc4n3)cnc2n1 ; Cn1cc2cc(-c3ccc4sc(C(=O)N5CCCC(F)(F)C5)cc4n3)cnc2n1 ; Cn1cc2cc(-c3ccc4sc(C(=O)N5CCOCC5)cc4n3)cnc2n1]

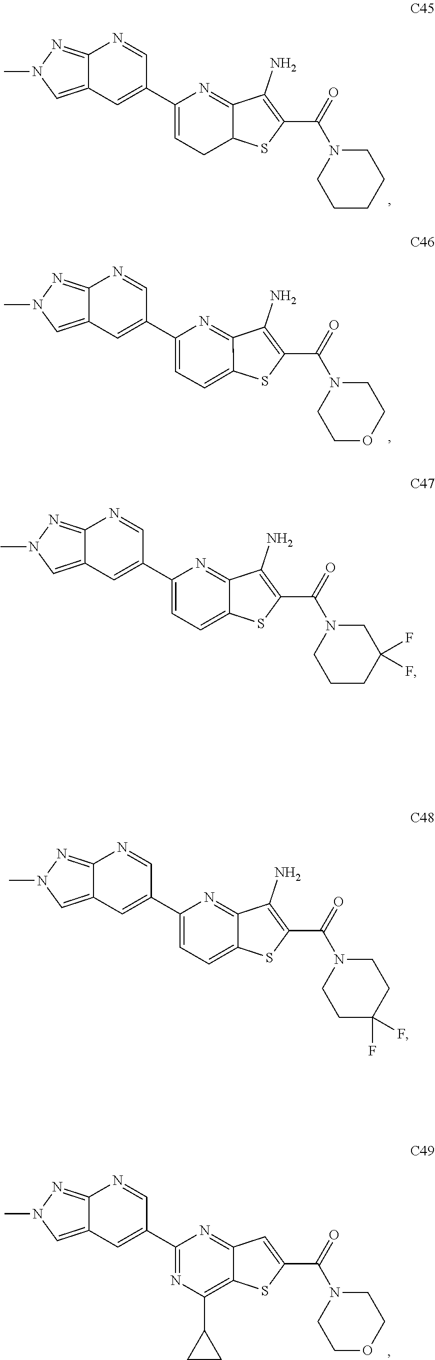 [SMILES: Cn1cc2cc(C3=CCC4SC(C(=O)N5CCCCC5)=C(N)C4=N3)cnc2n1 ; Cn1cc2cc(-c3ccc4sc(C(=O)N5CCOCC5)c(N)c4n3)cnc2n1 ; Cn1cc2cc(-c3ccc4sc(C(=O)N5CCCC(F)(F)C5)c(N)c4n3)cnc2n1 ; Cn1cc2cc(-c3ccc4sc(C(=O)N5CCC(F)(F)CC5)c(N)c4n3)cnc2n1 ; Cn1cc2cc(-c3nc(C4CC4)c4sc(C(=O)N5CCOCC5)cc4n3)cnc2n1]

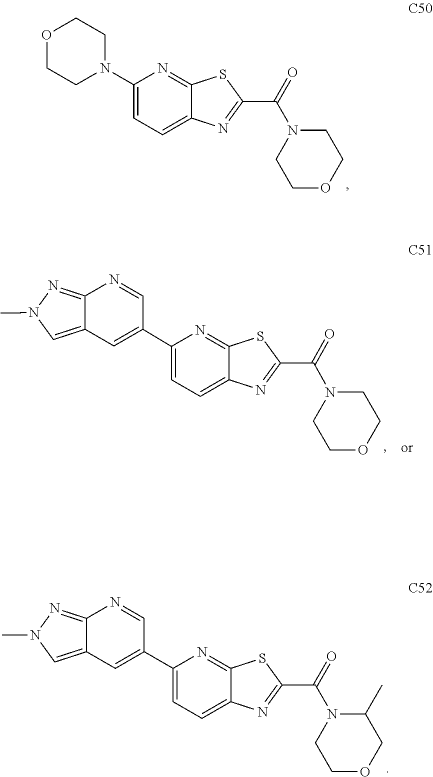

Provided herein as Embodiment 220 is the compound or salt of Embodiment 1, wherein the compound is:

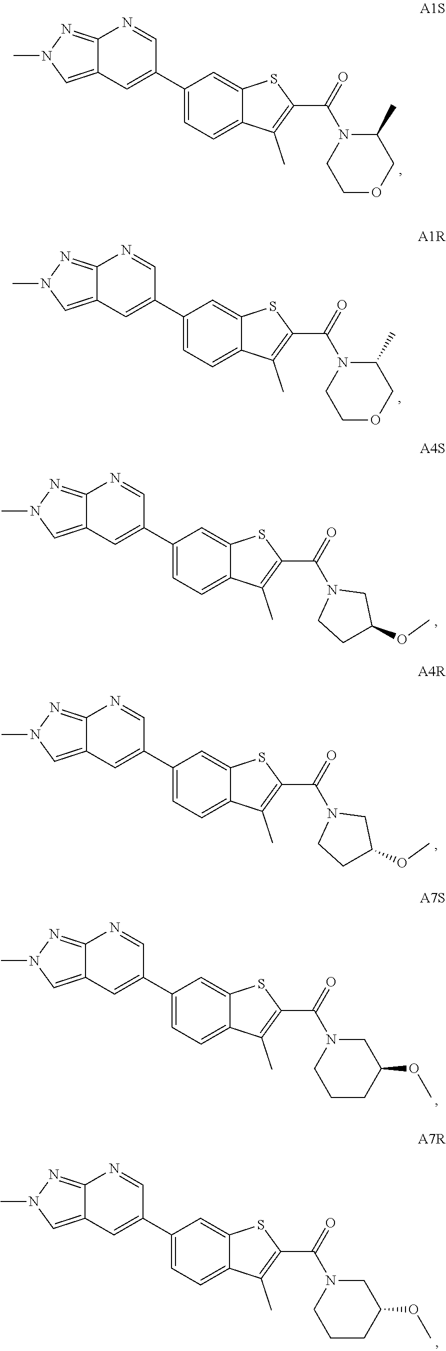

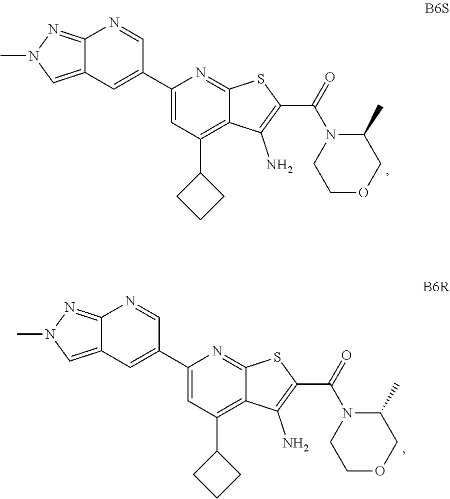

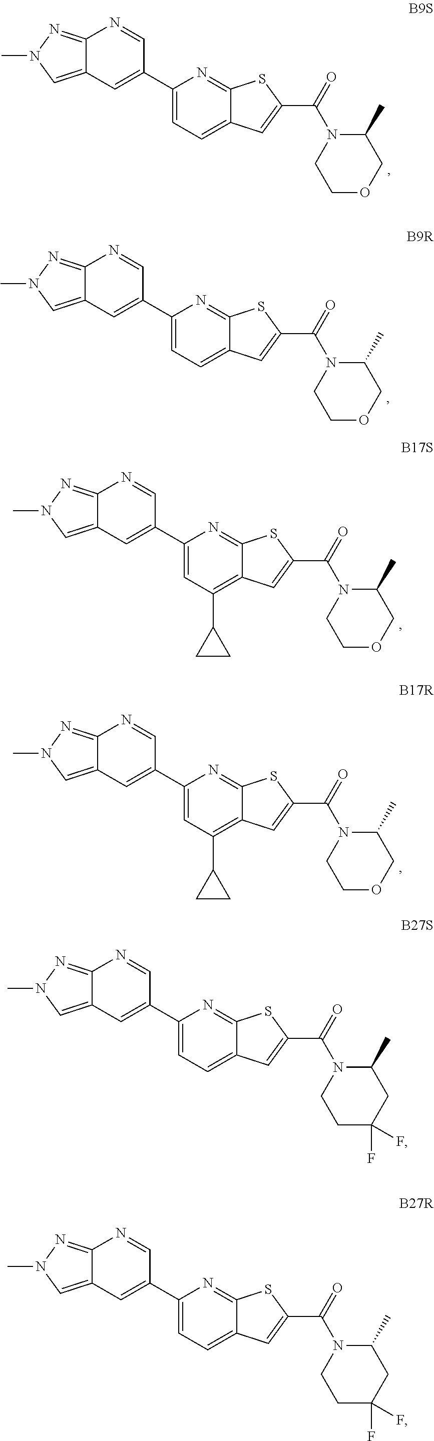

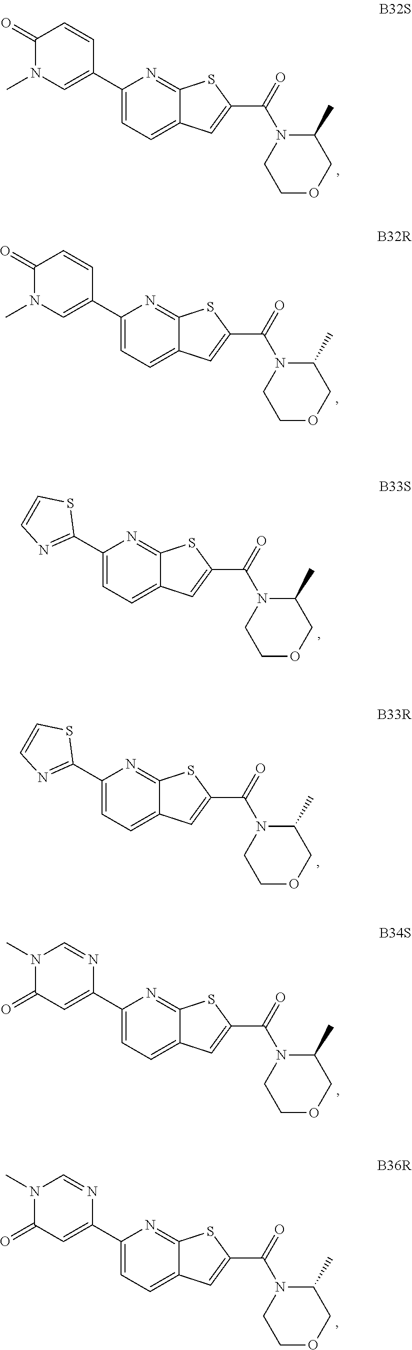

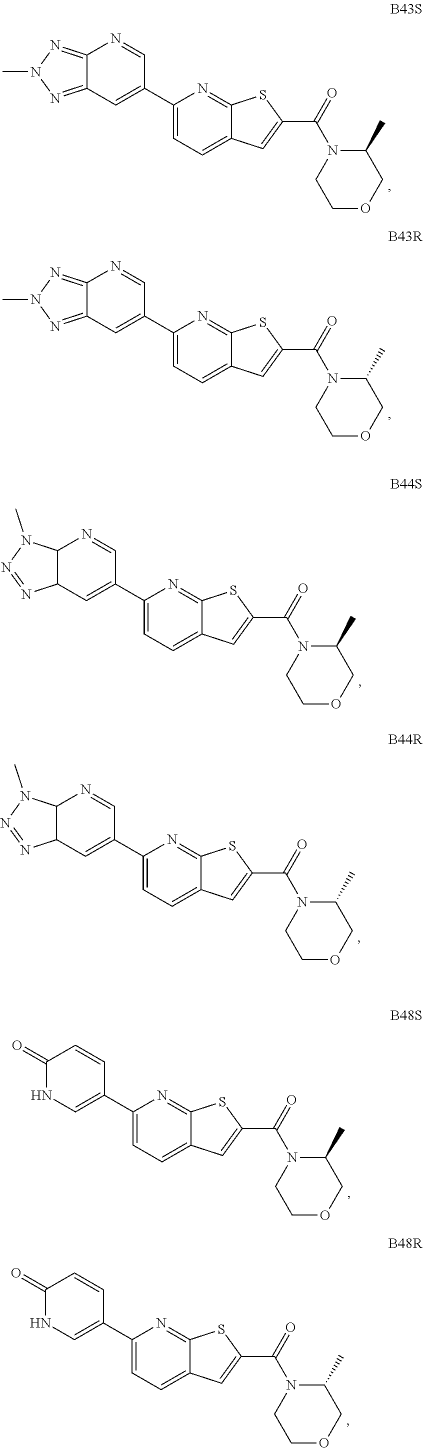

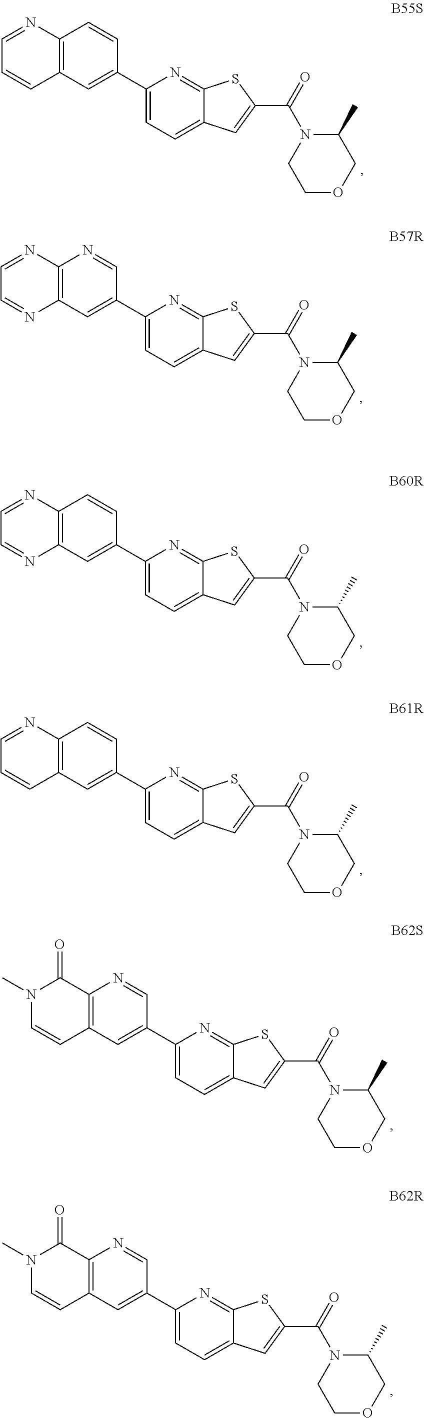

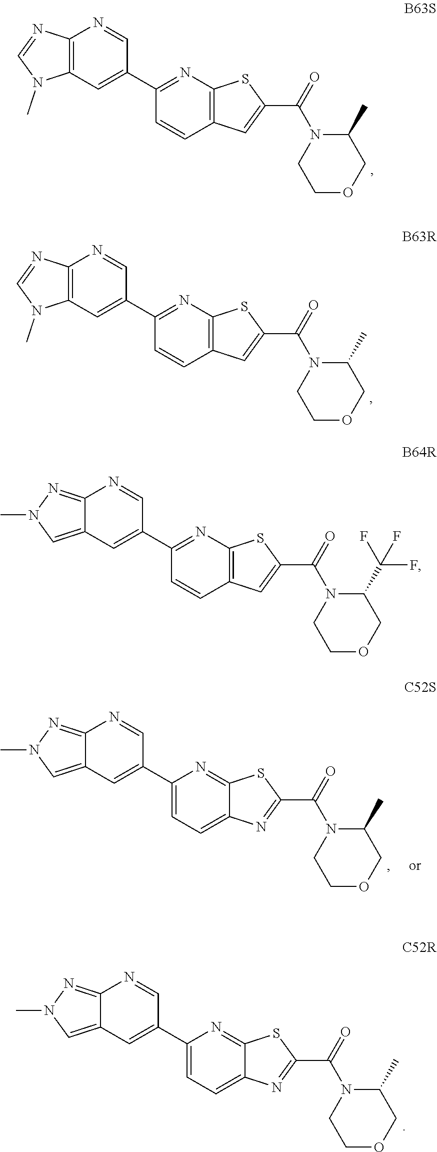

Provided herein as Embodiment 221 is the compound or salt of Embodiment 1, wherein the compound is:
(3-methyl-6-(2-methyl-2H-pyrazolo[3,4-b]pyridin-5-yl)-1-benzothiophen-2-yl)(3-methyl-4-morpholinyl)methanone;
(3-methyl-6-(2-methyl-2H-pyrazolo[3,4-b]pyridin-5-yl)-1-benzothiophen-2-yl)(4-morpholinyl)methanone;
(4,4-difluoro-1-piperidinyl)(3-methyl-6-(2-methyl-2H-pyrazolo[3,4-b]pyridin-5-yl)-1-benzothiophen-2-yl)methanone;
(3-methoxy-1-pyrrolidinyl)(3-methyl-6-(2-methyl-2H-pyrazolo[3,4-b]pyridin-5-yl)-1-benzothiophen-2-yl)methanone;
N-(2-methoxyethyl)-N,3-dimethyl-6-(2-methyl-2H-pyrazolo[3,4-b]pyridin-5-yl)-1-benzothiophene-2-carboxamide;
(2,2-dimethyl-4-morpholinyl)(3-methyl-6-(2-methyl-2H-pyrazolo[3,4-b]pyridin-5-yl)-1-benzothiophen-2-yl)methanone;
(3-methoxy-1-piperidinyl)(3-methyl-6-(2-methyl-2H-pyrazolo[3,4-b]pyridin-5-yl)-1-benzothiophen-2-yl)methanone;
(3-methyl-6-(5-pyrimidinyl)-1-benzothiophen-2-yl)(4-morpholinyl)methanone;
1-methyl-5-(3-methyl-2-(4-morpholinylcarbonyl)-1-benzothiophen-6-yl)-2(1H)-pyridinone;
(6-(2-((2-hydroxyethyl)amino)-5-pyrimidinyl)-3-methyl-1-benzothiophen-2-yl)(4-morpholinyl)methanone;
(3-methyl-6-(2-methyl-2H-indazol-5-yl)-1-benzothiophen-2-yl)(4-morpholinyl)methanone;
(3-amino-4-cyclobutyl-6-(2-methyl-2H-pyrazolo[3,4-b]pyridin-5-yl)thieno[2,3-b]pyridin-2-yl)(4-morpholinyl)methanone;
(3-amino-4-cyclobutyl-6-(2-methyl-2H-pyrazolo[3,4-b]pyridin-5-yl)thieno[2,3-b]pyridin-2-yl)(4,4-difluoro-1-piperidinyl)methanone;
(3-amino-4-cyclobutyl-6-(2-methyl-2H-pyrazolo[3,4-b]pyridin-5-yl)thieno[2,3-b]pyridin-2-yl)(1-piperidinyl)methanone;
(3-amino-4-cyclobutyl-6-(2-methyl-2H-pyrazolo[3,4-b]pyridin-5-yl)thieno[2,3-b]pyridin-2-yl)(3-methyl-4-morpholinyl)methanone;
(7-oxido-4-(2-propanyl)thieno[2,3-b]pyridin-2-yl)(1-piperidinyl)methanone;
(6-(2-methyl-2H-pyrazolo[3,4-b]pyridin-5-yl)thieno[2,3-b]pyridin-2-yl)(4-morpholinyl)methanone;
(3-methyl-4-morpholinyl)(6-(2-methyl-2H-pyrazolo[3,4-b]pyridin-5-yl)thieno[2,3-b]pyridin-2-yl)methanone;
(4-cyclopropyl-6-(2-methyl-2H-pyrazolo[3,4-b]pyridin-5-yl)thieno[2,3-b]pyridin-2-yl)(4-morpholinyl)methanone;
(4-cyclopropyl-6-(2-methyl-2H-pyrazolo[3,4-b]pyridin-5-yl)thieno[2,3-b]pyridin-2-yl)(3,3-difluoro-1-piperidinyl)methanone;
4-cyclopropyl-N-(2-methoxyethyl)-N-methyl-6-(2-methyl-2H-pyrazolo[3,4-b]pyridin-5-yl)thieno[2,3-b]pyridine-2-carboxamide;
(6-(2-methyl-2H-pyrazolo[3,4-b]pyridin-5-yl)-4-(1-methyl-1H-pyrazol-5-yl)thieno[2,3-b]pyridin-2-yl)(4-morpholinyl)methanone;
(6-(2-methyl-2H-pyrazolo[3,4-b]pyridin-5-yl)-4-(1-methyl-1H-pyrazol-5-yl)thieno[2,3-b]pyridin-2-yl)(4-thiomorpholinyl)methanone;
N-cyclobutyl-4-cyclopropyl-N-methyl-6-(2-methyl-2H-pyrazolo[3,4-b]pyridin-5-yl)thieno[2,3-b]pyridine-2-carboxamide;
(5-(2-methyl-2H-pyrazolo[3,4-b]pyridin-5-yl)thieno[2,3-b]pyridin-2-yl)(1-piperidinyl)methanone;
(4-cyclopropyl-6-(2-methyl-2H-pyrazolo[3,4-b]pyridin-5-yl)thieno[2,3-b]pyridin-2-yl)(3-methyl-4-morpholinyl)methanone;
2-methyl-2-propanyl 4-(5-(4-cyclopropyl-2-(1-piperidinylcarbonyl)thieno[2,3-b]pyridin-6-yl)-2-pyrimidinyl)-1-piperazinecarboxylate;
(4-cyclopropyl-6-(2-methyl-2H-pyrazolo[3,4-b]pyridin-5-yl)thieno[2,3-b]pyridin-2-yl)(4,4-difluoro-1-piperidinyl)methanone;
(4-cyclopropyl-6-(2-methyl-2H-pyrazolo[3,4-b]pyridin-5-yl)thieno[2,3-b]pyridin-2-yl)(4-(trifluoromethyl)-1-piperidinyl)methanone;
(6-(2-methyl-2H-pyrazolo[3,4-b]pyridin-5-yl)thieno[2,3-b]pyridin-2-yl)(4-(trifluoromethyl)-1-piperidinyl)methanone;
(4-cyclopropyl-6-(2-(1-piperazinyl)-5-pyrimidinyl)thieno[2,3-b]pyridin-2-yl)(1-piperidinyl)methanone;
(4-cyclopropyl-6-(2-methyl-2H-pyrazolo[3,4-b]pyridin-5-yl)thieno[2,3-b]pyridin-2-yl)(1-piperidinyl)methanone;
(4-cyclopropyl-6-(2-methyl-2H-pyrazolo[3,4-b]pyridin-5-yl)thieno[2,3-b]pyridin-2-yl)(1,4-oxazepan-4-yl)methanone;
(4,4-difluoro-1-piperidinyl)(6-(2-methyl-2H-pyrazolo[3,4-b]pyridin-5-yl)thieno[2,3-b]pyridin-2-yl)methanone;
(4,4-difluoro-2-methyl-1-piperidinyl)(6-(2-methyl-2H-pyrazolo[3,4-b]pyridin-5-yl)thieno[2,3-b]pyridin-2-yl)methanone;
2-methyl-6-(2-(1-piperidinylcarbonyl)thieno[2,3-b]pyridin-5-yl)-1(2H)-isoquinolinone;
(3,3-dimethyl-4-morpholinyl)(6-(2-methyl-2H-pyrazolo[3,4-b]pyridin-5-yl)thieno[2,3-b]pyridin-2-yl)methanone;
4-((6-(2-methyl-2H-pyrazolo[3,4-b]pyridin-5-yl)thieno[2,3-b]pyridin-2-yl)carbonyl)-3-morpholinecarbonitrile;
(2-methyl-4-morpholinyl)(6-(2-methyl-2H-pyrazolo[3,4-b]pyridin-5-yl)thieno[2,3-b]pyridin-2-yl)methanone;
1-methyl-5-(2-((3-methyl-4-morpholinyl)carbonyl)thieno[2,3-b]pyridin-6-yl)-2(1H)-pyridinone;
(3-methyl-4-morpholinyl)(6-(1,3-thiazol-2-yl)thieno[2,3-b]pyridin-2-yl)methanone;
3-methyl-6-(2-((3-methyl-4-morpholinyl)carbonyl)thieno[2,3-b]pyridin-6-yl)-4(3H)-pyrimidinone;
1-methyl-5-(2-(4-morpholinylcarbonyl)thieno[2,3-b]pyridin-6-yl)-2(1H)-pyridinone;
(3-methyl-4-morpholinyl)(6-(1,3-thiazol-2-yl)thieno[2,3-b]pyridin-2-yl)methanone;
3-methyl-6-(2-((3-methyl-4-morpholinyl)carbonyl)thieno[2,3-b]pyridin-6-yl)-4(3H)-pyrimidinone;
(6-(2-methyl-2H-pyrazolo[3,4-b]pyridin-5-yl)thieno[2,3-b]pyridin-2-yl)(8-oxa-3-azabicyclo[3.2.1]octan-3-yl)methanone;
4-morpholinyl(6-(1,3-thiazol-2-yl)thieno[2,3-b]pyridin-2-yl)methanone;
3-methyl-6-(2-(4-morpholinylcarbonyl)thieno[2,3-b]pyridin-6-yl)-4(3H)-pyrimidinone;
(2,2-dimethyl-4-morpholinyl)(6-(2-methyl-2H-pyrazolo[3,4-b]pyridin-5-yl)thieno[2,3-b]pyridin-2-yl)methanone;
5-(2-(4-morpholinylcarbonyl)thieno[2,3-b]pyridin-6-yl)-2(1H)-pyridinone;
(6-(2-methyl-2H-[1,2,3]triazolo[4,5-b]pyridin-6-yl)thieno[2,3-b]pyridin-2-yl)(4-morpholinyl)methanone;
(3-methyl-4-morpholinyl)(6-(2-methyl-2H-[1,2,3]triazolo[4,5-b]pyridin-6-yl)thieno[2,3-b]pyridin-2-yl)methanone;
(3-methyl-4-morpholinyl)(6-(3-methyl-3H-[1,2,3]triazolo[4,5-b]pyridin-6-yl)thieno[2,3-b]pyridin-2-yl)methanone;
(6-(2-methyl-2H-pyrazolo[3,4-b]pyridin-5-yl)thieno[2,3-b]pyridin-2-yl)(7-oxa-4-azaspiro[2.5]octan-4-yl)methanone;
(6-(2-methyl-2H-pyrazolo[3,4-b]pyridin-5-yl)thieno[2,3-b]pyridin-2-yl)(2-(trifluoromethyl)-4-morpholinyl)methanone;
(6-(2-methyl-2H-pyrazolo[3,4-b]pyridin-5-yl)thieno[2,3-b]pyridin-2-yl)(4-oxa-7-azaspiro[2.5]octan-7-yl)methanone;
5-(2-(3-methyl-4-morpholinyl)carbonyl)thieno[2,3-b]pyridin-6-yl)-2(1H)-pyridinone;
4-morpholinyl(6-(6-quinolinyl)thieno[2,3-b]pyridin-2-yl)methanone;
(6-(1-methyl-1H-imidazo[4,5-b]pyridin-6-yl)thieno[2,3-b]pyridin-2-yl)(4-morpholinyl)methanone;
2,8-dioxa-5-azaspiro[3.5]nonan-5-yl(6-(2-methyl-2H-pyrazolo[3,4-b]pyridin-5-yl)thieno[2,3-b]pyridin-2-yl)methanone;
(6-(2-methyl-2H-pyrazolo[3,4-b]pyridin-5-yl)thieno[2,3-b]pyridin-2-yl)(2-oxa-5-azabicyclo[2.2.2]octan-5-yl)methanone;
(6-(2-methyl-2H-pyrazolo[3,4-b]pyridin-5-yl)thieno[2,3-b]pyridin-2-yl)(2-oxa-5-azabicyclo[4.1.0]heptan-5-yl)methanone;
(6-(3-methyl-3H-[1,2,3]triazolo[4,5-b]pyridin-6-yl)thieno[2,3-b]pyridin-2-yl)(4-morpholinyl)methanone;
(3-methyl-4-morpholinyl)(6-(6-quinolinyl)thieno[2,3-b]pyridin-2-yl)methanone;
4-morpholinyl(6-(pyrido[2,3-b]pyrazin-7-yl)thieno[2,3-b]pyridin-2-yl)methanone;
(3-methyl-4-morpholinyl)(6-(pyrido[2,3-b]pyrazin-7-yl)thieno[2,3-b]pyridin-2-yl)methanone;
7-methyl-3-(2-(4-morpholinylcarbonyl)thieno[2,3-b]pyridin-6-yl)-1,7-naphthyridin-8(7H)-one;
(6-(2-methyl-2H-pyrazolo[3,4-b]pyridin-5-yl)thieno[2,3-b]pyridin-2-yl)(8-oxa-5-azaspiro[3.5]nonan-5-yl)methanone;
7-methyl-3-(2-((3-methyl-4-morpholinyl)carbonyl)thieno[2,3-b]pyridin-6-yl)-1,7-naphthyridin-8(7H)-one;
(6-(1-methyl-1H-imidazo[4,5-b]pyridin-6-yl)thieno[2,3-b]pyridin-2-yl)(3-methyl-4-morpholinyl)methanone;
(6-(2-methyl-2H-pyrazolo[3,4-b]pyridin-5-yl)thieno[2,3-b]pyridin-2-yl)(3-(trifluoromethyl)-4-morpholinyl)methanone;
(4-cyclopropyl-2-(2-methyl-2H-pyrazolo[3,4-b]pyridin-5-yl)thieno[2,3-d]pyrimidin-6-yl)(4-morpholinyl)methanone;
(4-cyclopropyl-2-(2-methyl-2H-pyrazolo[3,4-b]pyridin-5-yl)thieno[2,3-d]pyrimidin-6-yl)(1-pyrrolidinyl)methanone;
(4-cyclopropyl-2-(2-methyl-2H-pyrazolo[3,4-b]pyridin-5-yl)thieno[2,3-d]pyrimidin-6-yl)(3,3-difluoro-1-piperidinyl)methanone;
(4-cyclopropyl-2-(2-methyl-2H-pyrazolo[3,4-b]pyridin-5-yl)thieno[2,3-d]pyrimidin-6-yl)(1-piperidinyl)methanone;
(4-cyclopropyl-2-(2-methyl-2H-pyrazolo[3,4-b]pyridin-5-yl)thieno[2,3-d]pyrimidin-6-yl)(3-oxa-8-azabicyclo[3.2.1]octan-8-yl)methanone;
(2-(2-methyl-2H-pyrazolo[3,4-b]pyridin-5-yl)-4-(1-methyl-1H-pyrazol-5-yl)thieno[2,3-d]pyrimidin-6-yl)(4-morpholinyl)methanone;
(2-(2-methyl-2H-pyrazolo[3,4-b]pyridin-5-yl)-4-(1-methyl-1H-pyrazol-5-yl)thieno[2,3-d]pyrimidin-6-yl)(1-piperidinyl)methanone;
(4,4-difluoro-1-piperidinyl)(2-(2-methyl-2H-pyrazolo[3,4-b]pyridin-5-yl)-4-(1-methyl-1H-pyrazol-5-yl)thieno[2,3-d]pyrimidin-6-yl)methanone;
(4,4-difluoro-1-piperidinyl)(4-(1-methyl-1H-pyrazol-5-yl)-2-(2-methyl-2H-[1,2,3]triazolo[4,5-b]pyridin-6-yl)thieno[2,3-d]pyrimidin-6-yl)methanone;
6-(6-((4,4-difluoro-1-piperidinyl)carbonyl)-4-(1-methyl-1H-pyrazol-5-yl)thieno[2,3-d]pyrimidin-2-yl)-3-methyl-4(3H)-pyrimidinone;
(4,4-difluoro-1-piperidinyl)(4-(1-methyl-1H-pyrazol-5-yl)-2-(3-pyridinyl)thieno[2,3-d]pyrimidin-6-yl)methanone;
(4,4-difluoro-1-piperidinyl)(4-(1-methyl-1H-pyrazol-5-yl)-2-(5-pyrimidinyl)thieno[2,3-d]pyrimidin-6-yl)methanone;
(4,4-difluoro-1-piperidinyl)(2-(2-methoxy-5-pyrimidinyl)-4-(1-methyl-1H-pyrazol-5-yl)thieno[2,3-d]pyrimidin-6-yl)methanone;
5-(6-((4,4-difluoro-1-piperidinyl)carbonyl)-4-(1-methyl-1H-pyrazol-5-yl)thieno[2,3-d]pyrimidin-2-yl)-1-methyl-2(1H)-pyridinone;
5-(6-((4,4-difluoro-1-piperidinyl)carbonyl)-4-(1-methyl-H-pyrazol-5-yl)thieno[2,3-d]pyrimidin-2-yl)-2-pyrimidinecarbonitrile;
(4,4-difluoro-1-piperidinyl)(2-(2-(dimethylamino)-5-pyrimidinyl)-4-(1-methyl-1H-pyrazol-5-yl)thieno[2,3-d]pyrimidin-6-yl)methanone;
(4,4-difluoro-1-piperidinyl)(4-(1-methyl-1H-pyrazol-5-yl)-2-(2-methyl-5-pyrimidinyl)thieno[2,3-d]pyrimidin-6-yl)methanone;
(4,4-difluoro-1-piperidinyl)(4-(1-methyl-1H-pyrazol-5-yl)-2-(2-(trifluoromethyl)-5-pyrimidinyl)thieno[2,3-d]pyrimidin-6-yl)methanone;
(2-(2-cyclopropyl-5-pyrimidinyl)-4-(1-methyl-1H-pyrazol-5-yl)thieno[2,3-d]pyrimidin-6-yl)(4,4-difluoro-1-piperidinyl)methanone;
(4,4-difluoro-1-piperidinyl)(2-(1-methyl-1H-pyrazol-4-yl)-4-(1-methyl-JH-pyrazol-5-yl)thieno[2,3-d]pyrimidin-6-yl)methanone;
(4,4-difluoro-1-piperidinyl)(4-(1-methyl-1H-pyrazol-5-yl)-2-(1-(trifluoromethyl)-JH-pyrazol-4-yl)thieno[2,3-d]pyrimidin-6-yl)methanone;
(4,4-difluoro-1-piperidinyl)(2-(1-methyl-1H-pyrazol-3-yl)-4-(1-methyl-JH-pyrazol-5-yl)thieno[2,3-d]pyrimidin-6-yl)methanone;
N-(5-(6-((4,4-difluoro-1-piperidinyl)carbonyl)-4-(1-methyl-1H-pyrazol-5-yl)thieno[2,3-d]pyrimidin-2-yl)-2-pyrimidinyl)acetamide;
(4,4-difluoro-1-piperidinyl)(2-(1-methyl-1H-imidazol-5-yl)-4-(1-methyl-1H-pyrazol-5-yl)thieno[2,3-d]pyrimidin-6-yl)methanone;
(4,4-difluoro-1-piperidinyl)(4-(1-methyl-1H-pyrazol-5-yl)-2-(1,3-thiazol-5-yl)thieno[2,3-d]pyrimidin-6-yl)methanone;
(4,4-difluoro-1-piperidinyl)(4-(1-methyl-1H-pyrazol-5-yl)-2-(1,3-oxazol-5-yl)thieno[2,3-d]pyrimidin-6-yl)methanone;
(4,4-difluoro-1-piperidinyl)(2-(2-(4-methyl-1-piperazinyl)-5-pyrimidinyl)-4-(1-methyl-1H-pyrazol-5-yl)thieno[2,3-d]pyrimidin-6-yl)methanone;
(4,4-difluoro-1-piperidinyl)(4-(1-methyl-1H-pyrazol-5-yl)-2-(2-(4-morpholinyl)-5-pyrimidinyl)thieno[2,3-d]pyrimidin-6-yl)methanone;
5-(6-((4,4-difluoro-1-piperidinyl)carbonyl)-4-(1-methyl-1H-pyrazol-5-yl)thieno[2,3-d]pyrimidin-2-yl)-2(1H)-pyridinone;
(4,4-difluoro-1-piperidinyl)(2-(3H-imidazo[4,5-b]pyridin-6-yl)-4-(1-methyl-1H-pyrazol-5-yl)thieno[2,3-d]pyrimidin-6-yl)methanone;
(4,4-difluoro-1-piperidinyl)(2-(imidazo[1,2-a]pyridin-3-yl)-4-(1-methyl-1H-pyrazol-5-yl)thieno[2,3-d]pyrimidin-6-yl)methanone;
(4,4-difluoro-1-piperidinyl)(4-(1-methyl-1H-pyrazol-5-yl)-2-(4-pyridazinyl)thieno[2,3-d]pyrimidin-6-yl)methanone;
(4,4-difluoro-1-piperidinyl)(4-(1-methyl-1H-pyrazol-5-yl)-2-(2-pyrazinyl)thieno[2,3-d]pyrimidin-6-yl)methanone;
6-(6-((4,4-difluoro-1-piperidinyl)carbonyl)-4-(1-methyl-1H-pyrazol-5-yl)thieno[2,3-d]pyrimidin-2-yl)-2-methyl-1(2H)-isoquinolinone;
(4,4-difluoro-1-piperidinyl)(4-(1-methyl-1H-pyrazol-5-yl)-2-(1,3-thiazol-2-yl)thieno[2,3-d]pyrimidin-6-yl)methanone;
(4,4-difluoro-1-piperidinyl)(4-(1-methyl-1H-pyrazol-5-yl)-2-(1,3-oxazol-2-yl)thieno[2,3-d]pyrimidin-6-yl)methanone;
N-(5-(6-((4,4-difluoro-1-piperidinyl)carbonyl)-4-(1-methyl-JH-pyrazol-5-yl)thieno[2,3-d]pyrimidin-2-yl)-2-pyrimidinyl)methanesulfonamide;
5-(6-((4,4-difluoro-1-piperidinyl)carbonyl)-4-(1-methyl-1H-pyrazol-5-yl)thieno[2,3-d]pyrimidin-2-yl)-2(1H)-pyrimidinone;
(4,4-difluoro-1-piperidinyl)(3-(2-methyl-2H-pyrazolo[3,4-b]pyridin-5-yl)thieno[2,3-b]pyrazin-6-yl)methanone;
(4,4-difluoro-1-piperidinyl)(2-(2-methyl-2H-pyrazolo[3,4-b]pyridin-5-yl)thieno[2,3-b]pyrazin-6-yl)methanone;
(4,4-difluoro-1-piperidinyl)(5-(2-methyl-2H-pyrazolo[3,4-b]pyridin-5-yl)thieno[3,2-b]pyridin-2-yl)methanone;
(5-(2-methyl-2H-pyrazolo[3,4-b]pyridin-5-yl)thieno[3,2-b]pyridin-2-yl)(1-piperidinyl)methanone;
(3,3-difluoro-1-piperidinyl)(5-(2-methyl-2H-pyrazolo[3,4-b]pyridin-5-yl)thieno[3,2-b]pyridin-2-yl)methanone;
(5-(2-methyl-2H-pyrazolo[3,4-b]pyridin-5-yl)thieno[3,2-b]pyridin-2-yl)(4-morpholinyl)methanone;
(3-amino-5-(2-methyl-2H-pyrazolo[3,4-b]pyridin-5-yl)thieno[3,2-b]pyridin-2-yl)(1-piperidinyl)methanone;
(3-amino-5-(2-methyl-2H-pyrazolo[3,4-b]pyridin-5-yl)thieno[3,2-b]pyridin-2-yl)(4-morpholinyl)methanone;
(3-amino-5-(2-methyl-2H-pyrazolo[3,4-b]pyridin-5-yl)thieno[3,2-b]pyridin-2-yl)(3,3-difluoro-1-piperidinyl)methanone;
(3-amino-5-(2-methyl-2H-pyrazolo[3,4-b]pyridin-5-yl)thieno[3,2-b]pyridin-2-yl)(4,4-difluoro-1-piperidinyl)methanone;
(4-cyclopropyl-2-(2-methyl-2H-pyrazolo[3,4-b]pyridin-5-yl)thieno[3,2-d]pyrimidin-6-yl)(4-morpholinyl)methanone;
4-morpholinyl(5-(4-morpholinyl)[1,3]thiazolo[5,4-b]pyridin-2-yl)methanone;
(5-(2-methyl-2H-pyrazolo[3,4-b]pyridin-5-yl)[1,3]thiazolo[5,4-b]pyridin-2-yl)(4-morpholinyl)methanone; and
(3-methyl-4-morpholinyl)(5-(2-methyl-2H-pyrazolo[3,4-b]pyridin-5-yl)[1,3]thiazolo[5,4-b]pyridin-2-yl)methanone.

Provided herein as Embodiment 222 is the compound or salt of Embodiment 1, wherein the compound is:
(3-methyl-6-(2-methyl-2H-pyrazolo[3,4-b]pyridin-5-yl)-1-benzothiophen-2-yl)((3S)-3-methyl-4-morpholinyl)methanone;
(3-methyl-6-(2-methyl-2H-pyrazolo[3,4-b]pyridin-5-yl)-1-benzothiophen-2-yl)((3R)-3-methyl-4-morpholinyl)methanone;
((3R)-3-methoxy-1-pyrrolidinyl)(3-methyl-6-(2-methyl-2H-pyrazolo[3,4-b]pyridin-5-yl)-1-benzothiophen-2-yl)methanone;
((3S)-3-methoxy-1-pyrrolidinyl)(3-methyl-6-(2-methyl-2H-pyrazolo[3,4-b]pyridin-5-yl)-1-benzothiophen-2-yl)methanone;
((3S)-3-methoxy-1-piperidinyl)(3-methyl-6-(2-methyl-2H-pyrazolo[3,4-b]pyridin-5-yl)-1-benzothiophen-2-yl)methanone;
((3R)-3-methoxy-1-piperidinyl)(3-methyl-6-(2-methyl-2H-pyrazolo[3,4-b]pyridin-5-yl)-1-benzothiophen-2-yl)methanone;
(3-amino-4-cyclobutyl-6-(2-methyl-2H-pyrazolo[3,4-b]pyridin-5-yl)thieno[2,3-b]pyridin-2-yl)((3S)-3-methyl-4-morpholinyl)methanone;
(3-amino-4-cyclobutyl-6-(2-methyl-2H-pyrazolo[3,4-b]pyridin-5-yl)thieno[2,3-b]pyridin-2-yl)((3R)-3-methyl-4-morpholinyl)methanone;
((3R)-3-methyl-4-morpholinyl)(6-(2-methyl-2H-pyrazolo[3,4-b]pyridin-5-yl)thieno[2,3-b]pyridin-2-yl)methanone;
((3S)-3-methyl-4-morpholinyl)(6-(2-methyl-2H-pyrazolo[3,4-b]pyridin-5-yl)thieno[2,3-b]pyridin-2-yl)methanone;
(4-cyclopropyl-6-(2-methyl-2H-pyrazolo[3,4-b]pyridin-5-yl)thieno[2,3-b]pyridin-2-yl)((3S)-3-methyl-4-morpholinyl)methanone;
(4-cyclopropyl-6-(2-methyl-2H-pyrazolo[3,4-b]pyridin-5-yl)thieno[2,3-b]pyridin-2-yl)((3R)-3-methyl-4-morpholinyl)methanone;
((2R)-4,4-difluoro-2-methyl-1-piperidinyl)(6-(2-methyl-2H-pyrazolo[3,4-b]pyridin-5-yl)thieno[2,3-b]pyridin-2-yl)methanone;
((2S)-4,4-difluoro-2-methyl-1-piperidinyl)(6-(2-methyl-2H-pyrazolo[3,4-b]pyridin-5-yl)thieno[2,3-b]pyridin-2-yl)methanone;
((2R)-4,4-difluoro-2-methyl-1-piperidinyl)(6-(2-methyl-2H-pyrazolo[3,4-b]pyridin-5-yl)thieno[2,3-b]pyridin-2-yl)methanone;
((2S)-4,4-difluoro-2-methyl-1-piperidinyl)(6-(2-methyl-2H-pyrazolo[3,4-b]pyridin-5-yl)thieno[2,3-b]pyridin-2-yl)methanone;
(3R)-4-((6-(2-methyl-2H-pyrazolo[3,4-b]pyridin-5-yl)thieno[2,3-b]pyridin-2-yl)carbonyl)-3-morpholinecarbonitrile;
(3S)-4-((6-(2-methyl-2H-pyrazolo[3,4-b]pyridin-5-yl)thieno[2,3-b]pyridin-2-yl)carbonyl)-3-morpholinecarbonitrile;
((2R)-2-methyl-4-morpholinyl)(6-(2-methyl-2H-pyrazolo[3,4-b]pyridin-5-yl)thieno[2,3-b]pyridin-2-yl)methanone;
((2S)-2-methyl-4-morpholinyl)(6-(2-methyl-2H-pyrazolo[3,4-b]pyridin-5-yl)thieno[2,3-b]pyridin-2-yl)methanone;
1-methyl-5-(2-(((3S)-3-methyl-4-morpholinyl)carbonyl)thieno[2,3-b]pyridin-6-yl)-2(1H)-pyridinone;
1-methyl-5-(2-(((3R)-3-methyl-4-morpholinyl)carbonyl)thieno[2,3-b]pyridin-6-yl)-2(1H)-pyridinone;
((3S)-3-methyl-4-morpholinyl)(6-(1,3-thiazol-2-yl)thieno[2,3-b]pyridin-2-yl)methanone;
3-methyl-6-(2-(((3S)-3-methyl-4-morpholinyl)carbonyl)thieno[2,3-b]pyridin-6-yl)-4(3H)-pyrimidinone;
((3R)-3-methyl-4-morpholinyl)(6-(1,3-thiazol-2-yl)thieno[2,3-b]pyridin-2-yl)methanone;
3-methyl-6-(2-(((3R)-3-methyl-4-morpholinyl)carbonyl)thieno[2,3-b]pyridin-6-yl)-4(3H)-pyrimidinone;
(6-(2-methyl-2H-pyrazolo[3,4-b]pyridin-5-yl)thieno[2,3-b]pyridin-2-yl)((1R,5R)-8-oxa-3-azabicyclo[3.2.1]octan-3-yl)methanone;
(6-(2-methyl-2H-pyrazolo[3,4-b]pyridin-5-yl)thieno[2,3-b]pyridin-2-yl)((1R,5S)-8-oxa-3-azabicyclo[3.2.1]octan-3-yl)methanone;
(6-(2-methyl-2H-pyrazolo[3,4-b]pyridin-5-yl)thieno[2,3-b]pyridin-2-yl)((1S,5S)-8-oxa-3-azabicyclo[3.2.1]octan-3-yl)methanone;
((3S)-3-methyl-4-morpholinyl)(6-(2-methyl-2H-[1,2,3]triazolo[4,5-b]pyridin-6-yl)thieno[2,3-b]pyridin-2-yl)methanone;
((3R)-3-methyl-4-morpholinyl)(6-(2-methyl-2H-[1,2,3]triazolo[4,5-b]pyridin-6-yl)thieno[2,3-b]pyridin-2-yl)methanone;
((3R)-3-methyl-4-morpholinyl)(6-(3-methyl-3H-[1,2,3]triazolo[4,5-b]pyridin-6-yl)thieno[2,3-b]pyridin-2-yl)methanone;
((3S)-3-methyl-4-morpholinyl)(6-(3-methyl-3H-[1,2,3]triazolo[4,5-b]pyridin-6-yl)thieno[2,3-b]pyridin-2-yl)methanone;
(6-(2-methyl-2H-pyrazolo[3,4-b]pyridin-5-yl)thieno[2,3-b]pyridin-2-yl)((2R)-2-(trifluoromethyl)-4-morpholinyl)methanone;
(6-(2-methyl-2H-pyrazolo[3,4-b]pyridin-5-yl)thieno[2,3-b]pyridin-2-yl)((2S)-2-(trifluoromethyl)-4-morpholinyl)methanone;
5-(2-(((3S)-3-methyl-4-morpholinyl)carbonyl)thieno[2,3-b]pyridin-6-yl)-2(1H)-pyridinone;
(6-(2-methyl-2H-pyrazolo[3,4-b]pyridin-5-yl)thieno[2,3-b]pyridin-2-yl)((1R,4R)-2-oxa-5-azabicyclo[2.2.2]octan-5-yl)methanone;
(6-(2-methyl-2H-pyrazolo[3,4-b]pyridin-5-yl)thieno[2,3-b]pyridin-2-yl)((1R,4S)-2-oxa-5-azabicyclo[2.2.2]octan-5-yl)methanone;
(6-(2-methyl-2H-pyrazolo[3,4-b]pyridin-5-yl)thieno[2,3-b]pyridin-2-yl)((1S,4R)-2-oxa-5-azabicyclo[2.2.2]octan-5-yl)methanone;
(6-(2-methyl-2H-pyrazolo[3,4-b]pyridin-5-yl)thieno[2,3-b]pyridin-2-yl)((1S,4S)-2-oxa-5-azabicyclo[2.2.2]octan-5-yl)methanone;
(6-(2-methyl-2H-pyrazolo[3,4-b]pyridin-5-yl)thieno[2,3-b]pyridin-2-yl)((1R,6R)-2-oxa-5-azabicyclo[4.1.0]heptan-5-yl)methanone;
(6-(2-methyl-2H-pyrazolo[3,4-b]pyridin-5-yl)thieno[2,3-b]pyridin-2-yl)((1R,6S)-2-oxa-5-azabicyclo[4.1.0]heptan-5-yl)methanone;
(6-(2-methyl-2H-pyrazolo[3,4-b]pyridin-5-yl)thieno[2,3-b]pyridin-2-yl)((1S,6R)-2-oxa-5-azabicyclo[4.1.0]heptan-5-yl)methanone;
(6-(2-methyl-2H-pyrazolo[3,4-b]pyridin-5-yl)thieno[2,3-b]pyridin-2-yl)((1S,6S)-2-oxa-5-azabicyclo[4.1.0]heptan-5-yl)methanone;
5-(2-(((3R)-3-methyl-4-morpholinyl)carbonyl)thieno[2,3-b]pyridin-6-yl)-2(1H)-pyridinone;
((3S)-3-methyl-4-morpholinyl)(6-(6-quinolinyl)thieno[2,3-b]pyridin-2-yl)methanone;
((3S)-3-methyl-4-morpholinyl)(6-(pyrido[2,3-b]pyrazin-7-yl)thieno[2,3-b]9lyridine-2-yl)methanone;
((3R)-3-methyl-4-morpholinyl)(6-(pyrido[2,3-b]pyrazin-7-yl)thieno[2,3-b]pyridin-2-yl)methanone;
((3R)-3-methyl-4-morpholinyl)(6-(6-quinolinyl)thieno[2,3-b]pyridin-2-yl)methanone;
7-methyl-3-(2-(((3S)-3-methyl-4-morpholinyl)carbonyl)thieno[2,3-b]pyridin-6-yl)-1,7-naphthyridin-8(7H)-one;
(6-(1-methyl-1H-imidazo[4,5-b]pyridin-6-yl)thieno[2,3-b]pyridin-2-yl)((3S)-3-methyl-4-morpholinyl)methanone;
(6-(1-methyl-1H-imidazo[4,5-b]pyridin-6-yl)thieno[2,3-b]pyridin-2-yl)((3R)-3-methyl-4-morpholinyl)methanone;
7-methyl-3-(2-(((3R)-3-methyl-4-morpholinyl)carbonyl)thieno[2,3-b]pyridin-6-yl)-1,7-naphthyridin-8(7H)-one;
(6-(2-methyl-2H-pyrazolo[3,4-b]pyridin-5-yl)thieno[2,3-b]pyridin-2-yl)((3R)-3-(trifluoromethyl)-4-morpholinyl)methanone;
(6-(2-methyl-2H-pyrazolo[3,4-b]pyridin-5-yl)thieno[2,3-b]pyridin-2-yl)((3S)-3-(trifluoromethyl)-4-morpholinyl)methanone;
(4-cyclopropyl-2-(2-methyl-2H-pyrazolo[3,4-b]pyridin-5-yl)thieno[2,3-d]pyrimidin-6-yl)((1R,5S)-3-oxa-8-azabicyclo[3.2.1]octan-8-yl)methanone;
((3R)-3-methyl-4-morpholinyl)(5-(2-methyl-2H-pyrazolo[3,4-b]pyridin-5-yl)[1,3]thiazolo[5,4-b]pyridin-2-yl)methanone; or
((3S)-3-methyl-4-morpholinyl)(5-(2-methyl-2H-pyrazolo[3,4-b]pyridin-5-yl)[1,3]thiazolo[5,4-b]pyridin-2-yl)methanone.

Provided herein as Embodiment 223 is the compound or salt of Embodiment 1, wherein the compound is:

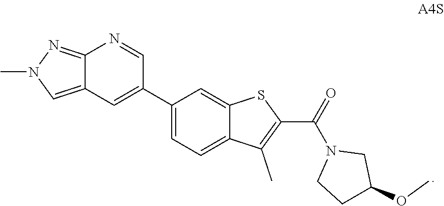

Provided herein as Embodiment 224 is the compound or salt of Embodiment 1, wherein the compound is:

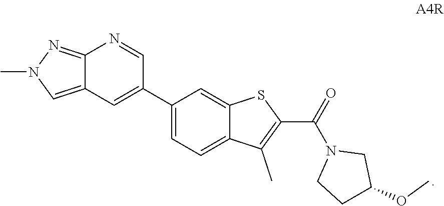

Provided herein as Embodiment 225 is the compound or salt of Embodiment 1, wherein the compound is:

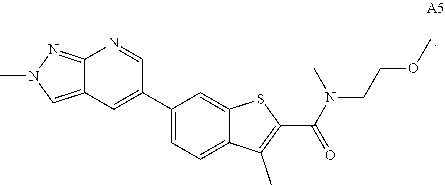

Provided herein as Embodiment 226 is the compound or salt of Embodiment 1, wherein the compound is:

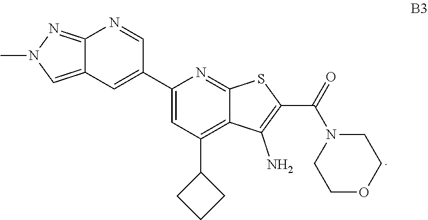 [SMILES: Cn1cc2cc(-c3cc(C4CCC4)c4c(N)c(C(=O)N5CCOCC5)sc4n3)cnc2n1]

Provided herein as Embodiment 227 is the compound or salt of Embodiment 1, wherein the compound is:

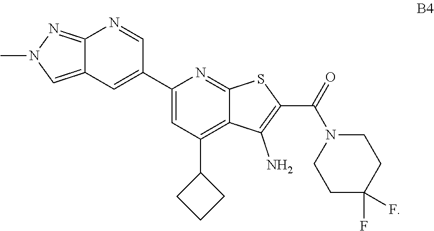 [SMILES: Cn1cc2cc(-c3cc(C4CCC4)c4c(N)c(C(=O)N5CCC(F)(F)CC5)sc4n3)cnc2n1]

Provided herein as Embodiment 228 is the compound or salt of Embodiment 1, wherein the compound is:

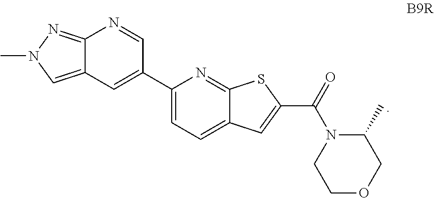

Provided herein as Embodiment 229 is the compound or salt of Embodiment 1 wherein the compound is:

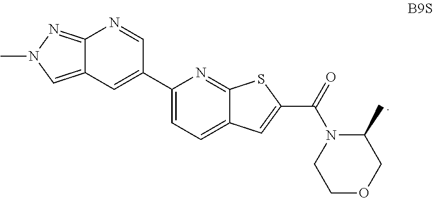

Provided herein as Embodiment 230 is the compound or salt of Embodiment 1, wherein the compound is:

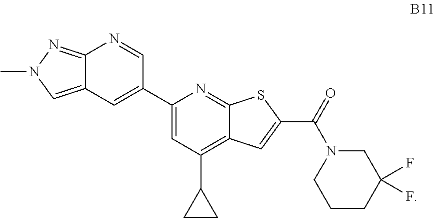 [SMILES: Cn1cc2cc(-c3cc(C4CC4)c4cc(C(=O)N5CCCC(F)(F)C5)sc4n3)cnc2n1]

Provided herein as Embodiment 231 is the compound or salt of Embodiment 1, wherein the compound is:

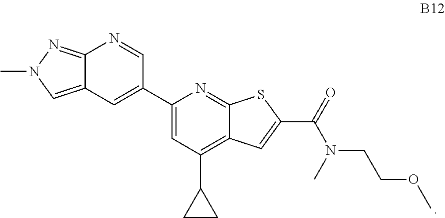

Provided herein as Embodiment 232 is the compound or salt of Embodiment 1, wherein the compound is:

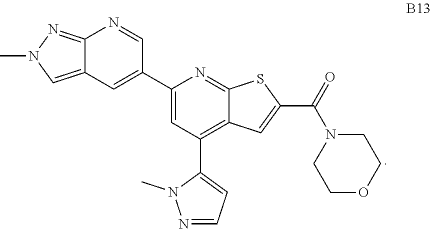 [SMILES: Cn1cc2cc(-c3cc(-c4ccnn4C)c4cc(C(=O)N5CCOCC5)sc4n3)cnc2n1]

Provided herein as Embodiment 233 is the compound or salt of Embodiment 1, wherein the compound is:

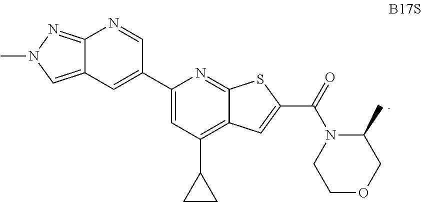

Provided herein as Embodiment 234 is the compound or salt of Embodiment 1, wherein the compound is:

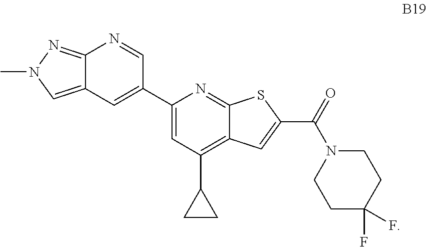 [SMILES: Cn1cc2cc(-c3cc(C4CC4)c4cc(C(=O)N5CCC(F)(F)CC5)sc4n3)cnc2n1]

Provided herein as Embodiment 235 is the compound or salt of Embodiment 1, wherein the compound is:

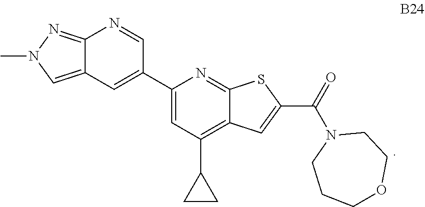 [SMILES: Cn1cc2cc(-c3cc(C4CC4)c4cc(C(=O)N5CCCOCC5)sc4n3)cnc2n1]

Provided herein as Embodiment 236 is the compound or salt of Embodiment 1, wherein the compound is:

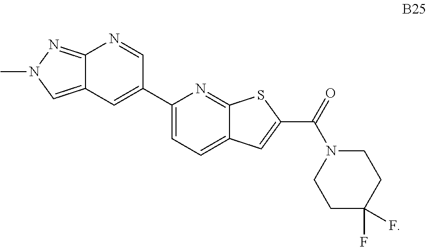 [SMILES: Cn1cc2cc(-c3ccc4cc(C(=O)N5CCC(F)(F)CC5)sc4n3)cnc2n1]

Provided herein as Embodiment 237 is the compound or salt of Embodiment 1, wherein the compound is:

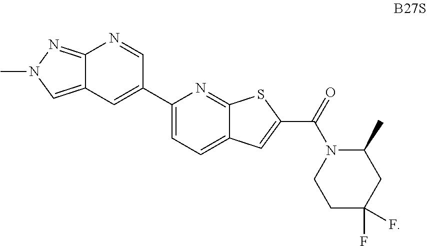 [SMILES: C[C@H]1CC(F)(F)CCN1C(=O)c1cc2ccc(-c3cnc4nn(C)cc4c3)nc2s1]

Provided herein as Embodiment 238 is the compound or salt of Embodiment 1, wherein the compound is:

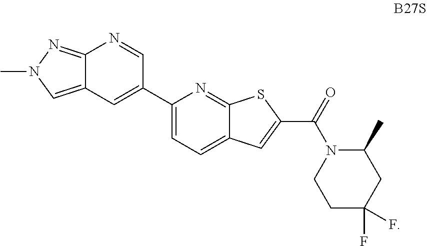 [SMILES: C[C@H]1CC(F)(F)CCN1C(=O)c1cc2ccc(-c3cnc4nn(C)cc4c3)nc2s1]

Provided herein as Embodiment 239 is the compound or salt of Embodiment 1, wherein the compound is:

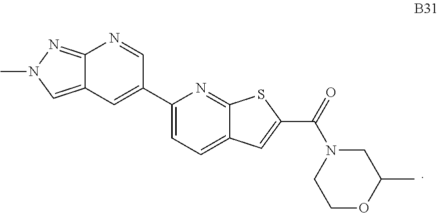

Provided herein as Embodiment 240 is the compound or salt of Embodiment 1, wherein the compound is:

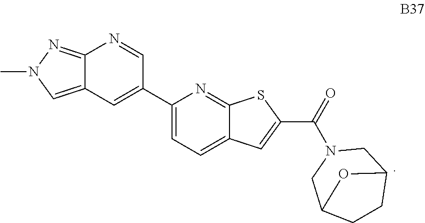 [SMILES: Cn1cc2cc(-c3ccc4cc(C(=O)N5CC6CCC(C5)O6)sc4n3)cnc2n1]

Provided herein as Embodiment 241 is the compound or salt of Embodiment 1, wherein the compound is:

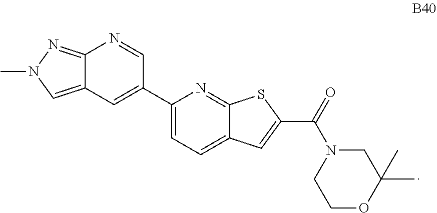

Provided herein as Embodiment 242 is the compound or salt of Embodiment 1, wherein the compound is:

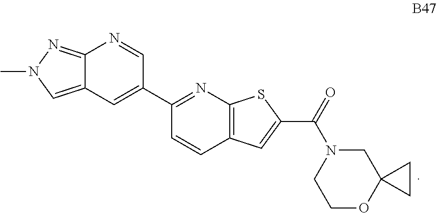 [SMILES: Cn1cc2cc(-c3ccc4cc(C(=O)N5CCOC6(CC6)C5)sc4n3)cnc2n1]

Provided herein as Embodiment 243 is the compound or salt of Embodiment 1, wherein the compound is:

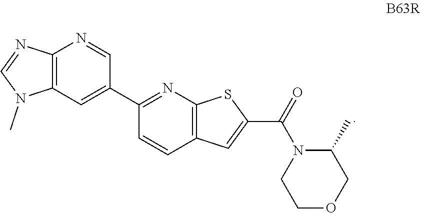 [SMILES: C[C@@H]1COCCN1C(=O)c1cc2ccc(-c3cnc4ncn(C)c4c3)nc2s1]

Provided herein as Embodiment 244 is the compound or salt of Embodiment 1, wherein the compound is:

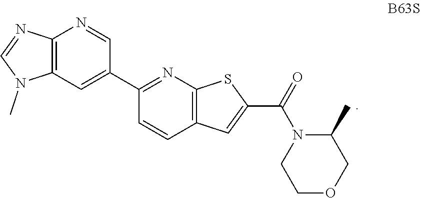 [SMILES: C[C@H]1COCCN1C(=O)c1cc2ccc(-c3cnc4ncn(C)c4c3)nc2s1]

Provided herein as Embodiment 245 is the compound or salt of Embodiment 1, wherein the compound is:

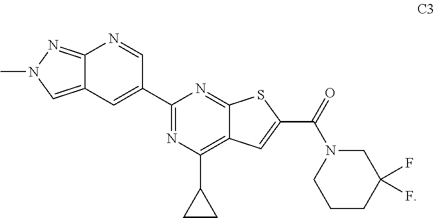 [SMILES: Cn1cc2cc(-c3nc(C4CC4)c4cc(C(=O)N5CCCC(F)(F)C5)sc4n3)cnc2n1]

Provided herein as Embodiment 246 is the compound or salt of Embodiment 1, wherein the compound is:

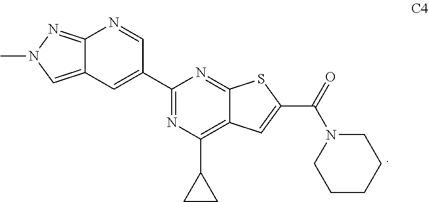 [SMILES: Cn1cc2cc(-c3nc(C4CC4)c4cc(C(=O)N5CCCCC5)sc4n3)cnc2n1]

Provided herein as Embodiment 247 is the compound or salt of Embodiment 1, wherein the compound is:

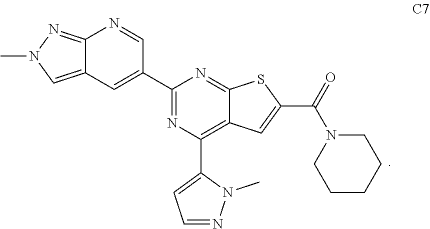 [SMILES: Cn1cc2cc(-c3nc(-c4ccnn4C)c4cc(C(=O)N5CCCCC5)sc4n3)cnc2n1]

Provided herein as Embodiment 248 is the compound or salt of Embodiment 1, wherein the compound is:

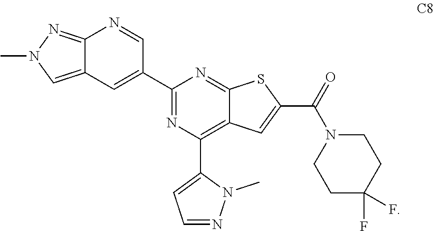 [SMILES: Cn1cc2cc(-c3nc(-c4ccnn4C)c4cc(C(=O)N5CCC(F)(F)CC5)sc4n3)cnc2n1]

Provided herein as Embodiment 249 is the compound or salt of Embodiment 1, wherein the compound is:

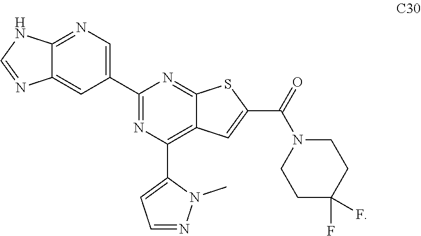 [SMILES: Cn1nccc1-c1nc(-c2cnc3[nH]cnc3c2)nc2sc(C(=O)N3CCC(F)(F)CC3)cc12]

Provided herein as Embodiment 250 is the compound or salt of Embodiment 1, wherein the compound is:

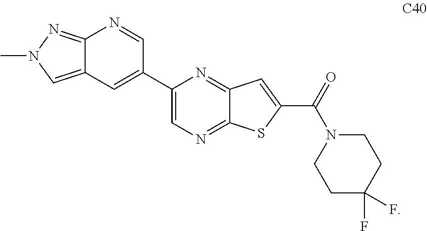 [SMILES: Cn1cc2cc(-c3cnc4sc(C(=O)N5CCC(F)(F)CC5)cc4n3)cnc2n1]

Provided herein as Embodiment 251 is the compound or salt of Embodiment 1, wherein the compound is:

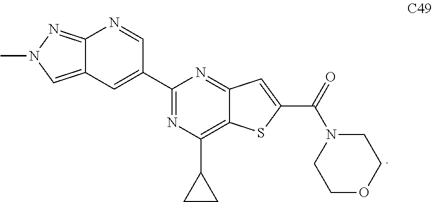 [SMILES: Cn1cc2cc(-c3nc(C4CC4)c4sc(C(=O)N5CCOCC5)cc4n3)cnc2n1]

Provided herein as Embodiment 252 is the compound or salt of Embodiment 1, wherein the compound is:

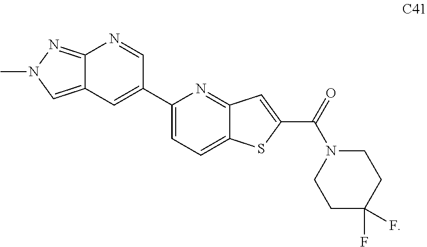 [SMILES: Cn1cc2cc(-c3ccc4sc(C(=O)N5CCC(F)(F)CC5)cc4n3)cnc2n1]

Provided herein as Embodiment 253 is the compound or salt of Embodiment 1, wherein the compound is:

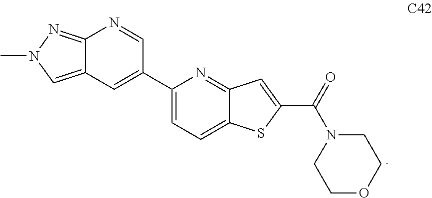 [SMILES: Cn1cc2cc(-c3ccc4sc(C(=O)N5CCOCC5)cc4n3)cnc2n1]

Provided herein as Embodiment 254 is the compound of any one of Embodiments 1 to 253.

The compounds or salts disclosed in Embodiments 1-254 may be stereoisomerically pure or stereoisomerically enriched.

If the stereochemistry of a structure or a portion of a structure disclosed herein is not explicitly shown (e.g., such as with dashed or bold lines), then the structure or portion of structure is either achiral or interpreted as being any of the possible stereoisomers of the structure or portion of the structure. If the stereochemistry of a structure or portion of a structure is explicitly shown, a single stereoisomer of the structure or portion of the structure is represented, with the understanding that the stereochemistry of the structure or portion of the structure may have been arbitrarily assigned.

The term “stereoisomer” or “stereoisomerically pure” compound as used herein refers to one stereoisomer (for example, geometric isomer, enantiomer, diastereomer and atropoisomer) of a compound that is substantially free of other stereoisomers of that compound. For example, a stereoisomerically pure compound having one chiral center will be substantially free of the mirror image enantiomer of the compound and a stereoisomerically pure compound having two chiral centers will be substantially free of other enantiomers or diastereomers of the compound.

A typical stereoisomerically enriched compound comprises greater than about 50% by weight of one stereoisomer of the compound and equal or less than about 50% by weight of other stereoisomers of the compound, greater than about 60% by weight of one stereoisomer of the compound and equal or less than about 40% by weight of other stereoisomers of the compound, greater than about 70% by weight of one stereoisomer of the compound and equal or less than about 30% by weight of other stereoisomers of the compound, greater than about 80% by weight of one stereoisomer of the compound and equal or less than about 20% by weight of other stereoisomers of the compound, greater than about 90% by weight of one stereoisomer of the compound and equal or less than about 10% by weight of the other stereoisomers of the compound, greater than about 95% by weight of one stereoisomer of the compound and equal or less than about 5% by weight of the other stereoisomers of the compound, greater than about 97% by weight of one stereoisomer of the compound and equal or less than about 3% by weight of other stereoisomers of the compound, greater than about 99% by weight of one stereoisomer of the compound and equal or less than about 1% by weight of other stereoisomers of the compound, or greater than about 99.9% by weight of one stereoisomer of the compound and equal or less than about 0.1% by weight of other stereoisomers of the compound.

This disclosure also encompasses the pharmaceutical compositions comprising stereoisomerically pure forms (or enriched forms) and the use of stereoisomerically pure forms (or enriched forms) of any compounds disclosed herein. Further, this disclosure also encompasses pharmaceutical compositions comprising mixtures of stereoisomers of any compounds disclosed herein and the use of said pharmaceutical compositions or mixtures of stereoisomers. These stereoisomers or mixtures thereof may be synthesized in accordance with methods well known in the art and methods disclosed herein. Mixtures of stereoisomers may be resolved using standard techniques, such as chiral columns or chiral resolving agents. See, for example, Jacques et al., Enantiomers, Racemates and Resolutions (Wiley-Interscience, New York, 1981); Wilen et al., Tetrahedron 33:2725; Eliel, Stereochemistry of Carbon Compounds (McGraw-Hill, NY, 1962); and Wilen, Tables of Resolving Agents and Optical Resolutions, page 268 (Eliel, Ed., Univ. of Notre Dame Press, Notre Dame, IN, 1972).

As known by those skilled in the art, certain compounds disclosed herein may exist in one or more tautomeric forms. Because one chemical structure may only be used to represent one tautomeric form, it will be understood that for convenience, referral to a compound of a given structural formula includes other tautomers of said structural formula. Accordingly, the scope of the instant disclosure is to be understood to encompass all tautomeric forms of the compounds disclosed herein. Exemplary tautomers include enol and imine forms, the keto and enamine forms, and geometric isomers and mixtures thereof. A tautomer is one of two or more structural isomers that exist in equilibrium and are readily converted from one isomeric form to another. This reaction results in the formal migration of a hydrogen atom accompanied by a switch of adjacent conjugated double bonds.

As discussed elsewhere herein, the compounds disclosed herein and the stereoisomers, tautomers, and isotopically-labelled forms thereof or a pharmaceutically acceptable salt of any of the foregoing may exist in solvated or unsolvated forms.

In several embodiments, the compound of Formula (I) (and/or any of Formulae (I), (Ia), (Ib), (Ic), (Id), (Ie), (If), (Ig), (Ih), (Ii), (Ij), (Ik), (Il), (Im), a 2-amido-5,6-fused-thieno-aryl, a 2-amido-5,6-fused-thieno-heteroaryl, a 2-amido-5,6-fused-thieno-heterocyclyl, or any other compound disclosed herein) can be characterized by its ability to inhibit the enzymatic activity of 15-PGDH (e.g., recombinant 15-PGDH) in an assay. In several embodiments, the assay is a cellular assay or a cell-free binding assay. In several embodiments, the compound of Formula (I) (and/or any of Formulae (I), (Ia), (Ib), (Ic), (Id), (Ie), (If), (Ig), (Ih), (Ii), (Ij), (Ik), (Il), (Im), a 2-amido-5,6-fused-thieno-aryl, a 2-amido-5,6-fused-thieno-heteroaryl, a 2-amido-5,6-fused-thieno-heterocyclyl, or any other compound disclosed herein) inhibits the enzymatic activity of 15-PGDH in the assay at an IC₅₀ of less than or equal to about: 1 μM, 250 nM, 50 nM, 20 nM, 10 nM, 5 nM, 2.5 nM 1 nM, 0.1 nM, 0.01 nM, 1 μM, or ranges including and/or spanning the aforementioned values. In several embodiments, the compound of Formula (I) (and/or any of Formulae (I), (Ia), (Ib), (Ic), (Id), (Ie), (If), (Ig), (Ih), (Ii), (Ij), (Ik), (Il), (Im), a 2-amido-5,6-fused-thieno-aryl, a 2-amido-5,6-fused-thieno-heteroaryl, a 2-amido-5,6-fused-thieno-heterocyclyl, or any other compound disclosed herein) inhibits the enzymatic activity of recombinant 15-PGDH at an IC₅₀ ranging from about 20 nM to about 0.01 pM, from about 20 nM to about 0.01 nM, from about 20 nM to about 0.1 nM, from about 20 nM to about 1 nM, from about 10 nM to about 0.01 pM, from about 10 nM to about 0.01 nM, from about 10 nM to about 0.1 nM, from about 10 nM to about 1 nM, from about 5 nM to about 0.01 pM, from about 5 nM to about 0.01 nM, from about 5 nM to about 0.1 nM, or from about 5 nM to about 1 nM.

In several embodiments, the IC₅₀ is measured using an assay as disclosed in the Examples. In several embodiments, the IC₅₀ for inhibition of 15-PGDH is measured using a cell-free binding assay. In several embodiments, recombinant 15-PGDH enzymatic assays are performed in a 25 μL volume of reaction buffer containing 50 mM Tris, pH 7.5, 0.01% Tween-20 and 100 μM DTT in a 384-well microtiter plate. In several embodiments, concentration-response experiments with test compounds are performed at 22 concentrations from 2-fold serial dilutions in DMSO. In several embodiments, the test compounds are pre-incubated with 15-PGDH for 15 minutes at room temperature. In several embodiments, thereafter, PGE2 and ß-NAD+ are added to initiate the 15-PGDH reaction. After 60 minutes at room temperature, the reaction is quenched and NADH signal was measured using a microtiter plate reader.

In several embodiments, the compound of Formula (I) (and/or any of Formulae (I), (Ia), (Ib), (Ic), (Id), (Ie), (If), (Ig), (Ih), (Ii), (Ij), (Ik), (Il), (Im), a 2-amido-5,6-fused-thieno-aryl, a 2-amido-5,6-fused-thieno-heteroaryl, a 2-amido-5,6-fused-thieno-heterocyclyl, or any other compound disclosed herein) can be characterized by its low inhibition of acetylcholinesterase (ACHE). In several embodiments, inhibition of ACHE may be measured using the method described in Ellman G. L., et al., A new and rapid colorimetric determination of acetylcholinesterase activity. Biochem Pharmacol. 1961; 7:88-95. In several embodiments, ACHE inhibition is measured by colorimetric detection of conversion of acetylthiocholine to thiocholine using a compound concentration of 10 μM under the Ellman conditions. In several embodiments, the substrate (acetylthiocholine; ATch) is provided at a concentration of 400 μM. In several embodiments, the ACHE enzyme is provided at a concentration of 1 mU. In several embodiments, dithiobisnotrobenzoate (DTNB) is provided at a concentration of 500 μM. In several embodiments, the reaction is carried out in a buffer of 8 mM NaH₂PO₄/Na₂HPO₄, 20 mM NaCl, 0.06% Triton and 0.8 mM EDTA and about 1 mU enzyme (ACHE). In several embodiments, the test compound is added to the buffer comprising enzyme. In several embodiments, the enzymatic reaction is initiated by addition of 400 μM of the substrate ATch and 500 μM DTNB. In this reaction ATch is transformed in thiocholine that will react with DTNB and forms an anion, 5-thio-2-nitrobenzoate that is yellow. In several embodiments, the absorbance is measured immediately (t=0) at λ=405 nm using a microplate reader. In several embodiments, the plate reader is an Envision, Perkin Elmer plate reader. In several embodiments, the this measurement allows to verify compound interference with the spectrophotometric detection at this wavelength. After 30 min incubation, a second measurement of the absorbance is made. The enzyme activity is determined by subtracting the signal measured at t=0 from that measured at t=30. In several embodiments, water is used as a control (substituted for the experimental compound). In several embodiments, a compound of Formula (I) (and/or any of Formulae (I), (Ia), (Ib), (Ic), (Id), (Ie), (If), (Ig), (Ih), (Ii), (Ij), (Ik), (Il), (Im), a 2-amido-5,6-fused-thieno-aryl, a 2-amido-5,6-fused-thieno-heteroaryl, a 2-amido-5,6-fused-thieno-heterocyclyl, or any other compound disclosed herein) resulted in no detectable inhibition of ACHE at a 10 μM test compound concentration. In several embodiments, a compound of Formula (I) (and/or any of Formulae (I), (Ia), (Ib), (Ic), (Id), (Ie), (If), (Ig), (Ih), (Ii), (Ij), (Ik), (Il), (Im), a 2-amido-5,6-fused-thieno-aryl, a 2-amido-5,6-fused-thieno-heteroaryl, a 2-amido-5,6-fused-thieno-heterocyclyl, or any other compound disclosed herein) resulted in no detectable inhibition of ACHE at a 5 μM test compound concentration. In several embodiments, a compound of Formula (I) (and/or any of Formulae (I), (Ia), (Ib), (Ic), (Id), (Ie), (If), (Ig), (Ih), (Ii), (Ij), (Ik), (Il), (Im), a 2-amido-5,6-fused-thieno-aryl, a 2-amido-5,6-fused-thieno-heteroaryl, a 2-amido-5,6-fused-thieno-heterocyclyl, or any other compound disclosed herein) resulted in no detectable inhibition of ACHE at a 2.5 μM test compound concentration. In several embodiments, a compound of Formula (I) (and/or any of Formulae (I), (Ia), (Ib), (Ic), (Id), (Ie), (If), (Ig), (Ih). (Ii), (Ij), (Ik). (Il), (Im), a 2-amido-5,6-fused-thieno-aryl, a 2-amido-5,6-fused-thieno-heteroaryl, a 2-amido-5,6-fused-thieno-heterocyclyl, or any other compound disclosed herein) resulted in no detectable inhibition of ACHE at a 1.0 μM test compound concentration. In several embodiments, at a concentration of test compound of 10 μM, the % inhibition of the enzyme is less than or equal to about 0% (undetectable), 0.05%, 0.1%, 0.5%, 0.75%, 1.0%, 2.0%, 5.0%, or ranges including and/or spanning the aforementioned values.

### Isotopically-Labelled Compounds

Further, the scope of the present disclosure includes all pharmaceutically acceptable isotopically-labelled compounds of the compounds disclosed herein, such as the compounds of Formula (I) (and/or any of Formulae (Ia), (Ib), (Ic), (Id), (Ie), (If), (Ig), (Ih), (Ii), (Ij), (Ik), (Il), (Im), a 2-amido-5,6-fused-thieno-heteroaryl, a 2-amido-5,6-fused-thieno-heterocyclyl, or any other compound disclosed herein) wherein one or more atoms are replaced by atoms having the same atomic number, but an atomic mass or mass number different from the atomic mass or mass number usually found in nature. Examples of isotopes suitable for inclusion in the compounds disclosed herein include isotopes of hydrogen, such as ²H (deuterium) and ³H (tritium), carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Certain isotopically-labelled compounds of Formula (I), for example, those incorporating a radioactive isotope, are useful in drug and/or substrate tissue distribution studies. The radioactive isotopes tritium (³H) and carbon-14 (¹⁴C) are particularly useful for this purpose in view of their ease of incorporation and ready means of detection. Substitution with isotopes such as deuterium (²H or D) may afford certain therapeutic advantages resulting from greater metabolic stability, for example, increased in vivo half-life or reduced dosage requirements, and hence may be advantageous in some circumstances. In several embodiments, the compounds herein are deuterated at metabolic hot spots (e.g., where certain hydrogen atoms of a structure are more metabolically labile than other hydrogens present on the molecule). Substitution with positron emitting isotopes, such as ¹¹C, ¹⁸F, ¹⁵O and ¹³N, can be useful in Positron Emission Topography (PET) studies, for example, for examining target occupancy. Isotopically-labelled compounds of the compounds disclosed herein can generally be prepared by conventional techniques known to those skilled in the art or by processes analogous to those described in the accompanying General Synthetic Schemes and Examples using an appropriate isotopically-labelled reagent in place of the non-labelled reagent previously employed.

### Solvates

As discussed above, the compounds disclosed herein and the stereoisomers and isotopically-labelled forms thereof or a pharmaceutically acceptable salt of any of the foregoing may exist in solvated or unsolvated forms. The term “solvate” as used herein refers to a molecular complex comprising a compound or a pharmaceutically acceptable salt thereof as described herein and a stoichiometric or non-stoichiometric amount of one or more pharmaceutically acceptable solvent molecules. If the solvent is water, the solvate is referred to as a “hydrate.” Accordingly, the scope of the instant disclosure is to be understood to encompass all solvents of the compounds disclosed herein and the stereoisomers, tautomers and isotopically-labelled forms thereof or a pharmaceutically acceptable salt of any of the foregoing.

### Formulation and Route of Administration

While it may be possible to administer a compound disclosed herein alone in the uses described, the compound administered normally will be present as an active ingredient in a pharmaceutical composition. Thus, in several embodiments, provided herein is a pharmaceutical composition comprising a compound disclosed herein in combination with one or more pharmaceutically acceptable excipients, such as diluents, carriers, adjuvants and the like, and, if desired, other active ingredients. In several embodiments, a pharmaceutical composition comprises a therapeutically effective amount of a compound disclosed herein.

The compound(s) disclosed herein may be administered by any suitable route in the form of a pharmaceutical composition adapted to such a route and in a dose effective for the treatment intended. The compounds and compositions presented herein may, for example, be administered orally, mucosally, topically, transdermally, rectally, pulmonarily, parentally, intranasally, intravascularly, intravenously, intraarterial, intraperitoneally, intrathecally, subcutaneously, sublingually, intramuscularly, intrasternally, vaginally or by infusion techniques, in dosage unit formulations containing conventional pharmaceutically acceptable excipients.

The pharmaceutical composition may be in the form of, for example, a tablet, chewable tablet, minitablet, caplet, pill, bead, hard capsule, soft capsule, gelatin capsule, granule, powder, lozenge, patch, cream, gel, sachet, microneedle array, syrup, flavored syrup, juice, drop, injectable solution, emulsion, microemulsion, ointment, aerosol, aqueous suspension, or oily suspension. The pharmaceutical composition is typically made in the form of a dosage unit containing a particular amount of the active ingredient.

Provided herein as Embodiment 255 is a pharmaceutical composition comprising the compound or salt of any one of Embodiments 1 to 254, and a pharmaceutically acceptable excipient.

Further, this disclosure encompasses pharmaceutical compositions comprising mixtures of any of the compounds disclosed herein and one or more other active agents disclosed herein.

### Use of Compounds and Compositions, Medicaments for Use, and Methods of Treatment

As discussed herein, the compounds described herein are to be understood to include all stereoisomers, tautomers, or pharmaceutically acceptable salts of any of the foregoing or solvates of any of the foregoing. Accordingly, the scope of the methods and uses provided in the instant disclosure is to be understood to encompass also methods and uses employing all such forms. Besides being useful for human treatment, the compounds provided herein may be useful for veterinary treatment of companion animals, exotic animals and farm animals, including mammals, rodents, and the like. For example, animals including horses, dogs, and cats may be treated with compounds provided herein.

In several embodiments, as disclosed elsewhere herein, a method of treating a patient is provided. In several embodiments, the method comprises administering a therapeutic amount of a 15-PGDH inhibitor compound (e.g., of any one of Formulae (I), (Ia), (Ib), (Ic), (Id), (Ie), (If), (Ig), (Ih), (Ii), (Ij), (Ik), (Il), (Im), a 2-amido-5,6-fused-thieno-aryl, a 2-amido-5,6-fused-thieno-heteroaryl, a 2-amido-5,6-fused-thieno-heterocyclyl, or any other compound disclosed herein) to a patient.

In several embodiments, the method comprises selecting a patient. In several embodiments, the patient is one suffering from a 15-PGDH mediated disease, disorder, or condition.

In several embodiments, after administration of a 15-PGDH inhibitor to the patient, the patient's response is measured. In several embodiments, a beneficial effect of the 15-PGDH inhibitors may be assessed by a reduction in one or more inflammatory biomarkers in a relevant sample from the subject. In several embodiments, the inflammatory biomarker may comprise or consist of one or more of cytokines or inflammatory cytokines (e.g., those associated with fibrosis). Such cytokines can include, for example, ′HHb, MIP2 (e.g., CCL3 or CCL4), IFN5, TGFP, TNFa, IL-6, MCP-1, IL2, and IL-10 in BAL fluid. Methods for measuring the amount of such biomarkers, include but are not limited to ELISAs. In several embodiments, the biomarker is an eicosanoid or a PGE2 metabolite, such as, prostaglandin E2 (PGE2), prostaglandin E metabolite (PGE-M; tetranor-PGEM), 15-keto prostaglandin E 2 (15-keto-PGE2), prostaglandin F2α PGF2α, 6-keto prostaglandin F1α (6-keto-PGF1α), prostaglandin D2 (PGD2), prostaglandin J2 (PGJ2), tetranor-PGE1 (TN-E), thromboxane B2 (TXB2), leukotriene B4 (LTB4), 15-hydroxyeicosatetraenoic acid (15-HETE), 12-hydroxyeicosatetraenoic acid (12-HETE), 8-hydroxyeicosatetraenoic acid (8-HETE), 5-hydroxyeicosatetraenoic acid (5-HETE), 17-HDA, 12,13-dihydroxy-9Z-octadecenoic acid (12, 13-DiHOME), 9,10-dihydroxy-9Z-octadecenoic acid 9,10-DiHOME, 14,15-dihydroxyeicosatrienoic acid (14,15-DHET), 11,12-dihydroxyeicosatrienoic acid (11,12-DHET), or combinations of the foregoing. In several embodiments, the methods disclosed herein may comprise reducing an amount of one or more biomarkers in a sample from the subject compared to control. In several embodiments, the methods disclosed herein may comprise reducing an amount of one or more biomarkers in a sample from the subject compared to the patient prior to treatment. In several embodiments, the methods disclosed herein may comprise increasing an amount of one or more biomarkers in a sample from the subject compared to control. In several embodiments, the methods disclosed herein may comprise increasing an amount of one or more biomarkers in a sample from the subject compared to the patient prior to treatment. Thus, the levels of these biomarkers may be measured before or after treatment with a 15-PDGH inhibitor as disclosed herein.

In several embodiments, as disclosed elsewhere herein, the patient is one suffering from a 15-PGDH mediated disease or disorder. In several embodiments, the 15-PGDH mediated disease or disorder is inflammatory bowel disease, ulcerative colitis, Crohn's disease, fibrotic disease disorder or condition. In several embodiments, the 15-PGDH mediated disease or disorder is atherosclerosis. In several embodiments, the 15-PGDH mediated disease or disorder is autoimmune disease. In several embodiments, the 15-PGDH mediated disease, disorder, or condition is systemic sclerosis, multifocal fibrosclerosis, nephrogenic systemic fibrosis, kidney fibrosis, glomerular sclerosis, renal tubulointerstitial fibrosis, progressive renal disease or diabetic nephropathy, cardiac fibrosis, pulmonary fibrosis, glomerulosclerosis pulmonary fibrosis, idiopathic pulmonary fibrosis, silicosis, asbestosis, interstitial lung disease, interstitial fibrotic lung disease, chemotherapy/radiation induced pulmonary fibrosis, endomyocardial fibrosis, deltoid fibrosis, pancreatitis, general fibrosis syndrome characterized by replacement of normal muscle tissue by fibrous tissue in varying degrees, retroperitoneal fibrosis, liver fibrosis, liver cirrhosis, chronic renal failure, myelofibrosis, bone marrow fibrosis, acute fibrosis, or organ specific fibrosis. In several embodiments, the 15-PGDH mediated disease, disorder, or condition is intestinal ischemia, ischemia, ischemic brain disease, ischemic heart disease, ischemic peripheral vascular disease, ischemic placenta, ischemic renal disease, ischemic vascular disease, ischemic-reperfusion injury, limb ischemia, lower extremity ischemia, myocardial ischemia, organ ischemia, peripheral ischemia, tissue ischemia, transient ischemic attack (TIA), and wounds to tissues or organs.

Provided herein as Embodiment 256 is the compound or salt of any one of Embodiments 1 to 254 or the pharmaceutical composition of Embodiment 255 for use as a medicament.

Provided herein as Embodiment 257 is the compound or salt of any one of Embodiments 1 to 254 or the pharmaceutical composition of Embodiment 255 for use in treating a 15-PGDH mediated disease or disorder.

Provided herein as Embodiment 258 is the compound or salt of any one of Embodiments 1 to 254 or the pharmaceutical composition of Embodiment 255 for use in treating inflammatory bowel disease.

Provided herein as Embodiment 259 is the compound or salt of any one of Embodiments 1 to 254 or the pharmaceutical composition of Embodiment 255 for use in treating ulcerative colitis.

Provided herein as Embodiment 260 is the compound or salt of any one of Embodiments 1 to 254 or the pharmaceutical composition of Embodiment 255 for use in treating Crohn's disease.

Provided herein as Embodiment 261 is the compound or salt of any one of Embodiments 1 to 254 or the pharmaceutical composition of Embodiment 255 for use in treating a fibrotic disease, disorder or condition.

Provided herein as Embodiment 262 is the compound, salt, or composition of Embodiment 261, wherein the fibrotic disease, disorder, or condition is characterized, in whole or in part, by the excess production of fibrous material, including excess production of fibrotic material within the extracellular matrix, or the replacement of normal tissue elements by abnormal, non-functional, and/or excessive accumulation of matrix-associated components.

Provided herein as Embodiment 263 is the compound, salt, or composition of Embodiment 261 or 262, wherein the fibrotic disease, disorder, or condition is systemic sclerosis, multifocal fibrosclerosis, nephrogenic systemic fibrosis, kidney fibrosis, glomerular sclerosis, renal tubulointerstitial fibrosis, progressive renal disease or diabetic nephropathy, cardiac fibrosis, pulmonary fibrosis, glomerulosclerosis pulmonary fibrosis, idiopathic pulmonary fibrosis, silicosis, asbestosis, interstitial lung disease, interstitial fibrotic lung disease, chemotherapy/radiation induced pulmonary fibrosis, endomyocardial fibrosis, deltoid fibrosis, pancreatitis, general fibrosis syndrome characterized by replacement of normal muscle tissue by fibrous tissue in varying degrees, retroperitoneal fibrosis, liver fibrosis, liver cirrhosis, chronic renal failure; myelofibrosis, bone marrow fibrosis, acute fibrosis, or organ specific fibrosis.

Provided herein as Embodiment 264 is the compound, salt, or composition of Embodiment 261 or 262, wherein the fibrotic disease, disorder, or condition is idiopathic pulmonary fibrosis.

Provided herein as Embodiment 265 is the compound, salt, or composition of Embodiment 261 or 262, wherein the fibrotic disease, disorder, or condition is kidney fibrosis.

Provided herein as Embodiment 266 is the compound, salt, or composition of Embodiment 261 or 262, wherein the fibrotic disease, disorder, or condition is liver fibrosis.

Provided herein as Embodiment 267 is the compound or salt of any one of Embodiments 1 to 254 or the pharmaceutical composition of Embodiment 255 for use in treating one or more of atherosclerotic cardiovascular disease, autoimmune disease, intestinal ischemia, ischemia, ischemic brain disease, ischemic heart disease, ischemic peripheral vascular disease, ischemic placenta, ischemic renal disease, ischemic vascular disease, ischemic-reperfusion injury, limb ischemia, lower extremity ischemia, myocardial ischemia, organ ischemia, peripheral ischemia, tissue ischemia, transient ischemic attack (TIA), and wounds to tissues or organs.

Provided herein as Embodiment 268 is a method of treating a 15-PGDH mediated disease, disorder, or condition in a subject in need thereof, the method comprising:
administering to the subject therapeutically an effective amount of the compound or salt of any one of Embodiments 1 to 254 or the pharmaceutical composition of Embodiment 255.

Provided herein as Embodiment 269 is a method of treating intestinal, gastrointestinal, or bowel disorders in a subject in need thereof, the method comprising:
administering to the subject a therapeutically effective amount of the compound or salt of any one of Embodiments 1 to 254 or the pharmaceutical composition of Embodiment 255.

Provided herein as Embodiment 270 is the method of Embodiment 268 or 269, wherein the disease or disorder comprises at least one of ulcerative colitis, inflammatory bowel disease, and Crohn's disease.

### Combinations

In several embodiments, the 15-PGDH inhibitor compounds disclosed herein (including those of Formula (I), other formulae disclosed herein, 2-amido-5,6-fused-thieno-aryls, 2-amido-5,6-fused-thieno-heterocyclyls, etc.) can be provided in combination with other therapeutic agents.

In several embodiments, a 15-PGDH inhibitor as disclosed herein is used in combination with a tumor necrosis factor (TNF) inhibitor (e.g., TNF-α inhibitor). In several embodiments, the TNF inhibitors can include, but are not limited to, anti-TNF alpha antibodies (e.g., infliximab, adalimumab, certolizumab pegol, and golimumab), receptor-construct fusion proteins (such as etanercept), or small molecules, such as, but not limited to, pomalidomide, thalidomide, lenalidomide and bupropion.

In several embodiments, a 15-PGDH inhibitor as disclosed herein is used in combination with a corticosteroid. In several embodiments, the corticosteroid is aclovate, alclometasone dipropionate, amcinafel, amcinafide, amcinonide, aristocort A, augmented betamethasone dipropionate, beclamethasone, beclopmethasone dipropionate, betamethasone, betamethasone benzoate, betamethasone-17-benzoate, betamethasone dipropionate, betamethasone sodium phosphate and acetate, betamethasone valerate, betamethasone-17-valerate, chloroprednisone, clobetasol propionate, clobetasone propionate, clocortelone, cordran, corticosterone, cortisol, cortisol acetate, cortisol cypionate, cortisol sodium phosphate, cortisol sodium succinate, cortisone, cortisone acetate, cortodoxone, cyclocort, deflazacort, defluprednate, descinolone, desonide, desowen, desoximetasone, desoxycorticosterone acetate, desoxycorticosterone pivalate, 11-desoxycortisol, dexamethasone, dexamethasone acetate, dexamethasone sodium phosphate, dichlorisone, diflorasone diacetate, dihydroxycortisone, diprolen, diprolene, diprosone, esters of betamethasone, florone, flucetonide, flucloronide, flucortolone, fludrocortisone, fludrocortisone acetate, flumethalone, flumethasone, flumethasone pivalate, flunisolide, fluocinolone acetonide, fluocinolone acetonide acetate, fluocinonide, fluorametholone, fluorocortisone, fluperolone, fluprednisolone, flurandrenolide, fluroandrenolone acetonide, fluticasone propionate, fuprednisolone, halcinonide, halobetasol propionate, halog, hydrocortamate, hydrocortisone, hydrocortisone acetate, hydrocortisone butyrate, hydrocortisone valerate, hydrocortisone-17-valerate, kenalog, lidex, locold, locorten, maxiflor, medrysone, meprednisone, methylprednisolone, 6α-methylprednisolone, methylprednisolone acetate, methylprednisolone sodium succinate, methylprednisone, mometasone furoate, paramethasone, paramethasone acetate, prednidone, prednisone, prednisolone, prednisolone acetate, prednisolone sodium phosphate, prednisolone sodium succinate, prednisolone tebutate, prednisone, psorcon, synalar, temovate, tetrahydrocortisol, topicort, topicort LP, triamcinolone, triamcinolone acetonide, triamcinolone diacetate, triamcinolone hexacotonide, tridesilone, valisone, westcort, or a combination of the foregoing.

In several embodiments, each constituent of the therapeutic combination can be administered in using the same route or a different route. For example, as disclosed elsewhere herein, the 15-PGDH inhibitors compounds disclosed herein may be administered by any suitable route in the form of a pharmaceutical composition adapted to such a route and in a dose effective for the treatment intended. Each constituent of the therapeutic combination can, for example, be administered orally, mucosally, topically, transdermally, rectally, pulmonarily, parentally, intranasally, intravascularly, intravenously, intraarterial, intraperitoneally, intrathecally, subcutaneously, sublingually, intramuscularly, intrasternally, vaginally or by infusion techniques, in dosage unit formulations containing conventional pharmaceutically acceptable excipients.

The individual therapeutics comprising the combination can be administered substantially simultaneously using any of the disclosed routes for any of the disclosed methods of treatment (e.g., methods of treating IBD, etc.). The individual therapeutics comprising the combination can be administered in a sequential manner using any of the disclosed routes for any of the disclosed methods of treatment (e.g., methods of treating IBD, etc.). The individual therapeutics comprising the combination may be administered together as part of the same composition or as different compositions.

It will be appreciated that, for a particular biologic (e.g., antibody or protein-based therapeutic), those therapeutic agents may be more suitably for administered by injection. Alternatively, small molecules (such as the 15-PGDH inhibitors, 5,6-fused bicyclic thieno-heterocyclyls, compounds of the any formula disclosed herein, etc.) may be suitable for injection or other administration methods (e.g., through other routes, including orally).

### Methods of Manufacture

The compounds provided herein can be synthesized according to the procedures described in this and the following sections. The synthetic methods described herein are merely exemplary, and the compounds disclosed herein may also be synthesized by alternate routes utilizing alternative synthetic strategies, as appreciated by persons of ordinary skill in the art, in view of the teachings of the present disclosure. It should be appreciated that the general synthetic procedures and specific examples provided herein are illustrative only and should not be construed as limiting the scope of the present disclosure in any manner. See, e.g., W. Friedrichsen, Furans, Thiophenes and Selenophenes with Fused Six-membered Heterocyclic Rings, in Comprehensive Heterocyclic Chemistry, 1984.

Generally, the compounds of Formula (I) (e.g., or of any one of Formulae (Ia), (Ib), (Ic), (Id), (Ie), (If), (Ig), (Ih), (Ii), (Ij), (Ik), (Il), (Im), a 2-amido-5,6-fused-thieno-heteroaryl, a 2-amido-5,6-fused-thieno-heterocyclyl, or any other compound disclosed herein) can be synthesized using one or more steps according to the following schemes and/or using one or more of the following intermediates. Any variables used in the following schemes in intermediates may be as defined elsewhere herein, unless otherwise noted. Thus, for example, where R³ (or R^{1a}, R^{1b}, or any other variable) is defined in one manner for Formula (I) and in another way for Formula (I-int-g), the definition for R³ (or R^{1a}, R^{1b}, or any other variable) in Formula (I) may be substituted for the definition of R³ (or R^{1a}, R^{1b}, or any other variable) in Formula (I-int-g) and vice versa. Likewise, the definitions for like variables in two different intermediates may be used interchangeably. Suitable reaction conditions, such as, solvent, reaction temperature, and reagents, for the Schemes discussed in this section, may be found in the examples provided herein.

Provided herein as Embodiment 271 is a method of manufacturing the compound or salt of Embodiment 1, the method comprising:
performing a Suzuki coupling between a compound of Formula (I-int-c.1)

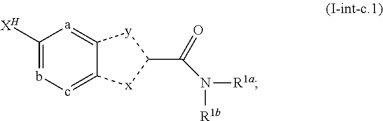

with a compound of Formula (B^{a}—R²):

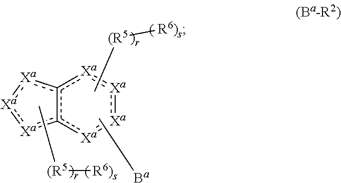

where
X^{H} is a halogen;
B^{a} is a boronic acid group or a boronic ester group;
“ [symbol] ” indicates a double bond or single bond may be present;
each instance of X^{a} independently is nitrogen, oxygen, or carbon;
each instance of s, where present, is an integer selected from 0, 1, 2, or 3;
each instance of r, where present, independently is an integer selected from 0, 1, 2, or 3, wherein where two instances of r occur on the same R² group, the total of both r values combined does not exceed 5;
each instance of R⁵, when present and attached to an N ring member, independently is C₁₋₆alkyl, C₁₋₆haloalkyl, C₃₋₆ cycloalkyl, or heterocyclyl having 3 to 10 members; and
each instance of R⁵, when present and attached to a C ring member, independently is oxo, CN, C₁₋₆ alkyl, C₃₋₆ cycloalkyl, C₁₋₆ haloalkyl, C₁₋₆ alkoxy, C(O)N(R⁶)₂, —N(R⁶)₂, —N(R⁶)(C₁₋₆ alkylene-OH), heterocyclyl having 3 to 10 members;
each instance of R⁶, where present, independently is —H, C₁₋₃ alkyl, C(O)C₁₋₃ alkyl, C(O)₂ C₁₋₃ alkyl, C₁₋₃ alkylene-OH, or S(O)ₙC₁₋₃alkyl; and
wherein a —N═ ring nitrogen can be bonded to an R⁵ to provide a cation.

### Intermediates

Several embodiments pertain to intermediates useful for synthesizing a compound of any one of Formulae (I), (Ia), (Ib), (Ic), (Id), (Ie), (If), (Ig), (Ih), (Ii), (Ij), (Ik), (Il), (Im), a 2-amido-5,6-fused-thieno-aryl, a 2-amido-5,6-fused-thieno-heteroaryl, a 2-amido-5,6-fused-thieno-heterocyclyl, or any other compound disclosed herein.

### Formula (I-int-a)

Several embodiments pertain to a compound of Formula (I-int-a) or a salt thereof:

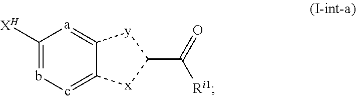

wherein the variables are as defined elsewhere herein. In several embodiments,

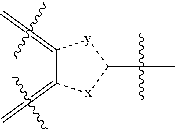

is

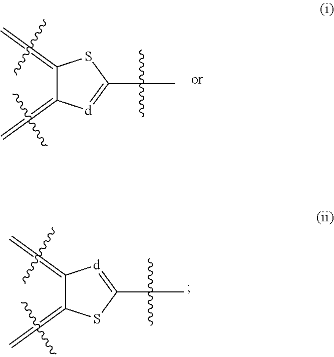

a is N or CH;
b is N or CH;
c is N, CH, or C—R³;
d is N, CH, or C—R⁴;
R³ is —H, C₁₋₆ alkyl, C₃₋₁₀ cycloalkyl, C₆₋₁₀ aryl, heteroaryl having 5 to 10 ring members, or heterocyclyl having 3 to 10 ring members;
   wherein R³ may be unsubstituted or substituted with one or more substituents each of which independently is halogen, —OH, oxo, C₁₋₃ alkyl, C₁₋₃ haloalkyl, C₁₋₃ alkoxy, C₁₋₃ alkylene-C₁₋₃ alkoxy, or C₁₋₆heteroalkyl;
R⁴ is —H or C₁₋₆ alkyl; and
R^{il} is C₁₋₆ alkoxy; and X^{H} represents a halogen atom.

In several embodiments, each X^{H} independently represents —Cl or Br. In several embodiments, X^{H} is —Cl. In several embodiments, R^{il} is ethoxy.

### Formula (I-int-b.1)

Several embodiments pertain to a compound of Formula (I-int-b.1) or a salt thereof:

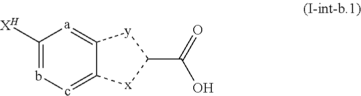

wherein the variables are as defined elsewhere herein. In several embodiments,

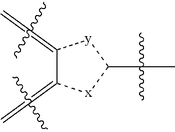

is

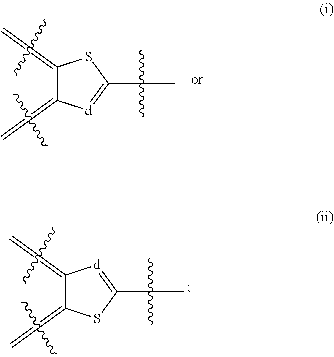

a is N or CH;
b is N or CH;
c is N, CH, or C—R³;
d is N, CH, or C—R⁴;
R³ is —H, C₁₋₆ alkyl, C₃₋₁₀ cycloalkyl, C₆₋₁₀ aryl, heteroaryl having 5 to 10 ring members, or heterocyclyl having 3 to 10 ring members;
   wherein R³ may be unsubstituted or substituted with one or more substituents each of which independently is halogen, —OH, oxo, C₁₋₃ alkyl, C₁₋₃ haloalkyl, C₁₋₃ alkoxy, C₁₋₃ alkylene-C₁₋₃ alkoxy, or C₁₋₆heteroalkyl;
R⁴ is —H or C₁₋₆ alkyl; and
X^{H} is halogen.

In several embodiments, each X^{H} independently represents —Cl or —Br. In several embodiments, X^{H} is —Cl.

### Formula (I-int-c.1)

Several embodiments pertain to a compound of Formula (I-int-c.1) or a salt thereof:

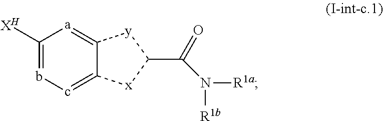

wherein the variables are as defined elsewhere herein. In several embodiments,

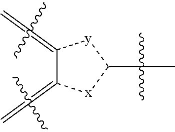

is

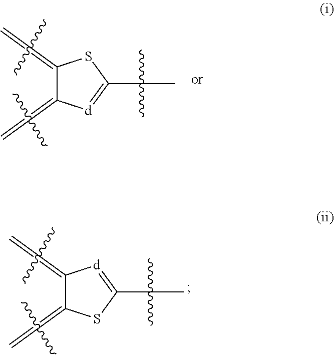

a is N or CH;
b is N or CH;
c is N, CH, or C—R³;
d is N, CH, or C—R⁴;
R^{1a} is —H, C₁₋₆ alkyl, C₁₋₆ haloalkyl, or C₁₋₆ heteroalkyl;
   wherein R^{1a} may be unsubstituted or substituted with one or more substituents, each of which independently is halogen, —OH, —N(R^{b})₂, C₁₋₃ alkyl, or C₁₋₃ alkoxy;
R^{1b} is C₁₋₆ alkyl, C₁₋₆ haloalkyl, C₁₋₆ heteroalkyl, C₃₋₁₀ cycloalkyl, or heterocyclyl having 3 to 10 ring members;
   wherein R^{1b} may be unsubstituted or substituted with one or more substituents, each of which independently is halogen, —OH, —N(R^{b})₂, C₁₋₃ alkyl, or C₁₋₃ alkoxy; or, alternatively, R^{1a} and R^{1b} together form a heterocyclyl having 3 to 10 ring members;
   wherein the heterocyclyl formed by R^{1a} and R^{1b} may be unsubstituted or substituted with one or more substituents, each of which independently is halogen, —OH, —CN, —N(R)₂, C₁₋₆ alkyl, C₁₋₆ haloalkyl, C₁₋₆ alkoxy, C₁₋₆ heteroalkyl, C₃₋₆ cycloalkyl, or heterocyclyl having 3 to 6 ring members;
R³ is —H, C₁₋₆ alkyl, C₃₋₁₀ cycloalkyl, C₆₋₁₀ aryl, heteroaryl having 5 to 10 ring members, or heterocyclyl having 3 to 10 ring members;
wherein R³ may be unsubstituted or substituted with one or more substituents each of which independently is halogen, —OH, oxo, —N(R^{b})₂, C₁₋₃ alkyl, C₁₋₃ haloalkyl, C₁₋₃ alkoxy, C₁₋₃ alkylene-C₁₋₃ alkoxy, or C₁₋₆ heteroalkyl;
R⁴ is —H, C₁₋₆ alkyl, or —N(R)₂;
   where each instance of R⁷, where present, independently is —H or C₁₋₆ alkyl;
   or, alternatively, two R⁷ substituents together form a heterocyclyl having 3 to 6 ring members; wherein R⁷ may be unsubstituted or substituted with one or more substituents each of which independently is —F, —OH, C₁₋₃ alkoxy;
wherein each instance of R^{b}, where present, independently is —H or C₁₋₆ alkyl; or, alternatively, two R^{b} substituents together form a heterocyclyl having 3 to 6 ring members; and
   wherein R^{b} may be unsubstituted or substituted with one or more substituents each of which independently is —F, —OH, C₁₋₃ alkoxy.

### Formula (I-int-b.2)

Several embodiments pertain to a compound of Formula (I-int-b.2) or a salt thereof:

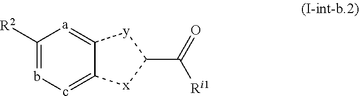

wherein the variables are as defined elsewhere herein. In several embodiments,

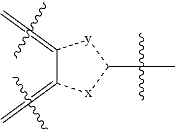

is

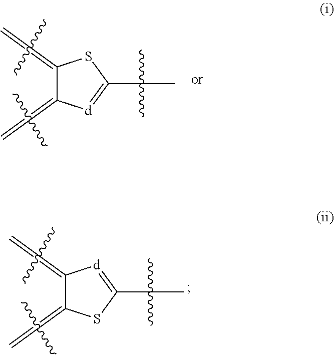

a is N or CH;
b is N or CH;
c is N, CH, or C—R³;
d is N, CH, or C—R⁴;
R² is heterocyclyl having 5 to 10 ring members, heteroaryl having 5 to 10 ring members;
   wherein R² may be unsubstituted or substituted with one or more instances of R⁵; wherein each instance of R⁵ independently is oxo, CN, C₁₋₆ alkyl, C₃₋₆ cycloalkyl, C₁₋₆ haloalkyl, C₁₋₆ alkoxy, C(O)N(R⁶)₂, —N(R⁶)₂, —N(R⁶)(C₁₋₆ alkylene-OH), or heterocyclyl having 3 to 10 members;
   wherein each instance of R⁵ may be unsubstituted or substituted one or more instances of R⁶.
   wherein each instance of R⁶ independently is —H, C₁₋₆ alkyl, C(O)C₁₋₆ alkyl, C(O)₂C₁₋₆ alkyl, C₁₋₆ alkylene-OH, or S(O)ₙC₁₋₆alkyl, where n is 0, 1, or 2;
   wherein R⁶ may be unsubstituted or substituted with one or more substituents each of which independently is —F, —OH, C₁₋₃ alkoxy;
R³ is —H, C₁₋₆ alkyl, C₃₋₁₀ cycloalkyl, C₆₋₁₀ aryl, heteroaryl having 5 to 10 ring members, or heterocyclyl having 3 to 10 ring members;
   wherein R³ may be unsubstituted or substituted with one or more substituents each of which independently is halogen, —OH, oxo, C₁₋₃ alkyl, C₁₋₃ haloalkyl, C₁₋₃ alkoxy, C₁₋₃ alkylene-C₁₋₃ alkoxy, or C₁₋₆heteroalkyl; R⁴ is —H or C₁₋₆ alkyl;
   wherein each instance of R^{b}, where present, independently is —H or C₁₋₆ alkyl;
or, alternatively, two R^{b} substituents together form a heterocyclyl having 3 to 6 ring members;
   wherein R^{b} may be unsubstituted or substituted with one or more substituents each of which independently is —F, —OH, C₁₋₃ alkoxy; and
Rⁱ¹ is C₁₋₆ alkoxy.

In several embodiments, Rⁱ¹ is ethoxy.

### Formula (I-int-c.2)

Several embodiments pertain to a compound of Formula (I-int-c.1) or a salt thereof:

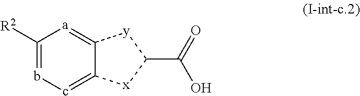

wherein the variables are as disclosed elsewhere herein (e.g., as disclosed for the compound of Formula (I-int.b.2).

### Formula (I-int-d)

Several embodiments pertain to a compound of Formula (I-int-d) or a salt thereof:

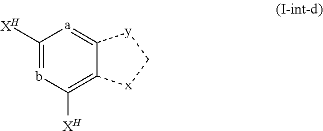

wherein the variables are as defined elsewhere herein. In several embodiments,

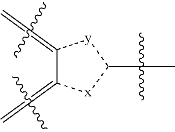

is

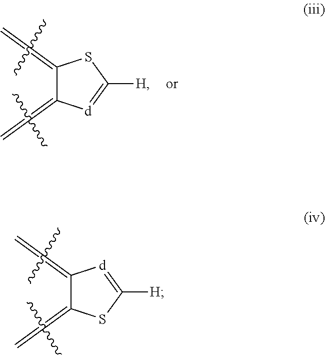

a is N or CH;
b is N or CH;
d is N, CH, or C—R;
R⁴ is —H or C₁₋₆ alkyl; and
each X^{H} independently represents a suitable halogen atom or a similar reactive group.

In several embodiments, each X^{H} independently represents —Cl or —Br. In several embodiments, X^{H} is Cl.

### Formula (I-int-e)

Several embodiments pertain to a compound of Formula (I-int-e) or a salt thereof:

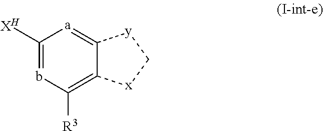

wherein the variables are as defined elsewhere herein. In several embodiments,

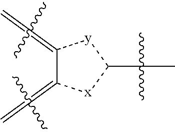

is

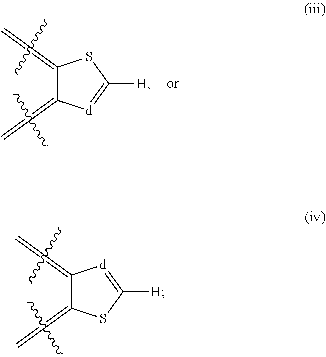

a is N or CH;
b is N or CH;
d is N, CH, or C—R⁴;
R³ is C₁₋₆ alkyl, C₃₋₁₀ cycloalkyl, C₆₋₁₀ aryl, heteroaryl having 5 to 10 ring members, or heterocyclyl having 3 to 10 ring members;
   wherein R³ may be unsubstituted or substituted with one or more substituents each of which independently is halogen, —OH, oxo, —N(R)₂, C₁₋₃ alkyl, C₁₋₃ haloalkyl, C₁₋₃ alkoxy, C₁₋₃ alkylene-C₁₋₃ alkoxy, or C₁₋₆heteroalkyl;
R⁴ is —H or C₁₋₆ alkyl;
wherein each instance of R^{b}, where present, independently is —H or C₁₋₆ alkyl;
or, alternatively, two R^{b} substituents together form a heterocyclyl having 3 to 6 ring members; and
   wherein R^{b} may be unsubstituted or substituted with one or more substituents each of which independently is —F, —OH, C₁₋₃ alkoxy.

### Formula (I-int-f)

Several embodiments pertain to a compound of Formula (I-int-f) or a salt thereof:

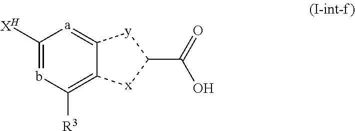

wherein the variables are as defined elsewhere herein. In several embodiments,

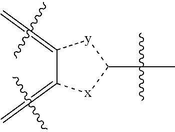

is

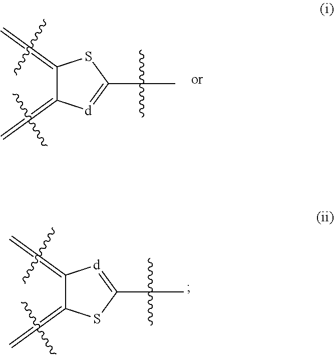

a is N or CH;
b is N or CH;
d is N, CH, or C—R;
R³ is C₁₋₆ alkyl, C₃₋₁₀ cycloalkyl, C₆₋₁₀ aryl, heteroaryl having 5 to 10 ring members, or heterocyclyl having 3 to 10 ring members;
   wherein R³ may be unsubstituted or substituted with one or more substituents each of which independently is halogen, —OH, oxo, —N(R)₂, C₁₋₃ alkyl, C₁₋₃ haloalkyl, C₁₋₃ alkoxy, C₁₋₃ alkylene-C₁₋₃ alkoxy, or C₁₋₆heteroalkyl;
R⁴ is —H or C₁₋₆ alkyl;
wherein each instance of R^{b}, where present, independently is —H or C₁₋₆ alkyl;
or, alternatively, two R^{b} substituents together form a heterocyclyl having 3 to 6 ring members; and
   wherein R^{b} may be unsubstituted or substituted with one or more substituents each of which independently is —F, —OH, C₁₋₃ alkoxy.

### Formula (I-int-g)

Several embodiments pertain to a compound of Formula (I-int-g) or a salt thereof:

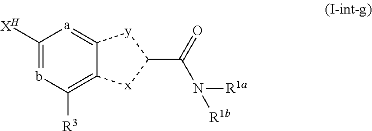

wherein the variables are as defined elsewhere herein. In several embodiments,

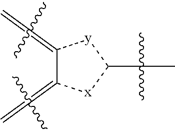

is

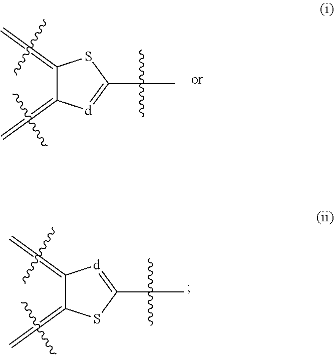

a is N or CH;
b is N or CH;
d is N, CH, or C—R⁴;
R^{1a} is —H. C₁₋₆ alkyl, C₁₋₆ haloalkyl, or C₁₋₆ heteroalkyl;
   wherein R^{1a} may be unsubstituted or substituted with one or more substituents, each of which independently is halogen, —OH, —N(R^{b})₂, C₁₋₃ alkyl, or C₁₋₃ alkoxy;
R^{1b} is C₁₋₆ alkyl, C₁₋₆ haloalkyl, C₁₋₆ heteroalkyl, C₃₋₁₀ cycloalkyl, or heterocyclyl having 3 to 10 ring members;
   wherein R^{1b} may be unsubstituted or substituted with one or more substituents, each of which independently is halogen, —OH, —N(R^{b})₂, C₁₋₃ alkyl, or C₁₋₃ alkoxy;
or, alternatively, R^{1a} and R^{1b} together form a heterocyclyl having 3 to 10 ring members;
   wherein the heterocyclyl formed by R^{1a} and R^{1b} may be unsubstituted or substituted with one or more substituents, each of which independently is halogen, —OH, —CN, —N(R)₂, C₁₋₆ alkyl. C₁₋₆ haloalkyl, C₁₋₆ alkoxy, C₁₋₆ heteroalkyl, C₃₋₆ cycloalkyl, or heterocyclyl having 3 to 6 ring members;
R³ is —H, C₁₋₆ alkyl, C₃₋₁₀ cycloalkyl, C₆₋₁₀ aryl, heteroaryl having 5 to 10 ring members, or heterocyclyl having 3 to 10 ring members;
   wherein R³ may be unsubstituted or substituted with one or more substituents each of which independently is halogen, —OH, oxo, —N(R^{b})₂, C₁₋₃ alkyl, C₁₋₃ haloalkyl, C₁₋₃ alkoxy, C₁₋₃ alkylene-C₁₋₃ alkoxy, or C₁₋₆heteroalkyl;
R⁴ is —H or C₁₋₆ alkyl;
wherein each instance of R^{b}, where present, independently is —H or C₁₋₆ alkyl;
or, alternatively, two R^{b} substituents together form a heterocyclyl having 3 to 6 ring members; and
   wherein R^{b} may be unsubstituted or substituted with one or more substituents each of which independently is —F, —OH, C₁₋₃ alkoxy.

### Formula (I-int-h) and Formula (I-int-h.1)

Several embodiments pertain to a compound of Formula (I-int-h) or a salt thereof:

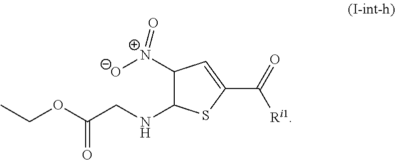

In several embodiments, Rⁱ¹ is C₁₋₆ alkoxy. In several embodiments, Rⁱ¹ is ethoxy (e.g., as shown in Formula (I-int-h.1)).

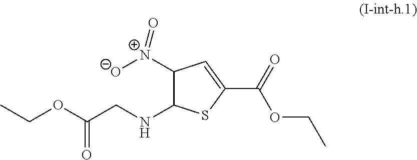 [SMILES: CCOC(=O)CNC1SC(C(=O)OCC)=CC1[N+](=O)[O-]]

### Formula (I-int-i) and Formula (I-int-i.1)

Several embodiments pertain to a compound of Formula (I-int-i) or a salt thereof.

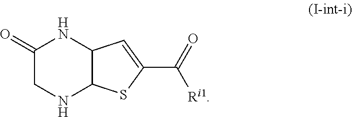

In several embodiments, Rⁱ¹ is C₁₋₆ alkoxy. In several embodiments, Rⁱ¹ is ethoxy (e.g., as shown in Formula (I-int-i.1)).

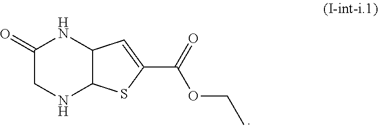

### Formula (I-int-j) and Formula (I-int-j.1)

Several embodiments pertain to a compound of Formula (I-int-j) or a salt thereof:

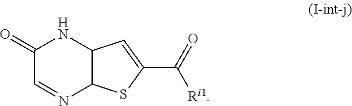

In several embodiments, the compound of Formula (I-int-j) is prepared by oxidizing the compound of Formula (I-int-i). In several embodiments, the compound of Formula (I-int-i) is oxidized in the presence of manganese dioxide. In several embodiments, Rⁱ¹ is C₁₋₆ alkoxy. In several embodiments, Rⁱ¹ is ethoxy as shown in Formula (I-int-j.1).

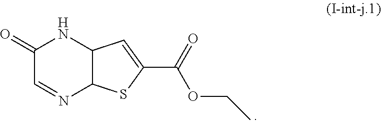

### Formula (I-int-k), Formula (I-int-k.1), and Formula (I-int-k.2)

Several embodiments pertain to a compound of Formula (I-int-k) or a salt thereof:

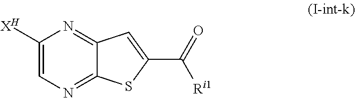

wherein the variables are as defined elsewhere herein. In several embodiments, X^{H} is a halogen. In several embodiments, X^{H} is —Cl or —Br. In several embodiments. X^{H} is —Cl. In several embodiments, Rⁱ¹ is C₁₋₆ alkoxy. In several embodiments, Rⁱ¹ is ethoxy as shown in Formula (I-int-k.1).

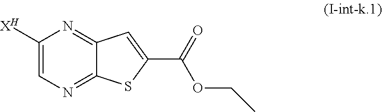

wherein the variables are as defined elsewhere herein. In several embodiments, X^{H} is —Cl and Rⁱ¹ as represented by Formula (I-int-k.2) or a salt thereof:

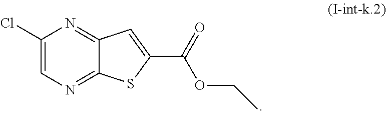

In several embodiments, the compound of Formula (I-int-k.2) is prepared by chlorinating the compound of Formula (I-int-j.1). In several embodiments, the compound of Formula (I-int-j.1) is chlorinated in the presence of a chlorinating agent (e.g., phenyl-phosphonic dichloride). In several embodiments, the compound of Formula (I-int-k.2) may be represented by a compound of Formula (I-int-a).

### Formula (I-int-i)

Several embodiments pertain to a compound of Formula (I-int-l) or a salt thereof:

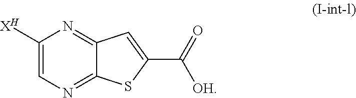

In several embodiments, the compound of Formula (I-int-l) is prepared by saponifying the compound of Formula (I-int-k). In several embodiments, the compound of Formula (I-int-k) is saponified in the presence of a strong base (e.g., NaOH). In several embodiments, the compound of Formula (I-int-l) may be represented by a compound of Formula (I-int-b). Several embodiments pertain to a compound of Formula (I-int-l.1) or a salt thereof:

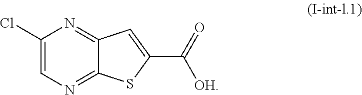 [SMILES: O=C(O)c1cc2nc(Cl)cnc2s1]

In several embodiments, the compound of Formula (I-int-l.1) is prepared by saponifying the compound of Formula (I-int-k.1). In several embodiments, the compound of Formula (I-int-k.1) is saponified in the presence of a strong base (e.g., NaOH). In several embodiments, the compound of Formula (I-int-l.1) may be represented by a compound of Formula (I-int-b).

### Formula (I-int-m)

Several embodiments pertain to a compound of Formula (I-int-m) or a salt thereof:

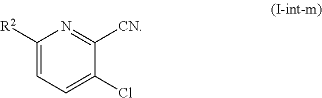

In several embodiments, R² is as disclosed elsewhere herein.

### Formula (I-int-n)

Several embodiments pertain to a compound of Formula (I-int-n) or a salt thereof:

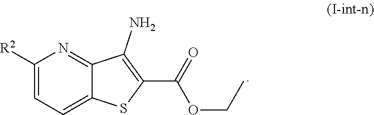

### Formula (I-int-o)

Several embodiments pertain to a compound of Formula (I-int-o) or a salt thereof:

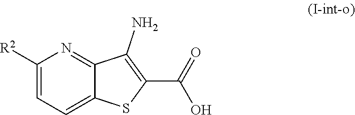

wherein the variables are as defined elsewhere herein.

### Preparation of Formula (I) Compounds

As illustrated in the schemes below, compounds of Formula (I) can be prepared using intermediate compounds as disclosed or combinations of any of the foregoing.

### Scheme 1.1 (Scheme 1a.1-1c.1)

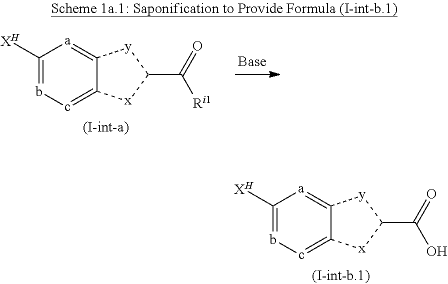

In several embodiments, the compound of Formula (I-int-a) is saponified using base (e.g., LiOH, NaOH, etc.). In several embodiments, the compound of Formula (I-int-a) is saponified using LiOH. In several embodiments, the reaction is performed in the presence of an appropriate solvent. In several embodiments, a compound of Formula (I-int-b.1) is provided. In several embodiments, the compound of Formula (I-int-b.1) is then subject to coupling conditions to provide a compound of Formula (I-int-c.1), as shown in Scheme 1b.1.

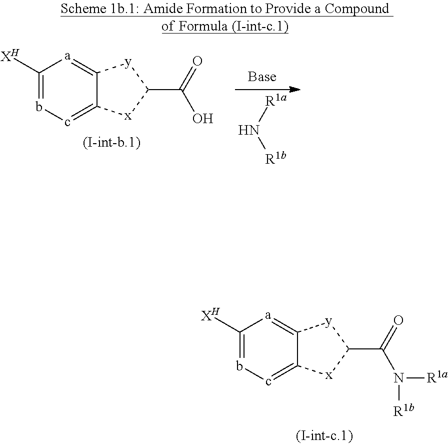

As illustrated in Scheme 1b.1, compounds of Formula (I-int-c.1) can be prepared by the reaction of an intermediate of Formula (I-int-b.1) with an amine of formula H—N(R^{1a})(R^{1b}). In several embodiments, the amide of Formula (I-int-c.1) is prepared in the presence of a coupling reagent (e.g., TBTU, DCC, DIC, EDC HCl, etc.). In several embodiments, the amide of Formula (I-int-c.1) is prepared in the presence of a base. In several embodiments, the base is an organic base. In several embodiments the base is TEA, DIPEA, etc.

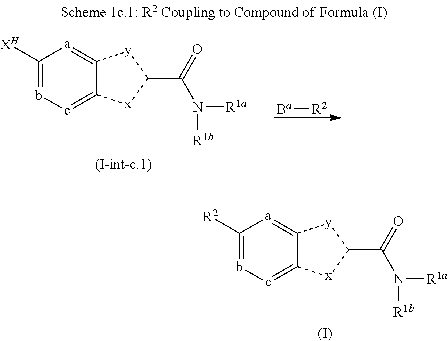

As illustrated in Scheme 1c.1, compounds of Formula (I) can be prepared by the reaction of an intermediate of Formula (I-int-c.1), in which X^{H} represents a suitable halogen atom (e.g., —Cl, —Br, etc.) or a similar reactive group (e.g., —OTf, —OTs, or —OMs) with an intermediate of B^{a}—R². In several embodiments, R² is as defined elsewhere herein (e.g., as in Formula (I), (Ia), (Ib), (Ic), (Id), (Ie), (If), (Ig), (Ih), (Ii), (Ij), (Ik), (Il), (Im), etc.). In several embodiments, B^{a} represents a suitable reactive moiety for cross-coupling reactions including, for example, Suzuki or Stille cross-coupling. In several embodiments, B^{a} is a boronic acid or a boronic acid ester (e.g., boronic pinacol ester) or a tributyl stannyl group. In several embodiments, a Suzuki coupling is performed using a palladium or similar transition metal catalyst together with appropriate ligands according to methods described in the literature and known to those skilled in the art.

### Scheme 1.2 (Scheme 1a.2-1c.2)

In an alternative embodiment to Scheme 1.1, the R² coupling can be performed on a compound of Formula (I-int-a) prior to saponification. Thereafter the intermediate by saponification, and amide formation. The conditions disclosed for Scheme 1.1 may be used for coinciding transformations in Scheme 1.2.

### Scheme 1a.2: R² Coupling to Compound of Formula (I-int-b.2)

In several embodiments, a compound of Formula (I-int-b.2) can be prepared by coupling R² to the compound of Formula (I-int-a). As disclosed elsewhere herein, Suzuki coupling or Stille coupling conditions may be used.

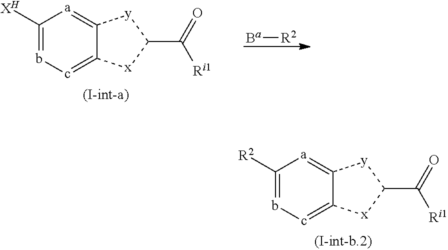

In several embodiments, this transformation can be performed using one or more conditions as disclosed in Scheme 1c.1. In several embodiments, the variables are as disclosed elsewhere herein (see, e.g., Scheme 1c.1).

### Scheme 1b.2: Saponification to Provide Formula (I-int-c.2)

In several embodiments, a compound of Formula (I-int-c.2) can be prepared by saponification of the compound of Formula (I-int-b.2) in the presence of base.

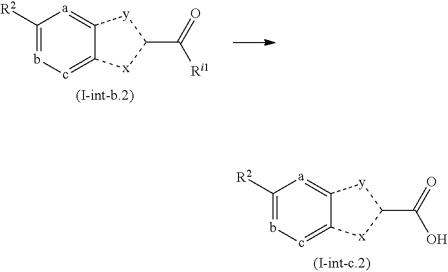

In several embodiments, this transformation can be performed using one or more conditions as disclosed in Scheme 1a.1.

### Scheme 1c.2: Amide Formation to Provide a Compound of Formula (I)

In several embodiments, a compound of Formula (I) can be prepared by coupling an amine with the compound of Formula (I-int-c.2) in the presence of a coupling reagent.

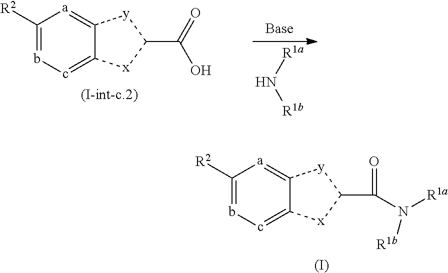

In several embodiments, this transformation can be performed using one or more conditions as disclosed in Scheme 1b.1.

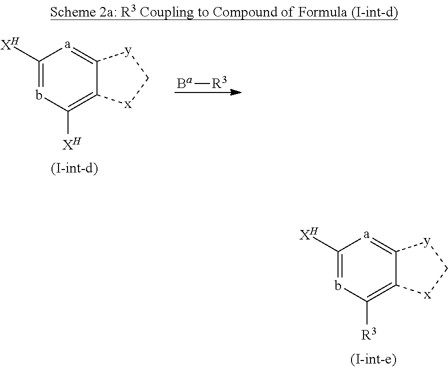

As illustrated in Scheme 2a, the intermediate of Formula (I-int-d) provides the intermediate of Formula (I-int-e) upon coupling with B^{a}—R³ (where B^{a} is as defined elsewhere herein and represents a suitable reactive moiety, such as a boronic acid or a boronic acid ester (e.g., boronic pinacol ester)) in a cross-coupling reaction (e.g., Suzuki). In several embodiments, a Suzuki coupling is performed using a palladium or similar transition metal catalyst together with appropriate ligands according to methods described in the literature and known to those skilled in the art.

### Scheme 2b: Lithiation and Carboxylation

In several embodiments, the intermediate of Formula (I-Int-e) is further subject to coupling conditions to provide a compound of Formula (I-Int-f), as shown in Scheme 2b.

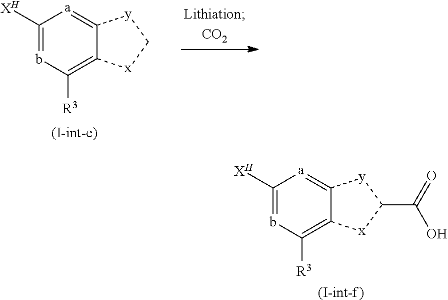

As illustrated in Scheme 2b, compounds of Formula (I-int-f) can be prepared by the reaction of an intermediate of Formula (I-int-e) with a lithiating agent (e.g., n-Bu-Li). In several embodiments, the lithiated Formula (I-int-e) compound is exposed to CO₂ to provide a compound of Formula (I-int-f).

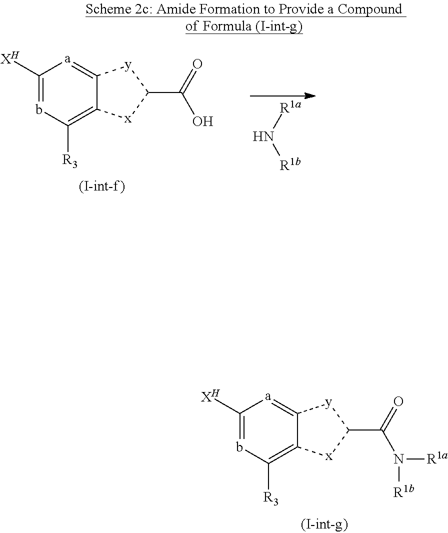

As illustrated in Scheme 2c, compounds of Formula (I-int-g) can be prepared by the reaction of an intermediate of Formula (I-int-f) with an amine of formula H—N(R^{1a})(R^{1b}), where R^{1a} and R^{1b} are as disclosed elsewhere herein. In several embodiments, the amide of Formula (I-int-c) is performed in the presence of a coupling reagent (e.g., TBTU, DCC, DIC, EDC HCl, etc.). In several embodiments, the amide of Formula (I-int-c) is performed in the presence of a base. In several embodiments, the base is an organic base. In several embodiments the base is TEA, DIPEA, etc.

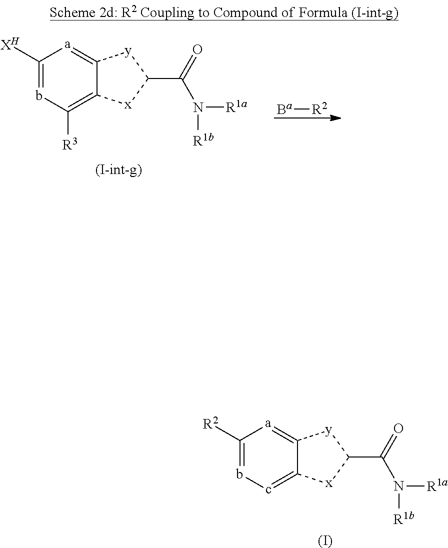

As illustrated in Scheme 2d, compounds of Formula (I) can be prepared by the reaction of an intermediate of Formula (I-int-g), in which X^{H} represents a suitable halogen atom (e.g., —Cl, —Br, etc.) or a similar reactive group, with an intermediate of B^{a}—R², where B^{a} represents a suitable reactive moiety, such as a boronic acid or a boronic acid ester (e.g., boronic pinacol ester) or a tributyl stannyl group, in a cross-coupling reaction (e.g., Suzuki or Stille). In several embodiments, R² is as defined elsewhere herein. In this embodiment, c is C—R³, where R³ is as defined elsewhere herein. In several embodiments, a Suzuki coupling is performed using a palladium or similar transition metal catalyst together with appropriate ligands according to methods described in the literature and known to those skilled in the art.

### Scheme 3

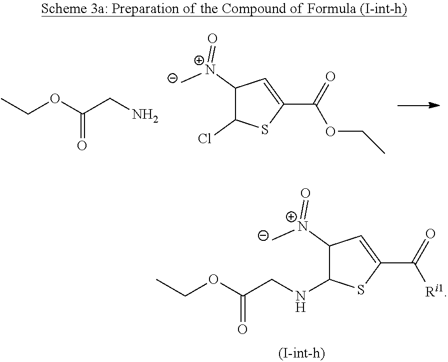

In several embodiments, a method of manufacturing a compound of Formula (I-int-h) is provided. In several embodiments, Formula (I-int-h) is prepared by performing an SNAr reaction using ethyl glycinate (e.g., the HCl salt of ethyl glycinate) and ethyl 5-chloro-4-nitro-4,5-dihydrothiophene-2-carboxylate. In several embodiments, the compound of Formula (I-int-h) is prepared in the presence of base (e.g., potassium carbonate).

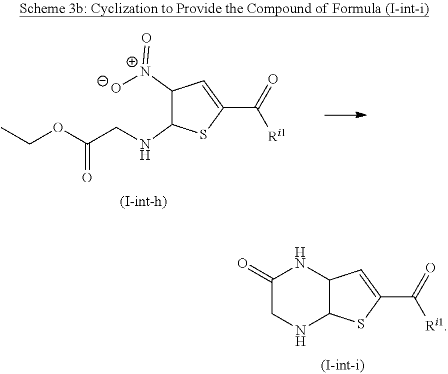

In several embodiments, the compound of Formula (I-int-i) is prepared by reducing the nitro group of Formula (I-int-h) in appropriate reducing conditions. In several embodiments, the compound of Formula (I-int-h) is reduced using Fe and AcOH.

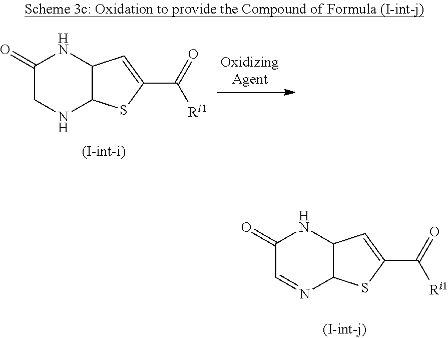

In several embodiments, the compound of Formula (I-int-j) is prepared by oxidizing the compound of Formula (I-int-j) in appropriate oxidizing conditions. In several embodiments, the compound of Formula (I-int-i) is oxidized in the presence of manganese dioxide.

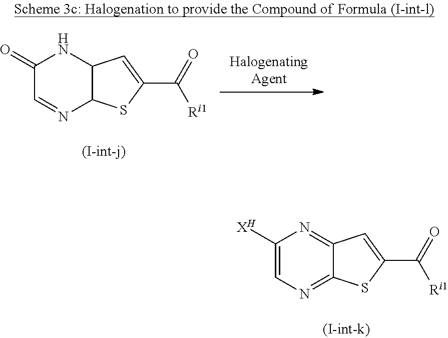

In several embodiments, a compound of Formula (I-int-k) is prepared by halogenating the compound of Formula (I-int-j). In several embodiments, the compound of Formula (I-int-j) is halogenated in the presence of a halogenating agent (e.g., phenyl-phosphonic dichloride). In several embodiments, the compound of Formula (I-int-k) may be represented by a compound of Formula (I-int-a).

As will be appreciated by a person of ordinary skill in the art, the compound of Formula I-int-k is also a compound falling under Formula (I-int-a). Thus, a compound of Formula (I) may be prepared using one or more of the steps shown for intermediate compound Formula (I-int-a) (e.g., one or more steps of Scheme 1.1 and/or Scheme 1.2). For example, in several embodiments, a compound of Formula (I-int-k) or Formula (I-int-k.1) may be saponified using conditions disclosed elsewhere herein to provide a compound of Formula (I-int-l) or Formula (I-int-l.1), respectively. In several embodiments, these compounds can then be coupled to R² using conditions disclosed elsewhere herein to provide compounds of Formula (I).

### Scheme 4

### Scheme 4a: R² Coupling to 3,6-Dihalo-Picolinonitrile

In several embodiments, as shown below, the compound of Formula (I-int-m) is prepared by coupling 3,6-dichloropicolinonitrile with an intermediate of B^{a}—R², where B^{a} represents a suitable reactive moiety, such as a boronic acid or a boronic acid ester (e.g., boronic pinacol ester) in a cross-coupling reaction (e.g., Suzuki). In several embodiments, a Suzuki coupling is performed using a palladium or similar transition metal catalyst together with appropriate ligands according to methods described in the literature and known to those skilled in the art.

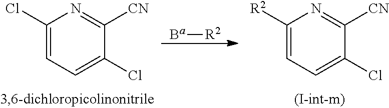

### Scheme 4b: Cyclization

In several embodiments, the compound of Formula (I-int-n) is prepared by cyclizing a compound of Formula (I-int-m) using for example ethyl 2-mercaptoacetate (or a C₁₋₆ mercaptoacetate).

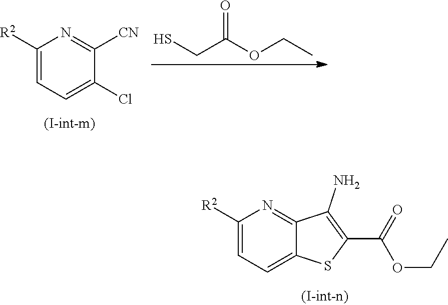

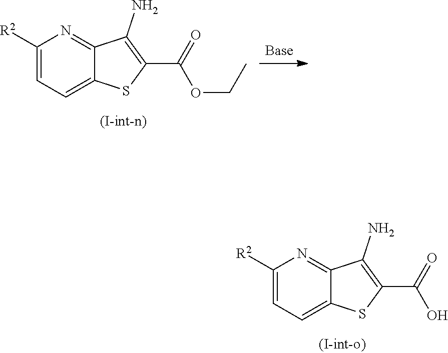

In several embodiments, a compound of Formula (I-int-n) may be saponified using conditions disclosed elsewhere herein to provide a compound of Formula (I-int-o). As will be appreciated by a person of ordinary skill in the art, the compound of Formula I-int-n is also a compound falling under Formula (I-int-b.2). Thus, a compound of Formula (I) may be prepared using one or more of the steps shown for intermediate compound Formula (I-int-b.2) (e.g., one or more steps of Scheme 1.2). In several embodiments, the compound of Formula (I-int-o) is prepared is prepared by saponifying the compound of Formula (I-int-n). In several embodiments, the compound of Formula (I-int-n) is saponified in the presence of a strong base (e.g., NaOH).

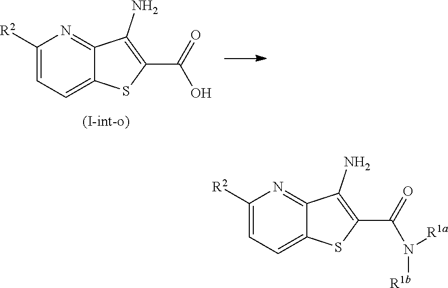

In several embodiments, a compound of Formula (I-int-o) (e.g., of Formula (I-int-c.2)) can then be coupled to H—N(R^{1a})(R^{1b}) using conditions disclosed elsewhere herein to provide falling under nFormula (I). In several embodiments, preparation of the amide of Formula (I) is performed in the presence of a coupling reagent (e.g., TBTU, DCC, DIC, EDC HCl, etc.). In several embodiments, the amide formation is performed in the presence of a base. In several embodiments, the base is an organic base. In several embodiments the base is TEA, DIPEA, etc.

### EXAMPLES

This section provides specific examples of compounds of Formula I and methods of making the same.

TABLE A
List of Abbreviations

| Ac | acetyl |
|---|---|
| MeCN or ACN | acetonitrile |
| AcOH | acetic acid |
| aq or aq. | aqueous |
| B₂pin₂ | bis(pinacolato)diboron |
| cataCXium A | [(di(1-adamantyl)- |
| Pd G3 | butylphosphine)-2-(2′-amino-1,1′- |
| | biphenyl)]palladium(II) methanesulfonate |
| Cy | cyclohexyl |
| DCM | dichloromethane |
| DIPEA | N-ethyl diisopropylamine (Hünig′s base) |
| DMF | N,N-dimethylformamide |
| DMSO | dimethyl sulfoxide |
| equiv. | equivalent |
| ESI or ES | electrospray ionization |
| Et | ethyl |
| EtOAc | ethyl acetate |
| EtOH | ethanol |
| g or gr | gram(s) |
| h | hour(s) |
| HATU | 1-[bis(dimethylamino)methylene]- |
| | 1H-1,2,3-triazolo[4,5- |
| | b]pyridinium 3-oxide hexafluorophosphate |
| HBpin | 4,4,5,5-tetramethyl-1,3,2-dioxaborolane |
| HPLC | high pressure liquid chromatography |
| iPr | iso-propyl |
| iPrOH or IPA | iso-propanol |
| KOAc | potassium acetate |
| LC MS, LCMS, | liquid chromatography mass spectroscopy |
| LC-MS or LC/MS | |
| LDA | lithium diisopropylamide |
| m/z | mass divided by charge |
| Me | methyl |
| MeOH | methanol |
| mg | milligrams |
| min | minutes |
| mL | milliliters |
| MS | mass spectra |
| MTBE | methyl tert-butyl ether |
| NaHMDS | sodium hexamethylsilazide |
| NMR | nuclear magnetic resonance |
| Pd(amphos)Cl₂ | bis(di-tert-butyl(4- |
| | dimethylaminophenyl)phosphine)dichlo- |
| | ropalladium(II) |
| Pd(dppf)Cl₂•DCM, | [1,1′- |
| Pd(dppf)Cl₂ | bis(diphenylphosphino)ferrocene]di- |
| | chloropalladium(II), |
| | complex with dichloromethane |
| Pd(PPh₃)₄ | tetrakis(triphenylphosphine)palladium(0) |
| Pd(dtbf)Cl₂ | [1,1′-bis(di-tert- |
| | butylphosphino)ferrocene]di- |
| | chloropalladium(II) |
| PEPPSI-iPr | [1,3-bis(2,6- |
| | diisopropylphenyl)imidazol-2-ylidene](3- |
| | chloropyridyl)palladium(II) dichloride |
| Pet ether | petroleum ether |
| Ph | phenyl |
| Pin | pinacolato |
| RP-HPLC | reverse phase high pressure |
| | liquid chromatography |
| RT or rt or r.t. | room temperature |
| sat. or satd. | saturated |
| SFC | supercritical fluid chromatography |
| TEA or Et₃N | triethylamine |
| TBTU | 2-(1H-benzotriazole-1-yl)- |
| | 1,1,3,3-tetramethylaminium |
| | tetrafluoroborate |
| temp | temperature |
| TFA | trifluoroacetic acid |
| THF | Tetrahydrofuran |
| UV | ultraviolet |

### Section 1: Intermediates

Provided in this section is the synthesis of various intermediates used to prepare compounds of Formula (I). All starting materials are either commercially available from Fisher Scientific or similar vendors, unless otherwise noted, or known in the art and may be synthesized by employing known procedures using ordinary skill.

### Intermediate 1: 2-methyl-5-(4,4,5,5-tetramethyl-1,3,2-dioxaborolan-2-yl)-2H-pyrazolo[3,4-b]pyridine

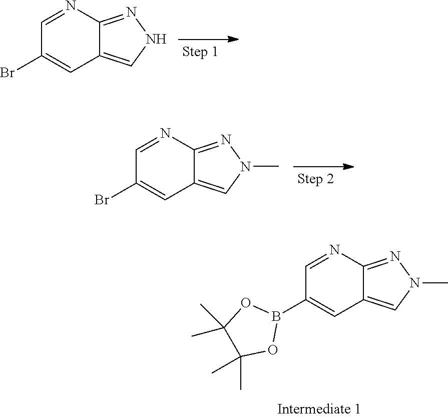

Step 1: 5-bromo-2-methyl-2H-pyrazolo[3,4-b]pyridine. To a mixture of 5-bromo-2H-pyrazolo[3,4-b]pyridine (50.0 g, 252 mmol) in THF (1600 mL) was added NaHMDS (379 mL, 379 mmol) at 0° C., After stirring for 0.5 h., MeI (47.4 mL, 757 mmol) was added at 0° C., The mixture was warmed to 25° C. and stirred for 4 h. The reaction mixture was quenched with H₂O (300 mL) at 0° C., then diluted with H₂O (200 mL), extracted with EtOAc (3×150 mL), washed with brine (150 mL), dried over Na₂SO₄, filtered and concentrated in vacuo. The crude mixture of isomers was absorbed onto a plug of silica gel and purified by column chromatography through a Redi-Sep pre-packed silica gel column (12 g), eluting with a gradient of 0% to 70% EtOAc in pet. ether, to provide 5-bromo-2-methyl-2H-pyrazolo[3,4-b]pyridine (8.00 g, 37.7 mmol, 74.7% yield) as a yellow solid. m/z (ESI): 212.0 (M+H)⁺. ¹H NMR (400 MHz, DMSO-d₆) δ 8.59 (d, J=2.38 Hz, 1H), 8.42 (s, 1H) 8.51 (d, J=2.38 Hz, 1H), 4.21 (s, 3H).

Step 2: 2-methyl-5-(4,4,5,5-tetramethyl-1,3,2-dioxaborolan-2-yl)-2H-pyrazolo[3,4-b]pyridine. To a solution of 5-bromo-2-methyl-2H-pyrazolo[3,4-b]pyridine (20.0 g, 94 mmol) in 1,4-dioxane (300 mL) was added KOAc (13.86 g, 141 mmol), B₂pin₂ (28.7 g, 113 mmol) and Pd(dppf)Cl₂-DCM adduct (3.85 g, 4.72 mmol). The mixture was stirred at 100° C. for 12 h under an atmosphere of N₂. The mixture was filtered through a celite plug and the filtrate was concentrated in vacuo. The reaction mixture was diluted with EtOAc (50 mL)/pet. ether (300 mL) and stirred at 20° C. for 15 min. The mixture was filtered and the filter cake was washed with pet. ether (3×300 mL). The filtrate was concentrated in vacuo and the solid was further dried under vacuum, to provide Intermediate 1 (24.0 g, 93 mmol, 98% yield) as a black solid. m/z (ESI): 178.1 (M-pinacol+2OH)⁺. ¹H NMR (400 MHz. DMSO-d₆) δ 8.72 (d, J 1.75 Hz, 1H), 8.53 (d, J=1.75 Hz, 1H), 8.47 (s, 1H), 4.21 (s, 3H), 1.32 (s, 12H).

### Intermediate 2: 6-(4,4,5,5-tetramethyl-1,3,2-dioxaborolan-2-yl)-1-((2-(trimethylsilyl)ethoxy)methyl)-1H-[1,2,3]triazolo[4,5-b]pyridine

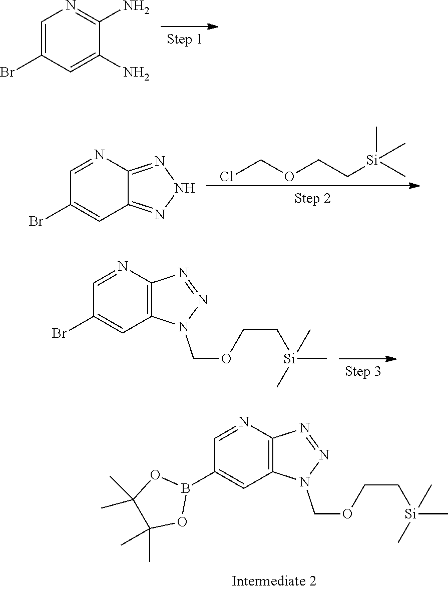

Step 1: 6-bromo-2H-[1,2,3]triazolo[4,5-b]pyridine. To a solution of 1N H₂SO₄ (150 mL) in H₂O (500 mL) was added 5-bromo-2,3-dihydropyridine-2,3-diamine (10.0 g, 52.6 mmol). The mixture was cooled to 0° C. and NaNO₂ (4.25 g, 61.6 mmol) in H₂O (120 mL) was added. Then, the mixture was stirred at 25° C. for 12 h under an atmosphere of N₂. The reaction mixture was poured into H₂O (80 mL), extracted with 2Me-THF (3×60 mL), washed with brine (50 mL), dried over Na₂SO₄, filtered and concentrated in vacuo to obtain the residue. The resulting residue was stirred with MTBE (50 mL) at 25° C. for 0.5 h. The suspension was filtered and the filter cake was dried under reduced pressure, to provide 6-bromo-2H-[1,2,3]triazolo[4,5-b]pyridine (8.00 g, 40.2 mmol, 76% yield) as a brown solid. m/z (ESI): 198.9 (M+H)⁺. ¹H NMR (400 MHz, DMSO-d₆) δ 8.78-8.79 (m, 2H).

Step 2: 6-bromo-1-((2-(trimethylsilyl)ethoxy)methyl)-1H-[1,2,3]triazolo[4,5-b]pyridine. To a solution of 6-bromo-1H-[1,2,3]triazolo[4,5-b]pyridine (1.000 g, 5.02 mmol) in THF (10 mL) at 0° C. was added NaH (0.181 g, 7.54 mmol). The mixture was stirred at 0° C. for 30 min. Then, (2-ethoxyethyl)trimethylsilyl chloride (1.103 g, 6.62 mmol) was added dropwise at 0° C. and stirring was continued for 1 h. The reaction mixture was diluted with H₂O (40 mL) and sat. NH₄Cl solution (30 mL), extracted with EtOAc (3×30 mL), washed with brine (30 mL), dried over Na₂SO₄, filtered and the filtrate was concentrated in vacuo. The crude material was absorbed onto a plug of silica gel and purified by column chromatography through a SEPAFLASH® pre-packed silica gel column (40 g), eluting with a gradient of 0% to 55% EtOAc in pet ether, to provide 6-bromo-1-((2-(trimethylsilyl)ethoxy)methyl)-1H-[1,2,3]triazolo[4,5-b]pyridine (650 mg, 1.97 mmol, 39% yield) as an oil. m/z (ESI): 328.9 (M+H)⁺. ¹H NMR (400 MHz, DMSO-d₆) δ 9.02 (d, J=2.0 Hz, 1H), 8.92 (d, J=2.0 Hz, 1H), 6.04 (s, 2H), 3.67 (t, J=8.0 Hz, 2H), 0.84 (t, J=8.0 Hz, 2H), −0.11 (s, 9H).

Step 3: 6-(4,4,5,5-tetramethyl-1,3,2-dioxaborolan-2-yl)-1-((2-(trimethylsilyl)ethoxy)methyl)-1H-[1,2,3]triazolo[4,5-b]pyridine. To a solution of B₂pin₂ (93.0 mg, 0.364 mmol) and 6-bromo-1-((2-(trimethylsilyl)ethoxy)methyl)-1H-[1,2,3]triazolo[4,5-b]pyridine (100 mg, 0.304 mmol) in 1,4-dioxane (1 mL) was added KOAc (44.7 mg, 0.456 mmol) and Pd(dppf)Cl₂-DCM adduct (12.6 mg, 0.015 mmol). The mixture was degassed with N₂, then stirred at 80° C. for 12 h. The solvent was removed in vacuo and the resulting crude boronate ester was used without further purification. m/z (ESI): 377.1 (M+H)⁺.

### Intermediate 3: 2-methyl-6-(4,4,5,5-tetramethyl-1,3,2-dioxaborolan-2-yl)-2H-[1,2,3]triazolo[4,5-b]pyridine

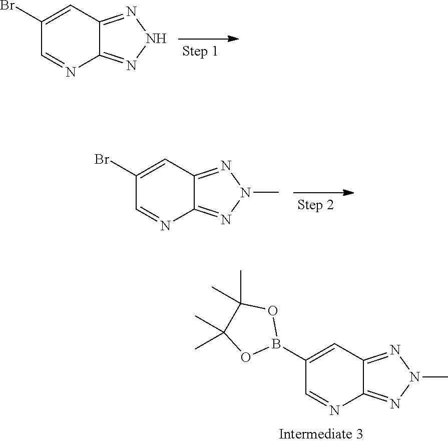

Step 1: 6-bromo-2-methyl-2H-[1,2,3]triazolo[4,5-b]pyridine. 6-bromo-2-methyl-2H-[1,2,3]triazolo[4,5-b]pyridine was prepared according to the literature procedure (Journal of Organic Chemistry (2018), 83(12), 6334-6353).

Step 2: 2-methyl-6-(4,4,5,5-tetramethyl-1,3,2-dioxaborolan-2-yl)-2H-[1,2,3]triazolo[4,5-b]pyridine. To a solution of 6-bromo-2-methyl-2H-[1,2,3]triazolo[4,5-b]pyridine (1.50 g, 7.04 mmol) in 1,4-dioxane (5 mL) was added B₂Pin₂ (3.580 g, 14.08 mmol), KOAc (1.175 g, 11.97 mmol), and Pd(dppf)Cl₂·DCM (0.575 g, 0.704 mmol). The mixture was stirred at 110° C. for 12 hr. The reaction mixture was diluted with H₂O (20 mL) and extracted with EtOAc (3×10 mL). The combined organic layers were washed with brine (15 mL), dried over Na₂SO₄, filtered and concentrated under reduced pressure to give a residue. The residue was purified by column chromatography (SiO₂, Pet ether/EtOAc=5/1 to 3/1) to afford Intermediate 3 as a yellow solid (1.60 g, 6.15 mmol, 87% yield). m/z (ESI): 261.0 (M+H)⁺

### Section 2—General Procedures

### Group 1

### Example 1-1: (3-methyl-6-(2-methyl-2H-pyrazolo[3,4-b]pyridin-5-yl)-1-benzothiophen-2-yl)(4-morpholinyl)methanone A2

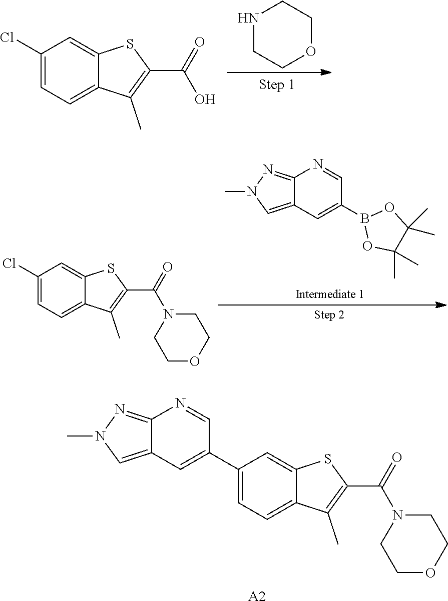

Step 1: (6-chloro-3-methylbenzo[b]thiophen-2-yl)(morpholino)methanone. To a stirred mixture of 6-chloro-3-methylbenzo[b]thiophene-2-carboxylic acid (400 mg, 1.77 mmol), TBTU (850 mg, 2.65 mmol) in DMF (30 mL). The resulting solution was cooled to 0° C., morpholine (184 mg, 2.12 mmol) and DIPEA (1.54 mL, 8.82 mmol) were added, and the resulting mixture was stirred at 0° C. for 1 h. The reaction mixture was poured into H₂O (40 mL). Then the mixture was extracted with EtOAc (3×30 mL). The combined organic layer was washed with brine (30 mL), dried over Na₂SO₄, filtered, and the filtrate was concentrated under reduced pressure to obtain the residue. The residue was purified by column chromatography (40 g SiO₂, Eluent of 0~28% EtOAc/pet ether with a flow rate of 80 mL/min) to obtain residue. (6-chloro-3-methylbenzo[b]thiophen-2-yl)(morpholino)methanone (500 mg, 1.690 mmol, 96% yield) was obtained as white solid. ¹H NMR (400 MHz, DMSO-d₆) δ 8.17 (d, J=1.6 Hz, 1l), 7.84 (d, J=8.4 Hz, 1H), 7.48 (dd, J=1.6, 8.4 Hz, 1H), 3.45-3.65 (m, 8H), 2.36 (s, 3H).

Step 2: (3-methyl-6-(2-methyl-2H-pyrazolo[3,4-b]pyridin-5-yl)benzo[b]thiophen-2-yl)(morpholino)methanone. A mixture of 2-methyl-5-(4,4,5,5-tetramethyl-1,3,2-dioxaborolan-2-yl)-2H-pyrazolo[3,4-b]pyridine (210 mg, 0.811 mmol), (6-chloro-3-methylbenzo[b]thiophen-2-yl)(morpholino)methanone (120 mg, 0.406 mmol), K₃PO₄ (172 mg, 0.811 mmol), Pd(dppf)Cl₂ (33.1 mg, 0.041 mmol) in THF (1 mL) and H₂O (0.2 mL) was degassed with N₂ 3 times. The mixture was stirred at 80° C. for 12 hr under an atmosphere of N₂. The mixture was poured into H₂O (20 ml). Then the mixture was extracted with EtOAc (6×20 mL), the organic layer was washed with brine (30 mL), dried over Na₂SO₄, filtered and the filtrate was concentrated under reduced pressure. The residue was purified first by flash silicagel chromatography (12 g SEPAFLASH® Silica Flash Column, Eluent of EtOAc/Pet Ether with a flow rate of 60 mL/min). The residue was further purified by prep-HPLC. Compound A2 (27.9 mg, 0.071 mmol, 18% yield) was obtained as white solid. m/z (ESI): 393.1 (M+H)⁺. ¹H NMR (400 MHz, DMSO-d₆) δ 9.00 (d, J=2.4 Hz, 1H), 8.53 (d, J=2.4 Hz, 1H), 8.50 (s, 1H), 8.41 (d, J=1.6 Hz, 1H), 7.93 (s, 1H), 7.86 (d, J=6.1 Hz, 1H), 4.235 (s, 3H), 3.63-3.55 (m, 8H), 2.55 (brs J=, 3H)

Compounds of Table 1-1 were Prepared Following the Procedure Described in Group A.1.

TABLE 1-1

| | Chemical Structure & | |
|---|---|---|
| Ex. # | Name | LCMS: (ESI + ve ion) m/z; NMR |
| A8 | 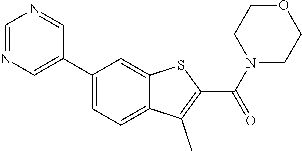 [SMILES: Cc1c(C(=O)N2CCOCC2)sc2cc(-c3cncnc3)ccc12] | 340.1; ¹H NMR (400 MHz, DMSO- d₆) δ 9.25 (s, 2H), 9.21 (s, 1H), 8.51 (d, J = 1.2 Hz, 1H), 7.98 (d, J= 8.0 Hz, 1H), 7.93 (t, J = 4.0 Hz, 1H), 3.63- 3.55 (m, 8H), 2.41 (s, 3H), |
| | (3-methyl-6-(5- | |
| | pyrimidinyl)-1- | |
| | benzothiophen-2-yl)(4- | |
| | morpholinyl)methanone | |
| A9 | 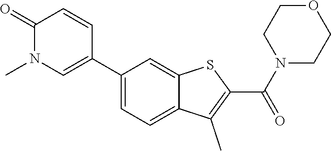 [SMILES: Cc1c(C(=O)N2CCOCC2)sc2cc(-c3ccc(=O)n(C)c3)ccc12] | 369.1; ¹H NMR (400 MHz, DMSO- d₆) δ 8.26 (d, J = 2.4 Hz, 1H), 8.21 (d, J = 1.6 Hz, 1H), 7.93 (d, J = 9.6 Hz, 1H), 7.86 (d, J = 8.4 Hz, 1H), 7.70 (t, J = 4.0 Hz, 1H), 6.51 (d, J = 9.6 Hz, 1H), 3.62 (s, 8H), 3.53 (s, 3H), 2.38 (s, 3H) |
| | 1-methyl-5-(3-methyl-2-(4- | |
| | morpholinylcarbonyl)-1- | |
| | benzothiophen-6-yl)-2(1H)- | |
| | pyridinone | |
| A10 | 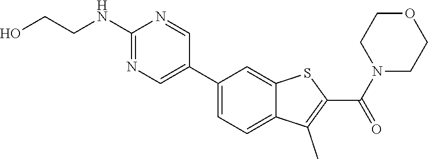 [SMILES: Cc1c(C(=O)N2CCOCC2)sc2cc(-c3cnc(NCCO)nc3)ccc12] | 399.2; ¹H NMR (400 MHz, DMSO- d₆) δ 2.38 (s, 3 H) 3.40 (br d, J = 6.13 Hz, 4 H) 3.52-3.56 (m, 4 H) 3.62 (br s, 4 H) 4.70 (t, J = 5.57 Hz, 1 H) 7.25 (t, J = 5.88 Hz, 1 H) 7.74 (dd, J = 8.44, 1.44 Hz, 1 H) 7.87 (d, J = 8.38 Hz, 1 H) 8.27 (d, J = 1.00 Hz, 1 H) 8.72 (s, 2 H) |
| | hydroxyethyl)amino)-5- | |
| | pyrimidinyl)-3-methyl-1- | |
| | benzothiophen-2-yl)(4- | |
| | morpholinyl)methanone | |
| A11 | 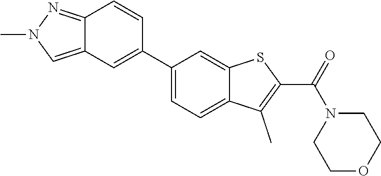 [SMILES: Cc1c(C(=O)N2CCOCC2)sc2cc(-c3ccc4nn(C)cc4c3)ccc12] | 392.2; ¹H NMR (400 MHz, DMSO- d₆) δ 8.41 (s, 1H), 8.33 (s, 1H), 8.07 (s, 1H), 7.89 (d, J = 8.0 Hz, 1H), 7.81 (d, J = 8.0 Hz, 1H), 7.96(t, J = 8.0 Hz, 2H), 4.20 (s, 3H), 3.63-3.56 (m, 8H), 2.40 (s, 3H) |
| | (3-methyl-6-(2-methyl-2H- | |
| | indazol-5-yl)-1- | |
| | benzothiophen-2-yl)(4- | |
| | morpholinyl)methanone | |

### Group 2

### Example 2-1: (4,4-difluoro-1-piperidinyl)(3-methyl-6-(2-methyl-2H-pyrazolo[3,4-b]pyridin-5-yl)-1-benzothiophen-2-yl)methanone A3

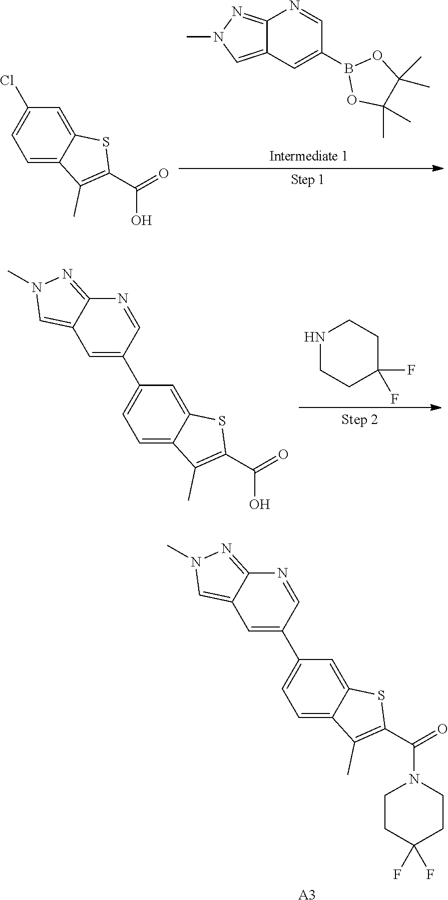

Step 1: 3-methyl-6-(2-methyl-2H-indazol-5-yl)benzo[b]thiophene-2-carboxylic acid. To a solution of ethyl 3-methyl-6-(2-methyl-2H-pyrazolo[3,4-b]pyridin-5-yl)benzo[b]thiophene-2-carboxylate (50 mg, 0.142 mmol) in tetrahydrofuran (1 mL), water (0.2 mL), and ethanol (0.1 mL) was added LiOH·H₂O (17.0 mg, 0.711 mmol) at 20° C. The mixture was stirred at 20° C. for 12 h. The reaction mixture was concentrated under reduced pressure. The resulting mixture was acidified to pH 2 with 0.1 N HCl. The aqueous phase was extracted with EtOAc (3×10 mL). The combined organics were dried over MgSO4, and concentrated to give the product as a gray solid (40 mg, 0.124 mmol, 87% yield). ¹H NMR (400 MHz, DMSO-d6) δ 8.98 (d, J=1.9 Hz, 111), 8.44-8.52 (m, 2H), 8.17 (s, 1H), 7.66-7.80 (m, 2H), 4.23 (s, 3H), 2.67 (s, 3H).

Step 2: (4,4-difluoropiperidin-1-yl)(3-methyl-6-(2-methyl-2H-pyrazolo[3,4-b]pyridin-5-yl)benzo[b]thiophen-2-yl)methanone. To a solution of 3-methyl-6-(2-methyl-2H-pyrazolo[3,4-b]pyridin-5-yl)benzo[b]thiophene-2-carboxylic acid (150 mg, 0.464 mmol) in DMF (2 mL) was added HATU (882 mg, 2.32 mmol) and DIPEA (0.405 mL, 2.32 mmol) at 0° C. The mixture was stirred at 0° C. for 2 min. Then 4,4-difluoropiperidine (56.2 mg, 0.464 mmol) was added to the mixture. The mixture was stirred at 20° C. for another 1 h The reaction mixture was diluted with H₂O (20 mL). Then the mixture was extracted with EtOAc (6×20 mL). The combined organic layer was washed with brine (30 mL), dried over Na₂SO₄, filtered and the filtrate was concentrated under reduced pressure. The residue was purified by prep-HPLC (Column: Waters Xbridge BEH C18100*30 mm*10 um; mobile phase: [H₂O (10 mM NH₄HCO₃)-ACN]; B %: 40%-80%, 8 min). Compound A3 (59.1 mg, 0.139 mmol, 30% yield) was obtained as a white solid. m/z (ESI): 427.2 (M+H)⁺; ¹H NMR (400 MHz, DMSO-d₆) δ 9.01 (d, J=2.4 Hz, 1H), 8.57 (m, 1H), 8.55 (s, 1H), 8.54 (s, 1H), 7.97 (d, J=4 Hz, 1H), 7.94 (s, 1H), 4.24 (s, 3H), 3.67 (br s, 4H), 2.42 (s, 3H), 2.06 (br s, 4H). ¹⁹F NMR (376 MHz, DMSO-d₆) δ −96.0 (s, 2F).

The compounds of Table 2-1 were prepared following the procedure described in Group 2.

TABLE 2-1

| | Chemical Structure & | | Reaction |
|---|---|---|---|
| Ex. # | Name | LCMS: (ESI + ve ion) m/z; NMR | Comments |
| A1S | 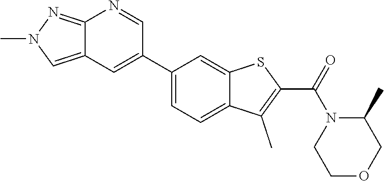 | 407.2; ¹H NMR (400 MHz, DMSO- d₆) δ 9.00 (d, J = 2.3 Hz, 1H), 8.55- 8.49 (m, 2H), 8.42 (d, J = 1.0 Hz, 1H), 7.96-7.92 (m, 1H), 7.89-7.85 (m, 1H), 4.24 (s, 4H), 3.85 (br d, J = 8.9 Hz, 1H), 3.75-3.54 (m, 3H), 3.48- 3.38 (m, 2H), 2.40 (s, 3H), 1.28 (d, J = 6.8 Hz, 3H) | |
| | (3-methyl-6-(2-methyl-2H- | | |
| | pyrazolo[3,4-b]pyridin-5- | | |
| | yl)-1-benzothiophen-2- | | |
| | yl)((3S)-3-methyl-4- | | |
| | morpholinyl)methanone | | |
| A1R | 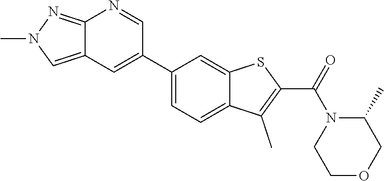 | 407.2; ¹H NMR (400 MHz, DMSO- d₆) δ 9.00 (d, J = 2.4 Hz, 1H), 8.53 (d, J = 2.4 Hz, 1H), 8.50 (s, 1H), 8.42 (s, 1H), 7.93 (d, J = 8.4 Hz, 1H), 7.88- 7.85 (m, 1H), 4.24 (s, 4H), 3.85 (br d, J = 8.9 Hz, 1H), 3.75-3.54 (m, 3H), 3.42-3.32 (m, 2H), 2.40 (s, 3H), 1.28 (d, J = 6.8 Hz, 3H) | |
| | (3-methyl-6-(2-methyl-2H- | | |
| | pyrazolo[3,4-b]pyridin-5- | | |
| | yl)-1-benzothiophen-2- | | |
| | yl)((3R)-3-methyl-4- | | |
| | morpholinyl)methanone | | |
| A4R | 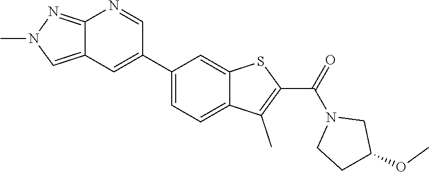 | 407.2; ¹H NMR (400 MHz, DMSO- d₆) δ 9.01 (d, J = 2.4 Hz, 1H), 8.54 (d, J = 2.4 Hz, 1H), 8.50 (s, 1H), 8.41 (s, 1H), 7.95-7.85 (m, 2H), 4.24 (s, 3H), 3.61 (m, 2H), 3.32-3.19 (m, 2H), 2.43 (s, 3H), 2.02 (s, 2H) | Confirmed by SFC: Chiralpak AD-3, 150 × 4.6 mm, I.D., 3 um; mobile phase: 60% EtOH (0.1% IPAm) using a flow rate of 2.5 mL/min |
| | ((3R)-3-methoxy-1- | | |
| | pyrrolidinyl)(3-methyl-6- | | |
| | (2-methyl-2H-pyrazolo[3,4- | | |
| | b]pyridin-5-yl)-1- | | |
| | benzothiophen-2- | | |
| | yl)methanone | | |
| A4S | 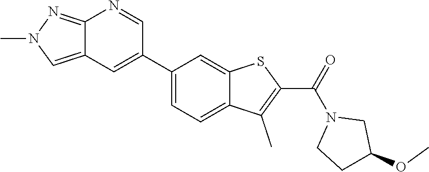 | 407.2; ¹H NMR (400 MHz, DMSO- d₆) δ 9.00 (d, J = 2.4Hz, 1H), 8.53 (d, J = 2.4 Hz, 1H), 8.50 (s, 1H), 8.41 (s, 1H), 7.93-7.85 (m, 2H), 4.23 (s, 3H), 3.96 (br d, J = 4.0 Hz, 1H), 3.60-3.53 (m, 1H), 3.44-3.19 (m, 2H), 2.42 (s, 3H), 2.02-1.96 (m, 2H) | Confirmed by SFC: Chiralpak AD-3, 150 × 4.6 mm, I.D., 3 um; mobile phase: 60% EtOH (0.1% IPAm) using a flow rate of 2.5 mL/min |
| | ((3S)-3-methoxy-1- | | |
| | pyrrolidinyl)(3-methyl-6- | | |
| | (2-methyl-2H-pyrazolo[3,4- | | |
| | b]pyridin-5-yl)-1- | | |
| | benzothiophen-2- | | |
| | yl)methanone | | |
| A5 | 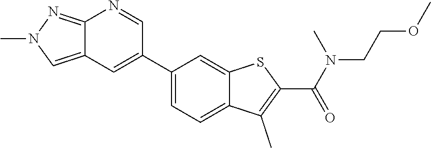 | 395.2; ¹H NMR (400 MHz, DMSO- d₆) δ 9.00 (d, J = 2.4 Hz, 1H), 8.53 (d, J = 10.0 Hz, 1H), 8.53 (s, 1H), 8.41- 8.38 (m, 2H), 4.50 (s, 3H), 3.75-3.39 (br m, 2H), 3.31-3.10 (br m, 2H), 3.05 (s, 3H), 2.32 (s. 3H) | |
| | N-(2-methoxyethyl)-N,3- | | |
| | dimethyl-6-(2-methyl-2H- | | |
| | pyrazolo[3,4-b]pyridin-5- | | |
| | yl)-1-benzothiophene-2- | | |
| | carboxamide | | |
| A6 | 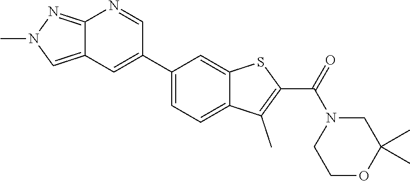 | 421.2; ¹H NMR (400 MHz, DMSO- d₆) δ 9.01 (s, 1H), 8.53 (d, J = 2.4 Hz, 1H), 8.50 (s, 1H), 8.41 (s, 1H), 7.98- 7.93 (m, 2H), 4.24 (s, 3H), 3.65 (br s, 2H), 3.53-3.49 (m, 2H), 3.40-3.33 (m, 2H), 2.45 (s, 3H), 1.15 (s, 6H) | |
| | (2,2-dimethyl-4- | | |
| | morpholinyl)(3-methyl-6- | | |
| | (2-methyl-2H-pyrazolo[3,4- | | |
| | b]pyridin-5-yl)-1- | | |
| | benzothiophen-2- | | |
| | yl)methanone | | |
| A7S | 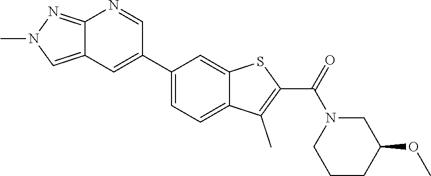 | 421.2; ¹H NMR (400 MHz, DMSO- d₆) δ 9.00 (d, J = 2.0 Hz, 1H), 8.58- 8.50 (m, 1H), 8.41 (s, 1H), 8.43 (s, 1H), 4.24 (s, 3H), 3.62-3.37 (m, 3H), 3.25-3.19 (m, 2H), 2.39 (s, 3H), 1.85 (br m, 1H), 1.70 (m, 2H), 1.42-1.37 (m, 1H) | Confirmed by SFC: Chiralpak AD-3, 150 × 4.6 mm, I.D., 3 um; mobile phase: 50% EtOH (0.1% IPAm) using a flow rate of 4 mL/min |
| | ((3S)-3-methoxy-1- | | |
| | piperidinyl)(3-methyl-6-(2- | | |
| | methyl-2H-pyrazolo[3,4- | | |
| | b]pyridin-5-yl)-1- | | |
| | benzothiophen-2- | | |
| | yl)methanone | | |
| A7R | 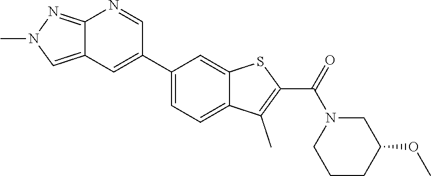 | 421.2; ¹H NMR (400 MHz, DMSO- d₆) δ 9.00 (d, J = 2.0 Hz, 1H), 8.53 (d, J = 2.4 Hz, 1H), 8.50 (s, 1H), 8.39 (d, J = 8.0 Hz, 1H), 7.87-7.85 (m, 2H), 4.24 (s, 3H), 3.73-3.37 (m, 3H), 3.20- 3.11 (m, 2H), 2.39 (s, 3H), 1.84 (br s, 1H), 1.68 (m, 2H), 1.44-1.35 (m, 1H) | Confirmed by SFC: Chiralpak AD-3, 150 × 4.6 mm, I.D., 3 um; mobile phase: 50% EtOH (0.1% IPAm) using a flow rate of 4 mL/min |
| | ((3R)-3-methoxy-1- | | |
| | piperidinyl)(3-methyl-6-(2- | | |
| | methyl-2H-pyrazolo[3,4- | | |
| | b]pyridin-5-yl)-1- | | |
| | benzothiophen-2- | | |
| | yl)methanone | | |

### Group 3

### Example 3-1: (3-amino-4-cyclobutyl-6-(2-methyl-2H-pyrazolo[3,4-b]pyridin-5-yl)thieno[2,3-b]pyridin-2-yl)(4,4-difluoro-1-piperidinyl)methanone B4

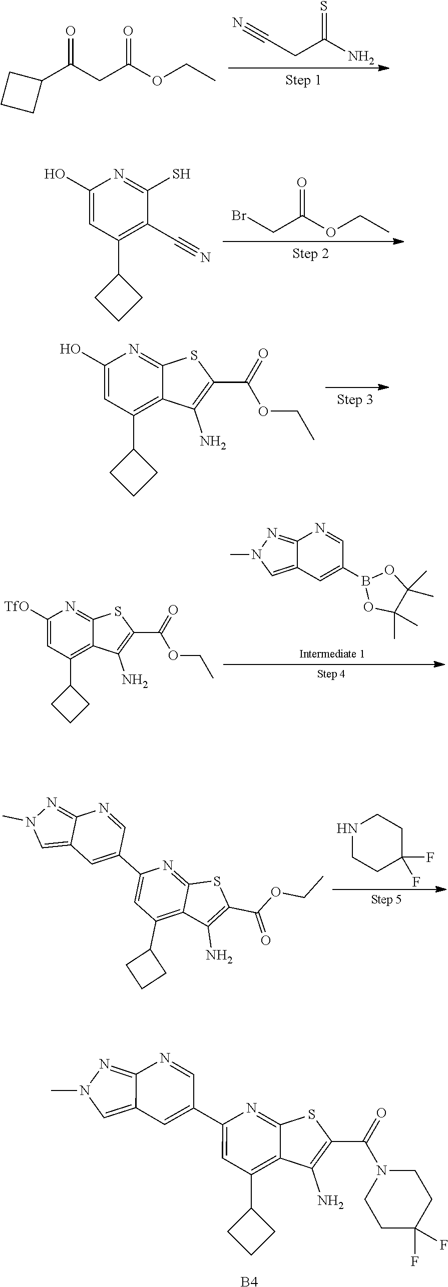

Step 1: 4-cyclobutyl-6-hydroxy-2-mercaptonicotinonitrile. To a solution of ethyl 3-cyclobutyl-3-oxo-propanoate (10.00 g, 58.75 mmol) in MeOH (100 mL) was added KOH (4.94 g, 88.1 mmol) and 2-cyanothioacetamide (8.83 g, 88.1 mmol). The mixture was stirred at 60° C. for 12 hr. The mixture was cooled to room temperature, and the product was collected by filtration. The solid was triturated with EtOAc at 20° C. for 30 min. The solid was collected and dried under reduced pressure. The crude product was used in the next step without further purification. 4-Cyclobutyl-6-hydroxy-2-mercaptonicotinonitrile (10.50 g, 49.38 mmol, 84% yield) was obtained as a white solid. 207.0 (M+H)⁺. ¹H NMR (400 MHz, DMSO-d₆) δ 10.34-10.58 (m, 1H), 5.36-5.44 (m, 1H), 3.22-3.30 (m, 1H), 2.17-2.26 (m, 2H), 1.98-1.85 (m, 3H), 1.67-1.76 (m, 1H).

Step 2: ethyl 3-amino-4-cyclobutyl-6-hydroxy-thieno[2,3-b]pyridine-2-carboxylate. To a solution of 4-cyclobutyl-6-hydroxy-2-sulfanyl-pyridine-3-carbonitrile (1.00 g, 4.85 mmol) in EtOH (10 mL) was added ethyl 2-bromoacetate (891 mg, 5.33 mmol, 0.590 mL) and NaOEt (990 mg, 14.54 mmol,). The reaction mixture was stirred at 80° C. for 2 hr. The reaction mixture was diluted with H₂O (500 mL). Then the mixture was extracted with EtOAc (3×300 mL). The combined organic layer was washed with brine (200 mL), dried over Na₂SO₄, filtered and the filtrate was concentrated under reduced pressure. The crude product was triturated with MTBE at 20° C. for 20 min, then the solid was dried under reduced pressure. Ethyl 3-amino-4-cyclobutyl-6-hydroxy-thieno[2,3-b]pyridine-2-carboxylate (1.00 g, 3.18 mmol, 66% yield) was obtained as a white solid. m/z (ESI): 293.1 (M+H)⁺. ¹H NMR (400 MHz, DMSO-d₆) δ 6.47 (br s, 2H), 5.93 (s, 1H), 4.13 (q, J=7.1 Hz, 2H), 3.88 (br t, J=8.2 Hz, 1H), 2.23-2.37 (m, 2H), 1.88-2.06 (m, 3H), 1.75 (br d, J=8.4 Hz, 1H), 1.22 (t, J=7.1 Hz, 3H)

Step 3: ethyl 3-amino-4-cyclobutyl-6-(trifluoromethylsulfonyloxy)thieno[2,3-b]pyridine-2-carboxylate. To the solution of ethyl 3-amino-4-cyclobutyl-6-hydroxy-thieno[2,3-b]pyridine-2-carboxylate (9.00 g, 30.8 mmol) in DCM (100 mL) was added DIPEA (7.96 g, 61.6 mmol, 10.72 mL). The mixture was cooled to −78° C. Then, Tf₂O (9.55 g, 33.9 mmol, 5.59 mL) was added under N₂ at −78° C. The mixture was stirred at −78° C. for 1 hr. The reaction mixture was diluted with H₂O (500 mL) and allowed to reach room temperature. The mixture was extracted with EtOAc (3×400 mL). The combined organic layer was washed with brine (300 mL), dried over Na₂SO₄, filtered and the filtrate was concentrated under reduced pressure. The residue was triturated with MTBE at 20° C. for 20 min, then the solid was dried under reduced pressure. The residue was purified by flash silica gel chromatography (40 g SEPAFLASH® Silica Flash Column, Eluent of 0~15% EtOAc/Pet ether gradient with a flow rate of 100 mL/min). Ethyl 3-amino-4-cyclobutyl-6-(trifluoromethylsulfonyloxy)thieno[2,3-b]pyridine-2-carboxylate was obtained as a yellow solid. m/z (ESI): 425.0 (M+H)⁺. ¹H NMR (400 MHz, DMSO-d₆) δ 7.45 (s, 1H), 6.90 (br s, 2H), 4.39 (quin, J=8.4 Hz, 1H), 4.29 (q, J=7.0 Hz, 2H), 2.41-2.48 (m, 2H), 2.26 (dt, J=2.3, 9.0 Hz, 2H), 2.00-2.13 (m, 1H), 1.77-1.89 (m, 1H), 1.29 (t, J=7.1 Hz, 3H)

Step 4: ethyl 3-amino-4-cyclobutyl-6-(2-methylpyrazolo[3,4-b]pyridin-5-yl)thieno[2,3-b]pyridine-2-carboxylate. To a mixture of ethyl 3-amino-4-cyclobutyl-6-(trifluoromethylsulfonyloxy)thieno[2,3-b]pyridine-2-carboxylate (3.20 g, 7.54 mmol) and (2-methylpyrazolo[3,4-b]pyridin-5-yl)boronic acid (1.60 g, 9.05 mmol) in dioxane (30 mL) and H₂O (6 mL) was added Pd(PPh₃)₄ (436 mg, 0.38 mmol) and K₂CO₃ (2.08 g, 15.08 mmol) under N₂. The reaction mixture was stirred at 60° C. for 12 hr. then diluted with H₂O (200 mL). The mixture was extracted with EtOAc (3×300 mL). The combined organic layer was washed with brine (100 mL), dried over Na₂SO₄, filtered and the filtrate was concentrated under reduced pressure. The residue was purified by flash silica gel chromatography (40 g SEPAFLASH® Silica Flash Column, Eluent of 0~100% EtOAc/Pet ether gradient with a flow rate of 120 mL/min). Ethyl 3-amino-4-cyclobutyl-6-(2-methylpyrazolo[3,4-b]pyridin-5-yl)thieno[2,3-b]pyridine-2-carboxylate (2.00 g, 4.91 mmol, 65% yield) was obtained as a yellow solid. m/z (ESI): 408.1 (M+H). ¹H NMR (400 MHz, DMSO-d₆) δ 9.42 (d, J=2.3 Hz, 1H), 9.05 (d, J=2.3 Hz, 1H), 8.57 (s, 1H), 7.91 (s, 1H), 6.86 (s, 2H), 4.41-4.34 (m, 1H), 4.27-4.32 (m, 2H), 4.25 (s, 3H), 2.35-2.47 (m, 4H), 2.17-2.03 (m, 1H), 1.83-1.94 (m, 1H), 1.31 (t, J=7.1 Hz, 3H).

Step 5: (3-amino-4-cyclobutyl-6-(2-methyl-2H-pyrazolo[3,4-b]pyridin-5-yl)thieno[2,3-b]pyridin-2-yl)(4,4-difluoropiperidin-1-yl)methanone. A solution of ethyl 3-amino-4-cyclobutyl-6-(2-methyl-2H-pyrazolo[3,4-b]pyridin-5-yl)thieno[2,3-b]pyridine-2-carboxylate (0.064 g, 0.157 mmol), and LiOH·H₂O (0.066 g, 1.57 mmol) in THF (1 mL), H₂O (0.5 mL), and MeOH (2 mL) was stirred at 60° C. for 2 hours. The reaction was concentrated under reduced pressure. The residue was taken up in DMF (1 mL) and treated with 4,4-difluoropiperidine (0.029 g, 0.029 mL, 0.236 mmol), TBTU (0.076 g, 0.236 mmol), and TEA (0.064 g, 0.088 mL, 0.628 mmol). The resulting solution was stirred at RT for 5 hours, concentrated and the residue purified by automated flash chromatography using 0-10% MeOH in DCM (product eluted at 4%) to afford Compound B4 (0.033 g, 0.068 mmol, Yield: 44%). m/z (ESI): 483.1 (M+H)⁺. ¹H NMR (400 MHz, chloroform-d) δ 9.41 (d, J=2.07 Hz, 1H) 8.73 (d, J=2.28 Hz, 1H) 8.01 (s, 1H) 7.65 (s, 1H) 5.94 (br s, 2H) 4.30 (s, 3H) 4.19 (t, J=8.40 Hz, 1H) 3.85-3.93 (m, 4H) 2.37-2.58 (m, 4H) 1.97-2.23 (m, 6H). ¹⁹F NMR (376 MHz, chloroform-d) δ −97.39 (s, 2 F).

The compounds of Table 3-1 were prepared following the procedure described for Group 3.

TABLE 3-1

| | Chemical Structure & | |
|---|---|---|
| Ex. # | Name | LCMS: (ESI + ve ion) m/z; NMR |
| B3 | 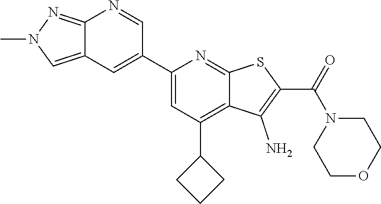 [SMILES: Cn1cc2cc(-c3cc(C4CCC4)c4c(N)c(C(=O)N5CCOCC5)sc4n3)cnc2n1] | 449.2; ¹H NMR (400 MHz, chloroform-d) δ 9.41 (d, J = 2.30 Hz, 1 H) 8.72 (d, J = 2.30 Hz, 1 H) 8.00 (s, 1 H) 7.65 (s, 1 H) 5.90 (s, 2 H) 4.19-4.31 (m, 4 H) 3.74-3.86 (m, 8 H) 2.38-2.58 (m, 4 H) 2.09-2.26 (m, 1 H) 1.95-2.09 (m, 1 H). |
| | (3-amino-4-cyclobutyl-6- | |
| | (2-methyl-2H-pyrazolo[3,4- | |
| | b]pyridin-5-yl)thieno[2,3- | |
| | b]pyridin-2-yl)(4- | |
| | morpholinyl)methanone | |
| B5 | 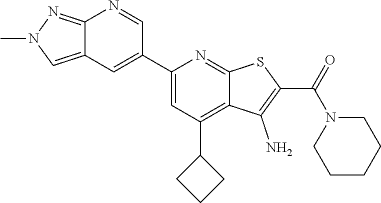 [SMILES: Cn1cc2cc(-c3cc(C4CCC4)c4c(N)c(C(=O)N5CCCCC5)sc4n3)cnc2n1] | 449.2; ¹H NMR (400 MHz, chloroform-d) δ 9.42 (d, J = 2.07 Hz, 1 H) 8.75 (d, J = 2.07 Hz, 1 H) 8.01 (s, 1 H) 7.67 (s, 1 H) 5.73 (br s, 2 H) 4.22-4.32 (m, 4 H) 3.75 (br d, J = 5.60 Hz, 4 H) 2.39-2.58 (m, 4 H) 2.11-2.26 (m, 1 H) 1.97-2.06 (m, 1 H) 1.57-1.81 (m, 6 H). |
| | (3-amino-4-cyclobutyl-6- | |
| | (2-methyl-2H-pyrazolo[3,4- | |
| | b]pyridin-5-yl)thieno[2,3- | |
| | b]pyridin-2-yl)(1- | |
| | piperidinyl)methanone | |
| B6S | 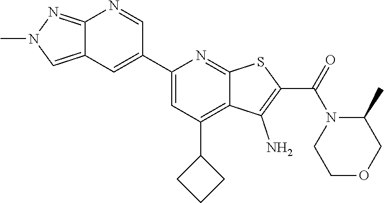 | 463.2; ¹H NMR (500 MHz, chloroform-d) δ 9.36 (d, J = 2.08 Hz, 1 H) 8.60 (d, J = 2.21 Hz, 1 H) 7.93 (s, 1 H) 7.56 (s, 1 H) 5.81 (s, 2 H) 4.56-4.63 (m, 1 H) 4.12-4.27 (m, 5 H) 3.92 (dd, J = 11.42, 3.11 Hz, 1 H) 3.63-3.74 (m, 2 H) 3.56 (td, J = 11.81, 2.60 Hz, 1 H) 3.39 (td, J = 12.91, 3.50 Hz, 1 H) 2.43-2.55 (m, 2 H) 2.31-2.42 (m, 2 H) 2.07-2.18 (m, 1 H) 1.92-2.00 (m, 1 H) 1.44 (d, J = 6.88 Hz, 3 H). |
| | (3-amino-4-cyclobutyl-6- | |
| | (2-methyl-2H-pyrazolo[3,4- | |
| | b]pyridin-5-yl)thieno[2,3- | |
| | b]pyridin-2-yl)((3S)-3- | |
| | methyl-4- | |
| | morpholinyl)methanone | |
| B6R | 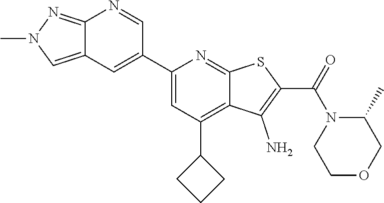 | 463.2; ¹H NMR (500 MHz, chloroform-d) δ 9.39 (d, J = 2.08 Hz, 1 H) 8.67 (d, J = 2.21 Hz, 1 H) 7.97 (s, 1 H) 7.61 (s, 1 H) 5.84 (br s, 2 H) 4.59-4.67 (m, 1 H) 4.17-4.32 (m, 5 H) 3.95 (dd, J = 11.35, 2.92 Hz, 1 H) 3.64-3.78 (m, 2 H) 3.58 (td, J = 11.77, 2.66 Hz, 1 H) 3.38-3.47 (m, 1 H) 2.35-2.56 (m, 4 H) 2.10-2.22 (m, 1 H) 1.95-2.05 (m, 1 H) 1.47 (d, J = 6.88 Hz, 3 H). |
| | NH2 (3-amino-4-cyclobutyl-6- | |
| | (2-methyl-2H-pyrazolo[3,4- | |
| | b]pyridin-5-yl)thieno[2,3- | |
| | b]pyridin-2-yl)((3R)-3- | |
| | methyl-4- | |
| | morpholinyl)methanone | |

### Group 4

### Example 4-1: (5-(2-methyl-2H-pyrazolo[3,4-b]pyridin-5-yl)thieno[2,3-b]pyridin-2-yl)(1-piperidinyl)methanone B16

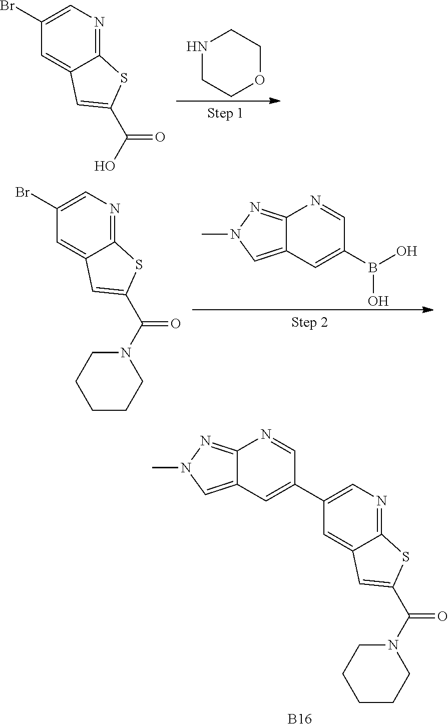

Step 1: (5-bromothieno[2,3-b]pyridin-2-yl)(piperidin-1-yl)methanone. A mixture of 5-bromothieno[2,3-b]pyridine-2-carboxylic acid (258 mg, 1.00 mmol), TEA (303 mg, 0.421 mL, 3.00 mmol), and TBTU (353 mg, 1.10 mmol) in DMF (5 mL) was treated with piperidine (85 mg, 0.099 mL, 1.00 mmol). The reaction mixture was stirred at 25° C. for 18 hr. The reaction mixture was diluted with H₂O (5 mL). The resulting precipitate was collected by filtration, washed with H₂O and dried to give (5-bromothieno[2,3-b]pyridin-2-yl)(piperidin-1-yl)methanone (175.8 mg, 0.541 mmol, 54% yield) as a tan solid. m/z (ESI): 325.0/327.0 (1:1) (M+H)⁺. ¹H NMR (400 MHz, DMSO-d₆) δ 8.74 (d, J=2.1 Hz, 1H), 8.61 (d, J=2.3 Hz, 1H), 7.62 (s, 1H), 3.57-3.68 (m, 4H), 1.63-1.72 (m, 2H), 1.59 (br d, J=4.8 Hz, 4H)

Step 2: (5-(2-methyl-2H-pyrazolo[3,4-b]pyridin-5-yl)thieno[2,3-b]pyridin-2-yl)(1-piperidinyl)methanone. A vial was charged with (5-bromothieno[2,3-b]pyridin-2-yl)(piperidin-1-yl)methanone (84 mg, 0.258 mmol), (2-methyl-2H-pyrazolo[3,4-b]pyridin-5-yl)boronic acid (68.6 mg, 0.387 mmol), anhydrous K₂CO₃ (107 mg, 0.775 mmol) and Pd(dppf)Cl₂ (18.90 mg, 0.026 mmol) and 1,4-dioxane (1.2 mL)/H₂O (0.3 mL). The vial was capped and stirred at 90° C. for 1.5 h. The reaction mixture was concentrated under reduced pressure, absorbed onto silica gel, and purified by chromatography through a Redi-Sep pre-packed silica gel column (12 g), eluting with a gradient of 0% to 10% MeOH in DCM, to obtain Compound B16 (65.8 mg, 0.174 mmol, 68% yield), as a white solid. m/z (ESI): 378.0 (M+H)⁺. ¹H NMR (400 MHz, DMSO-d₆) δ 9.03 (d, J=2.3 Hz, 1H), 9.01 (d, J=2.5 Hz, 1H), 8.68 (d, J=2.3 Hz, 1H), 8.61 (d, J=2.3 Hz, 1H), 8.54 (s, 1H), 7.71 (s, 1H), 4.26 (s, 3H), 3.62-3.70 (m, 4H), 1.68 (br d, J=5.4 Hz, 2H), 1.54-1.64 (m, 4H)

### Example 4-2: (4-cyclopropyl-6-(2-methyl-2H-pyrazolo[3,4-b]pyridin-5-yl)thieno[2,3-b]pyridin-2-yl)((3S)-3-methyl-4-morpholinyl)methanone B17S

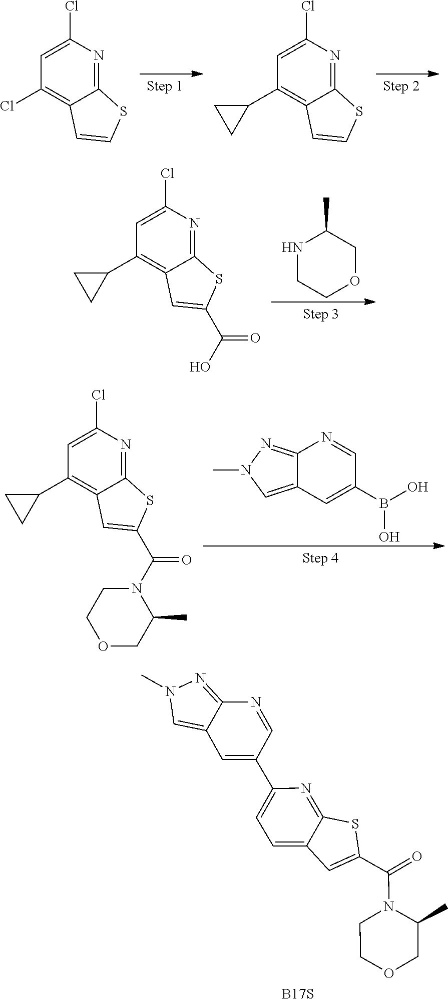

Step 1: 6-chloro-4-cyclopropylthieno[2,3-b]pyridine. To a stirring solution of 4,6-dichlorothieno[2,3-b]pyridine (1.50 g, 7.35 mmol) and PEPPSI-iPr catalyst (0.501 g, 0.735 mmol) in THF (5 mL) at 0° C. was added dropwise a 0.5M solution of cyclopropylzinc(II) bromide (22 mL, 11 mmol) in THF. The reaction was stirred at 0° C. for 30 min. The reaction was diluted with saturated ammonium chloride solution and extracted with EtOAc (25 mL). The organic phase was concentrated and the residue purified by chromatography through a Redi-Sep pre-packed silica gel column using 0-40% EtOAc in heptane to afford 6-chloro-4-cyclopropylthieno[2,3-b]pyridine (0.571 g, 2.72 mmol, 37% yield). m/z (ESI): 210.1 (M+H)⁺. ¹H NMR (400 MHz, chloroform-d) δ 7.68 (d, J=8.0 Hz, 1H), 7.59 (d, J=8.0 Hz, 1H), 6.81 (s, 1H), 2.30 (dt, J=5.0, 8.4 Hz, 1H), 1.15-1.29 (m, 2H), 0.89-1.01 (m, 2H).

Step 2: 6-chloro-4-cyclopropylthieno[2,3-b]pyridine-2-carboxylic acid. To a 50-mL pressure-release vial were added 6-chloro-4-cyclopropylthieno[2,3-b]pyridine (0.57 g, 2.72 mmol) and tetrahydrofuran (3 mL). The resulting solution was flushed with N₂ and cooled to −78° C. A 2.5M solution of n-butyllithium, in hexanes (1.305 mL, 3.26 mmol) was added dropwise. The resulting mixture was allowed to stir at −78° C. for 5 min under an atmosphere of N₂. A separate 50-mL pressure-release vial equipped with a cannula was charged with solid carbon dioxide (2.393 g, 54.4 mmol). A cannula was connected to the reaction vial, allowing the evolved CO₂ gass to bubble through the reaction mixture at −78° C. for 20 min. The reaction mixture was sparged with N₂ for 5 min to flush out any dissolved CO₂, then allowed to warm to 23° C. over the course of 30 minutes while sparging with N₂. The reaction mixture was then quenched by slow addition of sat. aq. NH₄Cl (5 mL) and transferred to a separatory funnel with EtOAc (30 mL), H₂O (10 mL), and 1 N aq. HCl (10 mL). The aqueous layer was extracted with EtOAc (30 mL), and the combined organics were dried and residue purified by automated flash column chromatography using 0-100% EtOAc/EtOH (3:1) in heptane to afford 6-chloro-4-cyclopropylthieno[2,3-b]pyridine-2-carboxylic acid (0.421 g. 1.659 mmol, Yield: 61%) as a white solid. m/z (ESI): 254.1 (M+H)⁺.

Step 3: (S)-(6-chloro-4-cyclopropylthieno[2,3-b]pyridin-2-yl)(3-methylmorpholino)methanone. A mixture of 6-chloro-4-cyclopropylthieno[2,3-b]pyridine-2-carboxylic acid (0.070 g, 0.276 mmol), (S)-3-methylmorpholine (0.033 g, 0.033 mL, 0.331 mmol), chloro-N,N,N′,N′-tetramethylformamidinium hexafluorophosphate (0.093 g, 0.331 mmol), and 1-methylimidazole (0.068 g, 0.066 mL, 0.828 mmol) in ACN (1 mL) was stirred at rt for 12 hours. The reaction was diluted with H₂O (10 mL) and extracted with EtOAc. The organic was concentrated to afford crude (S)-(6-chloro-4-cyclopropylthieno[2,3-b]pyridin-2-yl)(3-methylmorpholino)methanone. m/z (ESI): 337.2 (M+H)⁺.

Step 4: (S)-(4-cyclopropyl-6-(2-methyl-2H-pyrazolo[3,4-b]pyridin-5-yl)thieno[2,3-b]pyridin-2-yl)(3-methylmorpholino)methanone. The crude (S)-(6-chloro-4-cyclopropylthieno[2,3-b]pyridin-2-yl)(3-methylmorpholino)methanone was dissolved in dioxane (3 mL) and H₂O (1 mL) in a microwave vial and treated with [2-methyl-2H-pyrazolo[3,4-b]pyridin-5-yl]boronic acid (0.073 g, 0.414 mmol). Solid Pd(dppf)Cl₂ (0.040 g, 0.055 mmol), and K₂CO₃ (0.114 g, 0.828 mmol) were added. The vial was sparged with argon, capped and heated in microwave at 110° C. for 30 minutes. The reaction was concentrated under reduced pressure and the residue purified by automated flash column chromatography using 0-100% EtOAc/EtOH (3:1) in heptane to afford Compound B17S (0.082 g, 0.189 mmol, Yield: 69%). m/z (ESI): 434.1 (M+H)⁺. ¹H NMR (400 MHz, chloroform-d) δ 9.35 (d, J=2.30 Hz, 1H) 8.71 (d, J=2.09 Hz, 1H) 8.01 (s, 1H) 7.72 (s, 1H) 7.31 (s, 1H) 4.54 (br d, J=5.85 Hz, 1H) 4.29 (s, 3H) 4.15-4.24 (m, 1H) 3.97 (dd, J=11.50, 3.34 Hz, 1H) 3.68-3.78 (m, 2H) 3.44-3.63 (m, 2H) 2.33 (tt, J=8.44, 5.15 Hz, 1H) 1.47-1.50 (m, 2H) 1.19-1.32 (m, 3H) 0.96-1.08 (m, 2H).

### Alternate Conditions for Group 4

(1) 0.2 equiv. of cataCXium Pd G3 and K₃PO₄ was used as the catalyst and base, instead of Pd(dppf)Cl₂ and K₂CO₃. Toluene/H₂O were used as solvents, instead of 1,4-dioxane/H₂O

(2) 5 mol % Pd(PPh₃)₄ and Na₂CO₃ was used as the catalyst and base, instead of Pd(dppf)Cl₂ and K₂CO₃.

(3) Stille Cross-Coupling: To a stirring solution of product from Step 1 (1 equiv.) in 1,4-dioxane (1 mL) was added 3-methyl-6-(tributylstannyl)pyrimidin-4(3H)-one (413 mg, 1.035 mmol) and Pd(PPh₃)₄ (100 mg, 0.086 mmol). The mixture was stirred at 120° C. for 12 h under an atmosphere of N₂. The reaction mixture was quenched by addition of H₂O (20 mL), extracted with EtOAc (5×20 mL), washed with brine (15 mL), dried over Na₂SO₄, filtered and concentrated in vacuo. The crude material was absorbed onto a plug of silica gel and purified by column chromatography through a SEPAFLASH® pre-packed silica gel column (4 g), eluting with a gradient of 0% to 100% EtOAc in pet ether, to provide the product.

The compounds of Table 4-1 were prepared following the procedure described in Group 4.

TABLE 4-1

| | | | Reaction |
|---|---|---|---|
| Ex. # | Chemical Structure & Name | LCMS: (ESI + ve ion) m/z; NMR | Comments |
| B8 | 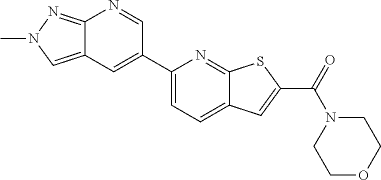 [SMILES: Cn1cc2cc(-c3ccc4cc(C(=O)N5CCOCC5)sc4n3)cnc2n1] | 380.1; ¹H NMR (400 MHz, chloroform-d) δ 9.42 (d, J = 2.09 Hz, 1 H) 8.78 (d, J = 2.09 Hz, 1 H) 8.17 (d, J = 8.57 Hz, 1 H) 8.03 (s, 1 H) 7.86 (d, J = 8.36 Hz, 1 H) 7.47 (s, 1 H) 4.31 (s, 3 H) 3.74-3.88 (m, 8 H). | |
| | (6-(2-methyl-2H-pyrazolo[3,4- | | |
| | b]pyridin-5-yl)thieno[2,3- | | |
| | b]pyridin-2-yl)(4- | | |
| | morpholinyl)methanone | | |
| B9R | 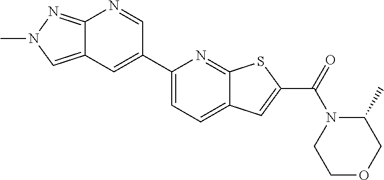 | 394.2; ¹H NMR (400 MHz, chloroform-d) δ 9.41 (s, 1 H) 8.76 (d, J = 1.88 Hz, 1 H) 8.15 (d, J = 8.36 Hz, 1 H) 8.02 (s, 1 H) 7.84 (d, J = 8.36 Hz, 1 H) 7.44 (s, 1 H) 4.44-4.58 (m, 1 H) 4.29 (s, 3 H) 4.17 (br d, J = 12.75 Hz, 1 H) 3.97 (br dd, J = 11.08, 2.51 Hz, 1 H) 3.67-3.79 (m, 2 H) 3.43-3.63 (m, 2 H) 1.48 (d, J = 6.90 Hz, 3 H). | Confirmed by SFC: Chiralpak IH-3, 100 × 4.6 mm, I.D., 3 um; mobile phase: 35% EtOH (0.1% IPAm) using a flow rate of 4 mL/min |
| | ((3R)-3-methyl-4- | | |
| | morpholinyl)(6-(2-methyl-2H- | | |
| | pyrazolo[3,4-b]pyridin-5- | | |
| | yl)thieno[2,3-b]pyridin-2- | | |
| | yl)methanone | | |
| B9S | 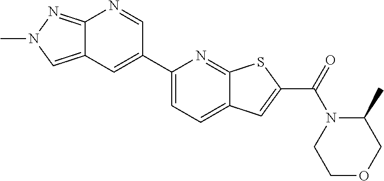 | 394.2; ¹H NMR (400 MHz, DMSO-d₆) δ 9.41 (d, J = 2.30 Hz, 1 H) 8.97 (d, J = 2.30 Hz, 1 H) 8.56 (s, 1 H) 8.42 (d, J = 8.36 Hz, 1 H) 8.20 (d, J = 8.36 Hz, 1 H) 7.76 (s, 1 H) 4.43 (br d, J = 4.60 Hz, 1 H) 4.25 (s, 3 H) 4.04 (br d, J = 11.08 Hz, 1 H) 3.89 (br d, J = 7.73 Hz, 1 H) 3.58-3.72 (m, 2 H) 3.39-3.52 (m, 2 H) 1.37 (d, J = 6.90 Hz, 3 H). | Confirmed by SFC: Chiralpak IH-3, 100 × 4.6 mm, I.D., 3 um; mobile phase: 35% EtOH (0.1% IPAm) using a flow rate of 4 mL/min |
| | ((3S)-3-methyl-4- | | |
| | morpholinyl)(6-(2-methyl-2H- | | |
| | pyrazolo[3,4-b]pyridin-5- | | |
| | yl)thieno[2,3-b]pyridin-2- | | |
| | yl)methanone | | |
| B10 | 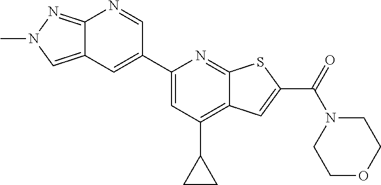 [SMILES: Cn1cc2cc(-c3cc(C4CC4)c4cc(C(=O)N5CCOCC5)sc4n3)cnc2n1] | 420.1; ¹H NMR (400 MHz, chloroform-d) δ 9.36 (d, J = 2.30 Hz, 1 H) 8.75 (d, J = 2.30 Hz, 1 H) 8.02 (s, 1 H) 7.76 (s, 1 H) 7.34 (s, 1 H) 4.31 (s, 3 H) 3.77-3.88 (m, 8 H) 2.31-2.39 (m, 1 H) 1.26-1.30 (m, 2 H) 0.99-1.09 (m, 2 H). | |
| | (4-cyclopropyl-6-(2-methyl- | | |
| | 2H-pyrazolo[3,4-b]pyridin-5- | | |
| | yl)thieno[2,3-b]pyridin-2-yl)(4- | | |
| | morpholinyl)methanone | | |
| B11 | 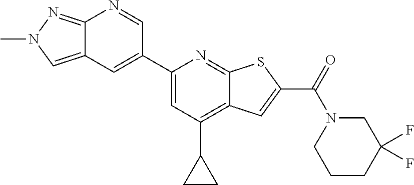 [SMILES: Cn1cc2cc(-c3cc(C4CC4)c4cc(C(=O)N5CCCC(F)(F)C5)sc4n3)cnc2n1] | 454.2; ¹H NMR (400 MHz, chloroform-d) δ 9.37 (s, 1 H) 8.76 (d, J = 2.09 Hz, 1 H) 8.02 (s, 1 H) 7.79 (s, 1 H) 7.35 (s, 1 H) 4.32 (s, 3 H) 4.00 (t, J = 11.08 Hz, 2 H) 3.76-3.83 (m, 2 H) 2.32-2.40 (m, 1 H) 2.17 (tt, J = 13.17, 6.48 Hz, 2 H) 1.90-1.98 (m, 2 H) 1.24-1.32 (m, 2 H) 0.99-1.10 (m, 2 H). ¹⁹F NMR (376 MHz, chloroform- d) δ −102.91 (s, 2 F). | |
| | (4-cyclopropyl-6-(2-methyl- | | |
| | 2H-pyrazolo[3,4-b]pyridin-5- | | |
| | yl)thieno[2,3-b]pyridin-2- | | |
| | yl)(3,3-difluoro-1- | | |
| | piperidinyl)methanone | | |
| B12 | 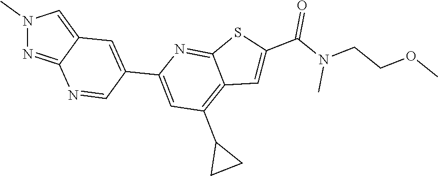 | 422.2; ¹H NMR (400 MHz, chloroform-d) δ 9.36 (d, J = 2.09 Hz, 1 H) 8.76 (d, J = 2.09 Hz, 1 H) 8.02 (s, 1 H) 7.85 (s, 1 H) 7.34 (s, 1 H) 4.31 (s, 3 H) 3.78-3.84 (m, 2 H) 3.71 (br d, J = 4.81 Hz, 2 H) 3.43 (s, 3 H) 3.22-3.38 (m, 3 H) 2.32-2.40 (m, 1 H) 1.22-1.31 (m, 2 H) 1.01-1.08 (m, 2 H). | |
| | 4-cyclopropyl-N-(2- | | |
| | methoxyethyl)-N-methyl-6-(2- | | |
| | methyl-2H-pyrazolo[3,4- | | |
| | b]pyridin-5-yl)thieno[2,3- | | |
| | b]pyridine-2-carboxamide | | |
| B13 | 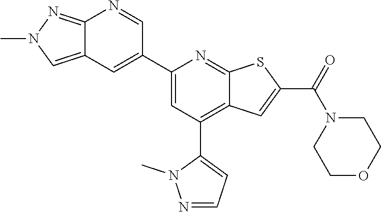 [SMILES: Cn1cc2cc(-c3cc(-c4ccnn4C)c4cc(C(=O)N5CCOCC5)sc4n3)cnc2n1] | 460.2; ¹H NMR (400 MHz, chloroform-d) δ 9.44 (d, J = 2.30 Hz, 1 H) 8.81 (d, J = 2.09 Hz, 1 H) 8.06 (s, 1 H) 7.81 (s, 1 H) 7.68 (d, J = 1.67 Hz, 1 H) 7.40 (s, 1 H) 6.55 (d, J = 1.67 Hz, 1 H) 4.32 (s, 3 H) 3.93 (s, 3 H) 3.78 (br d, J = 3.55 Hz, 8 H). | |
| | (6-(2-methyl-2H-pyrazolo[3,4- | | |
| | b]pyridin-5-yl)-4-(1-methyl- | | |
| | 1H-pyrazol-5-yl)thieno[2,3- | | |
| | b]pyridin-2-yl)(4- | | |
| | morpholinyl)methanone | | |
| B14 | 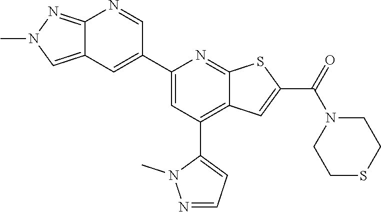 [SMILES: Cn1cc2cc(-c3cc(-c4ccnn4C)c4cc(C(=O)N5CCSCC5)sc4n3)cnc2n1] | 476.2; ¹H NMR (400 MHz, chloroform-d) δ 9.45 (d, J = 2.30 Hz, 1 H) 8.83 (d, J = 2.09 Hz, 1 H) 8.06 (s, 1 H) 7.82 (s, 1 H) 7.69 (d, J = 1.88 Hz, 1 H) 7.39 (s, 1 H) 6.56 (d, J = 1.88 Hz, 1 H) 4.33 (s, 3 H) 3.99-4.08 (m, 4 H) 3.94 (s, 3 H) 2.67-2.81 (m, 4 H) | |
| | (6-(2-methyl-2H-pyrazolo[3,4- | | |
| | b]pyridin-5-yl)-4-(1-methyl- | | |
| | 1H-pyrazol-5-yl)thieno[2,3- | | |
| | b]pyridin-2-yl)(4- | | |
| | thiomorpholinyl)methanone | | |
| B15 | 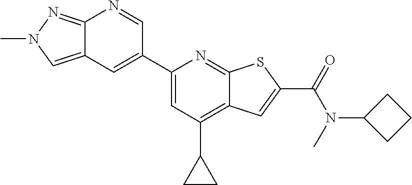 [SMILES: CN(C(=O)c1cc2c(C3CC3)cc(-c3cnc4nn(C)cc4c3)nc2s1)C1CCC1] | 418.2; ¹H NMR (400 MHz, chloroform-d) δ 9.30-9.38 (m, 1 H), 8.65-8.72 (m, 1 H), 8.00 (s, 1 H), 7.70 (s, 1 H), 7.29 (s, 1 H), 4.81 (br t, J = 8.15 Hz, 1 H), 4.27 (s, 3 H), 3.16 (s, 3 H), 2.25-2.38 (m, 3 H), 2.21 (br d, J = 7.94 Hz, 2 H), 1.53-1.88 (m, 2 H), 1.16-1.29 (m, 2 H), 0.93-1.06 (m, 2 H). | |
| | N-cyclobutyl-4-cyclopropyl-N- | | |
| | methyl-6-(2-methyl-2H- | | |
| | pyrazolo[3,4-b]pyridin-5- | | |
| | yl)thieno[2,3-b]pyridine-2- | | |
| | carboxamide | | |
| B17R | 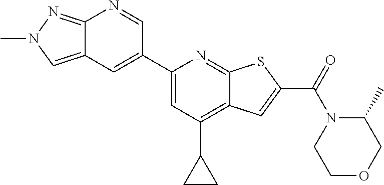 | 434.1; ¹H NMR (500 MHz, DMSO-d₆) δ 9.39 (d, J =2.32 Hz, 1 H) 8.96 (d, J = 2.32 Hz, 1 H) 8.52 (s, 1 H) 7.97 (s, 1 H) 7.55 (s, 1 H) 4.37-4.47 (m, 1 H) 4.24 (s, 3 H) 4.03 (br d, J = 10.51 Hz, 1 H) 3.88 (br d, J = 7.83 Hz, 1 H) 3.60-3.70 (m, 2 H) 3.42-3.52 (m, 2 H) 3.17 (d, J = 5.14 Hz, 1 H) 1.35 (d, J = 6.97 Hz, 3 H) 1.16-1.25 (m, 4 H). | Peak 1/SFC: DEAP 30 × 150 mm, mobile phase: 10%-40% MeOH using a flow rate of 100 mL/min |
| | (4-cyclopropyl-6-(2-methyl- | | |
| | 2H-pyrazolo[3,4-b]pyridin-5- | | |
| | yl)thieno[2,3-b]pyridin-2- | | |
| | yl)((3R)-3-methyl-4- | | |
| | morpholinyl)methanone | | |
| B18 | 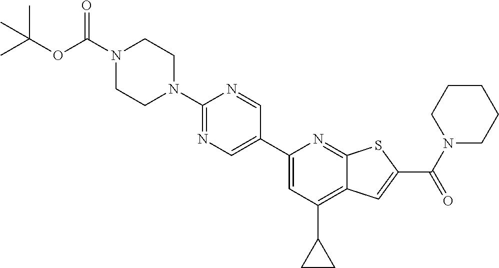 [SMILES: CC(C)(C)OC(=O)N1CCN(c2ncc(-c3cc(C4CC4)c4cc(C(=O)N5CCCCC5)sc4n3)cn2)CC1] | 549.0; ¹H NMR (400 MHz, DMSO-d₆) δ 9.14 (s, 2 H), 7.90 (s, 1 H), 7.40 (s, 1 H), 3.80-3.89 (m, 4 H), 3.66 (br d, J = 5.2 Hz, 4 H), 3.38-3.51 (m, 4 H), 2.53-2.61 (m, 1 H), 1.67 (br d, J = 3.7 Hz, 2 H), 1.60 (br d, J = 3.9 Hz, 4 H), 1.44 (s, 9 H), 1.07-1.23 (m, 4 H) | |
| | 2-methyl-2-propanyl 4-(5-(4- | | |
| | cyclopropyl-2-(1- | | |
| | piperidinylcarbonyl)thieno[2,3- | | |
| | b]pyridin-6-yl)-2-pyrimidinyl)- | | |
| | 1-piperazinecarboxylate | | |
| B19 | 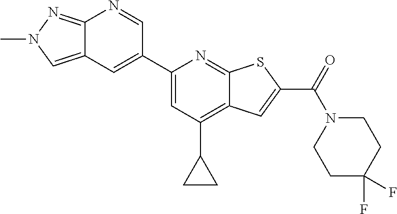 [SMILES: Cn1cc2cc(-c3cc(C4CC4)c4cc(C(=O)N5CCC(F)(F)CC5)sc4n3)cnc2n1] | 454.2; ¹H NMR (500 MHz, DMSO-d₆) δ 9.39 (d, J = 2.32 Hz, 1 H) 8.96 (d, J = 2.32 Hz, 1 H) 8.53 (s, 1 H) 8.03 (s, 1 H) 7.56 (s, 1 H) 4.24 (s, 3 H) 3.82 (br t, J = 5.75 Hz, 4 H) 2.31-2.38 (m, 1 H) 2.09-2.19 (m, 4 H) 1.16-1.26 (m, 4 H). | |
| | (4-cyclopropyl-6-(2-methyl- | | |
| | 2H-pyrazolo[3,4-b]pyridin-5- | | |
| | yl)thieno[2,3-b]pyridin-2- | | |
| | yl)(4,4-difluoro-1- | | |
| | piperidinyl)methanone | | |
| B20 | 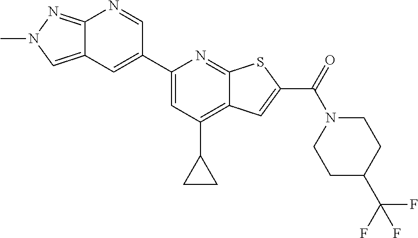 [SMILES: Cn1cc2cc(-c3cc(C4CC4)c4cc(C(=O)N5CCC(C(F)(F)F)CC5)sc4n3)cnc2n1] | 486.3; ¹H NMR (500 MHz, DMSO-d₆) δ 9.39 (d, J = 2.32 Hz, 1 H) 8.96 (d, J = 2.32 Hz, 1 H) 8.52 (s, 1 H) 7.99 (s, 1 H) 7.55 (s, 1 H) 4.34-4.51 (m, 2 H) 4.24 (s, 3 H) 3.22-3.30 (m, 1 H) 2.98-3.23 (m, 1 H) 2.71 (dtd, J = 12.15, 8.29, 8.29, 4.03 Hz, 1 H) 2.57-2.64 (m, 1 H) 1.93 (br d, J = 12.10 Hz, 2 H) 1.54 (qd, J = 12.51, 4.03 Hz, 2 H) 1.16-1.25 (m, 4 H) | |
| | (4-cyclopropyl-6-(2-methyl- | | |
| | 2H-pyrazolo[3,4-b]pyridin-5- | | |
| | yl)thieno[2,3-b]pyridin-2-yl)(4- | | |
| | (trifluoromethyl)-1- | | |
| | piperidinyl)methanone | | |
| B21 | 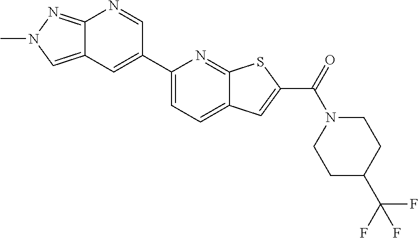 [SMILES: Cn1cc2cc(-c3ccc4cc(C(=O)N5CCC(C(F)(F)F)CC5)sc4n3)cnc2n1] | 446.0; ¹H NMR (500 MHz, DMSO-d₆) δ 9.40 (d, J = 2.32 Hz, 1 H) 8.96 (d, J = 2.32 Hz, 1 H) 8.55 (s, 1 H) 8.41 (d, J = 8.44 Hz, 1 H) 8.19 (d, J = 8.44 Hz, 1 H) 7.77 (s, 1 H) 4.32-4.51 (m, 2 H) 4.24 (s, 3 H) 3.25 (s, 1 H) 2.97-3.22 (m, 1 H) 2.66-2.77 (m, 1 H) 1.94 (br d, J = 11.98 Hz, 2 H) 1.53 (qd, J = 12.55, 4.16 Hz, 2 H). | |
| | (6-(2-methyl-2H-pyrazolo[3,4- | | |
| | b]pyridin-5-yl)thieno[2,3- | | |
| | b]pyridin-2-yl)(4- | | |
| | (trifluoromethyl)-1- | | |
| | piperidinyl)methanone | | |
| B23 | 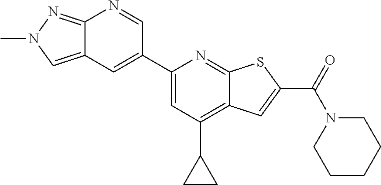 [SMILES: Cn1cc2cc(-c3cc(C4CC4)c4cc(C(=O)N5CCCCC5)sc4n3)cnc2n1] | 418.3; ¹H NMR (400 MHz, DMSO-d₆) δ 9.42 (d, J = 2.28 Hz, 1 H) 9.03 (d, J = 2.28 Hz, 1 H) 8.59 (s, 1 H) 7.96 (s, 1 H) 7.57 (s, 1 H) 4.25 (s, 3 H) 3.55-3.75 (m, 4 H) 2.54-2.63 (m, 1 H) 1.67 (br d, J = 4.56 Hz, 2 H) 1.61 (br d, J = 3.94 Hz, 4 H) 1.17-1.26 (m, 4 H). | |
| | (4-cyclopropyl-6-(2-methyl- | | |
| | 2H-pyrazolo[3,4-b]pyridin-5- | | |
| | yl)thieno[2,3-b]pyridin-2-yl)(1- | | |
| | piperidinyl)methanone | | |
| B24 | 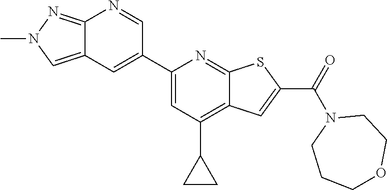 [SMILES: Cn1cc2cc(-c3cc(C4CC4)c4cc(C(=O)N5CCCOCC5)sc4n3)cnc2n1] | 434.2; ¹H NMR (400 MHz, chloroform-d) δ 9.32-9.38 (m, 1 H) 8.74-8.78 (m, 1 H) 8.01-8.07 (m, 1 H) 7.75-7.80 (m, 1 H) 7.31- 7.37 (m, 1 H) 4.31 (s, 3 H) 3.82- 3.95 (m, 9 H) 3.40-3.56 (m, 1 H) 2.32-2.41 (m, 1 H) 1.22-1.31 (m, 2 H) 1.00-1.08 (m, 2 H). | Step 3: Pd(dppf)Cl₂ was omitted |
| | (4-cyclopropyl-6-(2-methyl- | | |
| | 2H-pyrazolo[3,4-b]pyridin-5- | | |
| | yl)thieno[2,3-b]pyridin-2- | | |
| | yl)(1,4-oxazepan-4- | | |
| | yl)methanone | | |
| B25 | 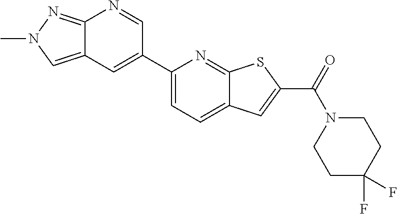 [SMILES: Cn1cc2cc(-c3ccc4cc(C(=O)N5CCC(F)(F)CC5)sc4n3)cnc2n1] | 414.3; ¹H NMR (400 MHz, chloroform-d) δ 9.43 (d, J = 1.87 Hz, 1 H) 8.82 (d, J = 2.07 Hz, 1 H) 8.20 (d, J = 8.50 Hz, 1 H) 8.05 (s, 1 H) 7.90 (d, J = 8.29 Hz, 1 H) 7.50 (s, 1 H) 4.32 (s, 3 H) 3.92 (br t, J = 5.60 Hz, 4 H) 2.04-2.20 (m, 4 H). ¹⁹F NMR (377 MHz, chloroform- d) δ −97.91 (s, 2F). | Alternate Condition (2) used |
| | (4,4-difluoro-1-piperidinyl)(6- | | |
| | (2-methyl-2H-pyrazolo[3,4- | | |
| | b]pyridin-5-yl)thieno[2,3- | | |
| | b]pyridin-2-yl)methanone | | |
| B27 | 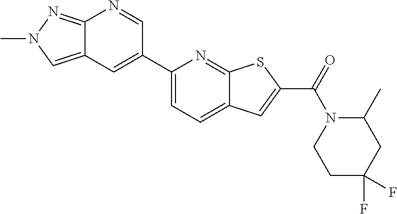 [SMILES: CC1CC(F)(F)CCN1C(=O)c1cc2ccc(-c3cnc4nn(C)cc4c3)nc2s1] | 428.2; ¹H NMR (400 MHz, chloroform-d) δ 9.43 (d, J = 2.09 Hz, 1 H) 8.81 (d, J = 2.30 Hz, 1 H) 8.19 (d, J = 8.36 Hz, 1 H) 8.04 (s, 1 H) 7.89 (d, J = 8.36 Hz, 1 H) 7.48 (s, 1 H) 4.95 (br t, J = 6.06 Hz, 1 H) 4.51 (br d, J = 12.12 Hz, 1 H) 4.32 (s, 3 H) 3.33-3.43 (m, 1 H) 1.97-2.24 (m, 4 H) 1.46 (dd, J = 7.11, 1.88 Hz, 3 H). 19F NMR (376 MHz, chloroform- d) 8-86.44--87.08 (d, 1 F)-94.74- -95.39 (d, 1 F). | Alternate Condition (2) used |
| | 4,4-difluoro-2-methyl-1- | | |
| | piperidinyl)(6-(2-methyl-2H- | | |
| | pyrazolo[3,4-b]pyridin-5- | | |
| | yl)thieno[2,3-b]pyridin-2- | | |
| | yl)methanone | | |
| B27R | 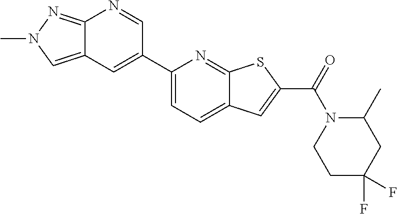 [SMILES: CC1CC(F)(F)CCN1C(=O)c1cc2ccc(-c3cnc4nn(C)cc4c3)nc2s1] | 428.2; ¹H NMR (400 MHz, chloroform-d) δ 9.43 (d, J = 2.28 Hz, 1 H) 8.82 (d, J = 2.28 Hz, 1 H) 8.20 (d, J = 8.50 Hz, 1 H) 8.06 (s, 1 H) 7.90 (d, J = 8.50 Hz, 1 H) 7.49 (s, 1 H) 4.95 (br s, 1 H) 4.52 (br d, J = 12.44 Hz, 1 H) 4.33 (s, 3 H) 3.38 (br t, J = 12.85 Hz, 1 H) 1.96- 2.24 (m, 4 H) 1.46 (dd, J = 7.26, 1.66 Hz, 3 H). ¹⁹F NMR (376 MHz, chloroform- d) δ −86.43-−87.08 (d, 1 F), −94.73-−95.38 (d, 1 F). | Alternate Condition (2) used Peak 2/SFC: Chiralpak AZ, 21 × 150 mm 5μm; mobile phase: 55% IPA using a flowrate of 80 mL/min |
| | ((2R)-4,4-difluoro-2-methyl-1- | | |
| | piperidinyl)(6-(2-methyl-2H- | | |
| | pyrazolo[3,4-b]pyridin-5- | | |
| | yl)thieno[2,3-b]pyridin-2- | | |
| | yl)methanone | | |
| B27S | 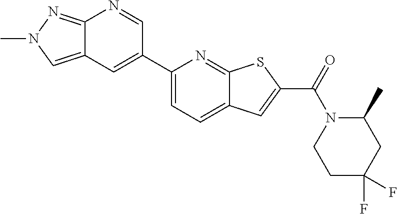 [SMILES: C[C@H]1CC(F)(F)CCN1C(=O)c1cc2ccc(-c3cnc4nn(C)cc4c3)nc2s1] | 428.2; ¹H NMR (400 MHz, chloroform-d) δ 9.43 (d, J = 2.28 Hz, 1 H) 8.82 (d, J = 2.07 Hz, 1 H) 8.20 (d, J = 8.50 Hz, 1 H) 8.05 (s, 1 H) 7.90 (d, J = 8.50 Hz, 1 H) 7.49 (s, 1 H) 4.95 (br s, 1 H) 4.52 (br d, J = 12.02 Hz, 1 H) 4.33 (s, 3 H) 3.38 (br t, J = 12.54 Hz, 1 H) 2.17- 2.25 (m, 1 H) 1.97-2.17 (m, 3 H) 1.46 (dd, J = 7.05, 1.66 Hz, 3 H). ¹⁹F NMR (376 MHz, chloroform- d) δ −86.43-−87.07 (d, 1 F), −94.73-−95.38 (d, 1 F) | Alternate Condition (2) used Peak 1/SFC: Chiralpak AZ, 21 × 150 mm 5μm; mobile phase: 55% IPA using a flowrate of 80 mL/min |
| | ((2S)-4,4-difluoro-2-methyl-1- | | |
| | piperidinyl)(6-(2-methyl-2H- | | |
| | pyrazolo[3,4-b]pyridin-5- | | |
| | yl)thieno[2,3-b]pyridin-2- | | |
| | yl)methanone | | |
| B28 | 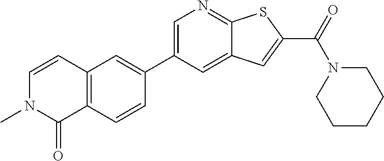 [SMILES: Cn1ccc2cc(-c3cnc4sc(C(=O)N5CCCCC5)cc4c3)ccc2c1=O] | 404.0; ¹H NMR (400 MHz, DMSO-d₆) δ 9.08 (d, J = 2.3 Hz, 1 H), 8.73 (d, J =2.3 Hz, 1 H), 8.34 (d, J = 8.4 Hz, 1 H), 8.10-8.16 (m, 1 H), 7.93 (d, J = 8.4 Hz, 1 H), 7.74 (s, 1 H), 7.55 (d, J = 6.8 Hz, 1 H), 6.68-6.74 (m, 1 H), 3.59-3.74 (m, 4 H), 3.54 (s, 3 H), 1.68 (br d, J = 4.6 Hz, 2 H), 1.61 (br d, J = 3.9 Hz, 4 H) | |
| | 2-methyl-6-(2-(1- | | |
| | piperidinylcarbonyl)thieno[2,3- | | |
| | b]pyridin-5-yl)-1(2H)- | | |
| | isoquinolinone | | |
| B29 | 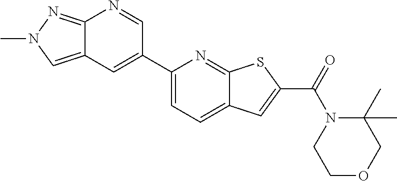 | 408.2; ¹H NMR (400 MHz, DMSO-d₆) δ 9.40 (d, J = 2.0 Hz, 1H), 8.97 (d, J = 2.0 Hz, 1H), 8.57 (s, 1H), 8.40 (d, J = 8.4 Hz, 1H), 8.20 (d, J = 8.5 Hz, 1H), 7.76 (s, 1H), 4.24 (s, 3H), 3.78 (br d, J = 4.8 Hz, 2H), 3.68 (br d, J = 4.9 Hz, 2H), 3.46 (s, 2H), 1.43 (s, 6H) | Alternate Condition (2) used |
| | (3,3-dimethyl-4- | | |
| | morpholinyl)(6-(2-methyl-2H- | | |
| | pyrazolo[3,4-b]pyridin-5- | | |
| | yl)thieno[2,3-b]pyridin-2- | | |
| | yl)methanone | | |
| B30 | 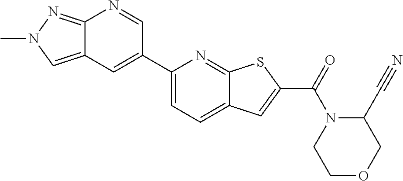 [SMILES: Cn1cc2cc(-c3ccc4cc(C(=O)N5CCOCC5C#N)sc4n3)cnc2n1] | 405.1; 1H NMR (400 MHz, DMSO-d₆) δ 9.41 (d, J = 2.1 Hz, 1H), 9.00 (d, J = 2.1 Hz, IH), 8.58 (s, 1H), 8.46 (br d, J = 8.5 Hz, 1H), 8.24 (br d, J = 8.5 Hz, 1H), 7.95 (s, 1H), 5.58 (br s, 1H), 4.29- 4.21 (m, 4H), 4.13-4.03 (m, 1H), 3.99-3.90 (m, 1H), 3.77 (br dd, J = 2.8, 12.3 Hz, 1H), 3.70-3.59 (m, 1H), 3.54-3.43 (m, 1H) | Alternate Condition (2) used |
| | 4-((6-(2-methyl-2H- | | |
| | pyrazolo[3,4-b ]pyridin-5- | | |
| | yl)thieno[2,3-b]pyridin-2- | | |
| | yl)carbonyl)-3- | | |
| | morpholinecarbonitrile | | |
| B31 | 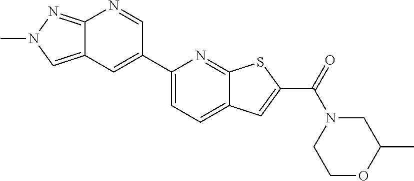 | 392.1; ¹H NMR (400 MHz, DMSO-d₆) δ 9.40 (d, J = 2.4 Hz, 1H), 8.98 (d, J = 2.3 Hz, 1H), 8.57 (s, 1H), 8.42 (d, J = 8.6 Hz, 1H), 8.20 (d, J = 8.5 Hz, 1H), 7.77 (s, 1H), 4.31-4.12 (m, 5H), 3.92-3.83 (m, 1H), 3.60-3.49 (m, 2H), 3.38- 3.32 (m, 2H), 1.17-1.08 (m, 3H) | Alternate Condition (2) used |
| | 2-methyl-4-morpholinyl)(6-(2- | | |
| | methyl-2H-pyrazolo[3,4- | | |
| | b]pyridin-5-yl)thieno[2,3- | | |
| | b]pyridin-2-yl)methanone | | |
| B32S | 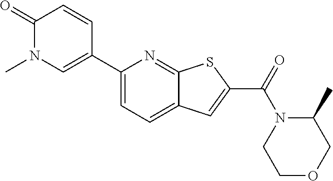 [SMILES: C[C@H]1COCCN1C(=O)c1cc2ccc(-c3ccc(=O)n(C)c3)nc2s1] | 370.0; ¹H NMR (400 MHz, DMSO-d₆) δ 8.68 (d, J = 2.4Hz, 1H), 8.33 (d, J = 8.0 Hz, 1H), 8.25 (dd, J = 2.4, 9.6 Hz, 1H), 7.94 (d, J = 8.4 Hz, 1H), 7.70 (s, 1H), 6.53 (d, J = 9.6 Hz, 1H), 4.41 (br d, J = 11.6 Hz, 1H), 4.03 (d, J = 11.6 Hz, 1H), 3.84-3.90 (m, 1H), 3.67 (d, J = 11.6 Hz, 1H), 3.61 (d, J = 2.8 Hz, 1H), 3.57 (s, 3H), 3.35- 3.50 (m, 2H), 1.35 (d, J = 6.8 Hz, 3H) | Alternate Condition (1) used Confirmed by SFC: Chiralpak AD-3, 50 × 4.6 mm, I.D., 3 um; mobile phase: 5%- 50% EtOH (0.1% IPAm, v/v) using a flow rate of 3.4 mL/min |
| | 1-methyl-5-(2-(((3S)-3-methyl- | | |
| | 4- | | |
| | morpholinyl)carbonyl)thieno[2, | | |
| | 3-b]pyridin-6-yl)-2(1H)- | | |
| | pyridinone | | |
| B32R | 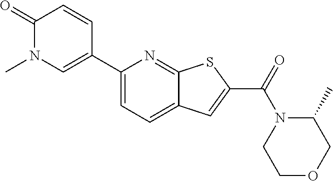 [SMILES: C[C@@H]1COCCN1C(=O)c1cc2ccc(-c3ccc(=O)n(C)c3)nc2s1] | 370.1; ¹H NMR (400 MHz, DMSO-d₆) δ 8.68 (d, J = 2.5 Hz, 1H), 8.33 (d, J = 8.6 Hz, 1H), 8.26 (dd, J = 2.6, 9.5 Hz, 1H), 7.94 (d, J = 8.6 Hz, 1H), 7.70 (s, 1H), 6.53 (d, J = 9.5 Hz, 1H), 4.41 (br d, J = 6.0 Hz, 1H), 4.03 (br d, J = 11.4 Hz, 1H), 3.84-3.92 (m, 1H), 3.64- 3.69 (m, 1H), 3.61 (d, J = 2.9 Hz, 1H), 3.57 (s, 3H), 3.39-3.50 (m, 2H), 1.35 (d, J = 6.8 Hz, 3H). | Alternate Condition (1) used Confirmed by SFC: Chiralpak AD-3, 50 × 4.6 mm, I.D., 3 um; mobile phase: 5%- 50% EtOH (0.1% IPAm, v/v) using a flow rate of 3.4 mL/min |
| | 1-methyl-5-(2-(((3R)-3-methyl- | | |
| | 4- | | |
| | morpholinyl)carbonyl)thieno[2, | | |
| | 3-b]pyridin-6-yl)-2(1H)- | | |
| | pyridinone | | |
| B33S | 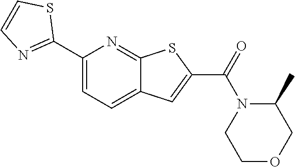 [SMILES: C[C@H]1COCCN1C(=O)c1cc2ccc(-c3nccs3)nc2s1] | 335.5; ¹H NMR (400 MHz, DMSO-d₆) δ 8.46 (d, J = 8.4 Hz, 1H), 8.26 (d, J = 8.3 Hz, IH), 8.05 (d, J = 3.1 Hz, 1H), 7.96 (d, J = 3.1 Hz, 1H), 7.81 (s, 1H), 4.40 (br d, J = 4.8 Hz, 1H), 4.10-3.96 (m, 1H), 3.91-3.81 (m, 1H), 3.72-3.64 (m, 1H), 3.63-3.57 (m, 1H), 3.51-3.36 (m, 2H), 1.35 (d, J = 6.9 Hz, 3H) | Alternate Condition (3) used Confirmed by SFC: Chiralpak AD-3, 150 × 4.6 mm, I.D., 3 um; mobile phase: 35% EtOH (0.1% IPAm) using a flow rate of 2.5 mL/min |
| | ((3S)-3-methyl-4- | | |
| | morpholinyl)(6-(1,3-thiazol-2- | | |
| | yl)thieno[2,3-b]pyridin-2- | | |
| | yl)methanone | | |
| B33R | 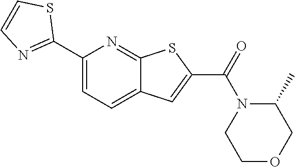 [SMILES: C[C@@H]1COCCN1C(=O)c1cc2ccc(-c3nccs3)nc2s1] | 346.1; ¹H NMR (400 MHz, DMSO-d₆) δ 8.46 (d, J = 8.4 Hz, 1H), 8.26 (d, J = 8.3 Hz, 1H), 8.05 (d, J = 3.1 Hz, 1H), 7.96 (d, J = 3.1 Hz, 1H), 7.81 (s, 1H), 4.40 (br d, J = 4.8 Hz, 1H), 4.10-3.96 (m, 1H), 3.91-3.81 (m, 1H), 3.72-3.64 (m, 1H), 3.63-3.57 (m, 1H), 3.51-3.36 (m, 2H), 1.35 (d, J = 6.9 Hz, 3H) | Alternate Condition (3) used Confirmed by SFC: Chiralpak AD-3, 150 × 4.6 mm, I.D., 3 um; mobile phase: 35% IPA (0.1% IPAm) using a flow rate of 2.5 mL/min |
| | ((3R)-3-methyl-4- | | |
| | morpholinyl)(6-(1,3-thiazol-2- | | |
| | yl)thieno[2,3-b]pyridin-2- | | |
| | yl)methanone | | |
| B34S | 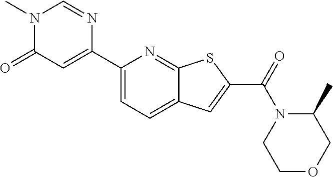 | 371.0; ¹H NMR (400 MHz, DMSO-d₆) δ 8.64 (s, 1H), 8.49- 8.44 (m, 1H), 8.40-8.35 (m, 1H), 7.79 (s, 1H), 7.30 (s, 1H), 4.39 (br s, 1H), 4.01 (br d, J = 10.3 Hz, 1H), 3.87 (br d, J = 8.0 Hz, 1H), 3.71-3.64 (m, 1H), 3.63-3.56 (m, 1H), 3.51-3.40 (m, 5H), 1.34 (d, J = 6.8 Hz, 3H) | Alternate Condition (3) used Confirmed by SFC: Chiralpak AD-3, 150 × 4.6 mm, I.D., 3 um; mobile phase: 40% IPA (0.1% IPAm) using a flow rate of 2.5 mL/min |
| | 3-methyl-6-(2-(((3S)-3-methyl-4- | | |
| | morpholinyl)carbonyl)thieno[2, | | |
| | 3-b]pyridin-6-yl)-4(3H)- | | |
| | pyrimidinone | | |
| B35 | 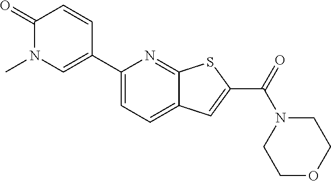 [SMILES: Cn1cc(-c2ccc3cc(C(=O)N4CCOCC4)sc3n2)ccc1=O] | 356.2; ¹H NMR (400 MHz, DMSO-d₆) δ 8.67 (d, J = 2.50 Hz, 1 H), 8.32 (d, J = 8.63 Hz, 1 H), 8.25 (dd, J = 9.57, 2.69 Hz, 1 H), 7.93 (d, J = 8.63 Hz, 1 H), 7.71 (s, 1 H), 6.53 (d, J = 9.51 Hz, 1 H), 3.63- 3.74 (m, 8 H), 3.57 (s, 3 H) | Alternate Condition (1) used |
| | 1-methyl-5-(2-(4- | | |
| | morpholinylcarbonyl)thieno[2,3- | | |
| | b]pyridin-6-yl)-2(1H)- | | |
| | pyridinone | | |
| B36R | 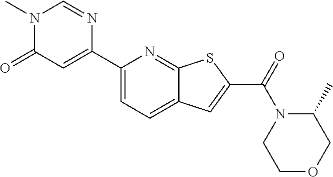 | 371.1; ¹H NMR (400 MHz, DMSO-d₆) δ 8.64 (s, 1H), 8.49- 8.44 (m, 1H), 8.40-8.35 (m, 1H), 7.79 (s, 1H), 7.30 (s, 1H), 4.39 (br s, 1H), 4.01 (br d, J = 10.3 Hz, 1H), 3.87 (br d, J = 8.0 Hz, 1H), 3.71-3.64 (m, 1H), 3.63-3.56 (m, 1H), 3.51-3.40 (m, 5H), 1.34 (d, J = 6.8 Hz, 3H). | Alternate Condition (3) used Confirmed by SFC: Chiralpak AD-3, 150 × 4.6 mm, I.D., 3 um; mobile phase: 40% IPA (0.1% IPAm) using a flow rate of 2.5 mL/min |
| | 3-methyl-6-(2-(((3R)-3-methyl-4- | | |
| | morpholinyl)carbonyl)thieno[2, | | |
| | 3-b]pyridin-6-yl)-4(3H)- | | |
| | pyrimidinone | | |
| B37 | 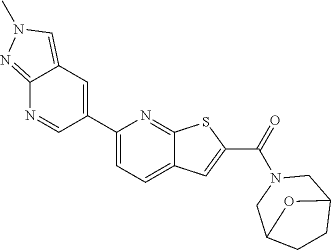 [SMILES: Cn1cc2cc(-c3ccc4cc(C(=O)N5CC6CCC(C5)O6)sc4n3)cnc2n1] | 406.1; ¹H NMR (400 MHz, DMSO-d₆) δ 9.40 (d, J = 2.4 Hz, 1H), 8.98 (d, J = 2.3 Hz, 1H), 8.57 (s, 1H), 8.41 (d, J = 8.5 Hz, 1H), 8.20 (d, J = 8.5 Hz, 1H), 7.75 (s, 1H), 4.44-4.32 (m, 2H), 4.24 (s, 3H), 4.16-3.78 (m, 2H), 3.35 (br d, J = 3.9 Hz, 1H), 3.30-3.22 (m, 1H), 1.90-1.81 (m, 2H), 1.77-1.67 (m, 2H) | Alternate Condition (2) used |
| | (6-(2-methyl-2H-pyrazolo[3,4- | | |
| | b]pyridin-5-yl)thieno[2,3- | | |
| | b]pyridin-2-yl)(8-oxa-3- | | |
| | azabicyclo[3.2.1]octan-3- | | |
| | yl)methanone | | |
| B38 | 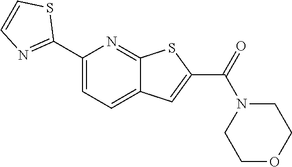 [SMILES: O=C(c1cc2ccc(-c3nccs3)nc2s1)N1CCOCC1] | 331.9; ¹H NMR (400 MHz, DMSO-d₆) δ 8.46 (d, J = 8.4 Hz, 1H), 8.26 (d, J = 8.4 Hz, 1H), 8.05 (d, J = 3.2 Hz, 1H), 7.95 (d, J = 3.2 Hz, 1H), 7.82 (s, 1H), 3.68- 3.70 (m, 8H | Alternate Condition (3) used |
| | 4-morpholinyl(6-(1,3-thiazol-2- | | |
| | yl)thieno[2,3-b]pyridin-2- | | |
| | yl)methanone | | |
| B39 | 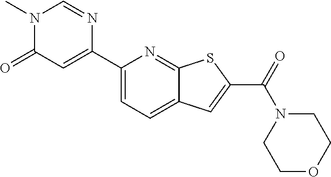 [SMILES: Cn1cnc(-c2ccc3cc(C(=O)N4CCOCC4)sc3n2)cc1=O] | 357.0; ¹H NMR (400 MHz, DMSO-d₆) δ 8.65 (s, 1H), 8.47 (d, J = 8.4 Hz, 1H), 8.38 (d, J = 8.4 Hz, 1H), 7.81 (s, 1H), 7.31 (s, 1H), 3.68-3.70 (m, 8H), 3.48 (s, 3H) | Alternate Condition (3) used |
| | 3-methyl-6-(2-(4- | | |
| | morpholinylcarbonyl)thieno[2,3- | | |
| | b]pyridin-6-yl)-4(3H)- | | |
| | pyrimidinone | | |
| B40 | 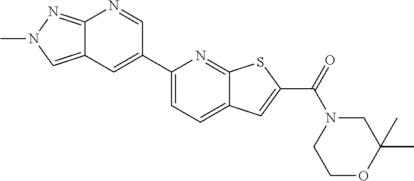 | 408.2; ¹H NMR (DMSO-d₆) δ: 9.40 (d, J = 2.3 Hz, 1H), 8.98 (d, J = 2.3 Hz, 1H), 8.57 (s, 1H), 8.42 (d, J = 8.5 Hz, 1H), 8.21 (d, J = 8.5 Hz, 1H), 7.75 (br s, 1H), 4.24 (s, 3H), 3.65-3.74 (m, 4H), 3.54 (s, 2H), 1.19 (s, 6H) | Alternate Condition (2) used |
| | (2,2-dimethyl-4- | | |
| | morpholinyl)(6-(2-methyl-2H- | | |
| | pyrazolo[3,4-b]pyridin-5- | | |
| | yl)thieno[2,3-b]pyridin-2- | | |
| | yl)methanone | | |
| B41 | 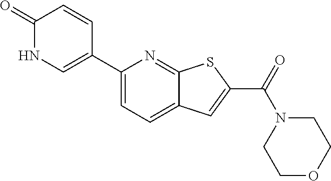 [SMILES: O=C(c1cc2ccc(-c3ccc(=O)[nH]c3)nc2s1)N1CCOCC1] | 341.9; ¹H NMR (DMSO-d₆) δ: = 12.04 (s, 1H), 8.30-8.26 (m, 3H), 7.94 (d, J = 8.4 Hz, 1H), 7.71 (s, 1H), 6.47 (d, J = 9.8 Hz, 1H), 3.70- 3.67 (m, 8H) | Alternate Condition (2) used |
| | 5-(2-(4- | | |
| | morpholinylcarbonyl)thieno[2,3- | | |
| | b]pyridin-6-yl)-2(1H)- | | |
| | pyridinone | | |
| B42 | 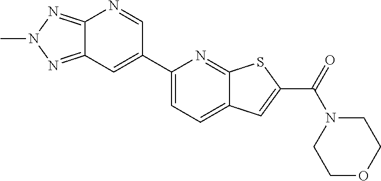 [SMILES: Cn1nc2cc(-c3ccc4cc(C(=O)N5CCOCC5)sc4n3)cnc2n1] | 381.0; ¹H NMR (400 MHz, DMSO-d₆) δ 9.60 (d, J = 2.0 Hz, 1H), 9.12 (d, J = 2.0 Hz, 1H), 8.47 (d, J = 8.4 Hz, 1H), 8.32 (d, J = 8.8 Hz, 1H), 7.80 (s, 1H), 4.60 (s, 3H), 3.65-3.73 (m, 8H). | Alternate Condition (1) was used |
| | (6-(2-methyl-2H- | | |
| | [1,2,3]triazolo[4,5-b]pyridin-6- | | |
| | yl)thieno[2,3-b]pyridin-2-yl)(4- | | |
| | morpholinyl)methanone | | |
| B43S | 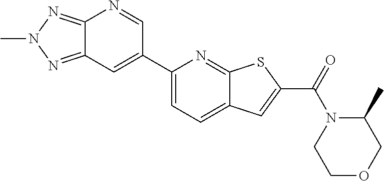 | 395.0; ¹H NMR (400 MHz, DMSO-d₆) δ 9.62 (d, J = 2.0 Hz, 1H), 9.14 (d, J = 2.0 Hz, 1H), 8.48 (d, J = 8.4 Hz, 1H), 8.33 (d, J = 8.8 Hz, 1H), 7.79 (s, 1H), 4.60 (s, 3H), 4.42 (br s, 1H), 4.04 (br d, J = 10.8 Hz, 1H), 3.89 (d, J = 8.0 Hz, 1H), 3.65-3.70 (m, 1H), 3.60-6.63 (m, 1H), 3.45-3.48 (m, 2H), 1.36 (d, J = 6.8 Hz, 3H) | Alternate Condition (1) was used |
| | ((3S)-3-methyl-4- | | |
| | morpholinyl)(6-(2-methyl-2H- | | |
| | [1,2,3]triazolo[4,5-b]pyridin-6- | | |
| | yl)thieno[2,3-b]pyridin-2- | | |
| | yl)methanone | | |
| B43R | 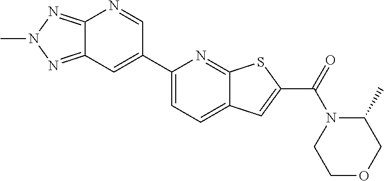 | 395.1; ¹H NMR (400 MHz, DMSO-d₆) δ 9.62 (d, J = 1.9 Hz, 1H), 9.14 (d, J = 2.0 Hz, 1H), 8.48 (d, J = 8.5 Hz, 1H), 8.33 (d, J = 8.4 Hz, 1H), 7.79 (s, 1H), 4.60 (s, 3H), 4.37-4.46 (m, 1H), 4.04 (br d, J = 11.6 Hz, 1H), 3.88 (br d, J = 7.6 Hz, 1H), 3.65-3.6570 (m, 1H), 3.59-3.64 (m, 1H), 3.42-3.51 (m, 2H), 1.36 (d, J = 6.8 Hz, 3H), 1.23 (br s, 1H), 1.05 (t, J = 6.9 Hz, 1H) | Alternate Condition (1) was used |
| | ((3R)-3-methyl-4- | | |
| | morpholinyl)(6-(2-methyl-2H- | | |
| | [1,2,3]triazolo[4,5-b]pyridin-6- | | |
| | yl)thieno[2,3-b]pyridin-2- | | |
| | yl)methanone | | |
| B44R | 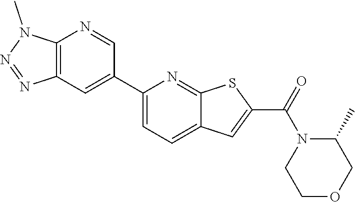 [SMILES: C[C@@H]1COCCN1C(=O)c1cc2ccc(-c3cnc4c(c3)nnn4C)nc2s1] | 395.1; ¹H NMR (400 MHz, DMSO-d₆) δ 9.59 (d, J = 1.8 Hz, 1H), 9.26 (d, J = 1.8 Hz, 1H), 8.48 (d, J = 8.5 Hz, 1H), 8.33 (d, J = 8.5 Hz, 1H), 7.78 (s, 1H), 4.41 (br d, J = 4.2 Hz, 1H), 4.36 (s, 3H), 4.04 (br d, J = 10.6 Hz, 1H), 3.88 (br d, J = 7.4 Hz, 1H), 3.71-3.65 (m, 1H), 3.64-3.58 (m, 1H), 3.52-3.42 (m, 2H), 1.36 (br d, J = 6.8 Hz, 3H) | Alternate Condition (2) used |
| | ((3R)-3-methyl-4- | | |
| | morpholinyl)(6-(3-methyl-3H- | | |
| | [1,2,3]triazolo[4,5-b ]pyridin-6- | | |
| | yl)thieno[2,3-b]pyridin-2- | | |
| | yl)methanone | | |
| B44S | 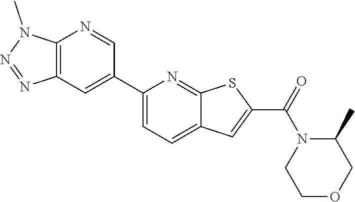 [SMILES: C[C@H]1COCCN1C(=O)c1cc2ccc(-c3cnc4c(c3)nnn4C)nc2s1] | 395.0; ¹H NMR (400 MHz, DMSO-d₆) δ 9.59 (d, J = 1.8 Hz, 1H), 9.26 (d, J = 1.8 Hz, 1H), 8.48 (d, J = 8.5 Hz, 1H), 8.33 (d, J = 8.5 Hz, 1H), 7.78 (s, 1H), 4.41 (br d, J = 4.2 Hz, 1H), 4.36 (s, 3H), 4.04 (br d, J = 10.6 Hz, 1H), 3.88 (br d, J = 7.4 Hz, 1H), 3.71-3.65 (m, 1H), 3.64-3.58 (m, 1H), 3.52-3.42 (m, 2H), 1.36 (br d, J = 6.8 Hz, 3H) | Alternate Condition (2) used |
| | ((3S)-3-methyl-4- | | |
| | morpholinyl)(6-(3-methyl-3H- | | |
| | [1,2,3]triazolo[4,5-b]pyridin-6- | | |
| | yl)thieno[2,3-b]pyridin-2- | | |
| | yl)methanone | | |
| B45 | 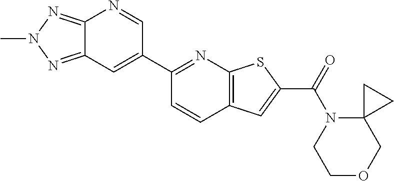 [SMILES: Cn1nc2cc(-c3ccc4cc(C(=O)N5CCOCC56CC6)sc4n3)cnc2n1] | 406.2; ¹H NMR (400 MHz, DMSO-d₆) δ: 9.40 (d, J = 2.3 Hz, 1H), 8.97 (d, J = 2.4 Hz, 1H), 8.57 (s, 1H), 8.43 (d, J = 8.5 Hz, 1H), 8.20 (d, J = 8.5 Hz, 1H), 7.80 (s, 1H), 4.24 (s, 3H), 3.85 (br s, 2H), 3.61-3.78 (m, 4H), 0.86-0.98 (m, 4H) | Alternate Condition (2) used |
| | (6-(2-methyl-2H-pyrazolo[3,4- | | |
| | b]pyridin-5-yl)thieno[2,3- | | |
| | b]pyridin-2-yl)(7-oxa-4- | | |
| | azaspiro[2.5]octan-4- | | |
| | yl)methanone | | |
| B46 | 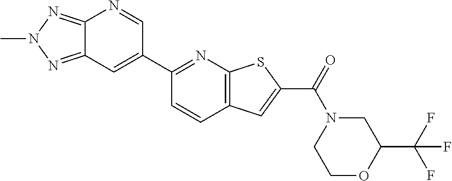 [SMILES: Cn1nc2cc(-c3ccc4cc(C(=O)N5CCOC(C(F)(F)F)C5)sc4n3)cnc2n1] | 448.1; ¹H NMR (400 MHz, DMSO-d₆) δ: 9.41 (d, J = 2.3 Hz, 1H), 8.98 (d, J = 2.3 Hz, 1H), 8.57 (s, 1H), 8.44 (d, J = 8.5 Hz, 1H), 8.22 (d, J = 8.5 Hz, 1H), 7.84 (s, 1H), 4.33-4.49 (m, 2H), 4.17-4.27 (m, 4H), 4.04 (br d, J = 12.3 Hz, 1H), 3.68-3.78 (m, 1H), 3.33-3.51 (m, 2H) | Alternate Condition (2) used |
| | (6-(2-methyl-2H-pyrazolo[3,4- | | |
| | b]pyridin-5-yl)thieno[2,3- | | |
| | b]pyridin-2-yl)(2- | | |
| | (trifluoromethyl)-4- | | |
| | morpholinyl)methanone | | |
| B47 | 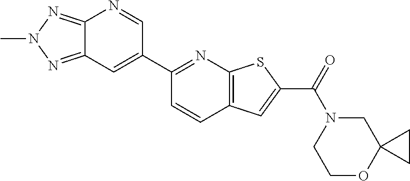 [SMILES: Cn1nc2cc(-c3ccc4cc(C(=O)N5CCOC6(CC6)C5)sc4n3)cnc2n1] | 406.2; ¹H NMR (400 MHz, DMSO-d₆) δ: 9.40 (d, J = 2.3 Hz, 1H), 8.97 (d, J = 2.3 Hz, 1H), 8.57 (s, 1H), 8.41 (d, J = 8.5 Hz, 1H), 8.20 (d, J = 8.5 Hz, 1H), 7.74 (br s, 1H), 4.24 (s, 3H), 3.81 (br s, 2H), 3.69-3.75 (m, 4H), 0.71-0.77 (m, 2H), 0.62 (br s, 2H) | Alternate Condition (2) used |
| | (6-(2-methyl-2H-pyrazolo[3,4- | | |
| | b]pyridin-5-yl)thieno[2,3- | | |
| | b]pyridin-2-yl)(4-oxa-7- | | |
| | azaspiro[2.5]octan-7- | | |
| | yl)methanone | | |
| B48S | 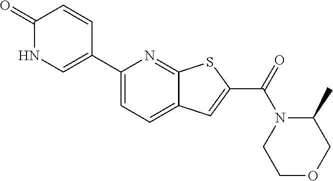 [SMILES: C[C@H]1COCCN1C(=O)c1cc2ccc(-c3ccc(=O)[nH]c3)nc2s1] | 355.9; 1H NMR (400 MHz, DMSO-d₆) δ 12.04 (br s, 1H), 8.33-8.24 (m, 3H), 7.94 (d, J = 8.6 Hz, 1H), 7.69 (s, 1H), 6.47 (d, J = 9.8 Hz, 1H), 4.40 (br d, J = 5.8 Hz, 1H), 4.02 (br d, J = 11.1 Hz, 1H), 3.91-3.82 (m, 1H), 3.69-3.64 (m, 1H), 3.62-3.56 (m, 1H), 3.49-3.37 (m, 2H), 1.34 (d, J = 6.8 Hz, 3H) | Alternate Condition (2) used Confirmed by SFC: Chiralpak AD-3, 50 × 4.6 mm, I.D., 3 um; mobile phase: 35% MeOH (0.1% IPAm) using a flow rate of 4 mL/min |
| | 5-(2-(((3S)-3-methyl-4- | | |
| | morpholinyl)carbonyl)thieno[2, | | |
| | 3-b]pyridin-6-yl)-2(1H)- | | |
| | pyridinone | | |
| B48R | 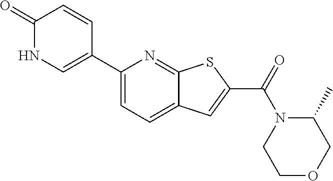 [SMILES: C[C@@H]1COCCN1C(=O)c1cc2ccc(-c3ccc(=O)[nH]c3)nc2s1] | 356.1; ¹H NMR (400 MHz, DMSO-d₆) δ 12.04 (br s, 1H), 8.33-8.24 (m, 3H), 7.94 (d, J = 8.6 Hz, 1H), 7.69 (s, 1H), 6.47 (d, J = 9.8 Hz, 1H), 4.40 (br d, J = 5.8 Hz, 1H), 4.02 (br d, J = 11.1 Hz, 1H), 3.91-3.82 (m, 1H), 3.69-3.64 (m, 1H), 3.62-3.56 (m, 1H), 3.49-3.37 (m, 2H), 1.34 (d, J = 6.8 Hz, 3H) | Alternate Condition (2) used Confirmed by SFC: Chiralpak AD-3, 50 × 4.6 mm, I.D., 3 um; mobile phase: 35% MeOH (0.1% IPAm) using a flow rate of 4 mL/min |
| | 5-(2-(((3R)-3-methyl-4- | | |
| | morpholinyl)carbonyl)thieno[2, | | |
| | 3-b ]pyridin-6-yl)-2(1H)- | | |
| | pyridinone | | |
| B49 | 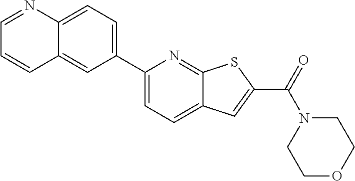 [SMILES: O=C(c1cc2ccc(-c3ccc4ncccc4c3)nc2s1)N1CCOCC1] | 375.9; ¹H NMR (400 MHz, DMSO-d₆) δ 8.95 (t, J = 2.0 Hz, 1H), 8.84 (d, J =1.8 Hz, 1H), 8.58- 8.53 (m, 2H), 8.46 (d, J = 8.4 Hz, 1H), 8.29 (d, J = 8.4 Hz, 1H), 8.15 (d, J = 8.8 Hz, 1H), 7.80 (s, 1H), 7.60 (t, J = 4.0 Hz, 1H), 5.75 (s, 1H), 3.71 (br d, J =10.8 Hz, 8H) | Alternate Condition (2) used |
| | 4-morpholinyl(6-(6- | | |
| | quinolinyl)thieno[2,3-b]pyridin- | | |
| | 2-yl)methanone | | |
| B50 | 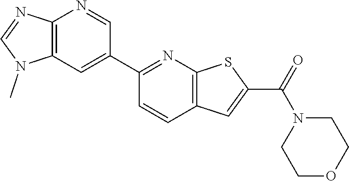 [SMILES: Cn1cnc2ncc(-c3ccc4cc(C(=O)N5CCOCC5)sc4n3)cc21] | 391.9; ¹H NMR (400 MHz, DMSO-d₆) δ 9.25 (d, J = 2.0 Hz, 1H), 8.80 (d, J = 2.0 Hz, 1H), 8.53 (s, 1H), 8.42 (d, J = 8.6 Hz, 1H), 8.26 (d, J = 8.4 Hz, 1H), 7.78 (s, 1H), 3.98 (s, 3H), 3.70 (br d, J = 10.0 Hz, 8H) | Alternate Condition (1) was used |
| | (6-(1-methyl-1H-imidazo[4,5- | | |
| | b]pyridin-6-yl)thieno[2,3- | | |
| | b]pyridin-2-yl)(4- | | |
| | morpholinyl)methanone | | |
| B51 | 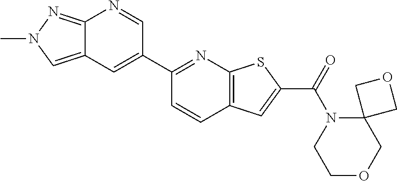 [SMILES: Cn1cc2cc(-c3ccc4cc(C(=O)N5CCOCC56COC6)sc4n3)cnc2n1] | 422.1; ¹H NMR (400 MHz, DMSO-d₆) δ 9.41 (d, J = 2.1 Hz, 1H), 8.99 (d, J = 2.3 Hz, 1H), 8.57 (s, 1H), 8.45 (d, J = 8.5 Hz, 1H), 8.23 (d, J = 8.6 Hz, 1H), 7.92 (s, 1H), 4.74 (d, J = 7.0 Hz, 2H), 4.42 (d, J = 7.0 Hz, 2H), 4.25 (s, 3H), 4.00 (s, 2H), 3.72 (br t, J = 4.4 Hz, 2H), 3.51 (t, J = 4.6 Hz, 2H) | Alternate Condition (2) used |
| | 2,8-dioxa-5- | | |
| | azaspiro[3.5]nonan-5-yl(6-(2- | | |
| | methyl-2H-pyrazolo[3,4- | | |
| | b]pyridin-5-yl)thieno[2,3- | | |
| | b]pyridin-2-yl)methanone | | |
| B52 | 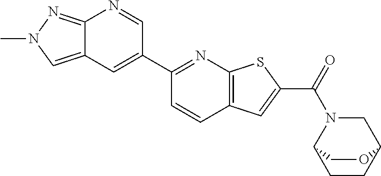 [SMILES: Cn1cc2cc(-c3ccc4cc(C(=O)N5C[C@H]6CC[C@@H]5CO6)sc4n3)cnc2n1] | 406.2; ¹H NMR (400 MHz, DMSO-d₆) δ 9.40 (d, J = 2.4 Hz, 1H), 8.93 (d, J = 2.3 Hz, 1H), 8.51 (s, 1H), 8.41 (d, J = 8.4 Hz, 1H), 8.15 (d, J = 8.4 Hz, 1H), 7.65-7.94 (m, 1H), 5.69 (s, 1H), 4.40 (br d, J = 6.4 Hz, 1H), 4.26 (s, 3H), 4.12 (br d, J = 9.3 Hz, 1H), 4.05 (br s, 1H), 3.97 (br d, J = 9.4 Hz, 1H), 3.62 (s, 2H), 2.04-2.13 (m, 2H), 1.91-1.99 (m, 1H), 1.78-1.85 (m, 1H) | Alternate Condition (1) was used |
| | (6-(2-methyl-2H-pyrazolo[3,4- | | |
| | b]pyridin-5-yl)thieno[2,3- | | |
| | b]pyridin-2-yl)(2-oxa-5- | | |
| | azabicyclo[2.2.2]octan-5- | | |
| | yl)methanone | | |
| B53 | 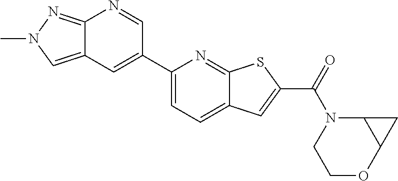 [SMILES: Cn1cc2cc(-c3ccc4cc(C(=O)N5CCOC6CC65)sc4n3)cnc2n1] | 392.1; ¹H NMR (400 MHz, DMSO-d₆) δ: 9.42 (d, J = 2.1 Hz, 1H), 8.99 (d, J = 2.1 Hz, 1H), 8.57 (s, 1H), 8.47 (br d, J = 8.5 Hz, 1H), 8.16-8.23 (m, 2H), 4.24 (s, 3H), 3.77-3.91 (m, 2H), 3.74 (br t, J = 8.4 Hz, 1H), 3.57-3.65 (m, 1H), 3.21 (br d, J = 5.0 Hz, 2H), 1.18 (br d, J = 6.1 Hz, 1H), 1.01-1.06 (m, 1H) | Alternate Condition (1) was used |
| | (6-(2-methyl-2H-pyrazolo[3,4- | | |
| | b]pyridin-5-yl)thieno[2,3- | | |
| | b]pyridin-2-yl)(2-oxa-5- | | |
| | azabicyclo[4.1.0]heptan-5- | | |
| | yl)methanone | | |
| B54 | 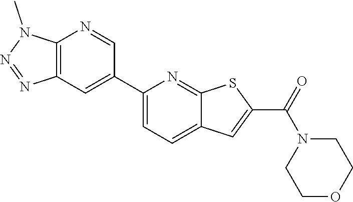 [SMILES: Cn1nnc2cc(-c3ccc4cc(C(=O)N5CCOCC5)sc4n3)cnc21] | 381.0; ¹H NMR (400 MHz, DMSO-d₆) δ 9.6 (d, J = 2.0 Hz, 1H), 9.26 (d, J = 2.0 Hz, 1H), 8.48 (d, J = 8.4 Hz, 1H), 8.33 (d, J = 8.4 Hz, 1H), 7.81 (s, 1H), 5.75 (s, 1H), 4.37 (s, 3H), 3.70 (br d, J = 8.8 Hz, 8H) | Alternate Condition (2) used |
| | (6-(3-methyl-3H- | | |
| | [1,2,3]triazolo[4,5-b]pyridin-6- | | |
| | yl)thieno[2,3-b]pyridin-2-yl)(4- | | |
| | morpholinyl)methanone | | |
| B55S | 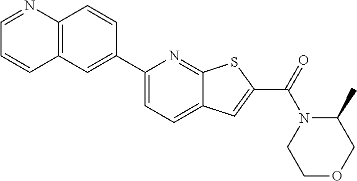 [SMILES: C[C@H]1COCCN1C(=O)c1cc2ccc(-c3ccc4ncccc4c3)nc2s1] | 390.0; ¹H NMR (400 MHz, DMSO-d₆) δ 8.95 (q, J = 2.0 Hz, 1H), 8.84 (d, J = 2.0 Hz, 1H), 8.59- 8.50 (m, 2H), 8.46 (d, J = 10.0 Hz, 1H), 8.46 (d, J = 8.8 Hz, 1H), 8.29 (d, J = 8.4 Hz, 1H), 8.15 (d, J = 8.4Hz, 1H). 7.78 (s, 1H), 7.61- 7.59 (m, 1H), 4.43 (br s, 1H), 4.05 (d, J = 10.0 Hz, 1H), 3.88 (d, J = 8.0 Hz, 1H), 3.69-3.60 (m, 2H), 3.46 (br d, J = 10.0 Hz, 2H), 1.36 | Alternate Condition (2) used Confirmed by SFC: Chiralpak IH-3, 100 × 4.6 mm, I.D., 3 um; mobile phase: 50% IPA (0.1% IPAm) using a flow rate of 4 mL/min |
| | | (d, J =7.2 Hz, 3H) | |
| | ((3S)-3-methyl-4- | | |
| | morpholinyl)(6-(6- | | |
| | quinolinyl)thieno[2,3-b]pyridin- | | |
| | 2-yl)methanone | | |
| B56 | 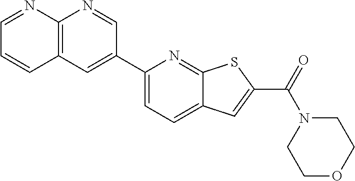 [SMILES: O=C(c1cc2ccc(-c3cnc4ncccc4c3)nc2s1)N1CCOCC1] | 377.9; ¹H NMR (400 MHz, DMSO-d₆) δ 9.98 (d, J = 2.0 Hz, 1H), 9.23-9.14 (m, 3H), 8.50 (q, J = 8.0 Hz, 2H), 7.82 (s, 1H), 3.71 (br d, J = 8.8 Hz, 8H) | Alternate Condition (1) was used |
| | 4-morpholinyl(6-(pyrido[2,3- | | |
| | b]pyrazin-7-yl)thieno[2,3- | | |
| | b]pyridin-2-yl)methanone | | |
| B57S | 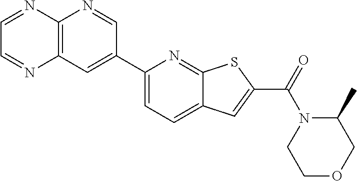 [SMILES: C[C@H]1COCCN1C(=O)c1cc2ccc(-c3cnc4nccnc4c3)nc2s1] | 329.0; ¹H NMR (400 MHz, DMSO-d₆) δ 9.97 (s, 1H), 9.17 (t, J = 11.0 Hz, 3H), 8.48 (q, J = 8.4 Hz, 1H), 5.75 (s, 1H), 4.41 (br d, J = 2.0 Hz, 1H), 4.04 (br d, J = 10.0 Hz, 1H), 3.89 (d, J = 8.0 Hz, 1H), 3.69-3.62 (m, 2H), 3.48-3.45 (m, 2H), 1.36 (d, J = 8.4 Hz, 3H) | Alternate Condition (1) was used Confirmed by SFC: Chiralpak IH-3, 50 × 4.6 mm, I.D., 3 um; mobile phase: 5%-50% IPA (0.1% IPAm, v/v) using a flow rate of 3.4 mL/min |
| | ((3S)-3-methyl-4- | | |
| | morpholinyl)(6-(pyrido[2,3- | | |
| | b]pyrazin-7-yl)thieno[2,3- | | |
| | b]pyridin-2-yl)methanone | | |
| B58 | 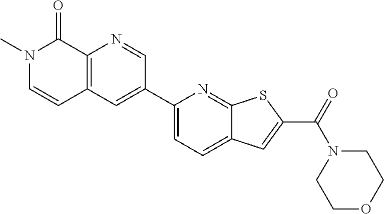 [SMILES: Cn1ccc2cc(-c3ccc4cc(C(=O)N5CCOCC5)sc4n3)cnc2c1=O] | 407.0; ¹H NMR (400 MHz, DMSO-d₆) δ 9.51 (d, J = 2.0 Hz, 1H), 8.86 (d, J = 1.6 Hz, 1H), 8.50 (d, J = 8.4 Hz, 1H), 8.32 (d, J = 8.4 Hz, 1H), 7.82 (s, 1H), 7.64 (d, J = 7.2 Hz, 1H), 6.76 (d, J = 7.2 Hz, 1H), 3.70 (br d, J = 9.2 Hz, 8H), 3.57 (s, 3H) | Alternate Condition (1) was used |
| | 7-methyl-3-(2-(4- | | |
| | morpholinylcarbonyl)thieno[2,3- | | |
| | b]pyridin-6-yl)-1,7- | | |
| | naphthyridin-8(7H)-one | | |
| B59 | 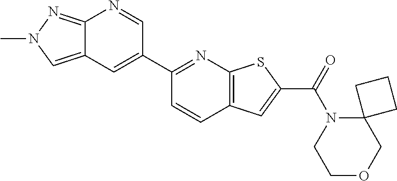 [SMILES: Cn1cc2cc(-c3ccc4cc(C(=O)N5CCOCC56CCC6)sc4n3)cnc2n1] | 425.5; ¹H NMR (400 MHz, DMSO-d₆) δ: 9.40 (d, J = 2.1 Hz, 1H), 8.97 (d, J = 2.3 Hz, 1H), 8.57 (s, 1H), 8.41 (d, J = 8.5 Hz, 1H), 8.20 (d, J = 8.5 Hz, 1H), 7.79 (s, 1H), 4.24 (s, 3H), 3.76 (s, 2H), 3.64-3.70 (m, 2H), 3.49-3.55 (m, 2H), 2.23-2.35 (m, 2H), 2.12-2.22 (m, 2H), 1.69-1.82 (m, 2H) | Alternate Condition (1) was used |
| | (6-(2-methyl-2H-pyrazolo[3,4- | | |
| | b]pyridin-5-yl)thieno[2,3- | | |
| | b]pyridin-2-yl)(8-oxa-5- | | |
| | azaspiro[3.5]nonan-5- | | |
| | yl)methanone | | |
| B60R | 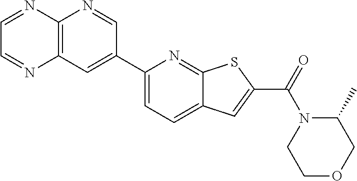 [SMILES: C[C@@H]1COCCN1C(=O)c1cc2ccc(-c3cnc4nccnc4c3)nc2s1] | 392.1; ¹H NMR (400 MHz, DMSO-d₆) δ 10.00 (d, J = 2.4 Hz, 1H), 9.25 (d, J = 2.4 Hz, 1H), 9.17 (dd, J = 1.5, 13.6 Hz, 2H), 8.55- 8.47 (m, 2H), 7.82 (s, 1H), 4.47- 4.36 (m, 1H), 4.10-3.99 (m, 1H), 3.89 (br d, J = 7.4 Hz, 1H), 3.71- 3.66 (m, 1H), 3.64-3.59 (m, 1H), 3.52-3.41 (m, 2H), 1.36 (d, J = 6.8 Hz, 3H) | Alternate Condition (1) was used Confirmed by SFC: Chiralpak IH-3, 50 × 4.6 mm, I.D., 3 um; mobile phase: 5%-50% IPA (0.1% IPAm, v/v) using a flow rate of 3.4 mL/min |
| | ((3R)-3-methyl-4- | | |
| | morpholinyl)(6-(pyrido[2,3- | | |
| | b ]pyrazin-7-yl)thieno[2,3- | | |
| | b]pyridin-2-yl)methanone | | |
| B61R | 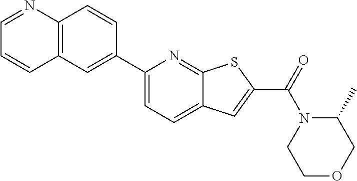 [SMILES: C[C@@H]1COCCN1C(=O)c1cc2ccc(-c3ccc4ncccc4c3)nc2s1] | 390.1; ¹H NMR (400 MHz, DMSO-d₆) δ 8.96 (dd, J = 1.5, 4.2 Hz, 1H), 8.86 (d, J = 1.8 Hz, 1H), 8.60 (dd, J = 2.0, 8.9 Hz, 1H), 8.55 (d, J = 8.2 Hz, 1H), 8.47 (d, J = 8.5 Hz, 1H), 8.30 (d, J = 8.5 Hz, 1H), 8.16 (d, J = 8.8 Hz, 1H), 7.78 (s, 1H), 7.61 (dd, J = 4.2, 8.3 Hz, 1H), 5.76 (s, 1H), 4.46-4.38 (m, 1H), 4.05 (br d, J = 11.3 Hz, 1H), 3.89 (br d, J = 7.7 Hz, 1H), 3.71-3.65 | Alternate Conditions (1) and (2) were used Confirmed by SFC: Chiralpak IH-3, 100 × 4.6 mm, I.D., 3 um; mobile phase: 50% IPA (0.1% IPAm, v/v) using a flow rate of 4 mL/min |
| | | (m, 1H), 3.64-3.58 (m, 1H), 3.46 | |
| | ((3R)-3-methyl-4- | (br d, J = 9.5 Hz, 2H), 1.36 (d, J = | |
| | morpholinyl)(6-(6- | 6.8 Hz, 3H) | |
| | quinolinyl)thieno[2,3-b]pyridin- | | |
| | 2-yl)methanone | | |
| B62S | 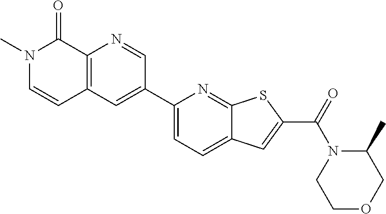 | 424.9; ¹H NMR (400 MHz, DMSO-d₆) δ 9.52 (s, 1H), 8.87 (s, 1H), 8.51 (d, J = 8.4 Hz, 1H), 8.32 (d, J = 8.4 Hz, 1H), 7.80 (s, 1H), 7.64 (d, J = 7.2 Hz, 1H), 6.76 (d, J = 7.2 Hz, 1H), 5.75 (s, 1H), 4.41 (br s, 1H), 4.03 (d, J = 9.6 Hz, 1H), 3.88 (d, J = 7.6 Hz, 1H), 3.66-3.62 (m, 2H), 3.57 (s, 3H), 3.46 (d, J = 8.0 Hz, 2H), 1.36 (d, J = 6.8 Hz, 3H) | Alternate Condition (1) was used Confirmed by SFC: Chiralpak AD-3, 100 × 4.6 mm, I.D., 3 um; mobile phase: 50% MeOH (0.1% IPAm, v/v) using a flow rate of 4 mL/min |
| | 7-methyl-3-(2-(((3S)-3-methyl-4- | | |
| | morpholinyl)carbonyl)thieno[2, | | |
| | 3-b]pyridin-6-yl)-1,7- | | |
| | naphthyridin-8(7H)-one | | |
| B62R | 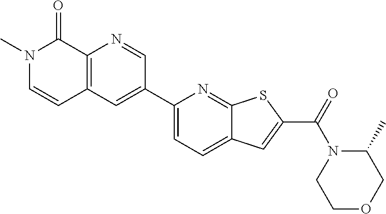 | 421.1; ¹H NMR (400 MHz, DMSO-d₆) δ 9.52 (s, J = 2.0 Hz, 1H), 8.87 (d, J = 2.4 Hz, 1H), 8.51 (d, J = 8.4 Hz, 1H), 8.33 (d, 8.8 Hz, 1H), 7.81 (s, 1H), 7.64 (d, J = 7.2 Hz, 1H), 6.76 (d, J = 8.4 Hz, 1H), 4.45-4.40 (m, 2H), 3.88 (d, J = 8.0 Hz, 1H), 3.69-3.62 (m, 1H), 3.57 (s, 3H), 3.74-3.45 (m, 2H), 1.36 (d, J = 7.2 Hz, 3H), 1.23 (s, 1H) | Alternate Condition (1) was used Confirmed by SFC: Chiralpak AD-3, 100 × 4.6 mm, I.D., 3 um; mobile phase: 50% MeOH (0.1% IPAm, v/v) using a flow rate of 4 mL/min |
| | 7-methyl-3-(2-(((3R)-3-methyl-4- | | |
| | morpholinyl)carbonyl)thieno[2, | | |
| | 3-b]pyridin-6-yl)-1,7- | | |
| | naphthyridin-8(7H)-one | | |
| B63S | 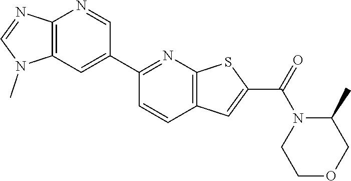 [SMILES: C[C@H]1COCCN1C(=O)c1cc2ccc(-c3cnc4ncn(C)c4c3)nc2s1] | 393.1; ¹H NMR (400 MHz, DMSO-d₆) δ 9.25 (d, J = 2.0 Hz, 1H), 8.80 (d, J = 2.0 Hz, 1H), 8.53 (s, 1H), 8.43 (d, J = 8.4 Hz, 1H), 8.27 (d, J = 8.4 Hz, 1H), 7.76 (s, 1H), 4.42 (br d, J = 5.2 Hz, 1H), 4.04 (d, J = 11.0 Hz, 1H), 3.98 (s, 3H), 3.88 (d, J = 7.6 Hz, 1H), 3.66- 3.69 (m, 1H), 3.61-3.63 (m, 1H), 3.41-3.47 (m, 2H), 1.36 (d, J = 6.8 Hz, 3H) | Alternate Condition (1) was used |
| | (6-(1-methyl-1H-imidazo[4,5- | | |
| | b]pyridin-6-yl)thieno[2,3- | | |
| | b]pyridin-2-yl)((3S)-3-methyl- | | |
| | 4-morpholinyl)methanone | | |
| B63R | 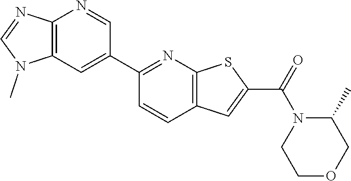 [SMILES: C[C@@H]1COCCN1C(=O)c1cc2ccc(-c3cnc4ncn(C)c4c3)nc2s1] | 394.2; ¹H NMR (400 MHz, DMSO-d₆) δ 9.25 (d, J = 2.0 Hz, 1H), 8.80 (d, J = 2.0 Hz, 1H), 8.54 (s, 1H), 8.44 (d, J = 8.5 Hz, 1H), 8.27 (d, J = 8.5 Hz, 1H), 7.77 (s, 1H), 4.42 (br d, J = 5.2 Hz, 1H), 4.05 (br d, J = 11.3 Hz, 1H), 3.98 (s, 3H), 3.89 (br d, J = 7.9 Hz, 1H), 3.66-3.71 (m, 1H), 3.59-3.64 (m, 1H), 3.51-3.41 (m, 2H), 1.36 (d, J = 6.8 Hz, 3H) | Alternate Condition (1) was used Confirmed by SFC: Chiralpak IH-3, 100 × 4.6 mm, I.D., 3 um; mobile phase: 5%- 50% MeOH (0.1% IPAm, v/v) using a flow rate of 3.4 mL/min |
| | (6-(1-methyl-1H-imidazo[4,5- | | |
| | b]pyridin-6-yl)thieno[2,3- | | |
| | b]pyridin-2-yl)((3R)-3-methyl- | | |
| | 4-morpholinyl)methanone | | |
| B64 | 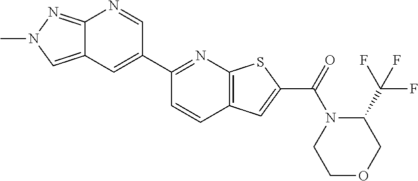 [SMILES: Cn1cc2cc(-c3ccc4cc(C(=O)N5CCOC[C@H]5C(F)(F)F)sc4n3)cnc2n1] | 448.2; ¹H NMR (400 MHz, DMSO-d₆) δ 9.41 (d, J = 2.0 Hz, 1H), 8.98 (d, J = 2.0 Hz, 1H), 8.57 (s, 1H), 8.43 (d, J = 8.8 Hz, 1H), 8.22 (d, J = 8.4 Hz, 1H), 7.92 (s, 1H), 5.21 (br s, 1H), 4.22 (s, 3H), 4.11-4.22 (m, 2H), 3.81-3.92 (m, 3H), 3.57 (t, J = 11.0 Hz, 1H). ¹⁹F NMR (377 MHz, DMSO-d₆) δ −68.76 (s, 3F) | |
| | (6-(2-methyl-2H-pyrazolo[3,4- | | |
| | b]pyridin-5-yl)thieno[2,3- | | |
| | b]pyridin-2-yl)(3- | | |
| | (trifluoromethyl)-4- | | |
| | morpholinyl)methanone | | |

### Group 5

### Example 5-1: (7-oxido-4-(2-propanyl)thieno[2,3-b]pyridin-2-yl)(1-piperidinyl)methanone B7

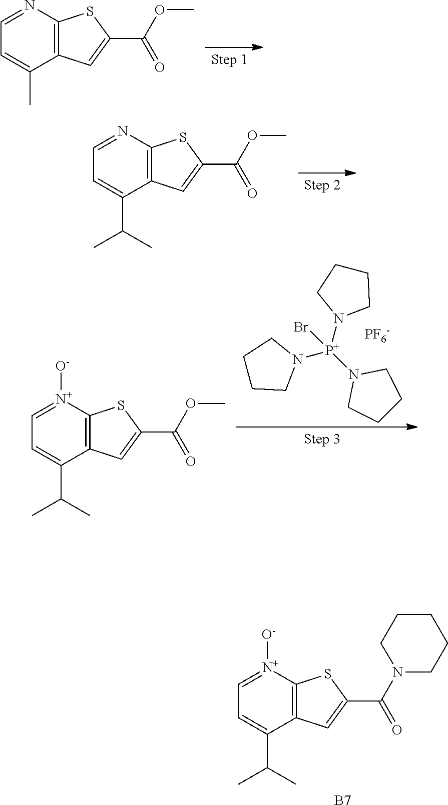

Step 1: 4-isopropyl-2-(methoxycarbonyl)thieno[2,3-b]pyridine. To a round-bottomed flask was charged with methyl 4-iodothieno[2,3-b]pyridine-2-carboxylate (0.500 g, 1.567 mmol), Pd(OAc)₂ (0.018 g, 0.078 mmol) and 2′-(dicyclohexylphosphino)-N2,N2,N6,N6-tetramethyl-[1,1′-biphenyl]-2,6-diamine (0.068 g, 0.157 mmol). The flask was evacuated and backfilled with N₂ three times. THF (8 mL) was introduced under nitrogen. The mixture was cooled in an ice bath. A 0.5M solution of 2-propylzinc bromide in tetrahydrofuran (3.76 mL, 1.880 mmol) was added slowly via a syringe under nitrogen. Upon completion of the addition, the ice bath was removed and the mixture was stirred at ambient temperature overnight. The mixture was cooled in an ice bath before being quenched with saturated ammonium chloride (0.3 mL) followed by MeOH (0.5 ml). The resulting mixture was stirred for 5 min, then concentration under reduced pressure. The residue was loaded in MeOH/DCM onto a silica gel precolumn (25 g) and subjected to automated flash column chromatography on a 40-g REDISEP GOLD® column eluting with (MeOH/DCM, 11 min 0 to 9%) to give methyl 4-isopropylthieno[2,3-b]pyridine-2-carboxylate (400 mg, 1.70 mmol, 108% crude yield) as a colorless oil. m/z (ESI): 236.20 (M+1)⁺. ¹H NMR (chloroform-d, 400 MHz) δ 8.50 (d, 1H, J=5.6 Hz), 8.27 (d, 1H, J=0.8 Hz), 7.62 (dd, 1H, J=0.8, 5.6 Hz), 3.98 (s, 3H), 3.6-3.7 (m, 1H), 1.43 (d, 6H, J=6.9 Hz).

Step 2: 4-isopropyl-2-(methoxycarbonyl)thieno[2,3-b]pyridine-7-oxide. To a stirred solution of methyl 4-isopropylthieno[2,3-b]pyridine-2-carboxylate (270 mg, 1.147 mmol) in DCM (8 mL) was added 3-chloroperoxybenzoic acid (277 mg, 1.606 mmol) in one portion as a solid. The resulting mixture was stirred at ambient temperature for 48 hours. The DCM solution was directly loaded onto a silica gel precolumn (25 g) and subjected to automated flash column chromatography on a 40 g REDISEP GOLD® column eluting with (EtOAc/EtOH, 3/1. v/v)/heptane (1 min at 0%, 12 min 0 to 60%, 2 min from 60 to 100%, and 6 min at 100%) to give 4-isopropyl-2-(methoxycarbonyl)thieno[2,3-b]pyridine 7-oxide (225 mg, 0.895 mmol, 78% yield) as an off-white solid. m/z (ESI): 252.20 (M+1)⁺. ¹H NMR (chloroform-d, 400 MHz) δ 8.2-8.3 (m, 2H), 7.55 (dd, 1H, J=0.7, 7.0 Hz), 4.2-4.4 (m, 1H), 3.98 (s, 3H), 1.55 (d, 6H, J=7.1 Hz).

Step 3: (7-oxido-4-(2-propanyl)thieno[2,3-b]pyridin-2-yl)(1-piperidinyl)methanone. A mixture of 4-isopropyl-2-(methoxycarbonyl)thieno[2,3-b]pyridine 7-oxide (20 mg, 0.080 mmol), bromotripyrrolidinophosphonium hexafluorophosphate (93 mg, 0.199 mmol), piperidine (172 mg, 0.20 mL, 2.020 mmol) and DIPEA (148 mg, 0.20 mL, 1.145 mmol) in THF (2 mL) in a 5-mL microwave reaction vessel was subjected to irradiation (15 h, 100° C.) The crude mixture was directly loaded onto a silica gel precolumn (25 g) and subjected to automated flash column chromatography on a 24-g REDISEP GOLD® column eluting with MeOH/DCM (1 min at 0 and 11 min from 0 to 20%) to give an impure product, which was dissolved in MeOH and subjected to preparative reverse-phase HPLC (Gemini™ Prep C18 10 μcolumn; Phenomenex, Torrance, CA; gradient elution of 10 to 95% MeCN in H₂O, where both solvents contain 0.1% TFA 15 min in a 15-min method) to give, after lyophilization, compound B7 (18 mg, 0.059 mmol, 74.3% yield) as an off-white solid. m/z (ESI): 305.0 (M+H)⁺. ¹H NMR (chloroform-d, 400 MHz) δ 8.49 (d, J=7.1 Hz, 1H), 7.84 (d, J=7.1 Hz, 1H), 7.79 (s, 1H), 4.1-4.4 (m, 1H), 3.69 (br s, 4H), 1.60-1.80 (m, 6H), 1.59 (d, J=6.9 Hz, 6H).

### Example 5-2: (4-cyclopropyl-6-(2-(1-piperazinyl)-5-pyrimidinyl)thieno[2,3-b]pyridin-2-yl)(1-piperidinyl)methanone B22

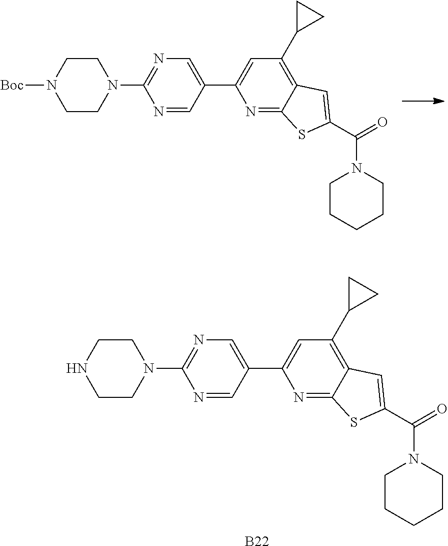 [SMILES: CC(C)(C)OC(=O)N1CCN(c2ncc(-c3cc(C4CC4)c4cc(C(=O)N5CCCCC5)sc4n3)cn2)CC1 ; O=C(c1cc2c(C3CC3)cc(-c3cnc(N4CCNCC4)nc3)nc2s1)N1CCCCC1 ; "B22"]

A solution of tert-butyl 4-(5-(4-cyclopropyl-2-(piperidine-1-carbonyl)thieno[2,3-b]pyridin-6-yl)pyrimidin-2-yl)piperazine-1-carboxylate (63.8 mg, 0.116 mmol) in DCM (1 mL) was treated with 4N HCl solution in 1,4-dioxane, (0.145 mL, 0.581 mmol). The reaction mixture was stirred at 25° C. for 18 h. The mixture was concentrated in vacuo to give Compound B22, as the hydrochloride salt (56.4 mg, 0.116 mmol, Yield: 100%) as a light yellow solid. m/z (ESI): 449.9 (M+H)⁺. 1H NMR (400 MHz, DMSO-d₆) δ 9.20 (s, 2H), 9.17 (br s, 2H), 7.92 (s, 1H), 7.42 (s, 1H), 4.03-4.10 (m, 4H), 3.66 (br d, J=5.4 Hz, 4H), 3.21 (br s, 4H), 2.53-2.60 (m, 1H), 1.67 (br d, J=4.4 Hz, 2H), 1.60 (br d, J=3.5 Hz, 4H), 1.11-1.27 (m, 4H)

### Group 6

### Example 6-1: (4,4-difluoro-1-piperidinyl)(4-(1-methyl-1H-pyrazol-5-yl)-2-(2-methyl-2H-[1,2,3]triazolo[4,5-b]pyridin-6-yl)thieno[2,3-d]pyrimidin-6-yl)methanone C9

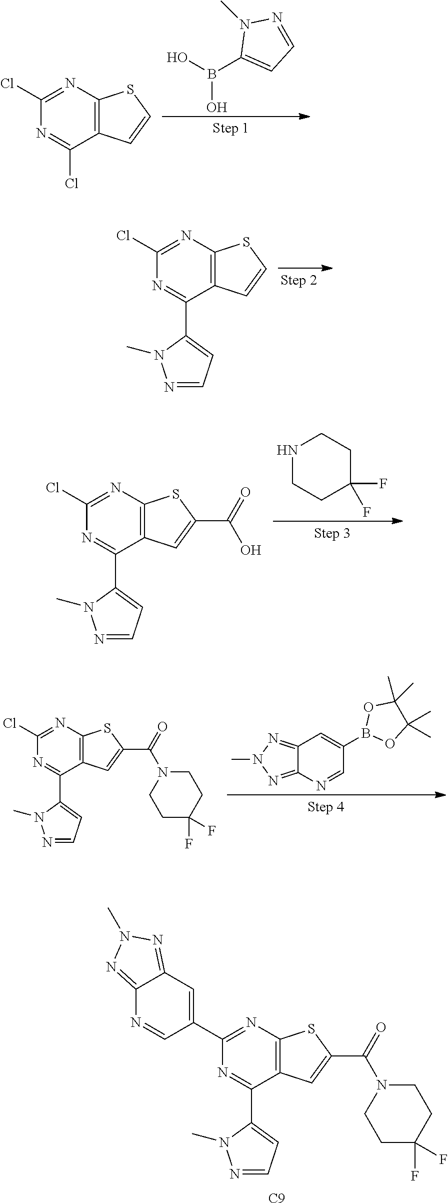

Step 1: 2-chloro-4-(1-methyl-1H-pyrazol-5-yl)thieno[2,3-d]pyrimidine. To a 50-mL pressure-release vial were added 2,4-dichlorothieno[2,3-d]pyrimidine (300 mg, 1.463 mmol), 1-methyl-1H-pyrazole-5-boronic acid (184 mg, 1.46 mmol), potassium phosphate (621 mg, 2.93 mmol), and cataCXium A Pd G3 (53.3 mg, 0.073 mmol). THF (1.32 mL) and H₂O (1.46 mL) were then added, and the resulting mixture was sparged gently with N₂ for 15 min and then heated to 70° C. After 16 h, the mixture was cooled to 23° C. The mixture was concentrated to dryness. The resulting crude residue was purified by flash chromatography (0 to 50% 3:1 EtOAc:EtOH in heptane gradient) to afford 2-chloro-4-(1-methyl-1Hpyrazol-5-yl)thieno[2,3-d]pyrimidine (138.6 mg, 0.553 mmol, Yield: 38%) as an off-white solid. m/z (ESI): 250.9 (M+H)⁺.

Step 2: 2-chloro-4-(1-methyl-1H-pyrazol-5-yl)thieno[2,3-d]pyrimidine-6-carboxylic acid. To a 50-mL pressure-release vial were added 2-chloro-4-(1-methyl-1H-pyrazol-5-yl)thieno[2,3-d]pyrimidine (138.6 mg, 0.553 mmol) and THF (5.5 mL). The resulting solution was flushed with nitrogen, cooled to −78° C., and n-butyllithium solution, 2.5 M in hexanes (0.243 mL, 0.608 mmol) was added dropwise. The resulting mixture was allowed to stir at −78° C. for 5 min under an atmosphere of N₂. A separate 50-mL pressure-release vial was charged with CO₂ solid (2.43 g, 55.3 mmol). A cannula was connected to the two vials in such a way that the subliming CO₂ could bubble through the reaction mixture at −78° C. After 20 min, the cooling bath was removed. The reaction mixture was allowed to warm to 23° C. over the course of 30 minutes while sparging with N₂ to flush out any dissolved CO₂. The reaction mixture was quenched by slow addition of sat. aq. NH₄Cl (5 mL) and transferred to a separatory funnel with EtOAc (30 mL), H₂O (10 mL), and 1 N aq. HCl (10 mL). The aq. layer was extracted with EtOAc (30 mL), and the combined organics were dried over Na₂SO₄, filtered, and concentrated in vacuo to afford 2-chloro-4-(1-methyl-1H-pyrazol-5-yl)thieno[2,3-d]pyrimidine-6-carboxylic acid (166.2 mg, 0.564 mmol, Crude yield: 102%) as a black solid. m/z (ESI): 294.85 (M+H)⁺. The material was used in the subsequent step without further purification.

Step 3: (2-chloro-4-(1-methyl-1H-pyrazol-5-yl)thieno[2,3-d]pyrimidin-6-yl)(4,4-difluoropiperidin-1-yl)methanone. To a 50-mL pressure-release vial were added 2-chloro-4-(1-methyl-1H-pyrazol-5-yl)thieno[2,3-d]pyrimidine-6-carboxylic acid (196.7 mg, 0.667 mmol), TBTU (321 mg, 1.001 mmol), and DMF (1.3 mL). The resulting solution was cooled to 0° C., 4,4-Difluoropiperidine (129 mg, 0.120 mL, 1.068 mmol) and DIPEA (431 mg, 0.583 mL, 3.34 mmol) were added, and the resulting mixture was allowed to stir at 0° C. After 2 h, the reaction mixture was transferred to a separatory funnel with EtOAc (30 mL) and the organic layer was washed with 1 M aq. LiCl (3×20 mL), dried over anhydrous Na₂SO₄, filtered, and concentrated to dryness. The resulting crude residue was purified by flash chromatography (0 to 100% EtOAc in heptane gradient) to afford (2-chloro-4-(1-methyl-1H-pyrazol-5-yl)thieno[2,3-d]pyrimidin-6-yl)(4,4-difluoropiperidin-1-yl)methanone (133.1 mg, 0.335 mmol, Yield: 50%) as a colorless foam. m/z (ESI): 398.05 (M+H)⁻.

Step 4: (4,4-difluoropiperidin-1-yl)(4-(1-methyl-1H-pyrazol-5-yl)-2-(2-methyl-2H-[1,2,3]triazolo[4,5-b]pyridin-6-yl)thieno[2,3-d]pyrimidin-6-yl)methanone. To a 10 mL pressure-release vial were added (2-chloro-4-(1-methyl-1H-pyrazol-5-yl)thieno[2,3-d]pyrimidin-6-yl)(4,4-difluoropiperidin-1-yl)methanone (41 mg, 0.103 mmol), 2-methyl-6-(4,4,5,5-tetramethyl-1,3,2-dioxaborolan-2-yl)-2H-[1,2,3]triazolo[4,5-b]pyridine (28.1 mg, 0.108 mmol), K₃PO₄ (43.8 mg, 0.206 mmol), and cataCXium A Pd G3 (3.75 mg, 5.15 mol). THF (0.9 mL) and H₂O (0.1 mL) were then added, and the resulting mixture was sparged gently with N₂ for 10 min and then heated to 70° C. After 16 h, the mixture was cooled to 23° C.) and concentrated to dryness. The resulting crude residue was purified by reverse-phase preparative HPLC using a Phenomenex Luna column, 5 micron, C8(2), 100 Å, 150×21.2 mm (10 to 60% gradient of 0.1% TFA/ACN in 0.1% TFA/H₂O) to afford Compound C9 (24.9 mg, 0.041 mmol, Yield: 40%) as an off-white solid after lyophilization. m/z (ESI): 496.2 (M+H)⁺. ¹H NMR (400 MHz, chloroform-d) δ 10.10 (d, J=1.9 Hz, 1H), 9.49 (d, J=1.9 Hz, 1H), 7.70-7.83 (m, 2H), 6.92 (d, J=2.1 Hz, 1H), 4.67 (s, 3H), 4.43 (s, 3H), 3.92 (brt, J=5.6 Hz, 4H), 2.05-2.21 (m, 4H).

### Alternate Conditions for Group C:

(1) K₂HPO₄ was used as the base, instead of K₃PO₄

(2) 5 mol % Pd(dtbf)Cl₂ was used as the catalyst, instead of cataCXium A Pd G3.

(3) Stille Cross-Coupling: To a stirring solution of product from Step 1 (1 equiv.) in 1,4-dioxane (1 mL) was added 3-methyl-6-(tributylstannyl)pyrimidin-4(3H)-one (413 mg, 1.035 mmol) and cataCXium A Pd G3 (8.05 mg, 0.011 mmol). The resulting mixture was sparged with N₂ for 10 min and then heated to 90° C. After 15 h, the mixture was allowed to cool to 23° C. and concentrated to dryness. The resulting crude residue was purified by reverse-phase preparative HPLC using a Phenomenex Luna column, 5 micron, C8(2), 100 Å, 150×21.2 mm (10 to 60% gradient of 0.1% TFA/ACN in 0.1% TFA/H₂O) to afford the product.

(4) Amide Formation with TBTU: To a stirring solution of product from Step 1 (1 equiv.), TBTU (46.3 mg, 0.144 mmol), and DMF (3 mL). To the resulting solution were added pyrrolidine (15.39 mg, 18.07 μL, 0.216 mmol) and DIPEA (93 mg, 126 μL, 0.721 mmol). The resulting mixture was allowed to stir at 23° C. After 30 min, the reaction mixture was transferred to a separatory funnel containing EtOAc (30 mL). The organic layer was washed with 1 M aq. LiCl (3×20 mL), dried over anhydrous Na₂SO₄, filtered, and concentrated to dryness. The resulting crude residue was purified by flash chromatography (0 to 100% 3:1 EtOAc:EtOH in heptane) to afford the product.

The compounds of Table 6-1 were prepared following the procedure described in Group 6.

TABLE 6-1

| | Chemical Structure & | | Reaction |
|---|---|---|---|
| Ex. # | Name | LCMS: (ESI + ve ion) m/z; NMR | Comments |
| C1 | 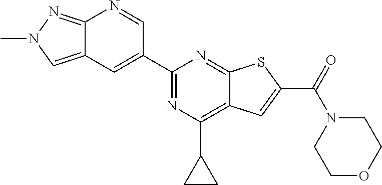 [SMILES: Cn1cc2cc(-c3nc(C4CC4)c4cc(C(=O)N5CCOCC5)sc4n3)cnc2n1] (4-cyclopropyl-2-(2- methyl-2H-pyrazolo[3,4- b]pyridin-5-yl)thieno[2,3- d]pyrimidin-6-yl)(4- morpholinyl)methanone | 421.2; ¹H NMR (400 MHz, chloroform-d) δ 9.84 (d, J = 2.1 Hz, 1 H), 9.15 (d, J = 2.3 Hz, 1 H), 8.04 (s, 1 H), 7.71 (s, 1 H), 4.31 (s, 3 H), 3.75-3.88 (m, 8 H), 2.40-2.51 (m, 1 H), 1.56 (dd, J = 4.4, 3.1 Hz, 2 H), 1.28-1.33 (m, 2 H) | Step 3 and Step 4 were performed in reverse order. Alternate Condition (4) was used |
| C2 | 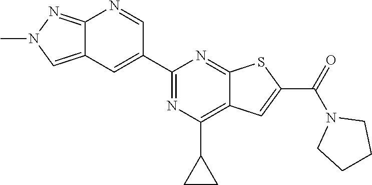 [SMILES: Cn1cc2cc(-c3nc(C4CC4)c4cc(C(=O)N5CCCC5)sc4n3)cnc2n1] (4-cyclopropyl-2-(2- methyl-2H-pyrazolo[3,4- b]pyridin-5-yl)thieno[2,3- d]pyrimidin-6-yl)(1- pyrrolidinyl)methanone | 405.1; ¹H NMR (500 MHz, chloroform-d) δ 9.84 (d, J = 2.2 Hz, 1 H), 9.16 (d, J = 2.2 Hz, 1 H), 8.04 (s, 1H), 7.93 (s, 1 H), 4.30 (s, 3 H), 3.90 (br t, J = 6.2 Hz, 2 H), 3.76 (br d, J = 6.5 Hz, 2 H), 2.42-2.53 (m, 1 H), 1.98-2.15 (m, 4 H), 1.56 (dd, J = 4.3, 3.0 Hz, 2 H), 1.28-1.33 (m, 2 H) | Step 3 and Step 4 were performed in reverse order. Alternate Condition (4) was used |
| C3 | 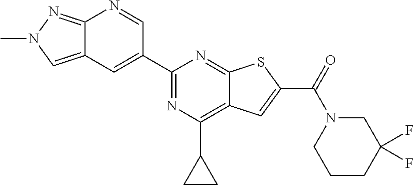 [SMILES: Cn1cc2cc(-c3nc(C4CC4)c4cc(C(=O)N5CCCC(F)(F)C5)sc4n3)cnc2n1] (4-cyclopropyl-2-(2- methyl-2H-pyrazolo[3,4- b]pyridin-5-yl)thieno[2,3- d]pyrimidin-6-yl)(3,3- difluoro-1- piperidinyl)methanone | 455.05; ¹H NMR (400 MHz, chloroform-d) δ 9.85 (d, J = 2.1 Hz, 1 H), 9.16 (d, J = 2.1 Hz, 1 H), 8.04 (s, 1 H), 7.75 (s, 1 H), 4.31 (s, 3 H), 3.99 (t, J = 11.2 Hz, 2 H), 3.74-3.83 (m, 2 H), 2.41-2.54 (m, 1 H), 2.17 (tt, J = 13.2, 6.5 Hz, 2 H), 1.88-2.01 (m, 2 H), 1.54-1.60 (m, 2 H), 1.24- 1.33 (m, 2 H); | |
| C4 | 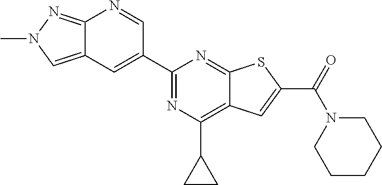 [SMILES: Cn1cc2cc(-c3nc(C4CC4)c4cc(C(=O)N5CCCCC5)sc4n3)cnc2n1] (4-cyclopropyl-2-(2- methyl-2H-pyrazolo[3,4- b]pyridin-5-yl)thieno[2,3- d]pyrimidin-6-yl)(1- piperidinyl)methanone | 419.2; ¹H NMR (400 MHz, chloroform-d) δ 9.85 (d, J = 2.1 Hz, 1 H), 9.15 (d, J = 2.1 Hz, 1 H), 8.04 (s, 1 H), 7.68 (s, 1 H), 4.31 (s, 3 H), 3.68-3.80 (m, 4 H), 2.38-2.53 (m, 1 H), 1.68-1.81 (m, 6 H), 1.56 (br dd, J = 4.5, 3.0 Hz, 2 H), 1.25-1.31 (m, 2 H) | |
| C5 | 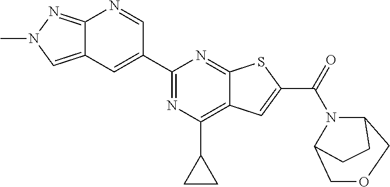 [SMILES: Cn1cc2cc(-c3nc(C4CC4)c4cc(C(=O)N5C6CCC5COC6)sc4n3)cnc2n1] (4-cyclopropyl-2-(2- methyl-2H-pyrazolo[3,4- b]pyridin-5-yl)thieno[2,3- d]pyrimidin-6-yl)((1R,5S)- 3-oxa-8- azabicyclo[3.2.1]octan-8- yl)methanone | 446.95; ¹H NMR (400 MHz, chloroform-d) δ 9.86 (d, J = 2.1 Hz, 1 H), 9.16 (d, J = 2.1 Hz, 1 H), 8.05 (s, 1 H), 7.87 (s, 1 H), 4.69 (br s, 2 H), 4.31 (s, 3 H), 3.91 (br d, J = 10.9 Hz, 2 H), 3.77 (d, J = 10.9 Hz, 2 H), 2.42-2.54 (m, 1 H), 2.04-2.19 (m, 4 H), 1.56-1.59 (m, 2 H), 1.31 (dq, J = 7.7, 3.7 Hz, 2 H) | |
| C6 | 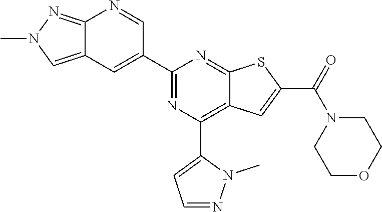 [SMILES: Cn1cc2cc(-c3nc(-c4ccnn4C)c4cc(C(=O)N5CCOCC5)sc4n3)cnc2n1] (2-(2-methyl-2H- pyrazolo[3,4-b]pyridin-5- yl)-4-(1-methyl-1H- pyrazol-5-yl)thieno[2,3- d]pyrimidin-6-yl)(4- morpholinyl)methanone | 461.2; ¹H NMR (400 MHz, chloroform-d) δ 9.90 (d, J = 2.1 Hz, 1 H), 9.26 (d, J = 2.1 Hz, 1 H), 8.10 (s, 1 H), 7.71 (s, 1 H), 7.69 (d, J = 1.9 Hz, 1 H), 6.87 (d, J = 1.9 Hz, 1 H), 4.40 (s, 3 H), 4.33 (s, 3 H), 3.81 (br d, J = 3.9 Hz, 8 H) | |
| C7 | 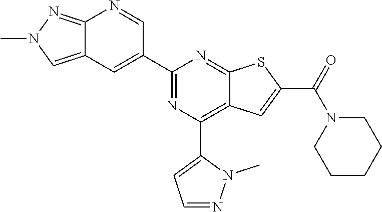 [SMILES: Cn1cc2cc(-c3nc(-c4ccnn4C)c4cc(C(=O)N5CCCCC5)sc4n3)cnc2n1] (2-(2-methyl-2H- pyrazolo[3,4-b]pyridin-5- yl)-4-(1-methyl-1H- pyrazol-5-yl)thieno[2,3- d]pyrimidin-6-yl)(1- piperidinyl)methanone | 459.1; ¹H NMR (400 MHz, chloroform-d) δ 9.92 (d, J = 2.1 Hz, 1 H), 9.27 (d, J = 2.1 Hz, 1 H), 8.11 (s, 1 H), 7.68 (d, J = 1.9 Hz, 1 H), 7.67 (s, 1 H), 6.88 (d, J = 1.9 Hz, 1 H), 4.40 (s, 3 H), 4.34 (s, 3 H), 3.73 (br d, J = 4.2 Hz, 4 H), 1.64-1.83 (m, 6 H) | |
| C8 | 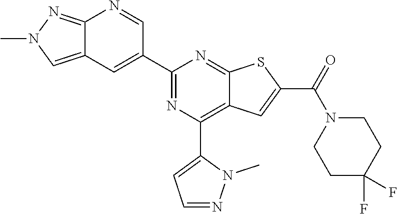 [SMILES: Cn1cc2cc(-c3nc(-c4ccnn4C)c4cc(C(=O)N5CCC(F)(F)CC5)sc4n3)cnc2n1] (4,4-difluoro-1- piperidinyl)(2-(2-methyl- 2H-pyrazolo[3,4-b]pyridin- 5-yl)-4-(1-methyl-1H- pyrazol-5-yl)thieno[2,3- d]pyrimidin-6- yl)methanone | 495.1; ¹H NMR (500 MHz, chloroform-d) δ 10.05 (s, 1 H), 9.50 (s, 1 H), 8.28 (s, 1 H), 7.73 (s, 2 H), 6.90 (d, J = 1.9 Hz, 1 H), 4.41 (s, 3 H), 4.39 (s, 3 H), 3.91 (br s, 4 H), 2.03-2.23 (m, 4 H) | |
| C10 | 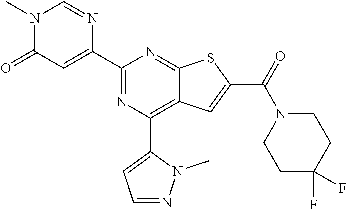 [SMILES: Cn1nccc1-c1nc(-c2cc(=O)n(C)cn2)nc2sc(C(=O)N3CCC(F)(F)CC3)cc12] 6-(6-((4,4-difluoro-1- piperidinyl)carbonyl)-4-(1- methyl-1H-pyrazol-5- yl)thieno[2,3-d]pyrimidin- 2-yl)-3-methyl-4(3H)- pyrimidinone | 472.5; ¹H NMR (500 MHz, chloroform-d) δ 8.54 (s, 1 H), 7.81 (s, 1 H), 7.70-7.79 (m, 2 H), 6.90 (d, J = 1.9 Hz, 1 H), 4.35 (s, 3 H), 3.89 (br s, 4 H), 3.69 (s, 3 H), 2.12 (br s, 4 H) | Alternate Condition (3) was used |
| C11 | 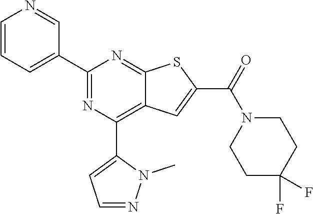 [SMILES: Cn1nccc1-c1nc(-c2cccnc2)nc2sc(C(=O)N3CCC(F)(F)CC3)cc12] (4,4-difluoro-1- piperidinyl)(4-(1-methyl- 1H-pyrazol-5-yl)-2-(3- pyridinyl)thieno[2,3- d]pyrimidin-6- yl)methanone | 441.1; ¹H NMR (400 MHz, DMSO- d₆) δ 9.65 (s, 1H), 8.88-8.72 (m, 2H), 7.96 (s, 1H), 7.73 (d, J = 1.3 Hz, 1H), 7.63 (dd, J = 4.8, 8.1 Hz, 1H), 7.21 (d, J = 1.4 Hz, 1H), 4.30 (s, 3H), 3.78 (br t, J = 5.3 Hz, 4H), 2.20-2.06 (m, 4H) | Alternate Condition (1) was used |
| C12 | 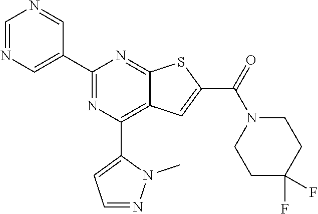 [SMILES: Cn1nccc1-c1nc(-c2cncnc2)nc2sc(C(=O)N3CCC(F)(F)CC3)cc12] (4,4-difluoro-1- piperidinyl)(4-(1-methyl- 1H-pyrazol-5-yl)-2-(5- pyrimidinyl)thieno[2,3- d]pyrimidin-6- yl)methanone | 442.1; ¹H NMR (400 MHz, DMSO- d₆) δ 9.76 (s, 2H), 9.39 (s, 1H), 7.98 (s, 1H), 7.74 (d, J = 2.1 Hz, 1H), 7.23 (d, J = 2.0 Hz, 1H), 4.36-4.23 (m, 3H), 3.78 (br t, J = 5.6 Hz, 4H), 2.21-2.03 (m, 4H) | Alternate Condition (1) was used |
| C13 | 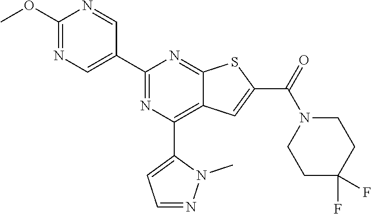 [SMILES: COc1ncc(-c2nc(-c3ccnn3C)c3cc(C(=O)N4CCC(F)(F)CC4)sc3n2)cn1] (4,4-difluoro-1- piperidinyl)(2-(2-methoxy- 5-pyrimidinyl)-4-(1- methyl-1H-pyrazol-5- yl)thieno[2,3-d]pyrimidin- 6-yl)methanone | 472.2; ¹H NMR (400 MHz, DMSO- d₆) δ 9.57 (s, 2H), 7.94 (s, 1H), 7.73 (d, J = 1.9 Hz, 1H), 7.20 (d, J = 2.0 Hz, 1H), 4.28 (s, 3H), 4.05 (s, 3H), 3.78 (br t, J = 5.4 Hz, 4H), 2.18-2.07 (m, 4H) | Alternate Condition (1) was used |
| C14 | 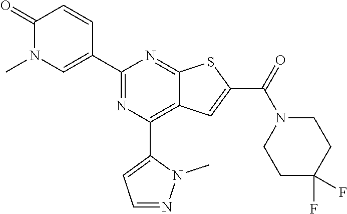 [SMILES: Cn1nccc1-c1nc(-c2ccc(=O)n(C)c2)nc2sc(C(=O)N3CCC(F)(F)CC3)cc12] 5-(6-((4,4-difluoro-1- piperidinyl)carbonyl)-4-(1- methyl-1H-pyrazol-5- yl)thieno[2,3-d]pyrimidin- 2-yl)-1-methyl-2(1H)- pyridinone | 471.1; ¹H NMR (400 MHz, DMSO- d₆) δ 8.93 (d, J = 2.3 Hz, 1H), 8.43 (dd, J = 2.4, 9.5 Hz, 1H), 7.86 (s, 1H), 7.71 (d, J = 1.9 Hz, 1H), 7.15 (d, J = 1.9 Hz, 1H), 6.56 (d, J = 9.5 Hz, 1H), 4.25 (s, 3H), 3.78 (br s, 4H), 3.62 (s, 3H), 2.17-2.06 (m, 4H) ¹⁹F NMR (377 MHz, DMSO-d₆) δ −95.82 (s, 1F) | Alternate Condition (1) was used |
| C15 | 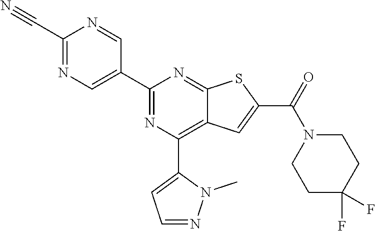 [SMILES: Cn1nccc1-c1nc(-c2cnc(C#N)nc2)nc2sc(C(=O)N3CCC(F)(F)CC3)cc12] 5-(6-((4,4-difluoro-1- piperidinyl)carbonyl)-4-(1- methyl-1H-pyrazol-5- yl)thieno[2,3-d]pyrimidin- 2-yl)-2- pyrimidinecarbonitrile | 467.1; ¹H NMR (400 MHz, DMSO- d₆) δ 9.96-9.87 (m, 2H), 8.01 (s, 1H), 7.75 (d, J = 2.0 Hz, 1H), 7.24 (d, J = 2.0 Hz, 1H), 4.29 (s, 3H), 3.78 (br t, J = 5.5 Hz, 4H), 2.19-2.08 (m, 4H), 2.07 (s, 1H) | Alternate Condition (1) was used |
| C16 | 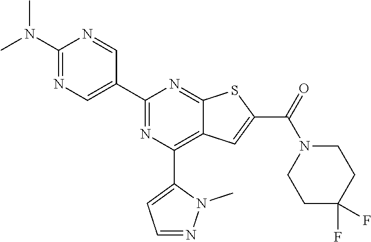 [SMILES: CN(C)c1ncc(-c2nc(-c3ccnn3C)c3cc(C(=O)N4CCC(F)(F)CC4)sc3n2)cn1] (4,4-difluoro-1- piperidinyl)(2-(2- (dimethylamino)-5- pyrimidinyl)-4-(1-methyl- 1H-pyrazol-5-yl)thieno[2,3- d]pyrimidin-6- yl)methanone | 485.1; ¹H NMR (400 MHz, DMSO- d₆) δ 9.33 (s, 2H), 7.88 (s, 1H), 7.71 (d, J = 2.0 Hz, 1H), 7.17 (d, J = 2.0 Hz), 1H), 4.25 (t, J = 6.0 Hz, 3H), 3.75 (m, 4H), 3.25 (s, 6H), 2.164- 2.081 (m, J = 6.6 Hz, 4H) | Alternate Condition (1) was used |
| C17 | 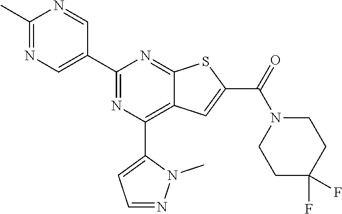 [SMILES: Cc1ncc(-c2nc(-c3ccnn3C)c3cc(C(=O)N4CCC(F)(F)CC4)sc3n2)cn1] (4,4-difluoro-1- piperidinyl)(4-(1-methyl- 1H-pyrazol-5-yl)-2-(2- methyl-5- pyrimidinyl)thieno[2,3- d]pyrimidin-6- yl)methanone | 456.1; ¹H NMR (400 MHz, DMSO- d₆) δ 9.64 (s, 2H), 8.03-7.92 (m, 1H), 7.73 (d, J = 2.0 Hz, 1H), 7.22 (d, J = 2.0 Hz, 1H), 4.29 (s, 3H), 3.78 (br t, J = 5.8 Hz, 4H), 2.75 (s, 3H), 2.19-2.05 (m, 4H) | Alternate Condition (1) was used |
| C18 | 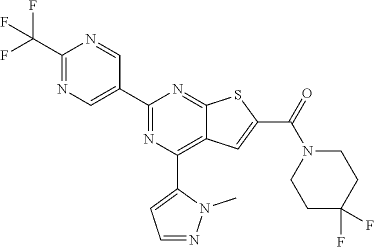 [SMILES: Cn1nccc1-c1nc(-c2cnc(C(F)(F)F)nc2)nc2sc(C(=O)N3CCC(F)(F)CC3)cc12] (4,4-difluoro-1- piperidinyl)(4-(1-methyl- 1H-pyrazol-5-yl)-2-(2- (trifluoromethyl)-5- pyrimidinyl)thieno[2,3- d]pyrimidin-6- yl)methanone | 510.1; ¹H NMR (400 MHz, DMSO- d₆) δ 9.99 (s, 2H), 8.02 (s, 1H), 7.75 (d, J = 2.1 Hz, 1H), 7.25 (d, J = 2.0 Hz, 1H), 4.30 (s, 3H), 3.78 (br t, J = 5.6 Hz, 4H), 2.20-2.07 (m, 4H) | Alternate Condition (1) was used |
| C19 | 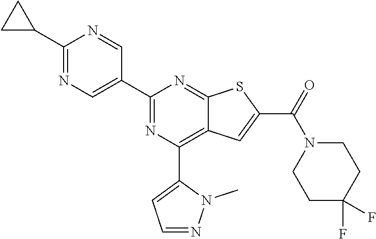 [SMILES: Cn1nccc1-c1nc(-c2cnc(C3CC3)nc2)nc2sc(C(=O)N3CCC(F)(F)CC3)cc12] (2-(2-cyclopropyl-5- pyrimidinyl)-4-(1-methyl- 1H-pyrazol-5-yl)thieno[2,3- d\|pyrimidin-6-yl)(4,4- difluoro-1- piperidinyl)methanone | 482.2; 1H NMR (400 MHz, DMSO- d₆) δ 9.63-9.54 (m, 2H), 7.96 (s, 1H), 7.73 (d, J = 2.1 Hz, 1H), 7.21 (d, J = 2.1 Hz, IH), 4.28 (s, 3H), 3.80-3.72 (m, 4H), 2.37-2.34 (m, 1H), 2.17-2.02 (m, 4H), 1.20-1.11 (m, 4H) | Alternate Condition (1) was used |
| C20 | 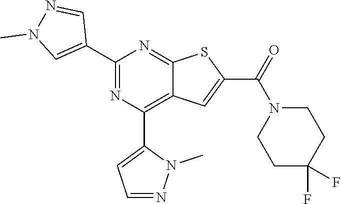 [SMILES: Cn1cc(-c2nc(-c3ccnn3C)c3cc(C(=O)N4CCC(F)(F)CC4)sc3n2)cn1] (4,4-difluoro-1- piperidinyl)(2-(1-methyl- 1H-pyrazol-4-yl)-4-(1- methyl-1H-pyrazol-5- yl)thieno[2,3-d]pyrimidin- 6-yl)methanone | 1H NMR (400 MHz, DMSO-d₆) δ = 8.57 (s, 1H), 8.19 (s, 1H), 7.85 (s, 1H), 7.69 (d, J = 2.1 Hz, 1H), 7.13- 7.16 (m, 1H), 4.25 (s, 3H), 3.94 (s, 3H), 3.78 (br t, J = 5.5 Hz, 4H), 2.07-2.16 (m, 4H). ¹⁹F NMR (377 MHz, DMSO-d₆) δ −95.82 (s, 2F) | Alternate Condition (1) was used |
| C21 | 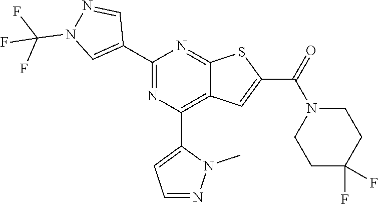 [SMILES: Cn1nccc1-c1nc(-c2cnn(C(F)(F)F)c2)nc2sc(C(=O)N3CCC(F)(F)CC3)cc12] (4,4-difluoro-1- piperidinyl)(4-(1-methyl- 1H-pyrazol-5-yl)-2-(1- (trifluoromethyl)-1H- pyrazol-4-yl)thieno[2,3- d]pyrimidin-6- yl)methanone | 498.1; ¹H NMR (400 MHz, DMSO- d₆) δ 9.35-9.30 (m, 1H), 8.70 (s, 1H), 7.91 (s, 1H), 7.71 (d, J = 2.1 Hz, 1H), 7.18 (d, J = 2.1 Hz, 1H), 4.26 (s, 3H), 3.78 (br t, J = 5.5 Hz, 4H), 2.17-2.05 (m, 4H) | Alternate Condition (1) was used |
| C22 | 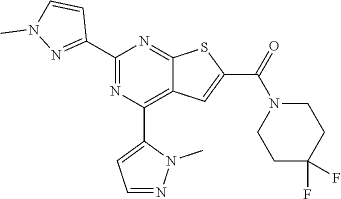 [SMILES: Cn1ccc(-c2nc(-c3ccnn3C)c3cc(C(=O)N4CCC(F)(F)CC4)sc3n2)n1] (4,4-difluoro-1- piperidinyl)(2-(1-methyl- 1H-pyrazol-3-yl)-4-(1- methyl-1H-pyrazol-5- yl)thieno[2,3-d]pyrimidin- 6-yl)methanone | 444.1; 1 H NMR (400 MHz, DMSO- d₆) δ 7.89 (s, 1H), 7.87 (d, J = 2.2 Hz, 1H), 7.70 (d, J = 2.1 Hz, 1H), 7.17 (d, J = 2.1 Hz, 1H), 7.07 (d, J = 2.2 Hz, 1H), 4.26 (s, 3H), 3.98 (s, 3H), 3.83-3.74 (m, 4H), 2.20-2.05 (m, 4H) | Alternate Condition (1) was used |
| C23 | 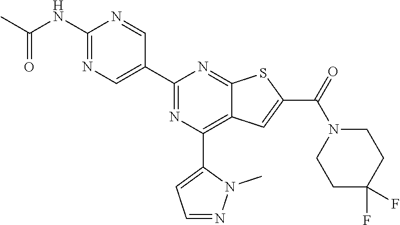 [SMILES: CC(=O)Nc1ncc(-c2nc(-c3ccnn3C)c3cc(C(=O)N4CCC(F)(F)CC4)sc3n2)cn1] N-(5-(6-((4,4-difluoro-1- piperidinyl)carbonyl)-4-(1- methyl-1H-pyrazol-5- yl)thieno[2,3-d]pyrimidin- 2-yl)-2- pyrimidinyl)acetamide | 499.1; ¹H NMR (400 MHz, DMSO- d₆) δ 10.93 (s, 1H), 9.62-9.53 (m, 2H), 7.96-7.93 (m, 1H), 7.73 (d, J = 2.0 Hz, 1H), 7.21 (d, J = 2.0 Hz, 1H), 4.29 (s, 3H), 3.78 (br t, J = 5.7 Hz, 4H), 2.26-2.26 (m, 1H), 2.26 (s, 3H), 2.16-2.07 (m, 4H) | |
| C24 | 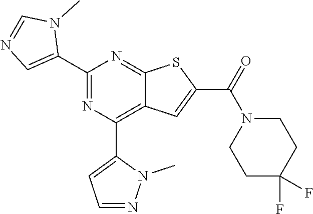 [SMILES: Cn1cncc1-c1nc(-c2ccnn2C)c2cc(C(=O)N3CCC(F)(F)CC3)sc2n1] (4,4-difluoro-1- piperidinyl)(2-(1-methyl- 1H-imidazol-5-yl)-4-(1- methyl-1H-pyrazol-5- yl)thieno[2,3-d]pyrimidin- 6-yl)methanone | 444.1; ¹H NMR (400 MHz, DMSO- d₆) δ 7.96-7.90 (m, 2H), 7.87-7.83 (m, 1H), 7.70 (s, 1H), 7.14 (s, 1H), 4.20 (s, 3H), 4.10 (s, 3H), 3.77 (br s, 4H), 2.16-2.07 (m, 4H) | Alternate Condition (1) was used |
| C25 | 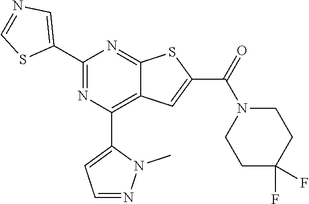 [SMILES: Cn1nccc1-c1nc(-c2cncs2)nc2sc(C(=O)N3CCC(F)(F)CC3)cc12] (4,4-difluoro-1- piperidinyl)(4-(1-methyl- 1H-pyrazol-5-yl)-2-(1,3- thiazol-5-yl)thieno[2,3- d]pyrimidin-6- yl)methanone | 469.0; ¹H NMR (400 MHz, DMSO- d₆) δ 9.32 (s, 1H), 8.82 (s, 1H), 7.93 (s, 1H), 7.73 (d, J = 2.0 Hz, 1H), 7.21 (d, J = 2.1 Hz, 1H), 4.26 (s, 3H), 3.78 (br t, J = 5.6 Hz, 4H), 2.16-2.07 (m, 4H) | Alternate Condition (1) was used |
| C26 | 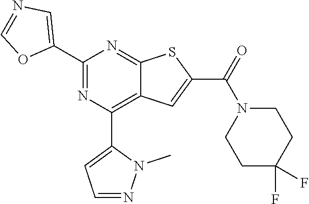 [SMILES: Cn1nccc1-c1nc(-c2cnco2)nc2sc(C(=O)N3CCC(F)(F)CC3)cc12] (4,4-difluoro-1- piperidinyl)(4-(1-methyl- 1H-pyrazol-5-yl)-2-(1,3- oxazol-5-yl)thieno[2,3- d]pyrimidin-6- yl)methanone | 431.0; ¹H NMR (400 MHz, DMSO- d₆) δ 8.71 (s, 1H), 8.18 (s, 1H), 7.94 (s, 1H), 7.72 (d, J = 1.9 Hz, 1H), 7.20 (d, J = 1.9 Hz, 1H), 4.25 (s, 3H), 3.78 (br t, J = 5.4 Hz, 4H), 2.17-2.06 (m, 4H) ¹⁹F NMR (377 MHz, DMSO-d₆) δ −95.85 (s, 1F) | |
| C27 | 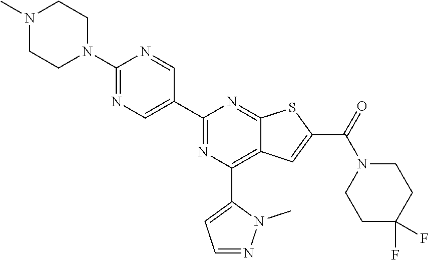 [SMILES: CN1CCN(c2ncc(-c3nc(-c4ccnn4C)c4cc(C(=O)N5CCC(F)(F)CC5)sc4n3)cn2)CC1] (4,4-difluoro-1- piperidinyl)(2-(2-(4- methyl-1-piperazinyl)-5- pyrimidinyl)-4-(1-methyl- 1H-pyrazol-5-yl)thieno[2,3- d]pyrimidin-6- yl)methanone | 540.2; ¹H NMR (400 MHz, DMSO- d₆) δ 9.33 (s, 2H), 7.88 (s, 1H), 7.71 (d, J = 2.0 Hz, 1H), 7.17 (d, J = 2.0 Hz, 1H), 4.26 (s, 1H), 3.88 (t, J = 3.1 Hz, 4H), 3.77 (t, J = 3.6 Hz, 4H), 2.40 (t, J = 4.8 Hz, 4H), 2.23 (s, 3H), 2.15- 2.07 (m, 4H) ¹⁹F NMR (376 MHz, DMSO-d₆) δ = −95.82 (s, 2F) | |
| C28 | 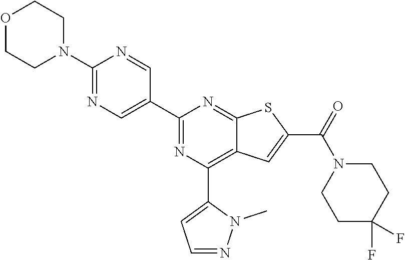 [SMILES: Cn1nccc1-c1nc(-c2cnc(N3CCOCC3)nc2)nc2sc(C(=O)N3CCC(F)(F)CC3)cc12] (4,4-difluoro-1- piperidinyl)(4-(1-methyl- 1H-pyrazol-5-yl)-2-(2-(4- morpholinyl)-5- pyrimidinyl)thieno[2,3- d]pyrimidin-6- yl)methanone | 527.2; ¹H NMR (400 MHz, DMSO- d₆) δ 9.35 (s, 2H), 7.88 (s, 1H), 7.70 (d, J = 2.0 Hz, 1H), 4.25 (s, 3H), 3.87 (t, J = 4.4 Hz, 4H), 3.77 (t, J = 5.4 Hz, 4H), 3.70 (t, J = 4.6 Hz, 4H), 2.16- 2.07 (m, J = 5.9 Hz, 4H) ¹⁹F NMR (376 MHz, DMSO-d₆) δ = −95.81 (s, 2F) | |
| C29 | 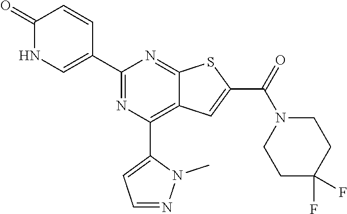 [SMILES: Cn1nccc1-c1nc(-c2ccc(=O)[nH]c2)nc2sc(C(=O)N3CCC(F)(F)CC3)cc12] 5-(6-((4,4-difluoro-1- piperidinyl)carbonyl)-4-(1- methyl-1H-pyrazol-5- yl)thieno[2,3-d]pyrimidin- 2-yl)-2(1H)-pyridinone | 457.1; ¹H NMR (400 MHz, DMSO- d₆) δ ppm 12.10 (br s, 1 H), 8.50 (br s, 1 H), 8.40-8.47 (m, 1 H), 7.86 (s, 1 H), 7.71 (s, 1 H), 7.16 (s, 1 H), 6.51 (d, J = 9.79 Hz, 1 H), 4.23 (s, 3 H), 3.77 (br s, 4 H), 2.10 (br dd, J = 11.61, 5.46 Hz, 4 H) ¹⁹F NMR (376 MHz, DMSO-d₆) δ ppm −95.82 (s, 2 F) | |
| C30 | 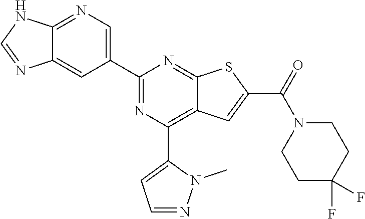 [SMILES: Cn1nccc1-c1nc(-c2cnc3[nH]cnc3c2)nc2sc(C(=O)N3CCC(F)(F)CC3)cc12] (4,4-difluoro-1- piperidinyl)(2-(3H- imidazo[4,5-b]pyridin-6- yl)-4-(1-methyl-1H- pyrazol-5-yl)thieno[2,3- d]pyrimidin-6- yl)methanone | 481.1; ¹H NMR (400 MHz, DMSO- d₆) δ 13.14 (br s, 1H), 9.53 (d, J = 1.5 Hz, 1H), 8.99 (s, 1H), 8.59 (s, 1H), 7.94 (s, 1H), 7.74 (d, J = 2.0 Hz, 1H), 7.21 (d, J = 2.1 Hz, 1H), 4.34 (s, 3H), 3.79 (br t, J = 5.6 Hz, 4H), 2.18-2.08 (m, 4H), 1.23 (s, 1H). | |
| C31 | 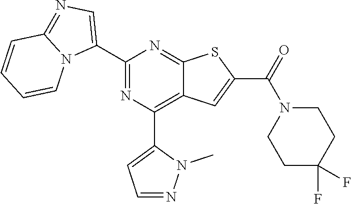 [SMILES: Cn1nccc1-c1nc(-c2cnc3ccccn23)nc2sc(C(=O)N3CCC(F)(F)CC3)cc12] (4,4-difluoro-1- piperidinyl)(2- (imidazo[1,2-a]pyridin-3- yl)-4-(1-methyl-1H- pyrazol-5-yl)thieno[2,3- d]pyrimidin-6- yl)methanone | 480.1; ¹H NMR (400 MHz, DMSO- d₆) δ 9.95 (d, J = 7.0 Hz, 1H), 8.70 (s, 1H), 7.88 (s, 1H), 7.84 (d, J = 9.0 Hz, 1H), 7.73 (d, J = 2.0 Hz, 1H), 7.58-7.52 (m, 1H), 7.35-7.27 (m, 1H), 7.19 (d, J = 2.0 Hz, 1H), 4.28 (s, 3H), 3.80 (br t, J = 5.5 Hz, 4H), 2.18-2.07 (m, 4H), 1.23 (br s, 1H), 1.05 (t, J = 7.0 Hz, 1H). | |
| C32 | 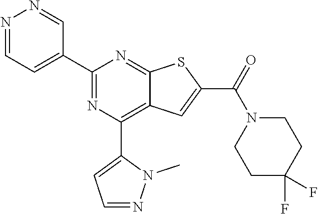 [SMILES: Cn1nccc1-c1nc(-c2ccnnc2)nc2sc(C(=O)N3CCC(F)(F)CC3)cc12] (4,4-difluoro-1- piperidinyl)(4-(1-methyl- 1H-pyrazol-5-yl)-2-(4- pyridazinyl)thieno[2,3- d]pyrimidin-6- yl)methanone | 483.1; ¹H NMR (400 MHz, DMSO- d₆) δ 10.15 (d, J = 0.8 Hz, 1H), 9.50 (t, J = 2.8 Hz, 1H), 8.60 (q, J = 2.4 Hz, 1H), 8.02 (s, 1H), 7.75 (d, J = 2.0 Hz, 1H), 7.24 (d, J = 2.0 Hz, 1H), 4.32 (s, 3H), 3.78 (t, J = 5.6 Hz, 4H), 2.18- 2.08 (m, 4H) ¹⁹F NMR (376 MHz, DMSO-d₆) δ = −95.85 (s, 1F) | |
| C33 | 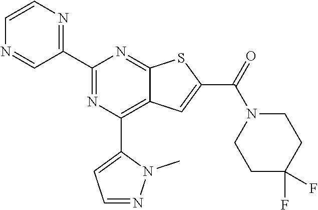 [SMILES: Cn1nccc1-c1nc(-c2cnccn2)nc2sc(C(=O)N3CCC(F)(F)CC3)cc12] (4,4-difluoro-1- piperidinyl)(4-(1-methyl- 1H-pyrazol-5-yl)-2-(2- pyrazinyl)thieno[2,3- d]pyrimidin-6- yl)methanone | 442.1; ¹H NMR (400 MHz, DMSO- d₆) δ 9.70 (s, 1H), 8.90 (d, J = 2.4 Hz, 1H), 8.84 (d, J = 2.4 Hz, 1H), 8.00 (s, 1H), 7.74 (d, J = 2.0 Hz, 1H), 7.23 (d, J = 2.0 Hz, 1H), 4.32 (s, 3H), 3.77- 3.80 (m, 4H), 2.10-2.17 (m, 4H). ¹⁹F NMR (377 MHz, DMSO-d₆) δ −95.85 (s, 2F) | |
| C34 | 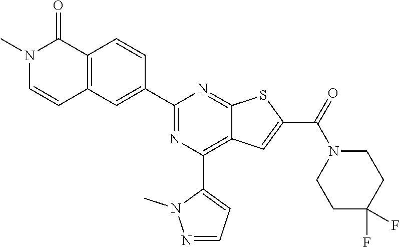 [SMILES: Cn1nccc1-c1nc(-c2ccc3c(=O)n(C)ccc3c2)nc2sc(C(=O)N3CCC(F)(F)CC3)cc12] 6-(6-((4,4-difluoro-1- piperidinyl)carbonyl)-4-(1- methyl-1H-pyrazol-5- yl)thieno[2,3-d]pyrimidin- 2-yl)-2-methyl-1(2H)- isoquinolinone | 521.2; ¹H NMR (400 MHz, DMSO- d₆) δ 8.82 (d, J = 1.4 Hz, 1H), 8.60 (dd, J = 1.6, 8.5 Hz, 1H), 8.40 (d, J = 8.6 Hz, 1H), 7.96 (s, 1H), 7.74 (d, J = 1.9 Hz, 1H), 7.56 (d, J = 7.3 Hz, 1H), 7.21 (d, J = 2.0 Hz, 1H), 6.89 (d, J = 7.4 Hz, 1H), 4.33 (s, 3H), 3.79 (br t, J = 5.5 Hz, 4H), 3.55 (s, 3H), 2.18-2.08 (m, 4H) ¹⁹F NMR (376 MHz, DMSO-d₆) δ −95.83 (s, 1F) | Alternate Condition (2) was used |
| C35 | 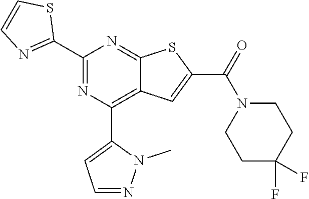 [SMILES: Cn1nccc1-c1nc(-c2nccs2)nc2sc(C(=O)N3CCC(F)(F)CC3)cc12] (4,4-difluoro-1- piperidinyl)(4-(1-methyl- 1H-pyrazol-5-yl)-2-(1,3- thiazol-2-yl)thieno[2,3- d]pyrimidin-6- yl)methanone | 447.0; ¹H NMR (400 MHz, DMSO- d₆) δ 8.15 (d, J = 3.2 Hz, 1H), 8.07 (d, J = 3.2 Hz, 1H), 7.99 (s, 1H), 7.74 (d, J = 2.0 Hz, 1H), 7.24 (d, J = 2.0 Hz, 1H), 4.30 (s, 3H), 3.78 (t, J = 5.6 Hz, 4H), 2.18-2.08 (m, 4H) ¹⁹F NMR (376 MHz, DMSO-d₆) δ −95.85 (s, 1F) | Alternate Condition (3) was used with Pd(PPh3)4 |
| C36 | 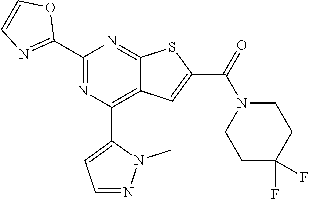 [SMILES: Cn1nccc1-c1nc(-c2ncco2)nc2sc(C(=O)N3CCC(F)(F)CC3)cc12] (4,4-difluoro-1- piperidinyl)(4-(1-methyl- 1H-pyrazol-5-yl)-2-(1,3- oxazol-2-yl)thieno[2,3- d]pyrimidin-6- yl)methanone | 431.1; ¹H NMR (400 MHz, DMSO- d₆) δ 8.46 (s, 1H), 8.01 (s, 1H), 7.73 (d, J = 2.0 Hz, 1H), 7.61 (s, 1H), 7.24 (d, J = 2.1 Hz, 1H), 4.29 (s, 3H), 3.78 (br t, J = 5.7 Hz, 4H), 2.19-.08 (m, 4H) | |
| C37 | 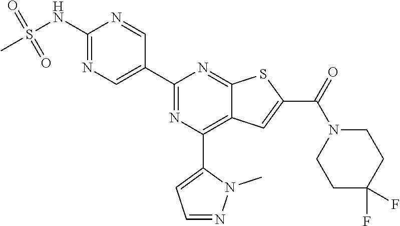 [SMILES: Cn1nccc1-c1nc(-c2cnc(NS(C)(=O)=O)nc2)nc2sc(C(=O)N3CCC(F)(F)CC3)cc12] N-(5-(6-((4,4-difluoro-1- piperidinyl)carbonyl)-4-(1- methyl-1H-pyrazol-5- yl)thieno[2,3-d]pyrimidin- 2-yl)-2- pyrimidinyl) methanesulfonamide | 535.1; . ¹H NMR (400 MHz, DMSO- d₆) δ 11.89-11.76 (m, 1H), 9.56 (s, 2H), 7.95 (s, 1H), 7.73 (d, J = 1.9 Hz, 1H), 7.21 (d, J = 2.0 Hz, 1H), 4.29 (s, 3H), 3.78 (br t, J = 5.5 Hz, 4H), 3.44 (s, 3H), 2.17-2.07 (m, 4H) | Alternate Condition (1) was used |
| C38 | 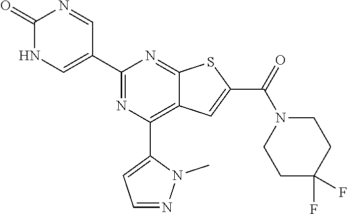 [SMILES: Cn1nccc1-c1nc(-c2cnc(=O)[nH]c2)nc2sc(C(=O)N3CCC(F)(F)CC3)cc12] 5-(6-((4,4-difluoro-1- piperidinyl)carbonyl)-4-(1- methyl-1H-pyrazol-5- yl)thieno[2,3-d]pyrimidin- 2-yl)-2(1H)-pyrimidinone | 458.1; ¹H NMR (400 MHz, DMSO- d₆) δ 9.20 (br s, 2H), 7.88 (s, 1H), 7.71 (d, J = 2.0 Hz, 1H), 7.17 (d, J = 2.1 Hz, 1H), 4.28-4.19 (m, 3H), 3.77 (br t, J = 5.4 Hz, 4H), 2.16-2.05 (m, 4H) | Alternate Condition (4) was used |

### Group 7

### Example 7-1: (4,4-difluoro-1-piperidinyl)(2-(2-methyl-2H-pyrazolo[3,4-b]pyridin-5-yl)thieno[2,3-b]pyrazin-6-yl)methanone C40

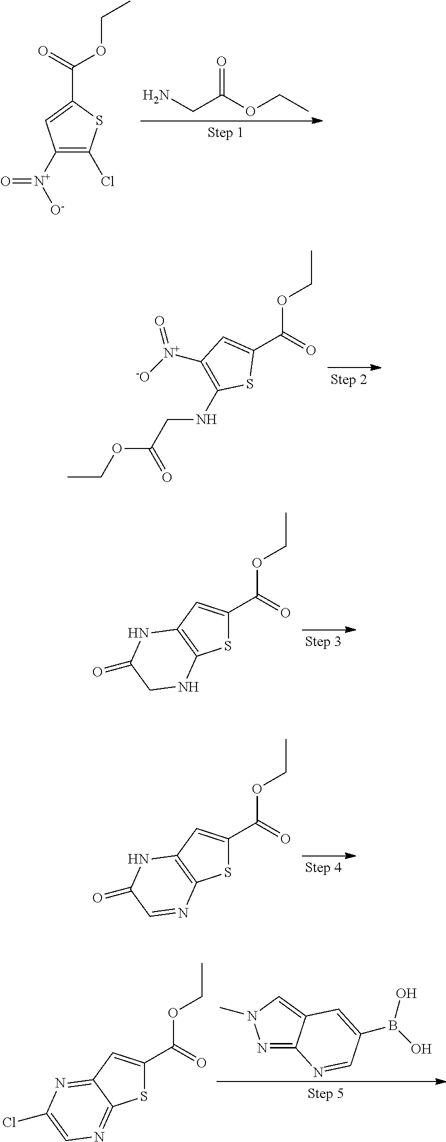

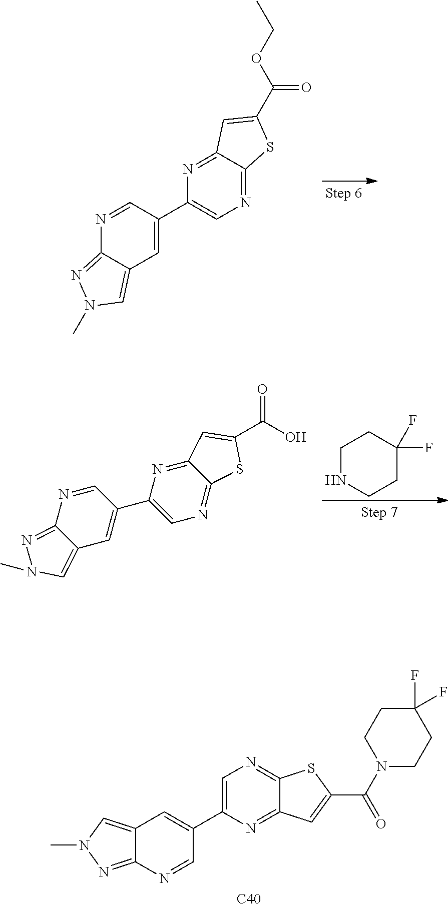 [SMILES: CCOC(=O)c1cc2nc(-c3cnc4nn(C)cc4c3)cnc2s1 ; Cn1cc2cc(-c3cnc4sc(C(=O)O)cc4n3)cnc2n1 ; FC1(F)CCNCC1 ; Cn1cc2cc(-c3cnc4sc(C(=O)N5CCC(F)(F)CC5)cc4n3)cnc2n1 ; "C40"]

Step 1: ethyl 5-((2-ethoxy-2-oxoethyl)amino)-4-nitrothiophene-2-carboxylate. To a 50-mL pressure-release vial were added ethyl 5-chloro-4-nitrothiophene-2-carboxylate (500 mg, 2.12 mmol), glycine ethyl ester hydrochloride (444 mg, 3.18 mmol), anhydrous K₂CO₃ (880 mg, 6.37 mmol), and ACN (1.7 mL). The resulting mixture was allowed to stir at 70° C. After 2 h the reaction was quenched by addition of sat. aq. NH₄Cl (10 mL), transferred to a separatory funnel with H₂O (10 mL) and EtOAc (20 mL), and extracted with EtOAc (20 mL). The combined organic phase was dried w/Na₂SO₄ and concentrated to dryness. The resulting crude residue was purified by automated flash chromatography (0 to 50% EtOAc in heptane) to afford ethyl 5-((2-ethoxy-2-oxoethyl)amino)-4-nitrothiophene-2-carboxylate (170.9 mg, 0.565 mmol, Yield: 27%) as a yellow solid. m/z (ESI): 303.0 (M+H)⁺

Step 2: ethyl 2-oxo-1,2,3,4-tetrahydrothieno[2,3-b]pyrazine-6-carboxylate. To a 50-mL pressure-release vial were added ethyl 5-((2-ethoxy-2-oxoethyl)amino)-4-nitrothiophene-2-carboxylate (170.9 mg, 0.565 mmol), iron powder (95 mg, 1.70 mmol), acetic acid (3.88 mL), and H₂O (0.65 mL). The resulting mixture was allowed to stir at 70° C. After 1 h the reaction mixture was filtered. The filtrate was frozen and lyophilized directly to afford crude ethyl 2-oxo-1,2,3,4-tetrahydrothieno[2,3-b]pyrazine-6-carboxylate as a brown solid. m/z (ESI): 226.9 (M+H)⁺. The material was used in the subsequent step without further purification.

Step 3: ethyl 2-oxo-1,2-dihydrothieno[2,3-b]pyrazine-6-carboxylate. To a 50-mL pressure-release vial were added ethyl 2-oxo-1,2,3,4-tetrahydrothieno[2,3-b]pyrazine-6-carboxylate (128 mg, 0.566 mmol), manganese(IV) oxide (492 mg, 5.66 mmol), and THF (5.7 mL). The resulting mixture was allowed to stir at 50° C. After 30 min the reaction mixture was filtered over a 1 cm pad of celite (20 mL EtOAc eluent). The filtrate was concentrated to afford ethyl 2-oxo-1,2-dihydrothieno[2,3-b]pyrazine-6-carboxylate as a brown solid. m/z (ESI): 224.95 (M+H)⁺. The material was used in the subsequent step without further purification.

Step 4: ethyl 2-chlorothieno[2,3-b]pyrazine-6-carboxylate. To a 50-mL pressure-release vial were added ethyl 2-oxo-1,2-dihydrothieno[2,3-b]pyrazine-6-carboxylate (127 mg, 0.566 mmol) and phenylphosphonic dichloride (3.31 g, 17.0 mmol). The resulting mixture was heated to 125° C. After 1.5 h the reaction mixture was cooled to 0° C. and quenched by addition of H₂O (5 mL). The resulting mixture was transferred to a separatory funnel with DCM (20 mL) and H₂O (10 mL), and extracted with DCM (2×20 mL). The resulting crude residue was purified by flash chromatography (0 to 40% EtOAc in heptane) to afford ethyl 2-chlorothieno[2,3-b]pyrazine-6-carboxylate (34.1 mg, 0.141 mmol, Yield: 25%) as an off-white solid. m/z (ESI): 242.95 (M+H)⁺.

Step 5: ethyl 2-(2-methyl-2H-pyrazolo[3,4-b]pyridin-5-yl)thieno[2,3-b]pyrazine-6-carboxylate. To a 25-mL pressure-release vial were added ethyl 2-chlorothieno[2,3-b]pyrazine-6-carboxylate (34.1 mg, 0.141 mmol), (2-methyl-2H-pyrazolo[3,4-b]pyridin-5-yl)boronic acid (27.4 mg, 0.155 mmol), anhydrous K₃PO₄ (59.7 mg, 0.281 mmol), and cataCXium A Pd G3 (10.23 mg, 0.014 mmol). 1,4-Dioxane (2.5 mL) and H₂O (0.3 mL) were added. The resulting mixture was sparged with N₂ for 10 min and then heated to 70° C. After 17 h, the mixture was cooled to 23° C. and concentrated to dryness. The resulting crude residue was purified by flash chromatography (0 to 100% 3:1 EtOAc:EtOH in heptane using a 10 g Biotage column) to afford ethyl 2-(2-methyl-2H-pyrazolo[3,4-b]pyridin-5-yl)thieno[2,3-b]pyrazine-6-carboxylate (37.8 mg, 0.111 mmol, Yield: 79%) as a light-yellow solid. m % z (ESI): 340.0 (M+H)⁺.

Step 6: 2-(2-methyl-2H-pyrazolo[3,4-b]pyridin-5-yl)thieno[2,3-b]pyrazine-6-carboxylic acid. To a 10-mL pressure-release vial were added ethyl 2-(2-methyl-2H-pyrazolo[3,4-b]pyridin-5-yl)thieno[2,3-b]pyrazine-6-carboxylate (37.8 mg, 0.111 mmol), 1,4-dioxane (0.56 mL), and MeOH (0.56 mL). To the resulting vigorously stirred suspension was added dropwise a 1 M aq. solution of NaOH (0.200 mL, 0.200 mmol). The resulting mixture was allowed to stir at 23° C. After 1 h the reaction was quenched by the addition of 1 NHCl (0.20 mL, 0.20 mmol). The resulting mixture was concentrated to dryness to afford crude 2-(2-methyl-2H-pyrazolo[3,4-b]pyridin-5-yl)thieno[2,3-b]pyrazine-6-carboxylic acid as an off-white solid. m/z (ESI): 312.0 (M+H)⁻. The material was used in the subsequent step without further purification.

Step 7: (4,4-difluoropiperidin-1-yl)(2-(2-methyl-2H-pyrazolo[3,4-b]pyridin-5-yl)thieno[2,3-b]pyrazin-6-yl)methanone 2,2,2-trifluoroacetate. To a 10-mL pressure-release vial were added 2-(2-methyl-2H-pyrazolo[3,4-b]pyridin-5-yl)thieno[2,3-b]pyrazine-6-carboxylic acid (34.7 mg, 0.111 mmol), TBTU (64.4 mg, 0.201 mmol), and DMF (2.2 mL). To the resulting suspension were added 4,4-difluoropiperidine (40.5 mg, 36.8 μL, 0.334 mmol) and DIPEA (144 mg, 195 μL, 1.12 mmol). The resulting mixture was allowed to stir at 23° C. After 15 min the reaction mixture was filtered. The filtrate was purified directly by reverse-phase preparative HPLC using a Phenomenex Luna column, 5 micron, C8(2), 100 Å, 150×21.2 mm (10 to 60% gradient of 0.1% TFA/ACN in 0.1% TFA/H₂O) to afford Compound C40 (26.3 mg, 0.050 mmol, Yield: 45%) as an off-white solid after lyophilization. m/z (ESI): 415.15 (M+H)⁺. ¹H NMR (400 MHz, chloroform-d) δ 9.54 (d, J=2.1 Hz, 1H), 9.11 (s, 1H), 8.86 (d, J=2.1 Hz, 1H), 8.15 (s, 1H), 7.70 (s, 1H), 4.36 (s, 3H), 3.94 (br t, J=5.8 Hz, 4H), 2.07-2.23 (m, 4H).

The compound of Table 7-1 was prepared following the procedure described in Group D.

TABLE 7-1

| | Chemical Structure & | |
|---|---|---|
| Ex. # | Name | LCMS: (ESI + ve ion) m/z; NMR |
| C39 | 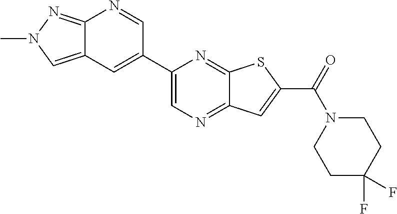 [SMILES: Cn1cc2cc(-c3cnc4cc(C(=O)N5CCC(F)(F)CC5)sc4n3)cnc2n1] (4,4-difluoro-1- piperidinyl)(3-(2-methyl- 2H-pyrazolo[3,4-b]pyridin- 5-yl)thieno[2,3-b]pyrazin- 6-yl)methanone | 415.0; ¹H NMR (400 MHz, chloroform-d) δ 9.61 (s, 1 H), 9.28 (s, 1 H), 9.04 (s, 1 H), 8.22 (s, 1 H), 7.71 (s, 1 H), 4.39 (s, 3 H), 3.90- 4.00 (m, 4 H), 2.10-2.20 (m, 4 H) |

### Group 8

### Example 8-1: (3-amino-5-(2-methyl-2H-pyrazolo[3,4-b]pyridin-5-yl)thieno[3,2-b]pyridin-2-yl)(1-piperidinyl)methanone C45

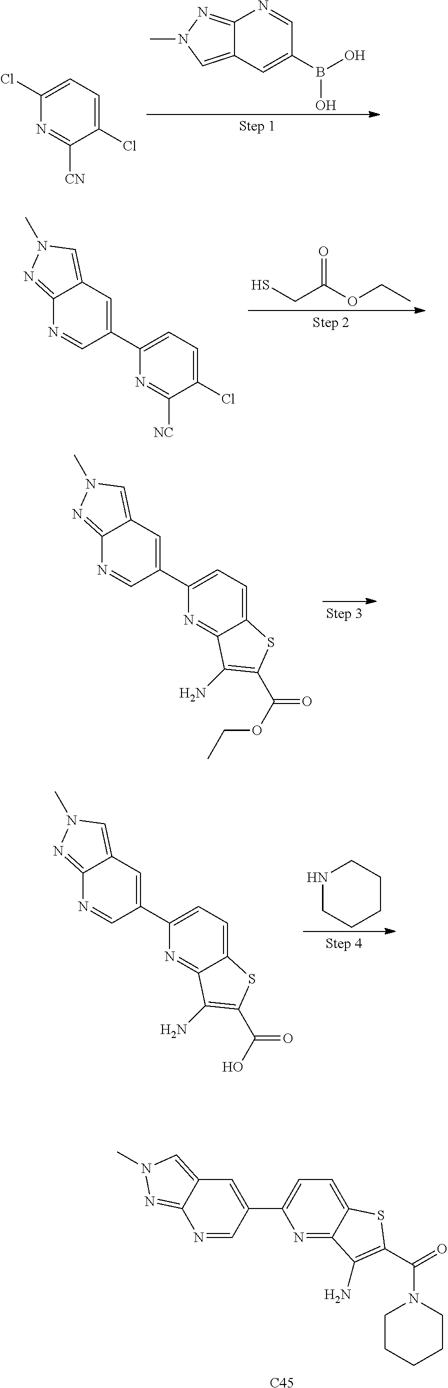

Step 1: 3-chloro-6-(2-methyl-2H-pyrazolo[3,4-b]pyridin-5-yl)picolinonitrile. A vial containing 3,6-dichloropicolinonitrile (0.500 g, 2.89 mmol), 2-methyl-5-(4,4,5,5-tetramethyl-1,3,2-dioxaborolan-2-yl)-2H-pyrazolo[3,4-b]pyridine (0.749 g, 2.89 mmol), anhydrous K₂CO₃ (0.799 g, 5.78 mmol) and Pd(dppf)Cl₂ (0.211 g, 0.289 mmol) was evacuated and backfilled with N₂ 3 times. 1,4-Dioxane (4 mL) and water (1 mL) were added via syringe. (The mixture was stirred at 110° C. for 12 hr under an atmosphere of N₂. The reaction mixture was poured into H₂O (10 mL) and extracted with EtOAc (3×10 mL). The combined organic layers were washed with brine 15 mL (15 mL), dried over Na₂SO₄, filtered and concentrated under reduced pressure to give 3-chloro-6-(2-methyl-2H-pyrazolo[3,4-b]pyridin-5-yl)picolinonitrile (1.10 g, 4.08 mmol, 71% yield) as brown oil. m/z (ESI): 270.1

Step 2: ethyl 3-amino-5-(2-methyl-2H-pyrazolo[3,4-b]pyridin-5-yl)thieno[3,2-b]pyridine-2-carboxylate. A mixture of 3-chloro-6-(2-methyl-2H-pyrazolo[3,4-b]pyridin-5-yl)picolinonitrile (1.00 g. 3.71 mmol), anhydrous K₂CO₃ (1.127 g, 8.16 mmol) and ethyl 2-mercaptoacetate (0.668 g, 5.56 mmol) in EtOH (20 mL) was degassed with N₂. The mixture was stirred at 80° C. for 3 hr under an atmosphere of N2. The reaction mixture was concentrated under reduced pressure to remove the solvent. The mixture was poured into H₂O (30 mL) and extracted with EtOAc (3×25 mL). The combined organic layers were washed with brine 15 mL (35 mL), dried over Na₂SO₄, filtered and concentrated under reduced pressure to give ethyl 3-amino-5-(2-methyl-2H-pyrazolo[3,4-b]pyridin-5-yl)thieno[3,2-b]pyridine-2-carboxylate (0.67 g, 1.896 mmol, 51% yield) as brown oil. m/z (ESI): 354.1

Step 3: 3-amino-5-(2-methyl-2H-pyrazolo[3,4-b]pyridin-5-yl)thieno[3,2-b]pyridine-2-carboxylic acid. A mixture of ethyl 3-amino-5-(2-methyl-2H-pyrazolo[3,4-b]pyridin-5-yl)thieno[3,2-b]pyridine-2-carboxylate (0.7 g, 1.981 mmol) and NaOH (0.119 g, 2.97 mmol) in EtOH (8 mL) and H₂O (2 mL) was stirred at 80° C. for 3 hr. The reaction mixture was concentrated under reduced pressure to give a residue. The residue was poured into H₂O (5 mL) and adjusted pH to 4 by addition of 1N HCl. Then the mixture was filtered to give 3-amino-5-(2-methyl-2H-pyrazolo[3,4-b]pyridin-5-yl)thieno[3,2-b]pyridine-2-carboxylic acid (0.62 g, 1.906 mmol, 84% yield) as a brown solid m/z (ESI): 325.9.

Step 4: (3-amino-5-(2-methyl-2H-pyrazolo[3,4-b]pyridin-5-yl)thieno[3,2-b]pyridin-2-yl)(piperidin-1-yl)methanone. To a solution of 3-amino-5-(2-methyl-2H-pyrazolo[3,4-b]pyridin-5-yl)thieno[3,2-b]pyridine-2-carboxylic acid (100 mg, 0.307 mmol) in DMF (0.5 mL) was added piperidine (31.4 mg, 0.369 mmol), HATU (140 mg, 0.369 mmol) and DIPEA (107 μL, 0.615 mmol) at 20° C. The mixture was stirred at 20° C. for 12 h. The mixture was directly purified by prep-HPLC (column: Waters Xbridge BEH C18 100*30 mm*10 um; mobile phase: H₂O (NH₄HCO₃)-ACN; B %: 20%-50%, 8 min). Compound C45 (0.0043 g, 10.79 μmol, 3.5% yield) was obtained as an off-white solid. m/z (ESI): 393.1 (M+H)⁺. ¹H NMR (400 MHz, DMSO-d₆) δ 9.62 (d, J=2.0 Hz, 1H), 9.05 (d, J=2.0 Hz, JH), 8.56 (s, 1H), 8.43 (d, J=8.8 Hz, 1H), 8.17 (d, J=8.4 Hz, 1H), 6.25 (s, 2H), 4.24 (s, 3H), 3.60 (t, J=4.8 Hz, 4H), 1.57-1.64 (m, 6H).

### Alternate Condition for Group E.1:

(1) To a solution of 5-(2-methyl-2H-pyrazolo[3,4-b]pyridin-5-yl)thieno[3,2-b]pyridine-2-carbonyl chloride (0.080 g, 0.24 mmol)) and piperidine (0.021 g, 0.24 mmol) in DCM (2 mL) was added Et₃N (0.068 mL, 0.49 mmol) at 20° C. The mixture was stirred at 20° C. for 12 h. The mixture was directly purified by prep-HPLC (column: Phenomenex C18 75*30 mm*3 um; mobile phase: H₂O (NH₃H₂O+NH₄HCO₃)-ACN; B %: 25%-60%, 8 min). to provide the product.

The compounds of Table 8-1 were prepared following the procedure described in Group 8.

TABLE 8-1

| | Chemical Structure & | | Reaction |
|---|---|---|---|
| Ex. # | Name | LCMS: (ESI + ve ion) m/z; NMR | Comments |
| C41 | 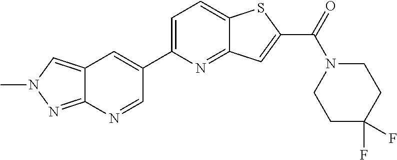 [SMILES: Cn1cc2cc(-c3ccc4sc(C(=O)N5CCC(F)(F)CC5)cc4n3)cnc2n1] (4,4-difluoro-1- piperidinyl)(5-(2-methyl- 2H-pyrazolo[3,4-b]pyridin- 5-yl)thieno[3,2-b]pyridin-2- yl)methanone | 414.1; ¹H NMR (500 MHz, chloroform-d) δ 9.42 (d, J = 1.9 Hz, 1 H), 8.73 (d, J = 1.9 Hz, 1 H), 8.30 (d, J = 8.6 Hz, 1 H), 8.04 (s, 1 H), 7.85 (d, J = 8.6 Hz, 1 H), 7.75 (s, 1 H), 4.32 (s, 3 H), 3.89-3.99 (m, 4 H), 2.05-2.18 (m, 4 H) | Alternative Condition (1) was used |
| C42 | 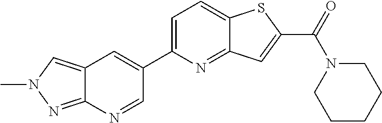 [SMILES: Cn1cc2cc(-c3ccc4sc(C(=O)N5CCCCC5)cc4n3)cnc2n1] (5-(2-methyl-2H- pyrazolo[3,4-b]pyridin-5- yl)thieno[3,2-b]pyridin-2- yl)(1- piperidinyl)methanone | 378.1; ¹H NMR (400 MHz, DMSO- d₆) δ 9.42 (d, J = 2.0 Hz, 1H), 8.94 (d, J = 2.0 Hz, 1H), 8.63 (d, J = 4.8 Hz, 1H), 8.57 (s, 1H), 8.14 (d, J = 8.8 Hz, 1H), 7.83 (s, 1H), 4.24 (s, 3H), 3.65 (t, J = 5.2 Hz, 4H), 1.67-1.59 (m, 6H) | Alternative Condition (1) was used |
| C43 | 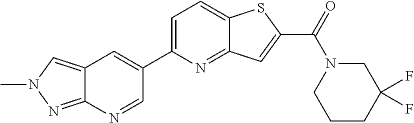 [SMILES: Cn1cc2cc(-c3ccc4sc(C(=O)N5CCCC(F)(F)C5)cc4n3)cnc2n1] (3,3-difluoro-1- piperidinyl)(5-(2-methyl- 2H-pyrazolo[3,4-b]pyridin- 5-yl)thieno[3,2-b]pyridin-2- yl)methanone | 414.1; ¹H NMR (400 MHz, DMSO- d₆) δ 9.42 (d, J = 2.4 Hz, 1H), 8.95 (d, J = 2.4 Hz, 1H), 8.65 (d, J -3.2 Hz, 1H), 8.57 (s, 1H), 8.17 (d, J = 8.8 Hz, 1H), 7.90 (s, 1H), 4.24 (s, 3H), 4.04 (t, J = 5.8 Hz, 2H), 3.74 (br s, 2H), 2.20-2.11 (m, 2H), 1.76 (br s, 2H) | Alternative Condition (1) was used |
| C44 | 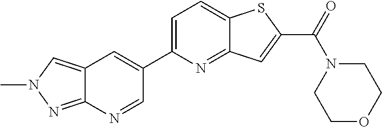 [SMILES: Cn1cc2cc(-c3ccc4sc(C(=O)N5CCOCC5)cc4n3)cnc2n1] (5-(2-methyl-2H- pyrazolo[3,4-b]pyridin-5- yl)thieno[3,2-b]pyridin-2- yl)(4- morpholinyl)methanone | 380.1; ¹H NMR (400 MHz, DMSO- d₆) δ 9.42 (d, J = 2.0 Hz, 1H), 8.93 (d, J = 2.0 Hz, 1H), 8.63 (d, J = 8.4 Hz, 1H), 8.57 (s, 1H), 8.15 (d, J = 8.8 Hz, 1H), 7.92 (s, 1H), 4.24 (s, 3H), 3.70 (d, J = 9.6 Hz, 8H) | Alternative Condition (1) was used |
| C46 | 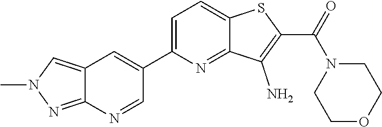 [SMILES: Cn1cc2cc(-c3ccc4sc(C(=O)N5CCOCC5)c(N)c4n3)cnc2n1] (3-amino-5-(2-methyl-2H- pyrazolo[3,4-b]pyridin-5- yl)thieno[3,2-b]pyridin-2- yl)(4- morpholinyl)methanone | 395.1; ¹H NMR (400 MHz, DMSO- d₆) δ 9.63 (d, J = 2.4 Hz, 1H), 9.06 (d, J = 2.4 Hz, 1H), 8.56 (s, 1H), 8.44 (d, J = 4.4 Hz, 1H), 8.18 (d, J = 8.8 Hz, 1H), 6.34 (s, 2H), 4.24 (s, 3H), 3.67- 3.63 (m, 8H) | |
| C47 | 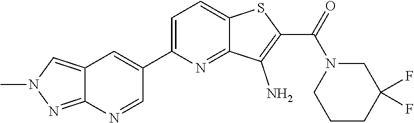 [SMILES: Cn1cc2cc(-c3ccc4sc(C(=O)N5CCCC(F)(F)C5)c(N)c4n3)cnc2n1] (3-amino-5-(2-methyl-2H- pyrazolo[3,4-b]pyridin-5- yl)thieno[3,2-b]pyridin-2- yl)(3,3-difluoro-1- piperidinyl)methanone | 429.1; ¹H NMR (400 MHz, DMSO- d₆) δ 9.66 (s, 1H), 9.12 (m, 1H), 8.59 (s, 1H), 8.46 (d, J = 8.4 Hz, 1H), 8.20 (d, J = 8.6 Hz, 1H), 4.24 (s, 3H), 3.93 (q, J = 7.9 Hz, 4H), 2.16-2.08 (m, 2H), 1.79-1.73 (m, 2H) | |
| C48 | 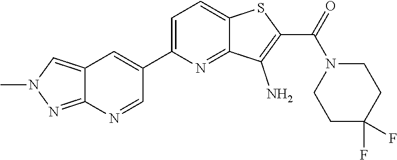 [SMILES: Cn1cc2cc(-c3ccc4sc(C(=O)N5CCC(F)(F)CC5)c(N)c4n3)cnc2n1] (3-amino-5-(2-methyl-2H- pyrazolo[3,4-b]pyridin-5- yl)thieno[3,2-b]pyridin-2- yl)(4,4-difluoro-1- piperidinyl)methanone | 429.1; ¹H NMR (400 MHz, DMSO- d₆) δ 9.63 (s, 1H), 9.06 (d, J = 2.0 Hz, 1H), 8.56 (s, 1H), 8.45 (d, J = 8.8 Hz, 1H), 8.18 (d, J = 8.4 Hz, 1H), 6.36 (s, 2H), 4.24 (s, 3H), 3.72 (t, J = 5.6 Hz, 4H), 2.14- 2.07 (4H) | |

### Group 9

### Example 9-1: (4-cyclopropyl-2-(2-methyl-2H-pyrazolo[3,4-b]pyridin-5-yl)thieno[3,2-d]pyrimidin-6-yl)(4-morpholinyl)methanone C49

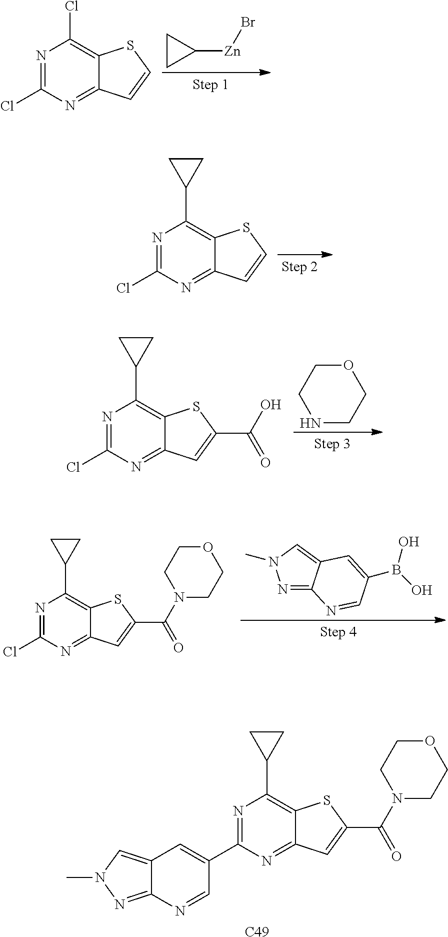

Step 1: 2-chloro-4-cyclopropylthieno[3,2-d]pyrimidine. To a stirring solution of 2,4-dichlorothieno[3,2-d]pyrimidine (500 mg, 2.44 mmol) and PEPPSI-iPr catalyst (166 mg, 0.244 mmol) in THF (4 mL) at 23° C. was added a 0.5 M solution of cyclopropylzinc(II) bromide in THF (9.75 mL, 4.88 mmol). The reaction mixture was allowed to stir at 23° C. After 17 h -d] the reaction mixture was diluted with sat. aq. NH₄Cl (5 mL), transferred to a separatory funnel with H₂O (20 mL) and EtOAc (40 mL). The aqueous phase was extracted with EtOAc (40 mL). The combined organics were dried with Na₂SO₄ and concentrated to dryness. The resulting crude residue was purified by flash chromatography (0 to 20% 3:1 EtOAc:EtOH in heptane) to afford 2-chloro-4-cyclopropylthieno[3,2-d]pyrimidine (133 mg, 0.632 mmol, Yield: 26%) as an off-white solid. m/z (ESI): 211.15 (M+H)⁺.

Step 2: 2-chloro-4-cyclopropylthieno[3,2-d]pyrimidine-6-carboxylic acid. To a 50-mL pressure-release vial were added 2-chloro-4-cyclopropylthieno[3,2-d]pyrimidine (133 mg, 0.632 mmol) and THF (6.3 mL). The resulting solution was flushed with nitrogen, cooled to −78° C., and n-butyllithium solution, 2.5 M in hexanes (0.278 mL, 0.695 mmol) was subsequently added dropwise. The resulting mixture was allowed to stir at −78° C. for 5 min under a N₂ atmosphere. A separate 50-mL pressure-release vial was charged with CO₂ solid (2.78 g, 63.2 mmol) and a cannula was connected in such a way that the subliming CO₂ could bubble through the reaction mixture at −78° C. After 20 min the cooling bath was removed. The reaction mixture was allowed to warm to 23° C. over the course of 30 minutes while sparging with N₂ to flush out any dissolved CO₂. The reaction mixture was then quenched by slow addition of sat. aq. NH₄Cl (5 mL) and transferred to a separatory funnel with EtOAc (30 mL), H₂O (10 mL), and 1 N aq. HCl (10 mL). The aq. layer was extracted with EtOAc (30 mL), and the combined organics were dried over Na₂SO₄, filtered, and concentrated in vacuo to afford 2-chloro-4-cyclopropylthieno[3,2-d]pyrimidine-6-carboxylic acid (160.6 mg, 0.631 mmol, Yield: 100%) as an off-white solid. m/z (ESI): 254.95 (M+H)⁺. The material was used in the subsequent step without further purification.

Step 3: (2-chloro-4-cyclopropylthieno[3,2-d]pyrimidin-6-yl)(morpholino)methanone. To a 50-mL pressure-release vial were added 2-chloro-4-cyclopropylthieno[3,2-d]pyrimidine-6-carboxylic acid (51.8 mg, 0.203 mmol), TBTU (85 mg, 0.264 mmol), and DMF (4 mL). The resulting solution was cooled to 0° C. Morpholine (28.4 mg, 28.5 μL, 0.325 mmol) and DIPEA (131 mg, 178 μL, 1.02 mmol) were added, and the resulting mixture was allowed to stir at 0° C. After 3 h the reaction mixture was transferred to a separatory funnel with EtOAc (30 mL) and the organic layer was washed with 1 M aq. LiCl (3×20 mL), dried over anhydrous Na₂SO₄, filtered, and concentrated to dryness under reduced pressure. The resulting crude residue was purified by flash chromatography (0 to 100% EtOAc in heptane gradient) to afford (2-chloro-4-cyclopropylthieno[3,2-d]pyrimidin-6-yl)(morpholino)methanone (48.2 mg, 0.149 mmol, Yield: 73%) as a colorless foam. m/z (ESI): 324.1 (M+H)⁺.

Step 4: (4-cyclopropyl-2-(2-methyl-2H-pyrazolo[3,4-b]pyridin-5-yl)thieno[3,2-d]pyrimidin-6-yl)(morpholino)methanone. To a 25-mL pressure-release vial were added (2-chloro-4-cyclopropylthieno[3,2-d]pyrimidin-6-yl)(morpholino)methanone (48.2 mg, 0.149 mmol), (2-methyl-2h-pyrazolo[3,4-b]pyridin-5-yl)boronic acid (31.6 mg, 0.179 mmol), anhydrous K₃PO₄ (95 mg, 0.447 mmol), and cataCXium A Pd G3 (5.42 mg, 7.44 mol). THF (1.3 mL) and H₂O (0.1 mL) were then added, and the resulting mixture was sparged with N₂ for 15 min. The mixture was heated to 70° C. After 17 h, the mixture was cooled to 23° C. and concentrated to dryness under reduced pressure. The resulting crude residue was purified by flash chromatography (0 to 100% 3:1 EtOAc:EtOH in heptane) to afford Compound C49 (44.3 mg, 0.105 mmol, Yield: 71%) as an off-white solid. m/z (ESI): 421.15 (M+H)⁺. ¹H NMR (400 MHz, chloroform-d) δ 9.85 (d, J=2.1 Hz, 1H), 9.13 (d, J=2.1 Hz, 1H), 8.04 (s, 1H), 7.64 (s, 1H), 4.31 (s, 3H), 3.83 (br s, 8H), 2.21-2.32 (m, 1H), 1.55-1.63 (m, 2H), 1.30-1.37 (m, 2H).

### Group 10

### Example 10-1: ((3R)-3-methyl-4-morpholinyl)(5-(2-methyl-2H-pyrazolo[3,4-b]pyridin-5-yl)[1,3]thiazolo[5,4-b]pyridin-2-yl)methanone C52R

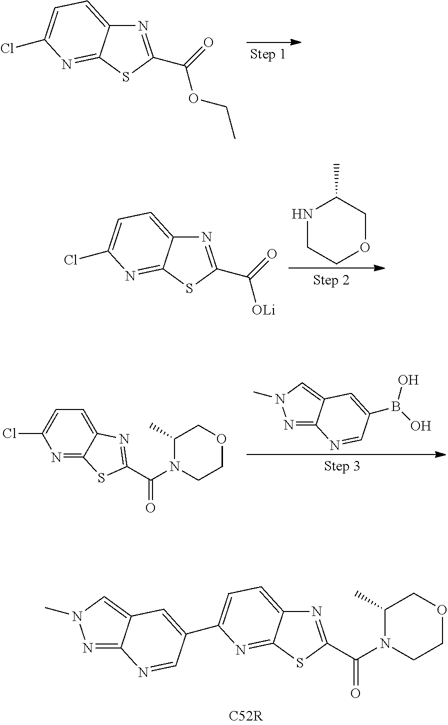

Step 1: lithium 5-chlorothiazolo[5,4-b]pyridine-2-carboxylate. To a solution of ethyl 5-chlorothiazolo[5,4-b]pyridine-2-carboxylate (0.300 g, 1.24 mmol) in THF (4 mL) and H₂O (1 mL) was added LiOH·H₂O (0.057 g, 1.36 mmol). The resulting mixture was stirred at rt for 4 hours. The reaction was concentrated and lyopholized to give crude lithium 5-chlorothiazolo[5,4-b]pyridine-2-carboxylate (0.275 g, 1.25 mmol, Crude yield: 101%). m/z (ESI): 215.0 (M+H)⁺. The crude material was used without further purification.

Step 2: (R)-(5-chlorothiazolo[5,4-b]pyridin-2-yl)(3-methylmorpholino)methanone. A mixture of lithium 5-chlorothiazolo[5,4-b]pyridine-2-carboxylate (0.100 g, 0.453 mmol), (R)-3-methyl-morpholine (0.055 g, 0.544 mmol), and PyBOP (0.283 g, 0.544 mmol) in N-methyl-2-pyrrolidinone (1 mL) was stirred at rt for 3 hours. The reaction was diluted with H₂O (10 mL) and extracted with EtOAc (2×20 mL). The combined organic phase was concentrated and the residue purified by automated flash column chromatography using 0-80% EtOAc/EtOH (3:1) in heptane (product eluted at 35%) to afford (R)-(5-chlorothiazolo[5,4-b]pyridin-2-yl)(3-methylmorpholino)methanone (0.048 g, 0.161 mmol, Yield: 35.6%). m/z (ESI): 298.0 (M+H)⁺.

Step 3: (R)-(5-(2-methyl-2H-pyrazolo[3,4-b]pyridin-5-yl)thiazolo[5,4-b]pyridin-2-yl)(3-methylmorpholino)methanone. A mixture of (R)-(5-chlorothiazolo[5,4-b]pyridin-2-yl)(3-methylmorpholino)methanone (0.048 g, 0.16 mmol, 129279-5-1), [2-methyl-2 h-pyrazolo[3,4-b]pyridin-5-yl]boronic acid (0.057 g, 0.32 mmol), Pd(dppf)Cl₂ (0.024 g, 0.032 mmol), and K₂CO₃ (0.067 g, 0.484 mmol, Oakwood) in 1,4-dioxane (2 mL) and H₂O (0.5 mL) contained in a microwave vial was capped and heated in microwave at 110° C. for 30 minutes. The reaction was concentrated and residue purified by automated flash column chromatography using 0-100% EtOAc/EtOH (3:1) in heptane (product eluted at 95%) to afford Compound C52R (0.028 g, 0.071 mmol, Yield: 44.0%). m/z (ESI): 395.0 (M+H)⁺. ¹H NMR (400 MHz, chloroform-d) δ 9.43 (br s, 1H) 8.83 (br s, 1H) 8.40 (br d, J=7.11 Hz, 1H) 7.94-8.13 (m, 2H) 5.19-5.73 (m, 1H) 4.39-4.94 (m, 1H) 4.24-4.49 (m, 2H) 3.93-4.18 (m, 1H) 3.75-3.90 (m, 2H) 3.37-3.74 (m, 2H) 1.59-2.28 (m, 1H) 1.43-1.60 (m, 3H).

The compounds of Table 9 were prepared following the procedure described in Group F.

TABLE 10-1

| | Chemical Structure & | | Reaction |
|---|---|---|---|
| Ex. # | Name | LCMS: (ESI + ve ion) m/z; NMR | Comments |
| C50 | 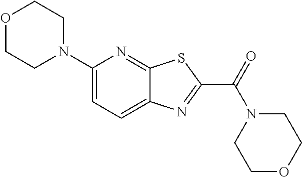 [SMILES: O=C(c1nc2ccc(N3CCOCC3)nc2s1)N1CCOCC1] 4-morpholinyl(5-(4- morpholinyl)[1,3]thiazolo [5,4-b]pyridin-2- yl)methanone | 335.2; ¹H NMR (400 MHz, chloroform-d) δ 8.04 (m, J = 9.20 Hz, 1 H) 6.83 (m, J = 9.20 Hz, 1 H) 4.52 (br s, 2 H) 3.76-3.92 (m, 10 H) 3.62-3.74 (m, 4 H) | |
| C51 | 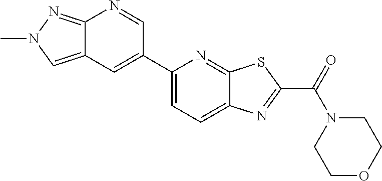 [SMILES: Cn1cc2cc(-c3ccc4nc(C(=O)N5CCOCC5)sc4n3)cnc2n1] (5-(2-methyl-2H- pyrazolo[3,4-b]pyridin-5- yl)[1,3]thiazolo[5,4- b]pyridin-2-yl)(4- morpholinyl)methanone | 381.1; ¹H NMR (400 MHz, DMSO- d₆) δ 9.43 (br s, 1 H) 9.02 (br s, 1 H) 8.55-8.68 (m, 2 H) 8.37 (br d, J = 7.94 Hz, 1 H) 4.35 (br s, 2 H) 4.26 (br s, 3 H) 3.75 (br s, 6 H). | Step 2: PyBOP was omitted |
| C52S | 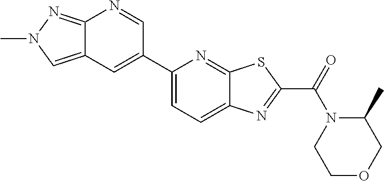 ((3S)-3-methyl-4- morpholinyl)(5-(2-methyl- 2H-pyrazolo[3,4-b]pyridin- 5-yl)[1,3]thiazolo[5,4- b]pyridin-2-yl)methanone | 395.1; ¹H NMR (400 MHz, DMSO- d₆) δ 9.34-9.47 (m, 1 H) 9.02 (br s, 1 H) 8.62 (s, 1 H) 8.59 (s, 1 H) 8.23- 8.48 (m, 1 H) 4.91-5.49 (m, 1 H) 4.47-4.68 (m, 1 H) 4.26 (br s, 3 H) 3.84-4.12 (m, 1 H) 3.46-3.79 (m, 4 H) 1.30-1.55 (m, 3 H) | |

### Section 3: Biochemical & Cellular Assays

### Example 11: Inhibition Study

The compound dose-response of its inhibition of 15-PGDH activity was determined in human recombinant 15-PGDH enzymatical assays. These assays measured 15-PGDH enzymatic activity by quantifying the amount of NADH produced during a 60-minute reaction initiated by the addition of PGE2 and NAD+. Under the conditions of these experiments, the increase of NADH signal upon oxidation of PGE2 and reduction of NAD+ was linearly proportional to 15-PGDH enzymatic activity. The recombinant human, mouse, rat and dog 15-PGDH enzymatic assays were performed in a 25 μL volume of reaction buffer containing 50 mM Tris, pH 7.5, 0.01% Tween-20 and 100 μM DTT in a 384-well microtiter plate. In concentration-response experiments with tested compounds, 22 concentrations from 2-fold serial dilutions in DMSO were pre-incubated with 15-PGDH for 15 minutes at room temperature. Then, PGE2 and ß-NAD+ were added to initiate the 15-PGDH reaction. After 60 minutes at room temperature, the reaction was quenched and NADH signal was measured using a microtiter plate reader.

The following data (Table 11-1) provides the IC50 of each compound for inhibiting enzymatic activity of recombinant 15-PGDH in the assay described above.

TABLE 11-1

| ID | PGDH-Lumi IC₅₀ |
|---|---|
| A1S | 0.217 |
| A1R | 0.273 |
| A2 | 0.163 |
| A3 | 0.0117 |
| A4R | 0.225 |
| A4S | 0.0195 |
| A5 | 0.0878 |
| A6 | 0.185 |
| A7S | 0.0664 |
| A7R | 0.0495 |
| A8 | 4.50 |
| A9 | 4.96 |
| A10 | 1.61 |
| A11 | 0.851 |
| B3 | 7.11E−4 |
| B4 | 3.87E−4 |
| B5 | 2.55E−4 |
| B6S | 5.54E−4 |
| B6R | 8.16E−4 |
| B7 | >100.0 |
| B8 | 0.00555 |
| B9R | 0.00639 |
| B9S | 0.00505 |
| B10 | 9.17E−4 |
| B11 | 3.17E−4 |
| B12 | 7.04E−4 |
| B13 | 0.00126 |
| B14 | 5.19E−4 |
| B15 | 4.75E−4 |
| B16 | 0.00951 |
| B17S | 0.00117 |
| B18 | 0.00158 |
| B17R | 0.00311 |
| B19 | 8.53E−4 |
| B20 | 0.00111 |
| B21 | 0.00145 |
| B22 | 0.00361 |
| B23 | 2.15E−4 |
| B24 | 4.28E−4 |
| B25 | 3.52E−4 |
| Not included | 0.00983 |
| B27 | 0.00170 |
| B28 | 0.0119 |
| B27R | 0.00151 |
| B27S | 0.00124 |
| B29 | 0.0836 |
| B30 | 0.212 |
| B31 | 0.00134 |
| B32S | 0.623 |
| B32R | 1.35 |
| B33S | 2.65 |
| B34S | 0.200 |
| B35 | 0.576 |
| B33R | 4.40 |
| B36R | 0.311 |
| B37 | 0.00238 |
| B38 | 1.33 |
| B39 | 0.128 |
| B40 | 0.00197 |
| B41 | 0.641 |
| B42 | 0.00633 |
| B43S | 0.0234 |
| B43R | 0.0403 |
| B44R | 0.0567 |
| B44S | 0.0542 |
| B45 | 0.0461 |
| B46 | 0.00260 |
| B47 | 7.12E−4 |
| B48S | 0.986 |
| B49 | 0.0778 |
| B50 | 0.00264 |
| B51 | 0.340 |
| B52 | 0.00175 |
| B53 | 0.00181 |
| B48R | 1.03 |
| B54 | 0.0161 |
| B55S | 0.144 |
| B56 | 0.00591 |
| B57S | 0.0103 |
| B58 | 0.00815 |
| B59 | 0.0464 |
| B60R | 0.0106 |
| B61R | 0.189 |
| B62S | 0.0392 |
| B63S | 0.00568 |
| B63R | 0.00517 |
| B62R | 4.26 |
| B64R | 0.0304 |
| C38 | 0.0160 |
| C39 | 0.00138 |
| C40 | 7.26E−4 |
| C41 | 4.29E−4 |
| C42 | 3.94E−4 |
| C43 | 3.75E−4 |
| C44 | 0.00451 |
| C45 | 6.79E−4 |
| C46 | 0.0201 |
| C47 | 0.00163 |
| C48 | 0.00353 |
| C49 | 0.00167 |
| C50 | 14.35 |
| C51 | 0.0179 |
| C52R | 0.017 |
| C52S | 0.0257 |

### Example 12: Cellular 15-PGDH Inhibition Study

This assay was used for the quantification of IL-1β induced Native Prostaglandin E2 (PGE2) produced by A549 cells (epithelial cells from lung carcinoma) in cell culture supernatants. Inhibition of 15-PGDH blocks PGE2 degradation and resulted in PGE2 accumulation over basal level. Briefly, A549 cells were treated with 2.5 ng/mL IL-1β (EC₈₀) and a 15-PGDH inhibitor for 24 hrs. PGE2 was detected in a competitive HTRF assay by using anti PGE2 antibody labeled with Europium cryptate, and PGE2 labeled with deuterium. The PGE2 present in the sample competes with the binding between the two HTRF detection solutions (reagents) and thereby prevents FRET from occurring. The specific signal is inversely proportional to the PGE2 concentration. From this binding data, IC50 values were calculated.

The following data (Table 12-1) provides the IC50 of each compound for inhibiting enzymatic activity in the assay described above.

TABLE 12-1

| ID | A549 Cell Assay IC50 |
|---|---|
| B3 | 0.00798 |
| B4 | 0.00185 |
| B5 | 7.29E−4 |
| B6S | 0.00507 |
| B6R | 0.0201 |
| B8 | 0.138 |
| B9R | 0.0405 |
| B9S | 0.082 |
| B10 | 0.0136 |
| B11 | 7.45E−4 |
| B12 | 0.0019 |
| B13 | 0.004 |
| B14 | 8.01E−4 |
| B15 | 0.00261 |
| B16 | 0.755 |
| B17S | 0.00863 |
| B18 | 2.55 |
| B17R | 0.081 |
| B19 | 3.79E−4 |
| B20 | 0.21 |
| B21 | 0.107 |
| B22 | 0.218 |
| B23 | 0.00118 |
| B24 | 9.59E−4 |
| B25 | 9.73E−4 |
| B27 | 0.0306 |
| B27R | 0.0393 |
| B27S | 0.00906 |
| B31 | 0.0285 |
| B37 | 0.0536 |
| B40 | 0.0104 |
| B42 | 0.123 |
| B46 | 0.0429 |
| B47 | 0.00916 |
| B63S | 0.0619 |
| B63R | 0.0598 |
| C39 | 0.133 |
| C40 | 0.00597 |
| C41 | 8.71E−5 |
| C42 | 0.00186 |
| C43 | 0.00199 |
| C44 | 0.102 |
| C45 | 0.108 |
| C49 | 0.00579 |

1. A compound of Formula (I) 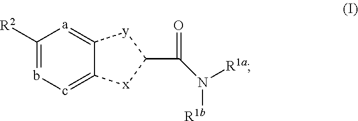
or a pharmaceutically acceptable salt of said compound;
wherein 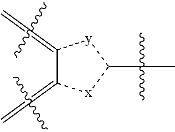
is 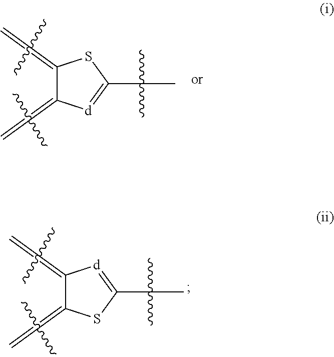
a is N, —N⁺—O⁻, or CH;
b is N or CH;
c is N, CH, or C—R³;
d is N, CH, or C—R⁴;
R^{1a} is —H, C₁₋₆ alkyl, C₁₋₆ haloalkyl, or C₁₋₆ heteroalkyl;
wherein R^{1a} may be unsubstituted or substituted with one or more substituents, each of which independently is halogen, —OH, —N(R^{b})₂, C₁₋₃ alkyl, or C₁₋₃ alkoxy;
R^{1b} is C₁₋₆ alkyl, C₁₋₆ haloalkyl, C₁₋₆ heteroalkyl, C₃₋₁₀ cycloalkyl, or heterocyclyl having 3 to 10 ring members;
wherein R^{1b} may be unsubstituted or substituted with one or more substituents, each of which independently is halogen, —OH, —N(R^{b})₂, C₁₋₃ alkyl, or C₁₋₃ alkoxy;
or, alternatively, R^{1a} and R^{1b} together form a heterocyclyl having 3 to 10 ring members;
wherein the heterocyclyl formed by R^{1a} and R^{1b} may be unsubstituted or substituted with one or more substituents, each of which independently is halogen, —OH, —CN, —N(R^{b})₂, C₁₋₆ alkyl, C₁₋₆ haloalkyl, C₁₋₆ alkoxy, C₁₋₆ heteroalkyl, C₃₋₆ cycloalkyl, or heterocyclyl having 3 to 6 ring members;
R² is heterocyclyl having 5 to 20 ring members or heteroaryl having 5 to 20 ring members, or alternatively, when a is —N⁺—O⁻, then R² is H;
wherein R² may be unsubstituted or substituted with one or more instances of R⁵;
wherein each instance of R⁵ independently is oxo, CN, halogen, —OH, C₁₋₆ alkyl, C₃₋₆ cycloalkyl, C₁₋₆ haloalkyl, C₁₋₆ alkoxy, C(O)N(R⁶)₂, —N(R⁶)₂, —N(R⁶)(C₁₋₆ alkylene-OH), or heterocyclyl having 3 to 10 members;
wherein each instance of R⁵ may be unsubstituted or substituted one or more instances of R⁶;
wherein each instance of R⁶ independently is —H, C₁₋₆ alkyl, C(O)C₁₋₆ alkyl, C(O)₂C₁₋₆ alkyl, C₁₋₆ alkylene-OH, or S(O)ₙC₁₋₆alkyl, where n is 0, 1, or 2;
wherein R⁶ may be unsubstituted or substituted with one or more substituents each of which independently is —F, —OH, C₁₋₃ alkoxy;
R³ is —H, C₁₋₆ alkyl, C₃₋₁₀ cycloalkyl, C₆₋₁₀aryl, heteroaryl having 5 to 10 ring members, or heterocyclyl having 3 to 10 ring members;
wherein R³ may be unsubstituted or substituted with one or more substituents each of which independently is halogen, —OH, oxo, —N(R^{b})₂, C₁₋₃ alkyl, C₁₋₃ haloalkyl, C₁₋₃ alkoxy, C₁₋₃ alkylene-C₁₋₃ alkoxy, or C₁₋₆heteroalkyl;
R⁴ is —H, C₁₋₆ alkyl, or —N(R⁷)₂ where each instance of R⁷, where present, independently is —H or C₁₋₆ alkyl; or, alternatively, two R⁷ substituents together form a heterocyclyl having 3 to 6 ring members;
wherein R⁷ may be unsubstituted or substituted with one or more substituents each of which independently is —F, —OH, C₁₋₃ alkyl, or C₁₋₃ alkoxy; and
wherein each instance of R^{b}, where present, independently is —H or C₁₋₆ alkyl; or, alternatively, two R^{b} substituents together form a heterocyclyl having 3 to 6 ring members;
wherein R^{b} may be unsubstituted or substituted with one or more substituents each of which independently is —F, —OH, C₁₋₃ alkyl, or C₁₋₃ alkoxy;
wherein, when R^{1a} is H, then a is CH, b is CH, c is C—R³, d is C—R⁴, R² is thienyl, R³ is phenyl, R⁴ is NH₂, R^{1b} is C₁₋₃ alkyl, wherein R^{1b} may be unsubstituted or substituted with C₁₋₃ alkoxy;
wherein, when R^{1a} is C₁₋₆ alkyl, C₁₋₆ haloalkyl, or C₁₋₆ heteroalkyl, then d is CH;
wherein when R² is triazolyl, tetrahydropyranyl, or oxetanyl, then R^{1b} is not a unsubstituted or substituted bridged [2.2.2]heterocyclyl with 8 ring members;
wherein, when R² is heteroaryl having 5 ring members or heterocyclyl having 4 to 6 ring members, then R^{1b} is not a unsubstituted or substituted bridged [2.2.2]heterocyclyl with 8 ring members;
wherein, when R^{1a} is H or Me and R^{1b} is C₁₋₃ alkyl or C₁₋₃ haloalkyl, then R³ is not unsubstituted or substituted morpholinyl;
wherein, when R^{1a} is H, R³ is not unsubstituted or substituted morpholinyl;
wherein, when R^{1a} and R^{1b} together provide unsubstituted or substituted piperidinyl or unsubstituted or substituted piperizinyl, then R³ is not unsubstituted or substituted morpholinyl.

2. (canceled)

3. The compound or salt of claim 1, wherein

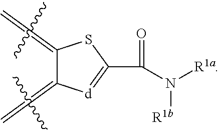

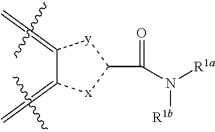

4. The compound or salt of claim 1, wherein

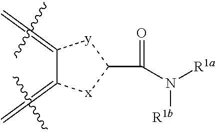

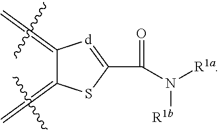

5. The compound or salt of claim 1, wherein the 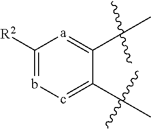
group is

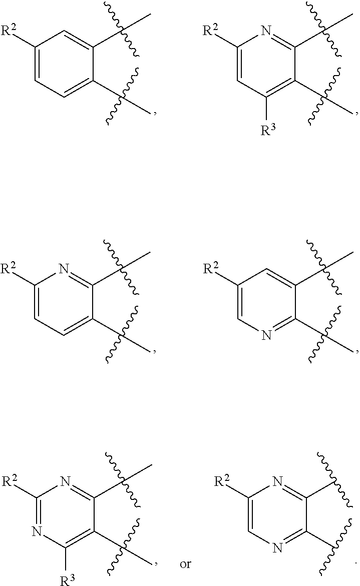

6. The compound or salt of claim 1, wherein R^{1a} is —H, unsubstituted or substituted C₁₋₃ alkyl, or unsubstituted or substituted C₁₋₃ haloalkyl.

7-15. (canceled)

16. The compound or salt of claim 1, wherein R^{1b} is unsubstituted or substituted alkyl.

17. (canceled)

18. The compound or salt of claim 1, wherein R^{1b} is unsubstituted or substituted cycloalkyl.

19-26. (canceled)

27. The compound or salt of claim 1, wherein R^{1a} and R^{1b} together form a 3 to 10 membered heterocyclyl.

28-31. (canceled)

32. The compound or salt of claim 1, wherein, when R^{1a} and R^{1b} together form a heterocyclyl that is substituted, each substituent of the heterocyclyl independently is halogen, —CN, C₁₋₆ alkyl, C₁₋₆ haloalkyl, C₁₋₆ alkoxy, C₁₋₆heteroalkyl, or C₃₋₆ cycloalkyl.

33. The compound or salt of claim 1, wherein R^{1a} and R^{1b} together form a heterocyclyl that is unsubstituted.

34. The compound or salt of claim 1, wherein, when R^{1a} and R^{1b} together form a heterocyclyl, the heterocyclyl is a bicyclic fused ring system.

35. The compound or salt of claim 1, wherein 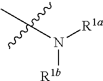
is

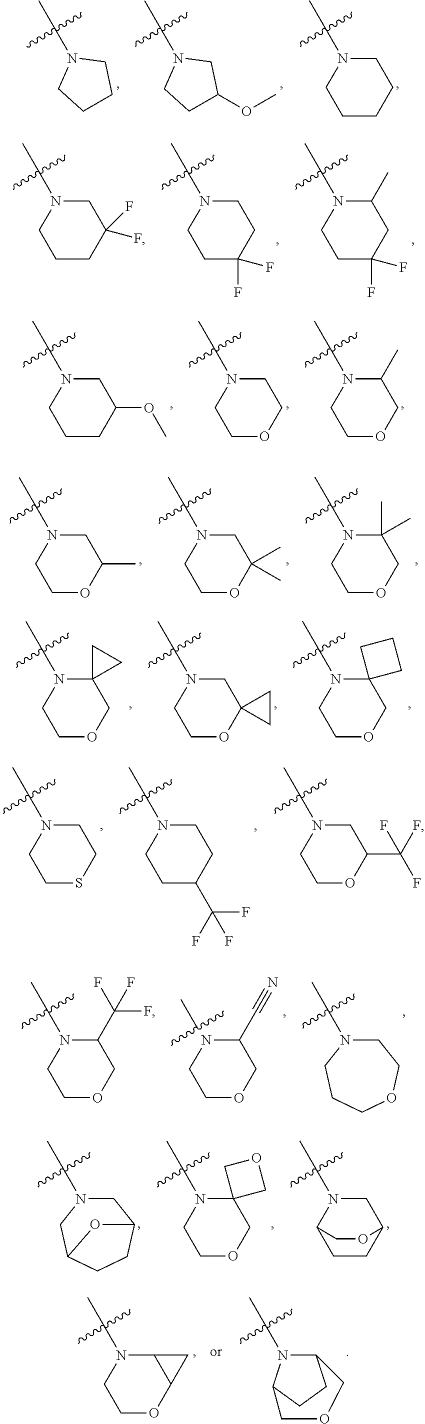

36. The compound or salt of claim 1, wherein R² is heteroaryl.

37-38. (canceled)

39. The compound or salt of claim 1, wherein R² is a fused bicyclic group.

40. (canceled)

41. The compound or salt of claim 1, wherein, each instance of R⁵ independently is oxo, CN, C₁₋₃ alkyl, C₃₋₆ cycloalkyl, C₁₋₃ haloalkyl, C₁₋₃ alkoxy, C(O)N(R⁶)₂, —N(R⁶)₂, —N(R⁶)(C₁₋₃ alkylene-OH), or heterocyclyl having 3 to 10 members.

42. (canceled)

43. The compound or salt of claim 1, wherein R³ is C₁₋₆ alkyl, C₃₋₁₀ cycloalkyl, C₆₋₁₀ aryl, heteroaryl having 5 to 10 ring members, or heterocyclyl having 3 to 10 ring members.

44. The compound or salt of claim 1, wherein R⁴ is —H.

45. The compound or salt of claim 1, wherein the compound is:

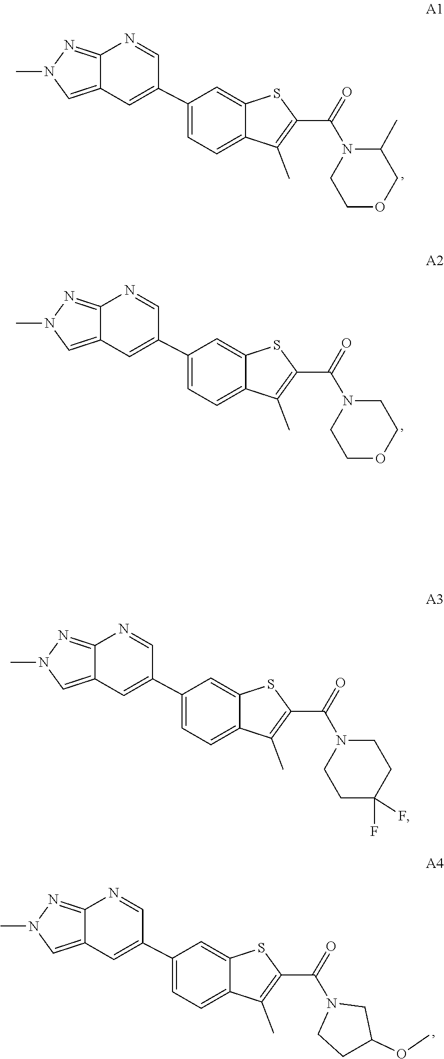

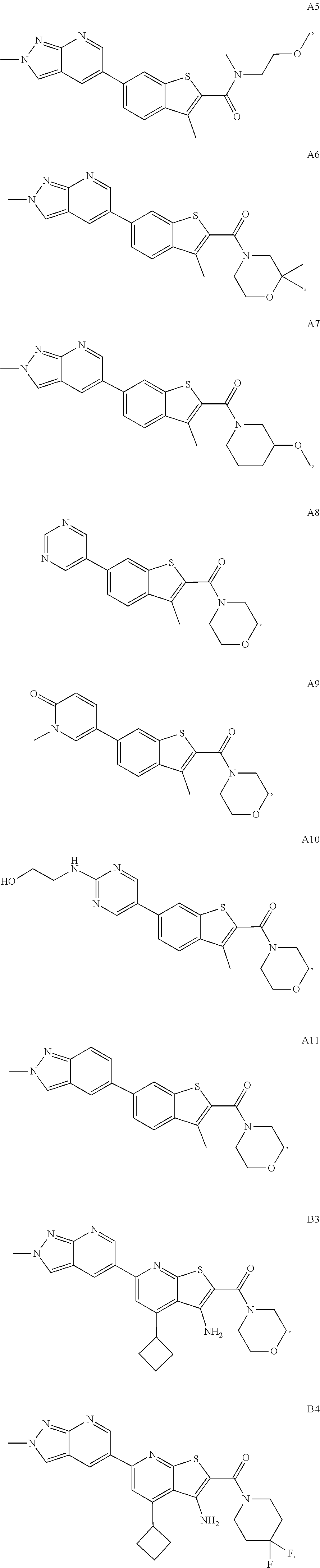

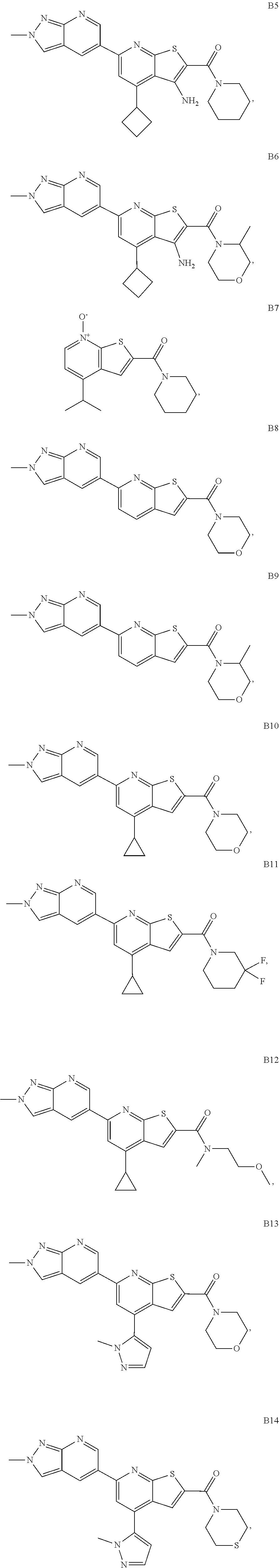

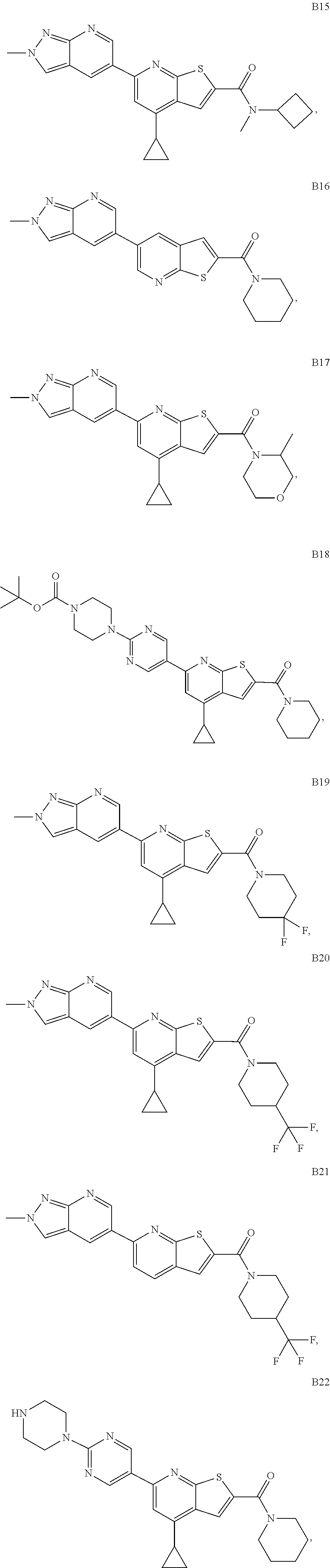

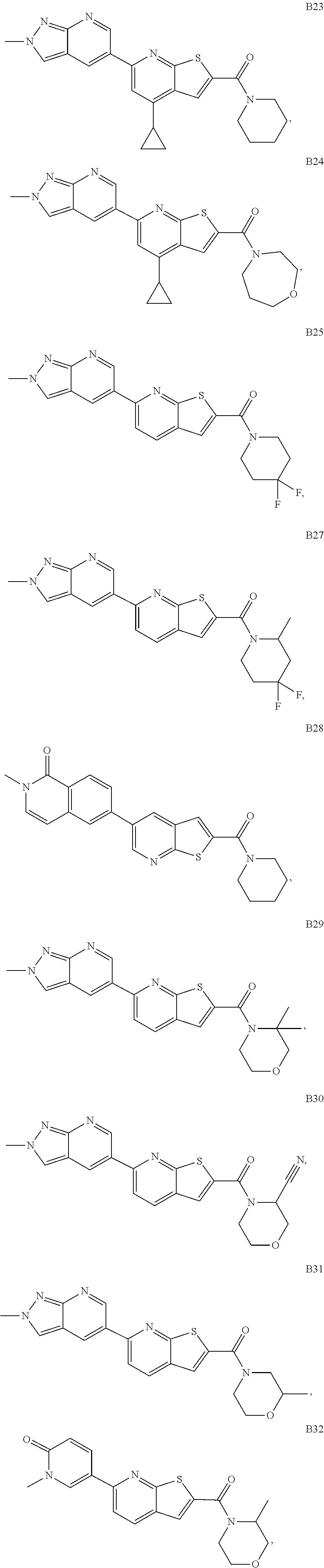

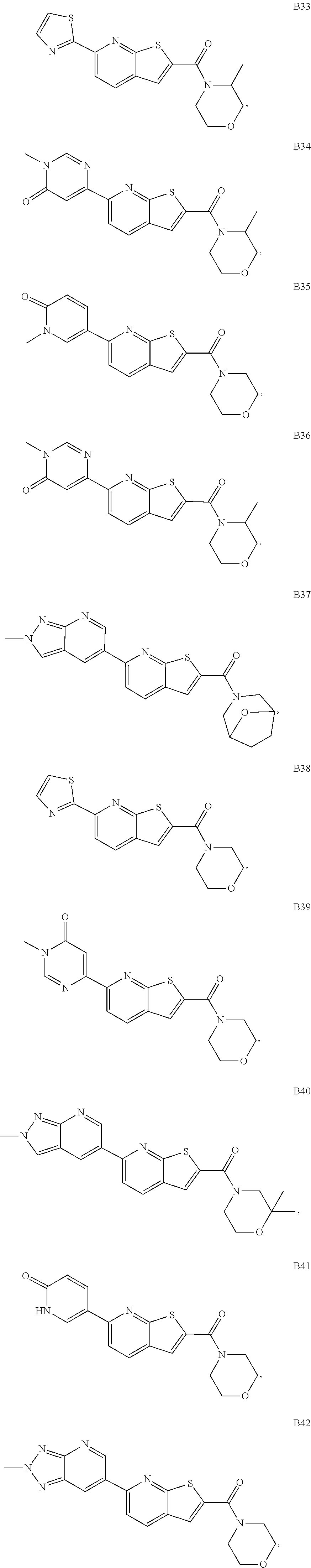

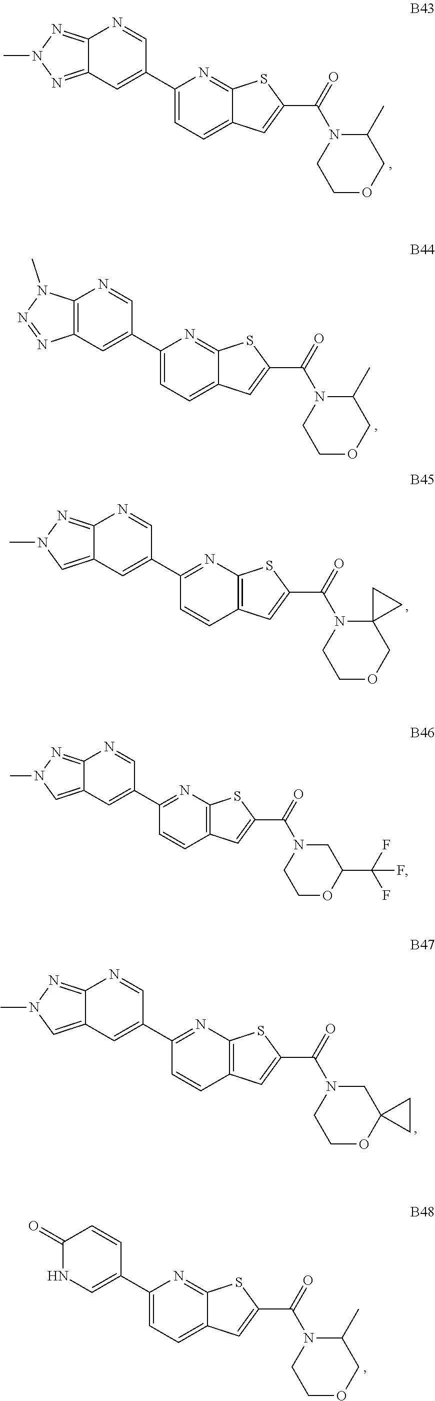

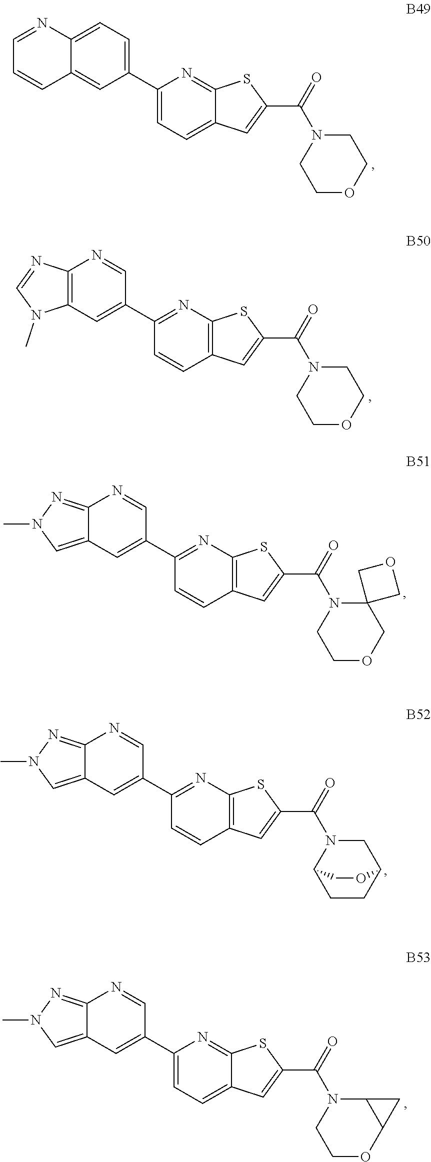 [SMILES: O=C(c1cc2ccc(-c3ccc4ncccc4c3)nc2s1)N1CCOCC1 ; Cn1cnc2ncc(-c3ccc4cc(C(=O)N5CCOCC5)sc4n3)cc21 ; Cn1cc2cc(-c3ccc4cc(C(=O)N5CCOCC56COC6)sc4n3)cnc2n1 ; Cn1cc2cc(-c3ccc4cc(C(=O)N5C[C@H]6CC[C@@H]5CO6)sc4n3)cnc2n1 ; Cn1cc2cc(-c3ccc4cc(C(=O)N5CCOC6CC65)sc4n3)cnc2n1]

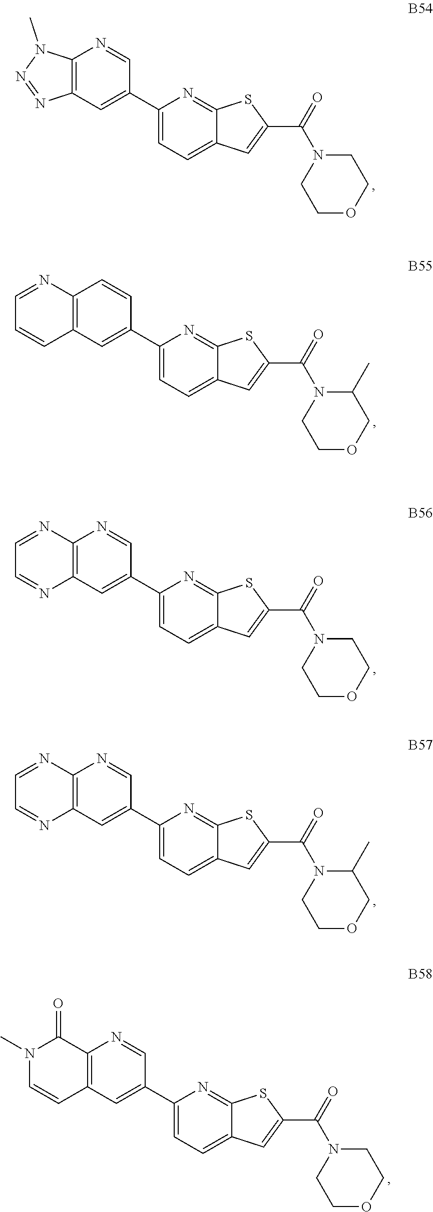

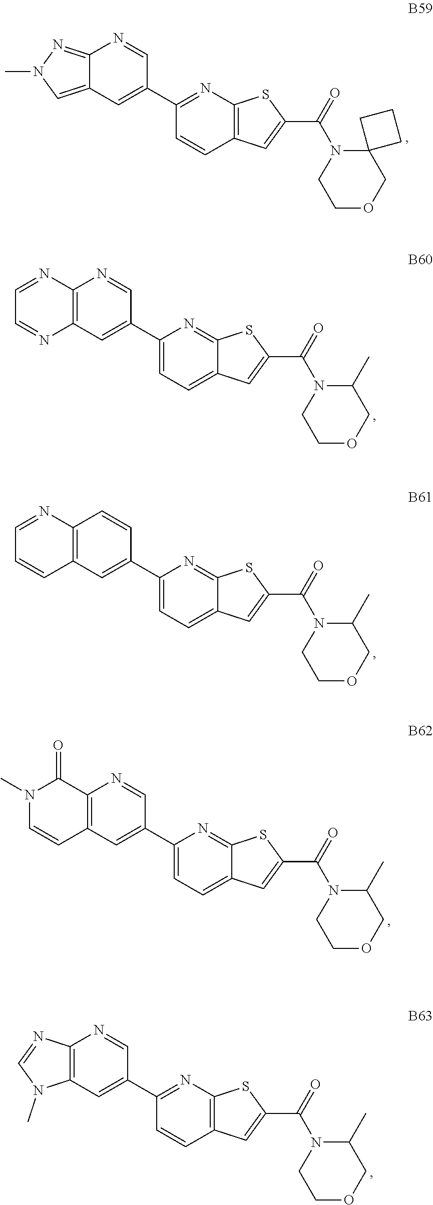

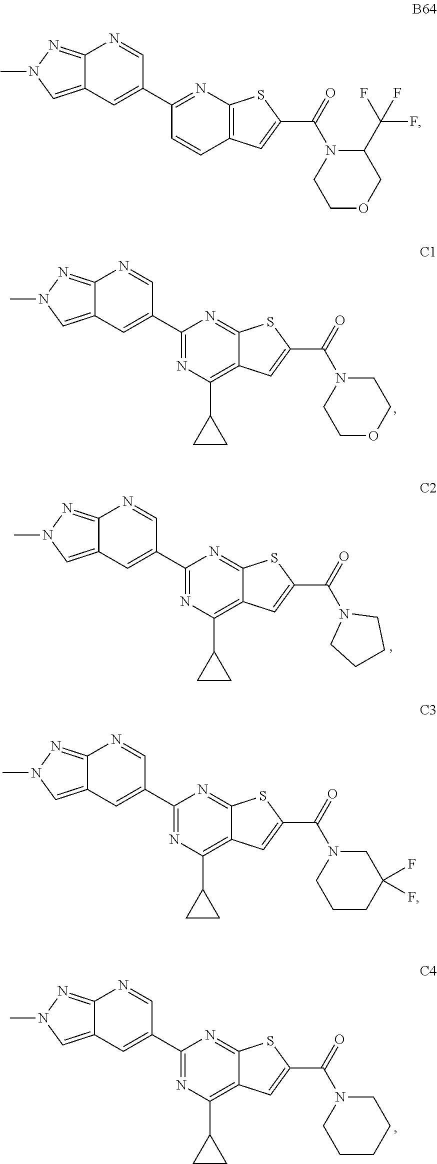 [SMILES: Cn1cc2cc(-c3ccc4cc(C(=O)N5CCOCC5C(F)(F)F)sc4n3)cnc2n1 ; Cn1cc2cc(-c3nc(C4CC4)c4cc(C(=O)N5CCOCC5)sc4n3)cnc2n1 ; Cn1cc2cc(-c3nc(C4CC4)c4cc(C(=O)N5CCCC5)sc4n3)cnc2n1 ; Cn1cc2cc(-c3nc(C4CC4)c4cc(C(=O)N5CCCC(F)(F)C5)sc4n3)cnc2n1 ; Cn1cc2cc(-c3nc(C4CC4)c4cc(C(=O)N5CCCCC5)sc4n3)cnc2n1]

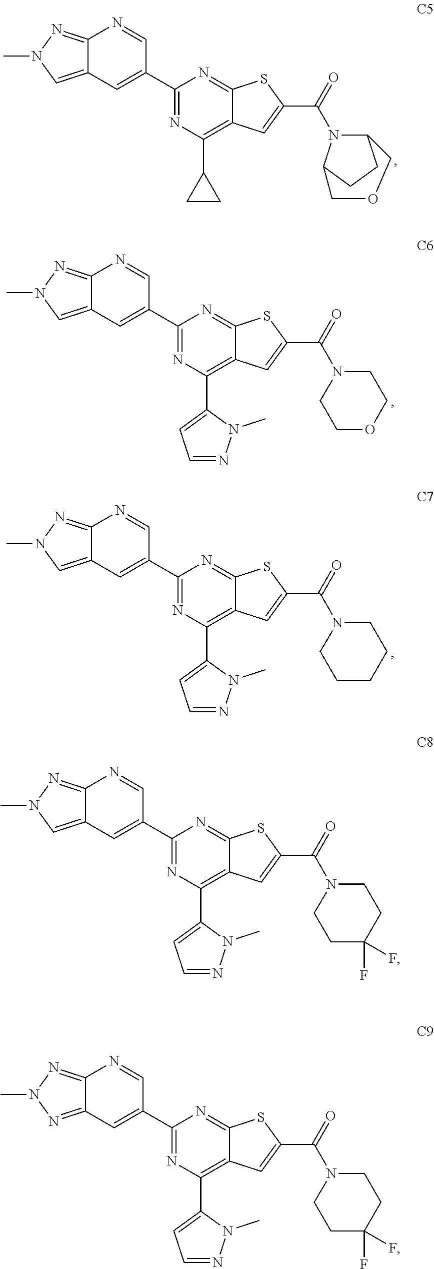 [SMILES: Cn1cc2cc(-c3nc(C4CC4)c4cc(C(=O)N5C6CCC5COC6)sc4n3)cnc2n1 ; Cn1cc2cc(-c3nc(-c4ccnn4C)c4cc(C(=O)N5CCOCC5)sc4n3)cnc2n1 ; Cn1cc2cc(-c3nc(-c4ccnn4C)c4cc(C(=O)N5CCCCC5)sc4n3)cnc2n1 ; Cn1cc2cc(-c3nc(-c4ccnn4C)c4cc(C(=O)N5CCC(F)(F)CC5)sc4n3)cnc2n1 ; Cn1nc2cc(-c3nc(-c4ccnn4C)c4cc(C(=O)N5CCC(F)(F)CC5)sc4n3)cnc2n1]

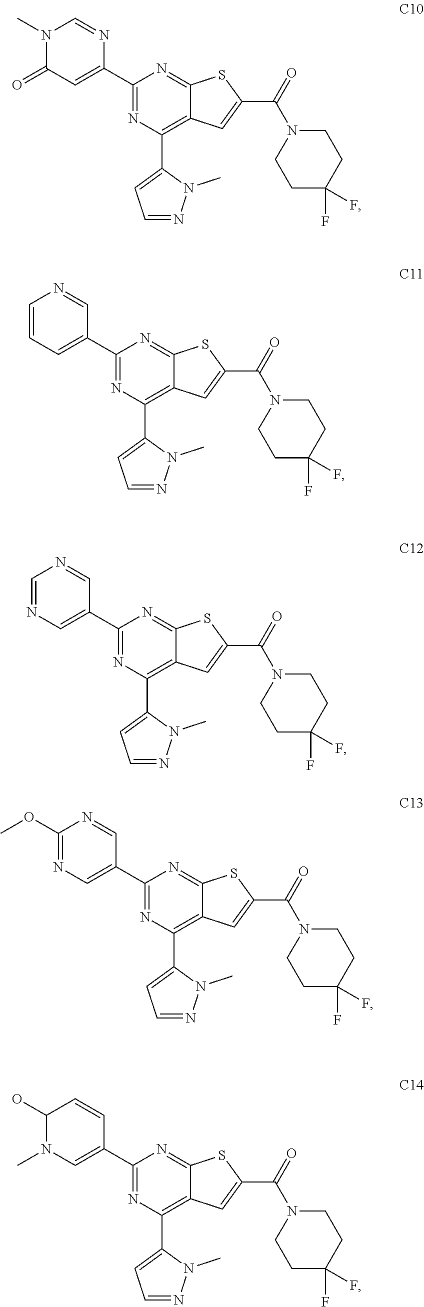 [SMILES: Cn1nccc1-c1nc(-c2cc(=O)n(C)cn2)nc2sc(C(=O)N3CCC(F)(F)CC3)cc12 ; Cn1nccc1-c1nc(-c2cccnc2)nc2sc(C(=O)N3CCC(F)(F)CC3)cc12 ; Cn1nccc1-c1nc(-c2cncnc2)nc2sc(C(=O)N3CCC(F)(F)CC3)cc12 ; COc1ncc(-c2nc(-c3ccnn3C)c3cc(C(=O)N4CCC(F)(F)CC4)sc3n2)cn1 ; CN1C=C(c2nc(-c3ccnn3C)c3cc(C(=O)N4CCC(F)(F)CC4)sc3n2)C=CC1O]

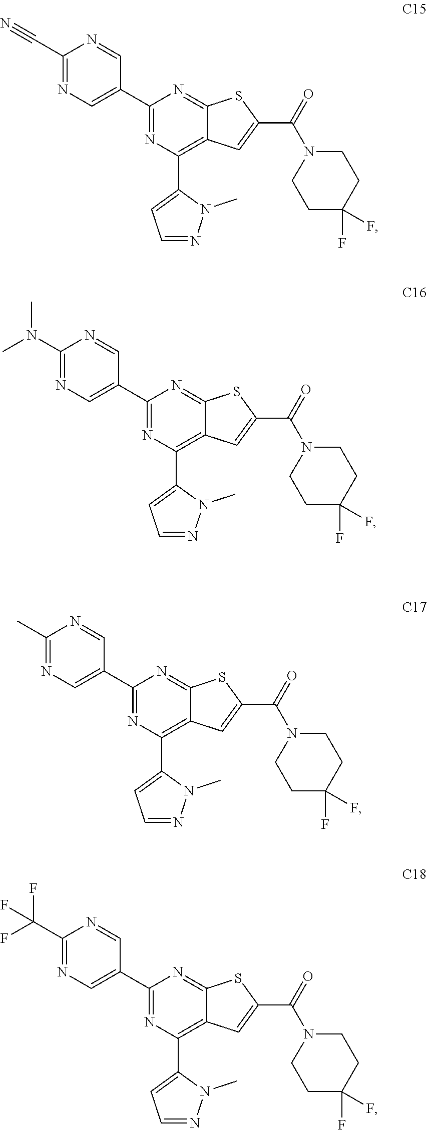 [SMILES: Cn1nccc1-c1nc(-c2cnc(C#N)nc2)nc2sc(C(=O)N3CCC(F)(F)CC3)cc12 ; CN(C)c1ncc(-c2nc(-c3ccnn3C)c3cc(C(=O)N4CCC(F)(F)CC4)sc3n2)cn1 ; Cc1ncc(-c2nc(-c3ccnn3C)c3cc(C(=O)N4CCC(F)(F)CC4)sc3n2)cn1 ; Cn1nccc1-c1nc(-c2cnc(C(F)(F)F)nc2)nc2sc(C(=O)N3CCC(F)(F)CC3)cc12]

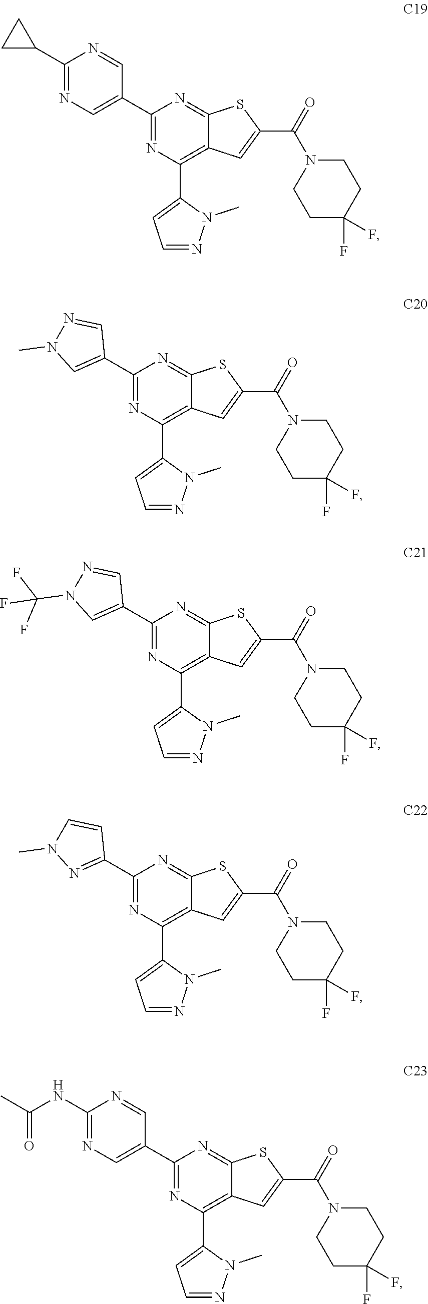 [SMILES: Cn1nccc1-c1nc(-c2cnc(C3CC3)nc2)nc2sc(C(=O)N3CCC(F)(F)CC3)cc12 ; Cn1cc(-c2nc(-c3ccnn3C)c3cc(C(=O)N4CCC(F)(F)CC4)sc3n2)cn1 ; Cn1nccc1-c1nc(-c2cnn(C(F)(F)F)c2)nc2sc(C(=O)N3CCC(F)(F)CC3)cc12 ; Cn1ccc(-c2nc(-c3ccnn3C)c3cc(C(=O)N4CCC(F)(F)CC4)sc3n2)n1 ; CC(=O)Nc1ncc(-c2nc(-c3ccnn3C)c3cc(C(=O)N4CCC(F)(F)CC4)sc3n2)cn1]

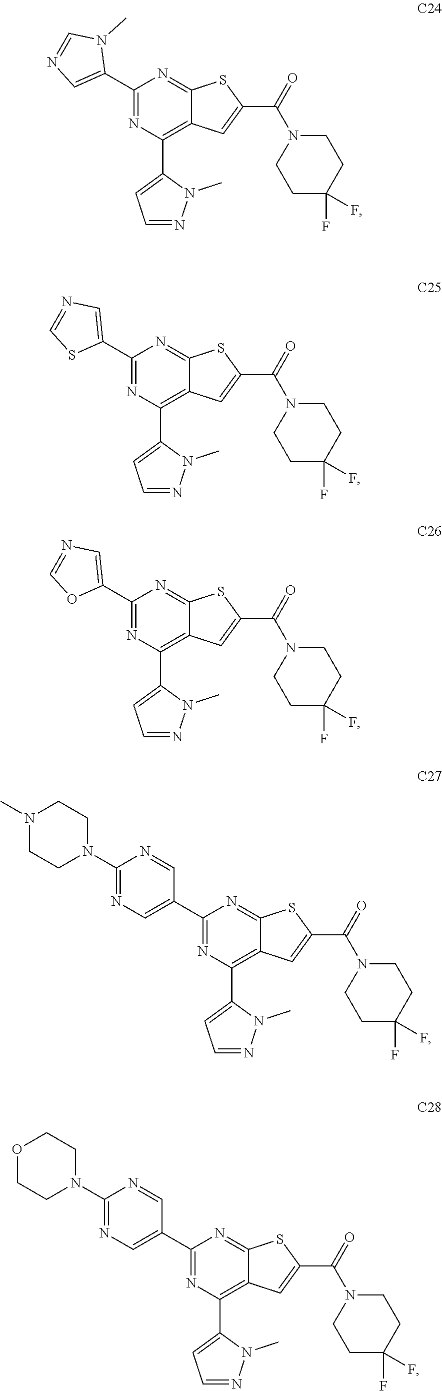 [SMILES: Cn1cncc1-c1nc(-c2ccnn2C)c2cc(C(=O)N3CCC(F)(F)CC3)sc2n1 ; Cn1nccc1-c1nc(-c2cncs2)nc2sc(C(=O)N3CCC(F)(F)CC3)cc12 ; Cn1nccc1-c1nc(-c2cnco2)nc2sc(C(=O)N3CCC(F)(F)CC3)cc12 ; CN1CCN(c2ncc(-c3nc(-c4ccnn4C)c4cc(C(=O)N5CCC(F)(F)CC5)sc4n3)cn2)CC1 ; Cn1nccc1-c1nc(-c2cnc(N3CCOCC3)nc2)nc2sc(C(=O)N3CCC(F)(F)CC3)cc12]

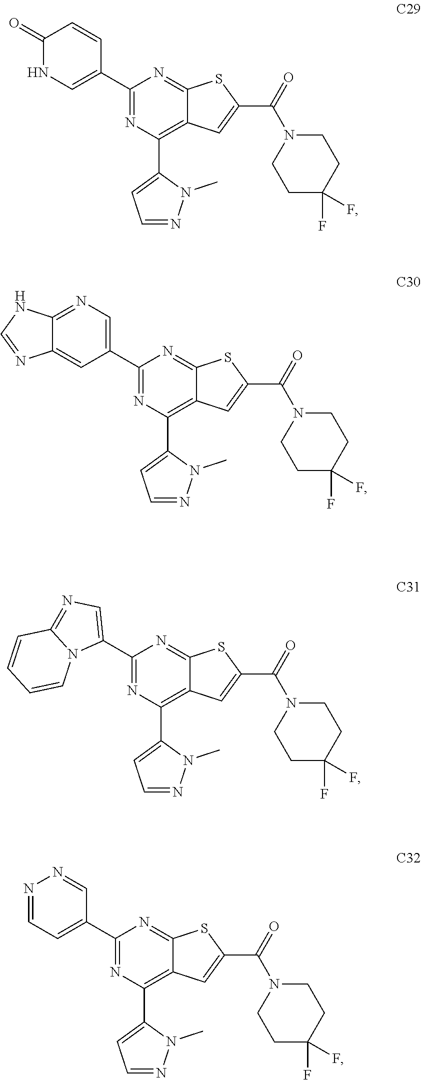 [SMILES: Cn1nccc1-c1nc(-c2ccc(=O)[nH]c2)nc2sc(C(=O)N3CCC(F)(F)CC3)cc12 ; Cn1nccc1-c1nc(-c2cnc3[nH]cnc3c2)nc2sc(C(=O)N3CCC(F)(F)CC3)cc12 ; Cn1nccc1-c1nc(-c2cnc3ccccn23)nc2sc(C(=O)N3CCC(F)(F)CC3)cc12 ; Cn1nccc1-c1nc(-c2ccnnc2)nc2sc(C(=O)N3CCC(F)(F)CC3)cc12]

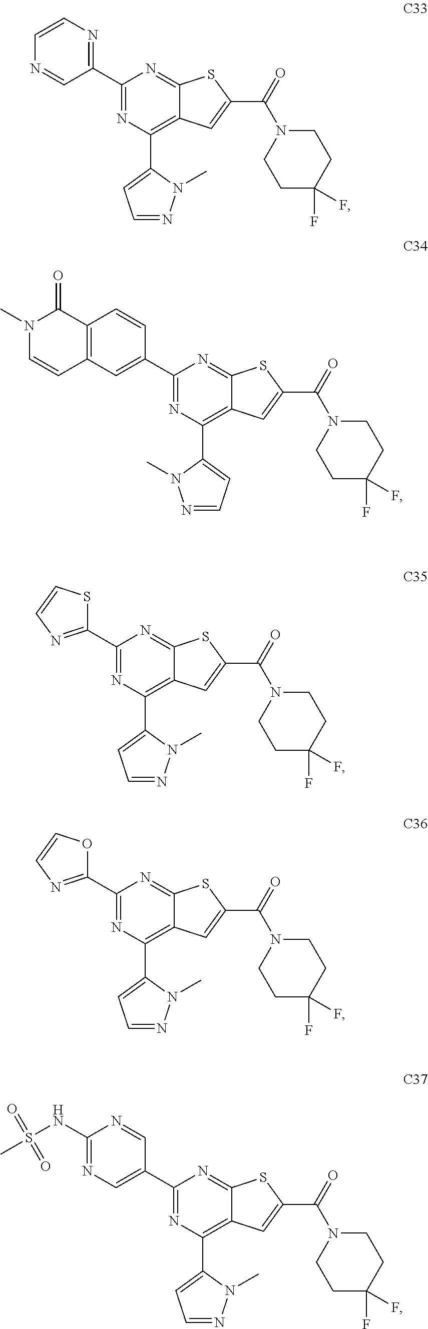 [SMILES: Cn1nccc1-c1nc(-c2cnccn2)nc2sc(C(=O)N3CCC(F)(F)CC3)cc12 ; Cn1nccc1-c1nc(-c2ccc3c(=O)n(C)ccc3c2)nc2sc(C(=O)N3CCC(F)(F)CC3)cc12 ; Cn1nccc1-c1nc(-c2nccs2)nc2sc(C(=O)N3CCC(F)(F)CC3)cc12 ; Cn1nccc1-c1nc(-c2ncco2)nc2sc(C(=O)N3CCC(F)(F)CC3)cc12 ; Cn1nccc1-c1nc(-c2cnc(NS(C)(=O)=O)nc2)nc2sc(C(=O)N3CCC(F)(F)CC3)cc12]

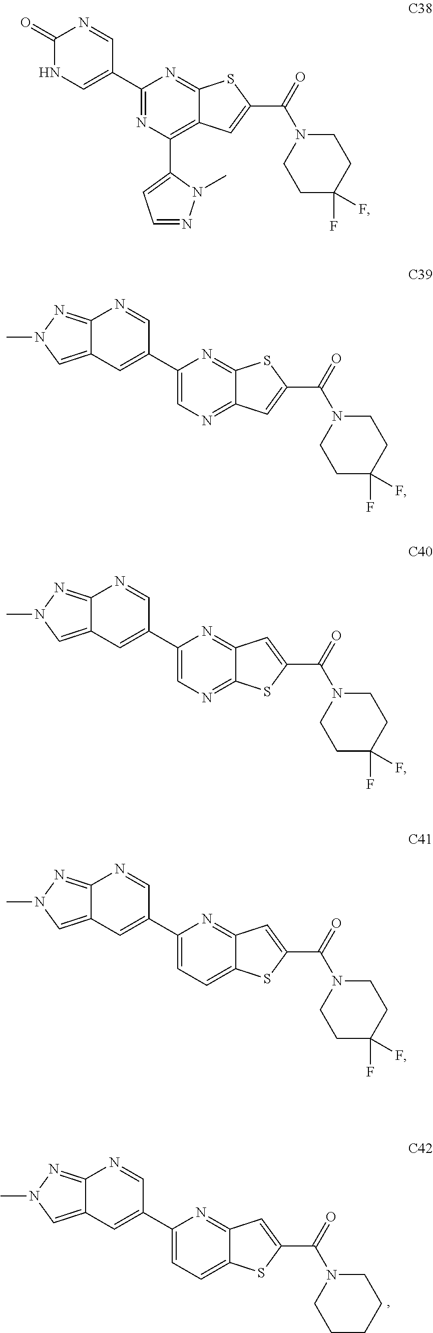 [SMILES: Cn1nccc1-c1nc(-c2cnc(=O)[nH]c2)nc2sc(C(=O)N3CCC(F)(F)CC3)cc12 ; Cn1cc2cc(-c3cnc4cc(C(=O)N5CCC(F)(F)CC5)sc4n3)cnc2n1 ; Cn1cc2cc(-c3cnc4sc(C(=O)N5CCC(F)(F)CC5)cc4n3)cnc2n1 ; Cn1cc2cc(-c3ccc4sc(C(=O)N5CCC(F)(F)CC5)cc4n3)cnc2n1 ; Cn1cc2cc(-c3ccc4sc(C(=O)N5CCCCC5)cc4n3)cnc2n1]

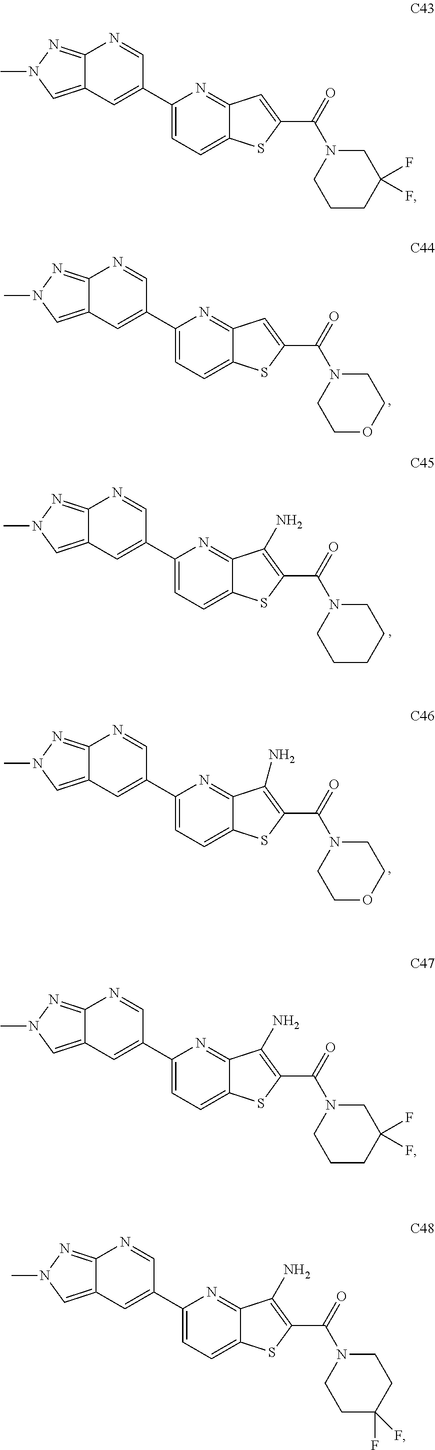 [SMILES: Cn1cc2cc(-c3ccc4sc(C(=O)N5CCCC(F)(F)C5)cc4n3)cnc2n1 ; Cn1cc2cc(-c3ccc4sc(C(=O)N5CCOCC5)cc4n3)cnc2n1 ; Cn1cc2cc(-c3ccc4sc(C(=O)N5CCCCC5)c(N)c4n3)cnc2n1 ; Cn1cc2cc(-c3ccc4sc(C(=O)N5CCOCC5)c(N)c4n3)cnc2n1 ; Cn1cc2cc(-c3ccc4sc(C(=O)N5CCCC(F)(F)C5)c(N)c4n3)cnc2n1 ; Cn1cc2cc(-c3ccc4sc(C(=O)N5CCC(F)(F)CC5)c(N)c4n3)cnc2n1]

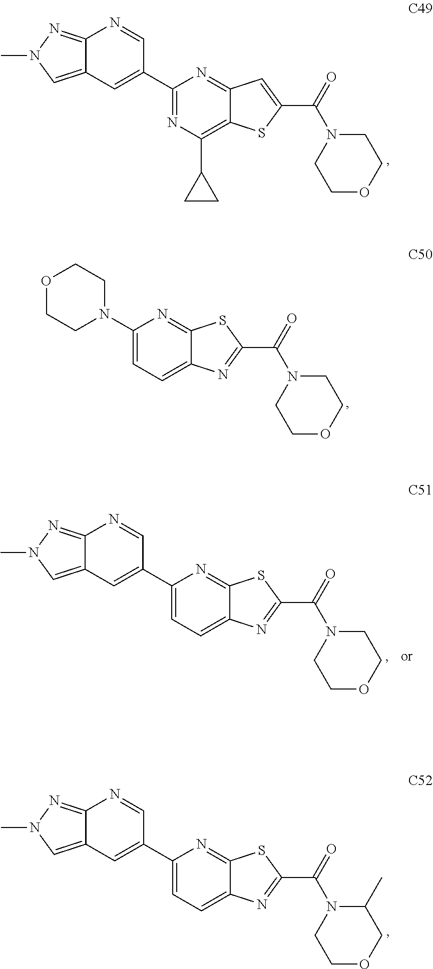

46. The compound or salt of claim 1, wherein the compound is:

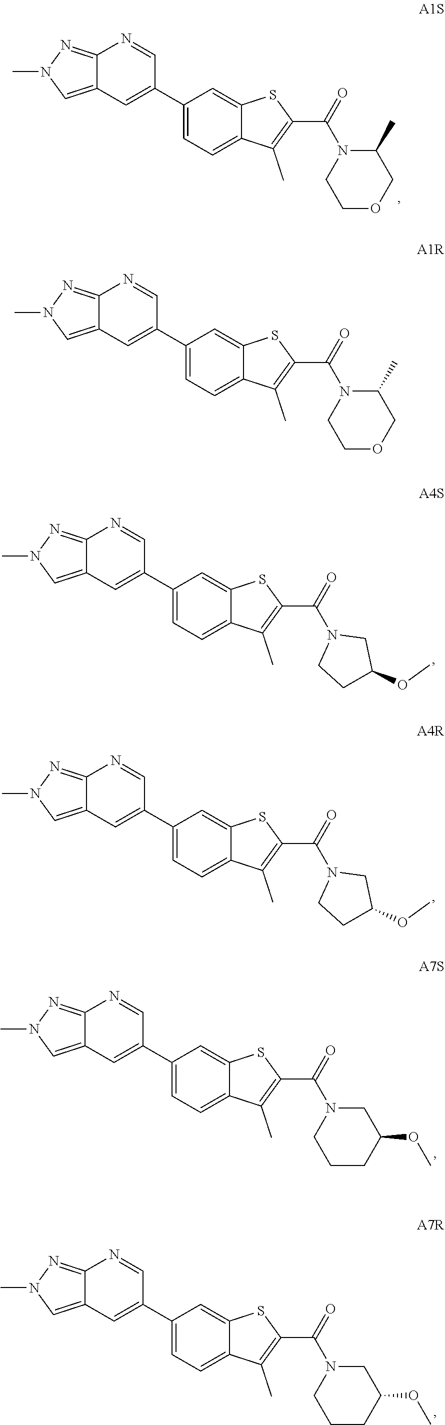

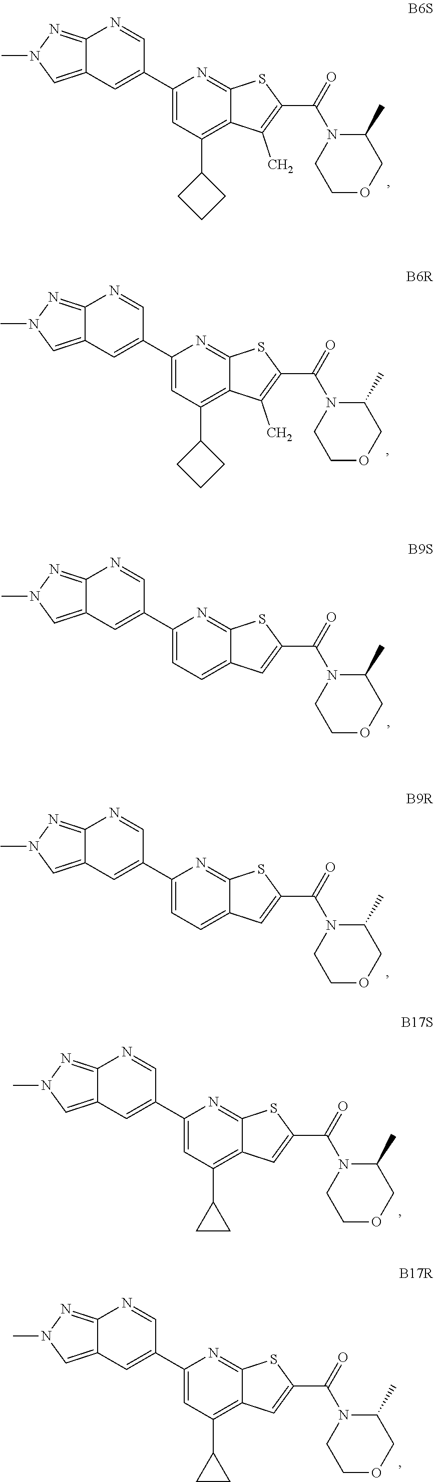

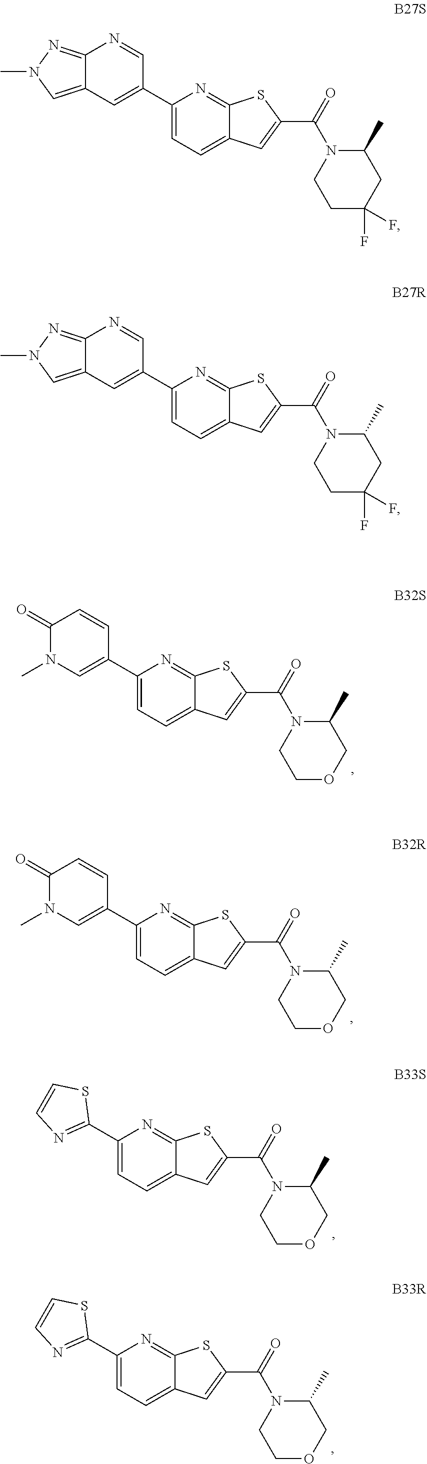 [SMILES: C[C@H]1CC(F)(F)CCN1C(=O)c1cc2ccc(-c3cnc4nn(C)cc4c3)nc2s1 ; C[C@@H]1CC(F)(F)CCN1C(=O)c1cc2ccc(-c3cnc4nn(C)cc4c3)nc2s1 ; C[C@H]1COCCN1C(=O)c1cc2ccc(-c3ccc(=O)n(C)c3)nc2s1 ; C[C@@H]1COCCN1C(=O)c1cc2ccc(-c3ccc(=O)n(C)c3)nc2s1 ; C[C@H]1COCCN1C(=O)c1cc2ccc(-c3nccs3)nc2s1 ; C[C@@H]1COCCN1C(=O)c1cc2ccc(-c3nccs3)nc2s1]

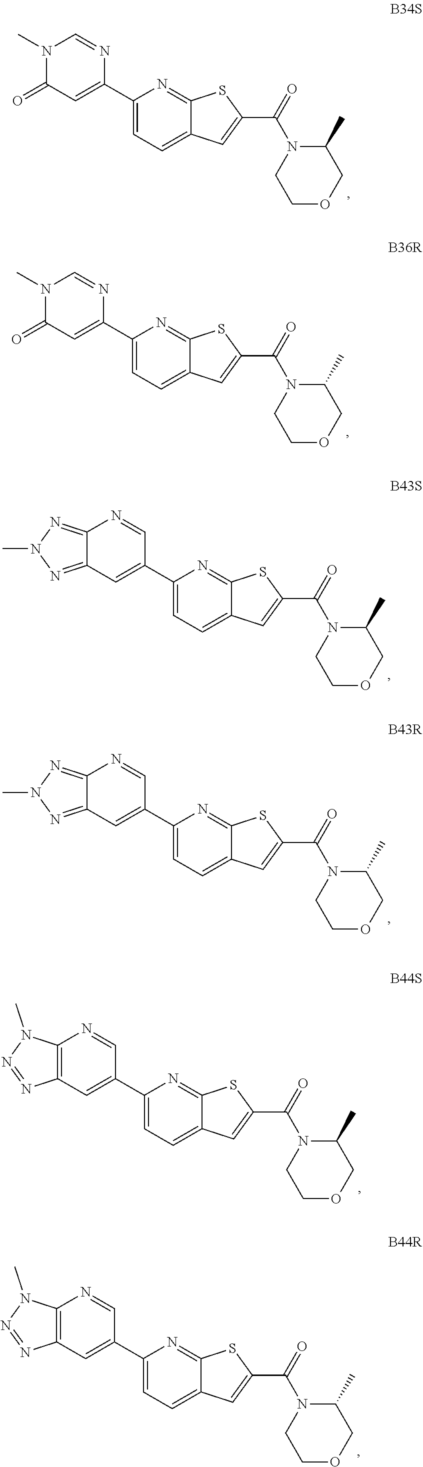

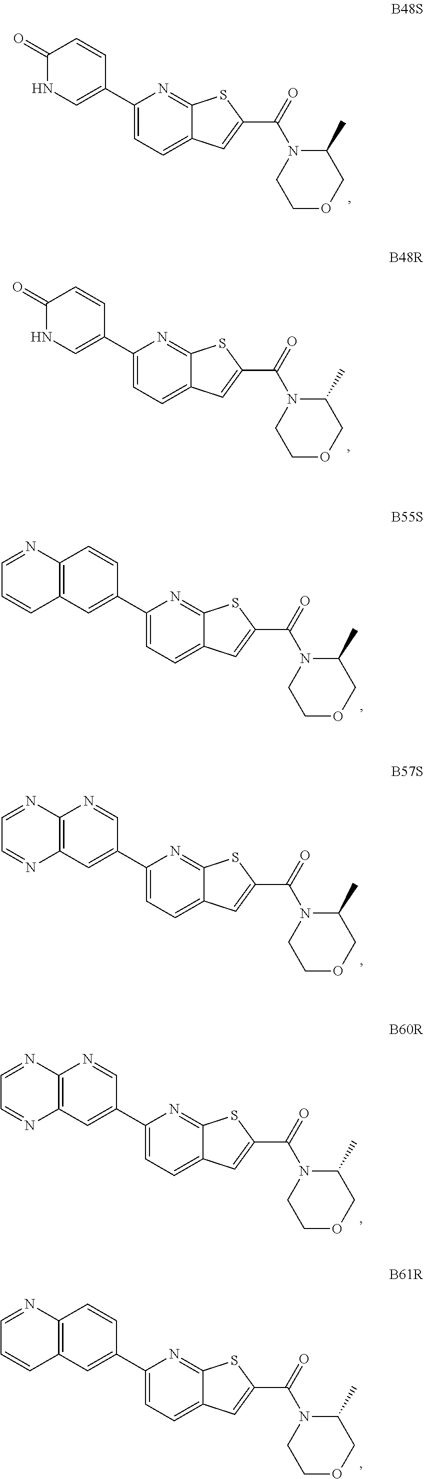 [SMILES: C[C@H]1COCCN1C(=O)c1cc2ccc(-c3ccc(=O)[nH]c3)nc2s1 ; C[C@@H]1COCCN1C(=O)c1cc2ccc(-c3ccc(=O)[nH]c3)nc2s1 ; C[C@H]1COCCN1C(=O)c1cc2ccc(-c3ccc4ncccc4c3)nc2s1 ; C[C@H]1COCCN1C(=O)c1cc2ccc(-c3cnc4nccnc4c3)nc2s1 ; C[C@@H]1COCCN1C(=O)c1cc2ccc(-c3cnc4nccnc4c3)nc2s1 ; C[C@@H]1COCCN1C(=O)c1cc2ccc(-c3ccc4ncccc4c3)nc2s1]

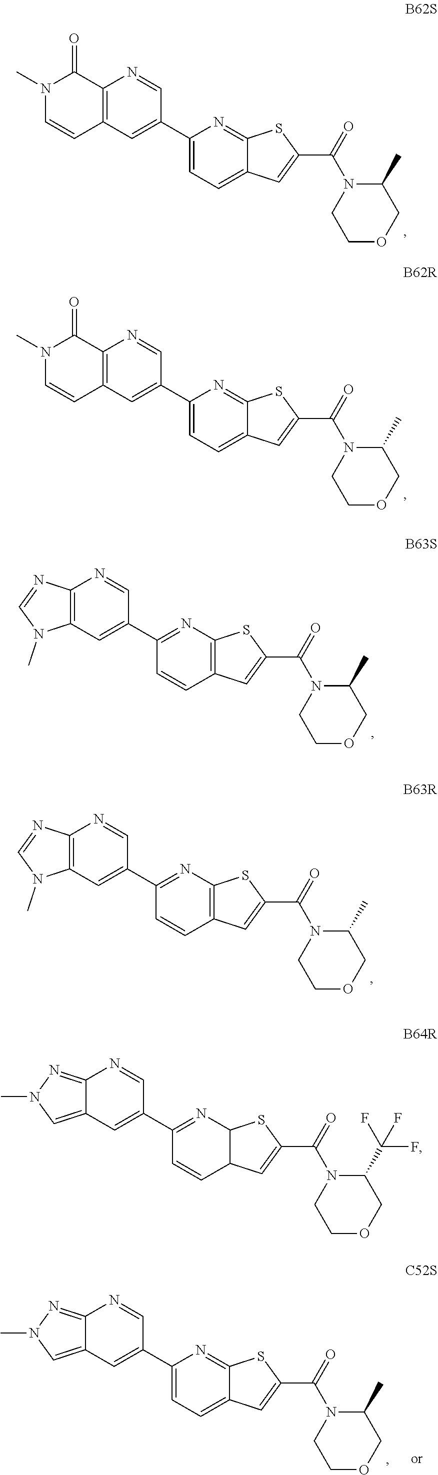

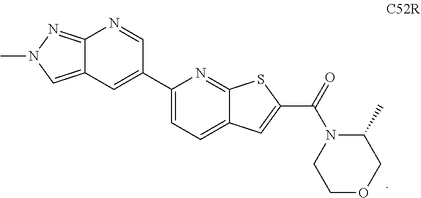

47. The compound or salt of claim 1, wherein the compound is:
(3-methyl-6-(2-methyl-2H-pyrazolo[3,4-b]pyridin-5-yl)-1-benzothiophen-2-yl)(3-methyl-4-morpholinyl)methanone;
(3-methyl-6-(2-methyl-2H-pyrazolo[3,4-b]pyridin-5-yl)-1-benzothiophen-2-yl)(4-morpholinyl)methanone;
(4,4-difluoro-1-piperidinyl)(3-methyl-6-(2-methyl-2H-pyrazolo[3,4-b]pyridin-5-yl)-1-benzothiophen-2-yl)methanone;
(3-methoxy-1-pyrrolidinyl)(3-methyl-6-(2-methyl-2H-pyrazolo[3,4-b]pyridin-5-yl)-1-benzothiophen-2-yl)methanone;
N-(2-methoxyethyl)-N,3-dimethyl-6-(2-methyl-2H-pyrazolo[3,4-b]pyridin-5-yl)-1-benzothiophene-2-carboxamide;
(2,2-dimethyl-4-morpholinyl)(3-methyl-6-(2-methyl-2H-pyrazolo[3,4-b]pyridin-5-yl)-1-benzothiophen-2-yl)methanone;
(3-methoxy-1-piperidinyl)(3-methyl-6-(2-methyl-2H-pyrazolo[3,4-b]pyridin-5-yl)-1-benzothiophen-2-yl)methanone;
(3-methyl-6-(5-pyrimidinyl)-1-benzothiophen-2-yl)(4-morpholinyl)methanone;
1-methyl-5-(3-methyl-2-(4-morpholinylcarbonyl)-1-benzothiophen-6-yl)-2(1H)-pyridinone;
(6-(2-((2-hydroxyethyl)amino)-5-pyrimidinyl)-3-methyl-1-benzothiophen-2-yl)(4-morpholinyl)methanone;
(3-methyl-6-(2-methyl-2H-indazol-5-yl)-1-benzothiophen-2-yl)(4-morpholinyl)methanone;
(3-amino-4-cyclobutyl-6-(2-methyl-2H-pyrazolo[3,4-b]pyridin-5-yl)thieno[2,3-b]pyridin-2-yl)(4-morpholinyl)methanone;
(3-amino-4-cyclobutyl-6-(2-methyl-2H-pyrazolo[3,4-b]pyridin-5-yl)thieno[2,3-b]pyridin-2-yl)(4,4-difluoro-1-piperidinyl)methanone;
(3-amino-4-cyclobutyl-6-(2-methyl-2H-pyrazolo[3,4-b]pyridin-5-yl)thieno[2,3-b]pyridin-2-yl)(1-piperidinyl)methanone;
(3-amino-4-cyclobutyl-6-(2-methyl-2H-pyrazolo[3,4-b]pyridin-5-yl)thieno[2,3-b]pyridin-2-yl)(3-methyl-4-morpholinyl)methanone;
(7-oxido-4-(2-propanyl)thieno[2,3-b]pyridin-2-yl)(1-piperidinyl)methanone;
(6-(2-methyl-2H-pyrazolo[3,4-b]pyridin-5-yl)thieno[2,3-b]pyridin-2-yl)(4-morpholinyl)methanone;
(3-methyl-4-morpholinyl)(6-(2-methyl-2H-pyrazolo[3,4-b]pyridin-5-yl)thieno[2,3-b]pyridin-2-yl)methanone;
(4-cyclopropyl-6-(2-methyl-2H-pyrazolo[3,4-b]pyridin-5-yl)thieno[2,3-b]pyridin-2-yl)(4-morpholinyl)methanone;
(4-cyclopropyl-6-(2-methyl-2H-pyrazolo[3,4-b]pyridin-5-yl)thieno[2,3-b]pyridin-2-yl)(3,3-difluoro-1-piperidinyl)methanone;
4-cyclopropyl-N-(2-methoxyethyl)-N-methyl-6-(2-methyl-2H-pyrazolo[3,4-b]pyridin-5-yl)thieno[2,3-b]pyridine-2-carboxamide;
(6-(2-methyl-2H-pyrazolo[3,4-b]pyridin-5-yl)-4-(1-methyl-1H-pyrazol-5-yl)thieno[2,3-b]pyridin-2-yl)(4-morpholinyl)methanone;
(6-(2-methyl-2H-pyrazolo[3,4-b]pyridin-5-yl)-4-(1-methyl-1H-pyrazol-5-yl)thieno[2,3-b]pyridin-2-yl)(4-thiomorpholinyl)methanone;
N-cyclobutyl-4-cyclopropyl-N-methyl-6-(2-methyl-2H-pyrazolo[3,4-b]pyridin-5-yl)thieno[2,3-b]pyridine-2-carboxamide;
(5-(2-methyl-2H-pyrazolo[3,4-b]pyridin-5-yl)thieno[2,3-b]pyridin-2-yl)(1-piperidinyl)methanone;
(4-cyclopropyl-6-(2-methyl-2H-pyrazolo [3,4-b]pyridin-5-yl)thieno[2,3-b]pyridin-2-yl)(3-methyl-4-morpholinyl)methanone;
2-methyl-2-propanyl 4-(5-(4-cyclopropyl-2-(1-piperidinylcarbonyl)thieno[2,3-b]pyridin-6-yl)-2-pyrimidinyl)-1-piperazinecarboxylate;
(4-cyclopropyl-6-(2-methyl-2H-pyrazolo[3,4-b]pyridin-5-yl)thieno[2,3-b]pyridin-2-yl)(4,4-difluoro-1-piperidinyl)methanone;
(4-cyclopropyl-6-(2-methyl-2H-pyrazolo[3,4-b]pyridin-5-yl)thieno[2,3-b]pyridin-2-yl)(4-(trifluoromethyl)-1-piperidinyl)methanone;
(6-(2-methyl-2H-pyrazolo[3,4-b]pyridin-5-yl)thieno[2,3-b]pyridin-2-yl)(4-(trifluoromethyl)-1-piperidinyl)methanone;
(4-cyclopropyl-6-(2-(1-piperazinyl)-5-pyrimidinyl)thieno[2,3-b]pyridin-2-yl)(1-piperidinyl)methanone;
(4-cyclopropyl-6-(2-methyl-2H-pyrazolo[3,4-b]pyridin-5-yl)thieno[2,3-b]pyridin-2-yl)(1-piperidinyl)methanone;
(4-cyclopropyl-6-(2-methyl-2H-pyrazolo[3,4-b]pyridin-5-yl)thieno[2,3-b]pyridin-2-yl)(1,4-oxazepan-4-yl)methanone;
(4,4-difluoro-1-piperidinyl)(6-(2-methyl-2H-pyrazolo[3,4-b]pyridin-5-yl)thieno[2,3-b]pyridin-2-yl)methanone;
(4,4-difluoro-2-methyl-1-piperidinyl)(6-(2-methyl-2H-pyrazolo[3,4-b]pyridin-5-yl)thieno[2,3-b]pyridin-2-yl)methanone;
2-methyl-6-(2-(1-piperidinylcarbonyl)thieno[2,3-b]pyridin-5-yl)-1(2H)-isoquinolinone;
(3,3-dimethyl-4-morpholinyl)(6-(2-methyl-2H-pyrazolo[3,4-b]pyridin-5-yl)thieno[2,3-b]pyridin-2-yl)methanone;
4-((6-(2-methyl-2H-pyrazolo[3,4-b]pyridin-5-yl)thieno[2,3-b]pyridin-2-yl)carbonyl)-3-morpholinecarbonitrile;
(2-methyl-4-morpholinyl)(6-(2-methyl-2H-pyrazolo[3,4-b]pyridin-5-yl)thieno[2,3-b]pyridin-2-yl)methanone;
1-methyl-5-(2-((3-methyl-4-morpholinyl)carbonyl)thieno[2,3-b]pyridin-6-yl)-2(1H)-pyridinone;
(3-methyl-4-morpholinyl)(6-(1,3-thiazol-2-yl)thieno[2,3-b]pyridin-2-yl)methanone;
3-methyl-6-(2-((3-methyl-4-morpholinyl)carbonyl)thieno[2,3-b]pyridin-6-yl)-4(3H)-pyrimidinone;
1-methyl-5-(2-(4-morpholinylcarbonyl)thieno[2,3-b]pyridin-6-yl)-2(1H)-pyridinone;
(3-methyl-4-morpholinyl)(6-(1,3-thiazol-2-yl)thieno[2,3-b]pyridin-2-yl)methanone;
3-methyl-6-(2-((3-methyl-4-morpholinyl)carbonyl)thieno[2,3-b]pyridin-6-yl)-4(3H)-pyrimidinone;
(6-(2-methyl-2H-pyrazolo[3,4-b]pyridin-5-yl)thieno[2,3-b]pyridin-2-yl)(8-oxa-3-azabicyclo[3.2.1]octan-3-yl)methanone;
4-morpholinyl(6-(1,3-thiazol-2-yl)thieno[2,3-b]pyridin-2-yl)methanone;
3-methyl-6-(2-(4-morpholinylcarbonyl)thieno[2,3-b]pyridin-6-yl)-4(3H)-pyrimidinone;
(2,2-dimethyl-4-morpholinyl)(6-(2-methyl-2H-pyrazolo[3,4-b]pyridin-5-yl)thieno[2,3-b]pyridin-2-yl)methanone;
5-(2-(4-morpholinylcarbonyl)thieno[2,3-b]pyridin-6-yl)-2(1H)-pyridinone;
(6-(2-methyl-2H-[1,2,3]triazolo[4,5-b]pyridin-6-yl)thieno[2,3-b]pyridin-2-yl)(4-morpholinyl)methanone;
(3-methyl-4-morpholinyl)(6-(2-methyl-2H-[1,2,3]triazolo[4,5-b]pyridin-6-yl)thieno[2,3-b]pyridin-2-yl)methanone;
(3-methyl-4-morpholinyl)(6-(3-methyl-3H-[1,2,3]triazolo[4,5-b]pyridin-6-yl)thieno[2,3-b]pyridin-2-yl)methanone;
(6-(2-methyl-2H-pyrazolo[3,4-b]pyridin-5-yl)thieno[2,3-b]pyridin-2-yl)(7-oxa-4-azaspiro[2.5]octan-4-yl)methanone;
(6-(2-methyl-2H-pyrazolo[3,4-b]pyridin-5-yl)thieno[2,3-b]pyridin-2-yl)(2-(trifluoromethyl)-4-morpholinyl)methanone;
(6-(2-methyl-2H-pyrazolo[3,4-b]pyridin-5-yl)thieno[2,3-b]pyridin-2-yl)(4-oxa-7-azaspiro[2.5]octan-7-yl)methanone;
5-(2-(3-methyl-4-morpholinyl)carbonyl)thieno[2,3-b]pyridin-6-yl)-2(1H)-pyridinone;
4-morpholinyl(6-(6-quinolinyl)thieno[2,3-b]pyridin-2-yl)methanone;
(6-(1-methyl-1H-imidazo[4,5-b]pyridin-6-yl)thieno[2,3-b]pyridin-2-yl)(4-morpholinyl)methanone;
2,8-dioxa-5-azaspiro[3.5]nonan-5-yl(6-(2-methyl-2H-pyrazolo[3,4-b]pyridin-5-yl)thieno[2,3-b]pyridin-2-yl)methanone;
(6-(2-methyl-2H-pyrazolo[3,4-b]pyridin-5-yl)thieno[2,3-b]pyridin-2-yl)(2-oxa-5-azabicyclo[2.2.2]octan-5-yl)methanone;
(6-(2-methyl-2H-pyrazolo[3,4-b]pyridin-5-yl)thieno[2,3-b]pyridin-2-yl)(2-oxa-5-azabicyclo[4.1.0]heptan-5-yl)methanone;
(6-(3-methyl-3H-[1,2,3]triazolo[4,5-b]pyridin-6-yl)thieno[2,3-b]pyridin-2-yl)(4-morpholinyl)methanone;
(3-methyl-4-morpholinyl)(6-(6-quinolinyl)thieno[2,3-b]pyridin-2-yl)methanone;
4-morpholinyl(6-(pyrido[2,3-b]pyrazin-7-yl)thieno[2,3-b]pyridin-2-yl)methanone;
(3-methyl-4-morpholinyl)(6-(pyrido[2,3-b]pyrazin-7-yl)thieno[2,3-b]pyridin-2-yl)methanone;
7-methyl-3-(2-(4-morpholinylcarbonyl)thieno[2,3-b]pyridin-6-yl)-1,7-naphthyridin-8(7H)-one;
(6-(2-methyl-2H-pyrazolo[3,4-b]pyridin-5-yl)thieno[2,3-b]pyridin-2-yl)(8-oxa-5-azaspiro[3.5]nonan-5-yl)methanone;
7-methyl-3-(2-((3-methyl-4-morpholinyl)carbonyl)thieno[2,3-b]pyridin-6-yl)-1,7-naphthyridin-8(7H)-one;
(6-(1-methyl-1H-imidazo[4,5-b]pyridin-6-yl)thieno[2,3-b]pyridin-2-yl)(3-methyl-4-morpholinyl)methanone;
(6-(2-methyl-2H-pyrazolo[3,4-b]pyridin-5-yl)thieno[2,3-b]pyridin-2-yl)(3-(trifluoromethyl)-4-morpholinyl)methanone;
(4-cyclopropyl-2-(2-methyl-2H-pyrazolo[3,4-b]pyridin-5-yl)thieno[2,3-d]pyrimidin-6-yl)(4-morpholinyl)methanone;
(4-cyclopropyl-2-(2-methyl-2H-pyrazolo[3,4-b]pyridin-5-yl)thieno[2,3-d]pyrimidin-6-yl)(1-pyrrolidinyl)methanone;
(4-cyclopropyl-2-(2-methyl-2H-pyrazolo[3,4-b]pyridin-5-yl)thieno[2,3-d]pyrimidin-6-yl)(3,3-difluoro-1-piperidinyl)methanone;
(4-cyclopropyl-2-(2-methyl-2H-pyrazolo[3,4-b]pyridin-5-yl)thieno[2,3-d]pyrimidin-6-yl)(1-piperidinyl)methanone;
(4-cyclopropyl-2-(2-methyl-2H-pyrazolo[3,4-b]pyridin-5-yl)thieno[2,3-d]pyrimidin-6-yl)(3-oxa-8-azabicyclo[3.2.1]octan-8-yl)methanone;
(2-(2-methyl-2H-pyrazolo[3,4-b]pyridin-5-yl)-4-(1-methyl-1H-pyrazol-5-yl)thieno[2,3-d]pyrimidin-6-yl)(4-morpholinyl)methanone;
(2-(2-methyl-2H-pyrazolo[3,4-b]pyridin-5-yl)-4-(1-methyl-1H-pyrazol-5-yl)thieno[2,3-d]pyrimidin-6-yl)(1-piperidinyl)methanone;
(4,4-difluoro-1-piperidinyl)(2-(2-methyl-2H-pyrazolo[3,4-b]pyridin-5-yl)-4-(1-methyl-1H-pyrazol-5-yl)thieno[2,3-d]pyrimidin-6-yl)methanone;
(4,4-difluoro-1-piperidinyl)(4-(1-methyl-1H-pyrazol-5-yl)-2-(2-methyl-2H-[1,2,3]triazolo[4,5-b]pyridin-6-yl)thieno[2,3-d]pyrimidin-6-yl)methanone;
6-(6-((4,4-difluoro-1-piperidinyl)carbonyl)-4-(1-methyl-1H-pyrazol-5-yl)thieno[2,3-d]pyrimidin-2-yl)-3-methyl-4(3H)-pyrimidinone;
(4,4-difluoro-1-piperidinyl)(4-(1-methyl-1H-pyrazol-5-yl)-2-(3-pyridinyl)thieno[2,3-d]pyrimidin-6-yl)methanone;
(4,4-difluoro-1-piperidinyl)(4-(1-methyl-1H-pyrazol-5-yl)-2-(5-pyrimidinyl)thieno[2,3-d]pyrimidin-6-yl)methanone;
(4,4-difluoro-1-piperidinyl)(2-(2-methoxy-5-pyrimidinyl)-4-(1-methyl-1H-pyrazol-5-yl)thieno[2,3-d]pyrimidin-6-yl)methanone;
5-(6-((4,4-difluoro-1-piperidinyl)carbonyl)-4-(1-methyl-1H-pyrazol-5-yl)thieno[2,3-d]pyrimidin-2-yl)-1-methyl-2(1H)-pyridinone;
5-(6-((4,4-difluoro-1-piperidinyl)carbonyl)-4-(1-methyl-1H-pyrazol-5-yl)thieno[2,3-d]pyrimidin-2-yl)-2-pyrimidinecarbonitrile;
(4,4-difluoro-1-piperidinyl)(2-(2-(dimethylamino)-5-pyrimidinyl)-4-(1-methyl-1H-pyrazol-5-yl)thieno[2,3-d]pyrimidin-6-yl)methanone;
(4,4-difluoro-1-piperidinyl)(4-(1-methyl-1H-pyrazol-5-yl)-2-(2-methyl-5-pyrimidinyl)thieno[2,3-d]pyrimidin-6-yl)methanone;
(4,4-difluoro-1-piperidinyl)(4-(1-methyl-1H-pyrazol-5-yl)-2-(2-(trifluoromethyl)-5-pyrimidinyl)thieno[2,3-d]pyrimidin-6-yl)methanone;
(2-(2-cyclopropyl-5-pyrimidinyl)-4-(1-methyl-1H-pyrazol-5-yl)thieno[2,3-d]pyrimidin-6-yl)(4,4-difluoro-1-piperidinyl)methanone;
(4,4-difluoro-1-piperidinyl)(2-(1-methyl-1H-pyrazol-4-yl)-4-(1-methyl-1H-pyrazol-5-yl)thieno[2,3-d]pyrimidin-6-yl)methanone;
(4,4-difluoro-1-piperidinyl)(4-(1-methyl-1H-pyrazol-5-yl)-2-(1-(trifluoromethyl)-1H-pyrazol-4-yl)thieno[2,3-d]pyrimidin-6-yl)methanone;
(4,4-difluoro-1-piperidinyl)(2-(1-methyl-1H-pyrazol-3-yl)-4-(1-methyl-1H-pyrazol-5-yl)thieno[2,3-d]pyrimidin-6-yl)methanone;
N-(5-(6-((4,4-difluoro-1-piperidinyl)carbonyl)-4-(1-methyl-1H-pyrazol-5-yl)thieno[2,3-d]pyrimidin-2-yl)-2-pyrimidinyl)acetamide;
(4,4-difluoro-1-piperidinyl)(2-(1-methyl-1H-imidazol-5-yl)-4-(1-methyl-1H-pyrazol-5-yl)thieno[2,3-d]pyrimidin-6-yl)methanone;
(4,4-difluoro-1-piperidinyl)(4-(1-methyl-1H-pyrazol-5-yl)-2-(1,3-thiazol-5-yl)thieno[2,3-d]pyrimidin-6-yl)methanone;
(4,4-difluoro-1-piperidinyl)(4-(1-methyl-1H-pyrazol-5-yl)-2-(1,3-oxazol-5-yl)thieno[2,3-d]pyrimidin-6-yl)methanone;
(4,4-difluoro-1-piperidinyl)(2-(2-(4-methyl-1-piperazinyl)-5-pyrimidinyl)-4-(1-methyl-1H-pyrazol-5-yl)thieno[2,3-d]pyrimidin-6-yl)methanone;
(4,4-difluoro-1-piperidinyl)(4-(1-methyl-1H-pyrazol-5-yl)-2-(2-(4-morpholinyl)-5-pyrimidinyl)thieno[2,3-d]pyrimidin-6-yl)methanone;
5-(6-((4,4-difluoro-1-piperidinyl)carbonyl)-4-(1-methyl-1H-pyrazol-5-yl)thieno[2,3-d]pyrimidin-2-yl)-2(1H)-pyridinone;
(4,4-difluoro-1-piperidinyl)(2-(3H-imidazo[4,5-b]pyridin-6-yl)-4-(1-methyl-1H-pyrazol-5-yl)thieno[2,3-d]pyrimidin-6-yl)methanone;
(4,4-difluoro-1-piperidinyl)(2-(imidazo[1,2-a]pyridin-3-yl)-4-(1-methyl-1H-pyrazol-5-yl)thieno[2,3-d]pyrimidin-6-yl)methanone;
(4,4-difluoro-1-piperidinyl)(4-(1-methyl-1H-pyrazol-5-yl)-2-(4-pyridazinyl)thieno[2,3-d]pyrimidin-6-yl)methanone;
(4,4-difluoro-1-piperidinyl)(4-(1-methyl-1H-pyrazol-5-yl)-2-(2-pyrazinyl)thieno[2,3-d]pyrimidin-6-yl)methanone;
6-(6-((4,4-difluoro-1-piperidinyl)carbonyl)-4-(1-methyl-1H-pyrazol-5-yl)thieno[2,3-d]pyrimidin-2-yl)-2-methyl-1(2H)-isoquinolinone;
(4,4-difluoro-1-piperidinyl)(4-(1-methyl-1H-pyrazol-5-yl)-2-(1,3-thiazol-2-yl)thieno[2,3-d]pyrimidin-6-yl)methanone;
(4,4-difluoro-1-piperidinyl)(4-(1-methyl-1H-pyrazol-5-yl)-2-(1,3-oxazol-2-yl)thieno[2,3-d]pyrimidin-6-yl)methanone;
N-(5-(6-((4,4-difluoro-1-piperidinyl)carbonyl)-4-(1-methyl-1H-pyrazol-5-yl)thieno[2,3-d]pyrimidin-2-yl)-2-pyrimidinyl)methanesulfonamide;
5-(6-((4,4-difluoro-1-piperidinyl)carbonyl)-4-(1-methyl-1H-pyrazol-5-yl)thieno[2,3-d]pyrimidin-2-yl)-2(1H)-pyrimidinone;
(4,4-difluoro-1-piperidinyl)(3-(2-methyl-2H-pyrazolo[3,4-b]pyridin-5-yl)thieno[2,3-b]pyrazin-6-yl)methanone;
(4,4-difluoro-1-piperidinyl)(2-(2-methyl-2H-pyrazolo[3,4-b]pyridin-5-yl)thieno[2,3-b]pyrazin-6-yl)methanone;
(4,4-difluoro-1-piperidinyl)(5-(2-methyl-2H-pyrazolo[3,4-b]pyridin-5-yl)thieno[3,2-b]pyridin-2-yl)methanone;
(5-(2-methyl-2H-pyrazolo[3,4-b]pyridin-5-yl)thieno[3,2-b]pyridin-2-yl)(1-piperidinyl)methanone;
(3,3-difluoro-1-piperidinyl)(5-(2-methyl-2H-pyrazolo[3,4-b]pyridin-5-yl)thieno[3,2-b]pyridin-2-yl)methanone;
(5-(2-methyl-2H-pyrazolo[3,4-b]pyridin-5-yl)thieno[3,2-b]pyridin-2-yl)(4-morpholinyl)methanone;
(3-amino-5-(2-methyl-2H-pyrazolo[3,4-b]pyridin-5-yl)thieno[3,2-b]pyridin-2-yl)(1-piperidinyl)methanone;
(3-amino-5-(2-methyl-2H-pyrazolo[3,4-b]pyridin-5-yl)thieno[3,2-b]pyridin-2-yl)(4-morpholinyl)methanone;
(3-amino-5-(2-methyl-2H-pyrazolo[3,4-b]pyridin-5-yl)thieno[3,2-b]pyridin-2-yl)(3,3-difluoro-1-piperidinyl)methanone;
(3-amino-5-(2-methyl-2H-pyrazolo[3,4-b]pyridin-5-yl)thieno[3,2-b]pyridin-2-yl)(4,4-difluoro-1-piperidinyl)methanone;
(4-cyclopropyl-2-(2-methyl-2H-pyrazolo[3,4-b]pyridin-5-yl)thieno[3,2-d]pyrimidin-6-yl)(4-morpholinyl)methanone;
4-morpholinyl(5-(4-morpholinyl)[1,3]thiazolo[5,4-b]pyridin-2-yl)methanone;
(5-(2-methyl-2H-pyrazolo[3,4-b]pyridin-5-yl)[1,3]thiazolo[5,4-b]pyridin-2-yl)(4-morpholinyl)methanone; or
(3-methyl-4-morpholinyl)(5-(2-methyl-2H-pyrazolo[3,4-b]pyridin-5-yl)[1,3]thiazolo[5,4-b]pyridin-2-yl)methanone.

48. The compound or salt of claim 1, wherein the compound is:
(3-methyl-6-(2-methyl-2H-pyrazolo[3,4-b]pyridin-5-yl)-1-benzothiophen-2-yl)((3S)-3-methyl-4-morpholinyl)methanone;
(3-methyl-6-(2-methyl-2H-pyrazolo[3,4-b]pyridin-5-yl)-1-benzothiophen-2-yl)((3R)-3-methyl-4-morpholinyl)methanone;
((3R)-3-methoxy-1-pyrrolidinyl)(3-methyl-6-(2-methyl-2H-pyrazolo[3,4-b]pyridin-5-yl)-1-benzothiophen-2-yl)methanone;
((3S)-3-methoxy-1-pyrrolidinyl)(3-methyl-6-(2-methyl-2H-pyrazolo[3,4-b]pyridin-5-yl)-1-benzothiophen-2-yl)methanone;
((3S)-3-methoxy-1-piperidinyl)(3-methyl-6-(2-methyl-2H-pyrazolo[3,4-b]pyridin-5-yl)-1-benzothiophen-2-yl)methanone;
((3R)-3-methoxy-1-piperidinyl)(3-methyl-6-(2-methyl-2H-pyrazolo[3,4-b]pyridin-5-yl)-1-benzothiophen-2-yl)methanone;
(3-amino-4-cyclobutyl-6-(2-methyl-2H-pyrazolo[3,4-b]pyridin-5-yl)thieno[2,3-b]pyridin-2-yl)((3S)-3-methyl-4-morpholinyl)methanone;
(3-amino-4-cyclobutyl-6-(2-methyl-2H-pyrazolo[3,4-b]pyridin-5-yl)thieno[2,3-b]pyridin-2-yl)((3R)-3-methyl-4-morpholinyl)methanone;
((3R)-3-methyl-4-morpholinyl)(6-(2-methyl-2H-pyrazolo[3,4-b]pyridin-5-yl)thieno[2,3-b]pyridin-2-yl)methanone;
((3S)-3-methyl-4-morpholinyl)(6-(2-methyl-2H-pyrazolo[3,4-b]pyridin-5-yl)thieno[2,3-b]pyridin-2-yl)methanone;
(4-cyclopropyl-6-(2-methyl-2H-pyrazolo[3,4-b]pyridin-5-yl)thieno[2,3-b]pyridin-2-yl)((3S)-3-methyl-4-morpholinyl)methanone;
(4-cyclopropyl-6-(2-methyl-2H-pyrazolo[3,4-b]pyridin-5-yl)thieno[2,3-b]pyridin-2-yl)((3R)-3-methyl-4-morpholinyl)methanone;
((2R)-4,4-difluoro-2-methyl-1-piperidinyl)(6-(2-methyl-2H-pyrazolo[3,4-b]pyridin-5-yl)thieno[2,3-b]pyridin-2-yl)methanone;
((2S)-4,4-difluoro-2-methyl-1-piperidinyl)(6-(2-methyl-2H-pyrazolo[3,4-b]pyridin-5-yl)thieno[2,3-b]pyridin-2-yl)methanone;
((2R)-4,4-difluoro-2-methyl-1-piperidinyl)(6-(2-methyl-2H-pyrazolo[3,4-b]pyridin-5-yl)thieno[2,3-b]pyridin-2-yl)methanone;
((2S)-4,4-difluoro-2-methyl-1-piperidinyl)(6-(2-methyl-2H-pyrazolo[3,4-b]pyridin-5-yl)thieno[2,3-b]pyridin-2-yl)methanone;
(3R)-4-((6-(2-methyl-2H-pyrazolo[3,4-b]pyridin-5-yl)thieno[2,3-b]pyridin-2-yl)carbonyl)-3-morpholinecarbonitrile;
(3S)-4-((6-(2-methyl-2H-pyrazolo[3,4-b]pyridin-5-yl)thieno[2,3-b]pyridin-2-yl)carbonyl)-3-morpholinecarbonitrile;
((2R)-2-methyl-4-morpholinyl)(6-(2-methyl-2H-pyrazolo[3,4-b]pyridin-5-yl)thieno[2,3-b]pyridin-2-yl)methanone;
((2S)-2-methyl-4-morpholinyl)(6-(2-methyl-2H-pyrazolo[3,4-b]pyridin-5-yl)thieno[2,3-b]pyridin-2-yl)methanone;
1-methyl-5-(2-(((3S)-3-methyl-4-morpholinyl)carbonyl)thieno[2,3-b]pyridin-6-yl)-2(1H)-pyridinone;
1-methyl-5-(2-(((3R)-3-methyl-4-morpholinyl)carbonyl)thieno[2,3-b]pyridin-6-yl)-2(1H)-pyridinone;
((3S)-3-methyl-4-morpholinyl)(6-(1,3-thiazol-2-yl)thieno[2,3-b]pyridin-2-yl)methanone;
3-methyl-6-(2-(((3S)-3-methyl-4-morpholinyl)carbonyl)thieno [2,3-b]pyridin-6-yl)-4(3H)-pyrimidinone;
((3R)-3-methyl-4-morpholinyl)(6-(1,3-thiazol-2-yl)thieno[2,3-b]pyridin-2-yl)methanone;
3-methyl-6-(2-(((3R)-3-methyl-4-morpholinyl)carbonyl)thieno[2,3-b]pyridin-6-yl)-4(3H)-pyrimidinone;
(6-(2-methyl-2H-pyrazolo[3,4-b]pyridin-5-yl)thieno[2,3-b]pyridin-2-yl)((1R,5R)-8-oxa-3-azabicyclo[3.2.1]octan-3-yl)methanone;
(6-(2-methyl-2H-pyrazolo[3,4-b]pyridin-5-yl)thieno[2,3-b]pyridin-2-yl)((1R,5S)-8-oxa-3-azabicyclo[3.2.1]octan-3-yl)methanone;
(6-(2-methyl-2H-pyrazolo[3,4-b]pyridin-5-yl)thieno[2,3-b]pyridin-2-yl)((1S,5S)-8-oxa-3-azabicyclo[3.2.1]octan-3-yl)methanone;
((3S)-3-methyl-4-morpholinyl)(6-(2-methyl-2H-[1,2,3]triazolo[4,5-b]pyridin-6-yl)thieno[2,3-b]pyridin-2-yl)methanone;
((3R)-3-methyl-4-morpholinyl)(6-(2-methyl-2H-[1,2,3]triazolo[4,5-b]pyridin-6-yl)thieno[2,3-b]pyridin-2-yl)methanone;
((3R)-3-methyl-4-morpholinyl)(6-(3-methyl-3H-[1,2,3]triazolo[4,5-b]pyridin-6-yl)thieno[2,3-b]pyridin-2-yl)methanone;
((3S)-3-methyl-4-morpholinyl)(6-(3-methyl-3H-[1,2,3]triazolo[4,5-b]pyridin-6-yl)thieno[2,3-b]pyridin-2-yl)methanone;
(6-(2-methyl-2H-pyrazolo[3,4-b]pyridin-5-yl)thieno[2,3-b]pyridin-2-yl)((2R)-2-(trifluoromethyl)-4-morpholinyl)methanone;
(6-(2-methyl-2H-pyrazolo[3,4-b]pyridin-5-yl)thieno[2,3-b]pyridin-2-yl)((2S)-2-(trifluoromethyl)-4-morpholinyl)methanone;
5-(2-(((3S)-3-methyl-4-morpholinyl)carbonyl)thieno[2,3-b]pyridin-6-yl)-2(1H)-pyridinone;
(6-(2-methyl-2H-pyrazolo[3,4-b]pyridin-5-yl)thieno[2,3-b]pyridin-2-yl)((1R,4R)-2-oxa-5-azabicyclo[2.2.2]octan-5-yl)methanone;
(6-(2-methyl-2H-pyrazolo[3,4-b]pyridin-5-yl)thieno[2,3-b]pyridin-2-yl)((1R,4S)-2-oxa-5-azabicyclo[2.2.2]octan-5-yl)methanone;
(6-(2-methyl-2H-pyrazolo[3,4-b]pyridin-5-yl)thieno[2,3-b]pyridin-2-yl)((1S,4R)-2-oxa-5-azabicyclo[2.2.2]octan-5-yl)methanone;
(6-(2-methyl-2H-pyrazolo[3,4-b]pyridin-5-yl)thieno[2,3-b]pyridin-2-yl)((1S,4S)-2-oxa-5-azabicyclo[2.2.2]octan-5-yl)methanone;
(6-(2-methyl-2H-pyrazolo[3,4-b]pyridin-5-yl)thieno[2,3-b]pyridin-2-yl)((1R,6R)-2-oxa-5-azabicyclo[4.1.0]heptan-5-yl)methanone;
(6-(2-methyl-2H-pyrazolo[3,4-b]pyridin-5-yl)thieno[2,3-b]pyridin-2-yl)((1R,6S)-2-oxa-5-azabicyclo[4.1.0]heptan-5-yl)methanone;
(6-(2-methyl-2H-pyrazolo[3,4-b]pyridin-5-yl)thieno[2,3-b]pyridin-2-yl)((1S,6R)-2-oxa-5-azabicyclo[4.1.0]heptan-5-yl)methanone;
(6-(2-methyl-2H-pyrazolo[3,4-b]pyridin-5-yl)thieno[2,3-b]pyridin-2-yl)((1S,6S)-2-oxa-5-azabicyclo[4.1.0]heptan-5-yl)methanone;
5-(2-(((3R)-3-methyl-4-morpholinyl)carbonyl)thieno[2,3-b]pyridin-6-yl)-2(1H)-pyridinone;
((3S)-3-methyl-4-morpholinyl)(6-(6-quinolinyl)thieno[2,3-b]pyridin-2-yl)methanone;
((3S)-3-methyl-4-morpholinyl)(6-(pyrido[2,3-b]pyrazin-7-yl)thieno[2,3-b]pyridine-2-yl)methanone;
((3R)-3-methyl-4-morpholinyl)(6-(pyrido[2,3-b]pyrazin-7-yl)thieno[2,3-b]pyridin-2-yl)methanone;
((3R)-3-methyl-4-morpholinyl)(6-(6-quinolinyl)thieno[2,3-b]pyridin-2-yl)methanone;
7-methyl-3-(2-(((3S)-3-methyl-4-morpholinyl)carbonyl)thieno[2,3-b]pyridin-6-yl)-1,7-naphthyridin-8(7H)-one;
(6-(1-methyl-1H-imidazo[4,5-b]pyridin-6-yl)thieno[2,3-b]pyridin-2-yl)((3S)-3-methyl-4-morpholinyl)methanone;
(6-(1-methyl-1H-imidazo[4,5-b]pyridin-6-yl)thieno[2,3-b]pyridin-2-yl)((3R)-3-methyl-4-morpholinyl)methanone;
7-methyl-3-(2-(((3R)-3-methyl-4-morpholinyl)carbonyl)thieno[2,3-b]pyridin-6-yl)-1,7-naphthyridin-8(7H)-one;
(6-(2-methyl-2H-pyrazolo[3,4-b]pyridin-5-yl)thieno[2,3-b]pyridin-2-yl)((3R)-3-(trifluoromethyl)-4-morpholinyl)methanone;
(6-(2-methyl-2H-pyrazolo[3,4-b]pyridin-5-yl)thieno[2,3-b]pyridin-2-yl)((3S)-3-(trifluoromethyl)-4-morpholinyl)methanone;
(4-cyclopropyl-2-(2-methyl-2H-pyrazolo[3,4-b]pyridin-5-yl)thieno[2,3-d]pyrimidin-6-yl)((1R,5S)-3-oxa-8-azabicyclo[3.2.1]octan-8-yl)methanone;
((3R)-3-methyl-4-morpholinyl)(5-(2-methyl-2H-pyrazolo[3,4-b]pyridin-5-yl)[1,3]thiazolo[5,4-b]pyridin-2-yl)methanone; or
((3S)-3-methyl-4-morpholinyl)(5-(2-methyl-2H-pyrazolo[3,4-b]pyridin-5-yl)[1,3]thiazolo[5,4-b]pyridin-2-yl)methanone.

49. (canceled)

50. A pharmaceutical composition comprising the compound or salt of claim 1 and a pharmaceutically acceptable excipient.

51. A method of treating a 15-PGDH mediated disease, disorder, or condition in a subject in need thereof, the method comprising administering to the subject an effective amount of the compound or salt of claim 1.

52-55. (canceled)

56. A method of manufacturing the compound or salt of claim 1, the method comprising:
reacting a compound of Formula (I-int-c.1) 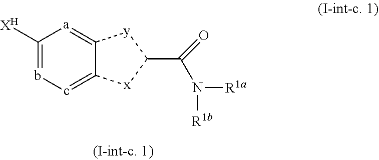
with a compound of Formula (B^{a}—R²): 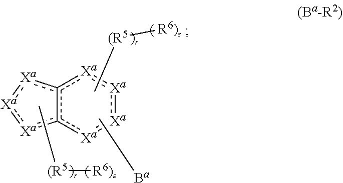
where
X^{H} is a halogen;
B^{a} is a boronic acid group or a boronic ester group;
“ [symbol] ” indicates a double bond or single bond may be present;
each instance of X^{a} independently is nitrogen, oxygen, or carbon;
each instance of s, where present, is an integer selected from 0, 1, 2, or 3;
each instance of r, where present, independently is an integer selected from 0, 1, 2, or 3, wherein where two instances of r occur on the same R² group, the total of both r values combined does not exceed 5;
each instance of R⁵, when present and attached to an N ring member, independently is C₁₋₆alkyl, C₁₋₆haloalkyl, C₃₋₆ cycloalkyl, or heterocyclyl having 3 to 10 members; and
each instance of R⁵, when present and attached to a C ring member, independently is oxo, CN, C₁₋₆ alkyl, C₃₋₆ cycloalkyl, C₁₋₆ haloalkyl, C₁₋₆ alkoxy, C(O)N(R⁶)₂, —N(R⁶)₂, —N(R⁶)(C₁₋₆ alkylene-OH), heterocyclyl having 3 to 10 members;
each instance of R⁶, where present, independently is —H, C₁₋₃ alkyl, C(O)C₁₋₃ alkyl, C(O)₂ C₁₋₃ alkyl, C₁₋₃ alkylene-OH, or S(O)ₙC₁₋₃alkyl, where n is 0, 1, or 2; and
wherein a —N═ ring nitrogen can be bonded to an R⁵ to provide a cation.
